(19) United States
(12) Patent Application Publication
(10) Pub. No.: US 2013/0330358 A1
(43) Pub. Date: Dec. 12, 2013

(54) CRYSTAL STRUCTURE AND PEPTIDE INHIBITORS OF HAUSP DEUBIQUITINASE

(71) Applicants: Jae Jung, Los Angeles, CA (US); Hye-Ra Lee, Los Angeles, CA (US); Myung Hee Kim, Daejeon (KR); Tae-Kwang Oh, Daejeon (KR); Jung-Won Hwang, Daejeon (KR)

(72) Inventors: Jae Jung, Los Angeles, CA (US); Hye-Ra Lee, Los Angeles, CA (US); Myung Hee Kim, Daejeon (KR); Tae-Kwang Oh, Daejeon (KR); Jung-Won Hwang, Daejeon (KR)

(73) Assignee: University of Southern California

(21) Appl. No.: 14/000,849

(22) PCT Filed: Feb. 22, 2012
(86) PCT No.: PCT/US12/26198

§ 371 Date: Aug. 22, 2013

Related U.S. Application Data
(60) Provisional application No. 61/445,452, filed on Feb. 22, 2011.
(60) Provisional application No. 61/454,839, filed on Mar. 21, 2011.

(51) Int. Cl. A61K 39/42; A61K 38/55

(52) U.S. Cl. CPC A61K 39/42; A61K 38/55
USPC 424/159.1; 530/300; 536/23.72; 514/19.3; 530/389.4; 435/375; 435/188; 435/69.1; 435/23; 435/354; 435/353; 435/352; 435/325; 435/350; 435/351; 435/363; 435/366; 703/11

(57) ABSTRACT

Two vIRF4 (Kaposi's-sarcoma-associated-herpesvirus vIRF4) peptides, vif1, corresponding to aa202-216 of vIRF4, and vif2, corresponding to aa220-236 of vIRF4, are potent and selective HAUSP antagonists. The vif1 and vif2 peptides robustly suppress HAUSP DUB enzymatic activity, ultimately leading to p53-mediated anti-cancer activity. The vif1 and vif2 peptides, along with their homologues, are useful in treating cancer through regulation of p53 activity in a cancer cell. Also disclosed is the crystalline structure of vIRF4-HAUSP TRAF domain complex. The structure is useful in computer aided drug design for identifying an agent that interacts with and inhibits HAUSP, resulting in p53 medicated cell cycle arrest of cancer cells.

### CROSS REFERENCE TO RELATED APPLICATIONS

This application claims the benefit under 35 U.S.C. §119(e) of U.S. Provisional Application Ser. No. 61/445,452, filed Feb. 22, 2011, and U.S. Provisional Application Ser. No. 61/454,839, filed Mar. 21, 2011, the contents of both which are incorporated by reference in their entireties.

### FIELD OF INVENTION

The present disclosure generally relates to compositions and methods and for preventing or treating cancer. It also relates to computer aided design for agents useful in interacting with HAUSP (Herpes virus-associated ubiquitin-specific protease), inhibiting HAUSP activity.

### BACKGROUND

Throughout this application, several technical publications are referenced by an Arabic numeral. The complete bibliographic citation for each reference is found immediately preceding the claims. The contents of each publication so referenced and the publications referenced within the specification are hereby incorporated into the present disclosure to more fully describe the state of the art to which this invention pertains.

p53 is a key regulator of a wide range of cellular activities, including cell cycle regulation, apoptosis, response to DNA damage, differentiation, and angiogenesis. p53 responds to DNA damage and other cellular stresses, such as viral infections, by inducing cell cycle arrest or apoptosis as well as playing an important role in tumor suppression. In order to circumvent host scrutiny, viruses employ their products to disrupt and overcome p53-mediated irreversible cell cycle arrest and apoptosis that are parts of the overall host surveillance mechanisms to block viral replication and dissemination.

p53 is negatively regulated by murine double minute 2 (MDM2) to maintain its low levels under normal conditions. It has been well established that MDM2, an oncogenic E3 ligase, is the major negative regulator of p53, which it modulates in two ways. First, MDM2 interaction masks the transactivation domain of p53, resulting in interfering with the transcriptional activity of p53. Second, MDM2 promotes the ubiquitin-mediated degradation of p53.

HAUSP (Herpes virus-associated ubiquitin-specific protease) is a ubiquitin specific protease or a deubiquitylating enzyme that cleaves ubiquitin from its substrates. HAUSP plays pivotal roles in the stability of p53 and MDM2, raising HAUSP as a potential therapeutic target for tuning p53-mediated anti-tumor activity. HAUSP is most widely known as a direct antagonist of MDM2. Normally, p53 levels are kept low in part due to MDM2-mediated ubiquitylation and degradation of p53. Interestingly, in response to oncogenic insults, HAUSP can deubiquitinate p53 and protect p53 from MDM2-mediated degradation of p53 in response to stress. It was also reported, however, that HAUSP is required for p53 destabilization and disruption of HAUSP stabilizes p53.

### SUMMARY

It is discovered herein that two vIRF4 (Kaposi's-sarcoma-associated-herpesvirus vIRF4) peptides, vif1, corresponding to aa202-216 of vIRF4, and vif2, corresponding to aa220-236 of vIRF4, are potent and selective HAUSP antagonists. It is further demonstrated that vif1 and vif2 peptides robustly suppress HAUSP DUB enzymatic activity, ultimately leading to p53-mediated anti-cancer activity. Therefore, the vif1 and vif2 peptides, along with their homologues, are useful in treating cancer, through regulation of p53 activity in a cancer cell.

Thus, one embodiment of the present disclosure provides a purified, isolated or recombinant vIRF4 peptide fragment, wherein the fragment comprises, or alternatively consists essentially of, or yet alternatively consists of, one or more amino acid sequence of the group: vIRF4 aa 153-256; vIRF4 aa 608-758; vIRF4 aa 202-208; vIRF4 aa 211-216; vIRF4 aa 202-216 (vif1); vIRF4 aa 209-216; vIRF4 aa 153-216; or vIRF4 aa 217-236; and vIRF4 aa 220-236 (vif2), or a biological equivalent of each thereof.

In another embodiment, the present disclosure provides a purified, isolated or recombinant vIRF4 peptide comprising, or alternatively consisting essentially of, or yet further consisting of at least two non-contiguous vIRF4 peptide fragments described above.

Another embodiment of the present disclosure provides a purified, isolated or recombinant retro-inverso peptide of any of the above vIRF4 peptides or peptide fragments.

Any of the above peptides can further comprise, or alternatively consist essentially of, or yet further consist of, a cell penetrating domain, which for example, can comprise a HIV TAT peptide.

In further embodiments, the present disclosure also provides polynucleotides encoding the peptides, of the present disclosure, antibodies that specifically bind to the peptides of the present disclosure, and compositions comprising the peptides or polynucleotides of the present disclosure.

Yet another embodiment of the present disclosure provides a method of increasing or inducing apoptosis in a cell, comprising contacting the cell with an effective amount of one or more of any of the above vIRF4 peptide fragments, polynucleotides or compositions, thereby increasing or inducing apoptosis in the cell.

Also provided is a method of increasing p53 activity in a cell with functional p53, comprising contacting the cell with an effective amount of one or more of any of the above vIRF4 peptide fragments, polynucleotides or compositions, thereby increasing p53 activity in the cell.

Further provided is a method of increasing MDM2 activity in a cell, comprising contacting the cell with an effective amount of one or more of any of the above vIRF4 peptide fragments, polynucleotides or compositions, thereby increasing MDM2 activity in the cell.

Still further, provided is a method of decreasing HAUSP activity in a cell, comprising contacting the cell with an effective amount of one or more of any of the above vIRF4 peptide fragments, polynucleotides or compositions, thereby decreasing HAUSP activity in the cell.

In yet another embodiment, the present disclosure provides a method of inhibiting enzyme substrate interaction between p53 and MDM2 in a cell, comprising contacting the cell with an effective amount of one or more of any of the above vIRF4 peptide fragments, polynucleotides or compositions, thereby inhibiting enzyme substrate interaction between p53 and MDM2 in the cell.

Provided also is a method of competitively blocking substrate binding of the TRAF domain of HAUSP in a cell, comprising contacting the cell with an effective amount of one or more of any of the above vIRF4 peptide fragments, polynucleotides or compositions, thereby competitively blocking substrate binding of the TRAF domain of HAUSP in the cell.

Still further provided is a method for suppressing deubiquitination activity of HAUSP in a cell comprising contacting the cell with an effective amount of one or more of any of the above vIRF4 peptide fragments, polynucleotides or compositions, thereby suppressing deubiquitination activity of HAUSP in a cell.

In one embodiment, the present disclosure provides a method of inhibiting the growth of a cancer cell, comprising contacting the cell with an effective amount of one or more of any of the above vIRF4 peptide fragments, polynucleotides or compositions, thereby inhibiting the growth of the cancer cell.

Another embodiment provides a method for tuning p53-mediated anti-tumor activity in a cell with functional p53, comprising contacting the cell with an effective amount of one or more of any of the above vIRF4 peptide fragments, polynucleotides or compositions, thereby tuning p53-mediated anti-tumor activity in a cell.

Thus, in one embodiment, the present disclosure provides a computer-implemented method for identifying an agent that binds HAUSP. The method comprises positioning a three-dimensional structure of a candidate agent against a three-dimensional structure of a HAUSP fragment, which three-dimensional structure of the HAUSP fragment is based on X, Y and Z atomic structure coordinates determined from a crystalline form of the HAUSP fragment. Interaction of the candidate agent with the HAUSP fragment at two or more, or alternatively three or more, or four or more, or five or more, or six or more, or all seven HAUSP amino acids selected from R104, R152, R153, 5155, D164, W165 or G166 identifies that candidate agent binds HAUSP.

Throughout the disclosure, the locations of the amino acids in HAUSP refer to those in human HAUSP, the sequence of which is provided in SEQ ID NO: 4. It would be readily appreciated by one of skill in the art that for a different HAUSP sequence, either a human variant, or HAUSP sequence from a different species, the corresponding locations of these amino acids can be readily obtained by methods known in the art including, for example, sequence alignment. Accordingly, in the present disclosure, a HAUSP sequence encompasses the human HAUSP sequence represented by SEQ ID NO. 4 and HAUSP variants and HAUSP sequences from other species.

A HAUSP amino acid, further, also encompasses its equivalents. An equivalent of a HAUSP amino acid, in one aspect, is any amino acid in a different HAUSP sequence that corresponds to the one recited for SEQ ID NO: 4. In another aspect, an equivalent of a HAUSP amino acid is a substitute amino acid at the same location in a HAUSP, or a corresponding location in a different HAUSP sequence, provided that the substitute amino acid is biologically similar to the one being substituted, in terms of size, hydrophilicity, or charge.

The present disclosure, in Table 3, for instance, also provides the X, Y and Z atomic structure coordinates that are determined from a crystalline form of a vIRF4-HAUSP TRAF domain complex. Alternatively, the X, Y and Z atomic structure coordinates can be determined from a free HAUSP TRAF domain as it is demonstrated here that the structure of a free HAUSP TRAF domain is similar to that of a HAUSP TRAF domain in a vIRF4-HAUSP TRAF domain complex. An exemplary set of X, Y and Z atomic structure coordinates of a free HAUSP TRAF domain are provided in Protein Data Bank (PDB) Accession No.: 2F1W.

In another embodiment, the three-dimensional structure of the HAUSP fragment is based on X, Y and Z atomic structure coordinates of a HAUSP catalytic domain. The interaction of the candidate agent with the HAUSP fragment at two or more, or alternatively all three HAUSP amino acids selected from C223, D481 or H464 identifies the candidate agent as suitable for inhibiting the activity of HAUSP. As it is further shown that N218, N226, D295, D482 or H456 of the HAUSP catalytic domain are also involved in the vif2-HAUSP catalytic domain binding, interaction of the candidate agent at one or more, or alternatively two or more, or three or more, or four or move, or all five of these amino acids further indicates that the candidate agent binds HAUSP. The X, Y and Z atomic structure coordinates of the HAUSP catalytic domain, for example, can be found in Protein Data Bank (PDB) Accession No.: 2F1Z.

In yet another embodiment, an agent that binds HAUSP is an agent or a mixture of agents that interact, as shown with any of the above methods, with both the HAUSP TRAF domain and the HAUSP catalytic domain.

A candidate agent can further analyzed for its ability bind to HAUSP and/or inhibit HAUSP activity in an in vitro or in vivo assay. Such a candidate agent can be a small molecule, a polypeptide, an antibody, an antibody fragment, or a combination or mixture of two or more such agents, without limitation.

The above methods are also useful in identifying an agent that interacts with HAUSP, or an agent that inhibits the activity of HAUSP, suitable for inhibiting cell growth, promoting cell cycle arrest, promoting apoptosis or promoting cell death.

Also provided, in one embodiment, is an agent suitable for inhibiting the activity of HAUSP, for interacting with HAUSP, or suitable for inhibiting cell growth, promoting cell cycle arrest, promoting apoptosis or promoting cell death, that is identified by any of the above methods.

Computer apparatus and non-transitory storage medium useful in carrying out the methods of the present disclosure are also provided in this disclosure. Also provided is a non-transitory computer medium comprising the crystalline structure of the vIRF4-HAUSP TRAF domain complex, as provided in Table 3, or its equivalents.

### BRIEF DESCRIPTION OF THE FIGURES

FIG. 1a-d. Structural basis for the interaction between HAUSP and vIRF4. (a) Silver stained purified V5-vIRF4 complexes. Arrows, HAUSP; asterisks, V5-vIRF4 (b) Ribbon representation of the vIRF4-HAUSP TRAF domain complex. The viral peptide (S202 to M216) bound to the TRAF domain is represented in dark gray. The β6 and β7 strands of the TRAF domain are indicated. (c) Decisive interactions between vIRF4 and HAUSP TRAF domain. Residues in vIRF4 and TRAF are displayed by gray and white carbon atoms, respectively. Hydrogen bonds are depicted as gray short-dashed lines. (d) Superimposition of target binding peptides onto HAUSP TRAF domain. The peptides include vIRF4, p53, MDM2, MDM2, MDM4, and EBNA1. Residues in vIRF4 are labeled in gray. The consensus sequence motif is shown and the most conserved residues are circled.

FIG. 2a-g. Bilateral interaction of vIRF4 with HAUSP and effect of this interaction on HAUSP DUB enzymatic activity. (a) NMR analysis of interaction between vIRF4 peptide and HAUSP TRAF-catalytic domain. The backbone amide region of the 2D ¹H-¹⁵N correlation spectra of vIRF4¹⁵³⁻²⁵⁶ in the presence of an equimolar amount of HAUSP⁶²⁻²⁰⁵ (light gray contours) or HAUSP⁶²⁻⁵⁶⁰ (dark gray contours). ¹H-¹⁵N spectrum of free vIRF4¹⁵³⁻²⁵⁶ is represented in black contours. Signal changes of vIRF4¹⁵³⁻²⁵⁶ observed upon the binding of HAUSP⁶²⁻²⁰⁵ are denoted by dark gray triangles, and additional changes detected upon the binding of HAUSP⁶²⁻⁵⁶⁰ are indicated by light gray triangles. Residues close to vIRF4 Trp²³² are indicated by light gray arrows. (b) The Trp²³² backbone assignment. Superposition of the ¹H-¹⁵N correlation spectra of free vIRF4^{153 256} (black) and vIRF4 W232A¹⁵³⁻²⁵⁶ (gray) for comparison. Residues located close to Trp²³² were identified by the comparison of the two spectra (light gray arrows). The assigned Trp²³² backbone is indicated by a gray arrow. (c) Signal changes of the tryptophan ε-NH protons upon interaction with HAUSP⁶²⁻²⁰⁵ (light gray) or HAUSP⁶²⁻⁵⁶⁰ (dark gray). Free vIRF4¹⁵³⁻²⁵⁶ is represented in black contours. Assignments of the tryptophan side chain and backbone signals are described in the FIGS. 11 and 12. (d) Proposed molecular interaction scheme between HAUSP and two different vIRF4 derived peptides. This model is based on the vIRF4-TRAF complex structure from the present study and the HAUSP structure containing the TRAF and catalytic domains (PDB accession code 2F1Z). The vIRF4²⁰²⁻²¹⁶ peptide is displayed a magenta loop, while the vIRF4²¹⁷⁻²³⁶ peptide is depicted as magenta short dashed line. Catalytic triad (red) is highlighted in the catalytic site. The ubiquitin binding pocket is marked by the black dashed circle. See text for description. (e) Effect of vif1/2 peptides on HAUSP DUB activity toward ubiquitin chains. Purified recombinant HAUSP alone or HAUSP pre-incubated with vif1 or vif2 peptide for 5 min at 37° C. was incubated with K48-Ub₃₋₇ chains for the indicated times at 37° C. Products were analyzed by immunoblotting (IB) with an anti-ubiquitin antibody. Right: time-course measuring the appearance of cleaved mono- and di-ubiquitin reaction products were determined by semi-quantification of IB shown on the left. (f) Effect of vif1/2 peptides on HAUSP DUB activity toward ubiquitinated MDM2. Human recombinant purified MDM2 was incubated with purified E1, E2, and ubiquitin for 2 h at 37° C. prior to the deubiquitination assay. HAUSP pre-incubated with increasing concentrations of each peptide or HAUSP alone was then incubated with ubiquitinated MDM2 for 1 h at 37° C. HAUSP DUB enzymatic activity toward ubiquitinated MDM2 was observed by IB with anti-MDM2 antibody. (g) In vivo effect of TAT-vif1/2 peptides on HAUSP DUB activity. At 24 h post-transfection with vector or Flag-tagged HAUSP, 293T cells were treated with 100 μM of TAT, TAT-vif1, or TAT-vif2 for an additional 12 h, followed by IP with an anti-Flag agarose beads and elution with Flag peptide. Purified HAUSP complexes were incubated with K48-Ub₃₋₇ chains for the indicated intervals and IB with an anti-ubiquitin antibody. One percent of the IP complex was used as the input.

FIG. 3a-g Inhibition of HAUSP function by vif1 or vif2 peptide activates p53-mediated anti tumor activity in vivo. (a) Growth inhibition of PELs induced by vif1/2 peptides. BC3, VG1, BCBL-1, and BJAB cells were treated with 100 μM TAT, TAT-vif1, or TAT-vif2 peptide for the indicated periods of time. The results were quantified as mean±s.d. of the combined results from three independent experiments; Data are mean±s. e. m.; n=200-300 cells from three independent experiments. *P<0.01 and **P<0.001. A Beckman Coulter Z2 Particle Count and size analyzer (BC Z2 CS analyzer) and trypan blue staining were used to determine cell death and for cell growth analysis. (b) vif1/2-induced cell cycle arrest of PELs. Asynchronously growing VG1 cells were treated with 100 μM peptide (TAT, TAT-vif1, or TAT-vif2 peptide) or 10 μM Nutlin-3a for 48 h. Cells were pulse-labeled with BrdU and analyzed for DNA content by flow cytometry. BrdU incorporation during the S phase is quantified as percentage of stained cells. The sub-G₁ populations in TAT-vif2 peptide treated VG1 cells are denoted by arrow. Data are culled from 3 independent experiments. (c) vif1/2-induced cell death of PELs. Apoptosis in VG1 cells was assessed at 48 h after treatment with 10 μM Nutlin-3a or 100 μM of each peptide by Annexin V/PI staining and measured by flow cytometry analysis. Lower left quadrants represent viable cells (Annexin V- and PI-negative); lower right quadrants represent early apoptotic cells (Annexin V-positive, PI-negative) demonstrating cytoplasmic membrane integrity; upper right quadrants represent non-viable, late apoptotic cells (Annexin V- and PI positive). Numbers indicate the percentage of cells in each quadrant. (d) Effect of vif1/2 peptides on p53 and its transcription target protein levels. VG1 and BJAB cells were treated with the same dose as used in (b) and (c) for 6 h and aliquots of cell lysates containing 10 mg of protein were analyzed by IB with the indicated antibody. (e) vif1/2-induced tumor suppression in vivo. NOD/SCID mice received an injection of 5×10⁶ BCBL-1-Luc cells, followed by intraperitoneal injection with 1 mg of TAT, TAT-vif1, and TAT-vif2 peptide for two weeks. Tumors were measured by in vivo bioluminescence imaging. (f and g) Combination therapy of vif1 and vif2 peptides. (f) BCBL-1 cells were treated with 25 μM of the indicated peptide for the indicated periods of time, followed by trypan blue staining for cell death analysis or cell number counting for cell growth. The results were quantified as mean±s.d. of the combined results from three independent experiments; Data are mean±s. e. m.; n=200-300 cells from three independent experiments. *P<0.05 and **P<0.01. (g) After establishment of tumors in NOD/SCID mice, TAT-vif1 and TAT-vif2 peptide were injected together for two weeks and tumors were measured by in vivo bioluminescence imaging.

FIG. 4. TRAF-like domain of HAUSP and vIRF4 (aa153-256) are responsible for their interaction. Schematic representation of the plasmid constructs. Left schematic describes HAUSP constructs. TRAF denotes the TRAF like domain, DUB denotes the de-ubiquitinase enzymatic domain. Right schematic depicts vIRF4 constructs; amino—terminal DNA—binding domain (DB), proline rich domain (PRD), and transactivation domain (TA) of cellular IRFs. (a and b) Communoprecipitation (Co-IP) of vIRF4 with the wt or several HAUSP mutants. 293T cells were transfected with the indicated HAUSP constructs along with vIRF4, followed by IP with an anti-V5 antibody and IB with an anti-Flag antibody. 1% of the whole cell lysate (WCL) was used as the input. (c and d) Co-IP of HAUSP with wt or several vIRF4 mutants. 293T cells were transfected with the indicated vIRF4 constructs along with HAUSP, followed by IP with an anti-V5 antibody and IB with an anti-Flag antibody. 1% of the WCL was used as the input. (e) At 48 h post-transfection with several GST-vIRF4 mutants along with HAUSP, 293T cells were used for GST pulldown, followed by IB with anti-Flag antibody.

FIG. 5. Typical isothermal titration calorimetric measurements of the interactions between the HAUSP TRAF domain and the vIRF4 protein derivatives or other peptides. Purified proteins and synthesized peptides were reconstituted in 150 mM NaCl and 10 mM HEPES (pH 7.0). The calorimetric assays were performed using a VPITC system. All experiments were carried out with a stirring speed of 300 rpm at 20° C., and the thermal power was recorded every 10 s. Data were analyzed using the ORIGIN software package (version 7.0). In each panel, the raw data are displayed in the upper FIG., and the integrated injection heats are displayed in the lower panel. Each titration against the HAUSP TRAF domain is indicated in each panel.

FIG. 6. The electron density map (Fo-Fc) showing viral peptide was calculated prior to inclusion of the peptide in the complex structure model and is contoured at 3.0σ.

FIG. 7. Surface representation of the TRAF domain-vIRF4 peptide complex. The HAUSP TRAF domain (in gray) forms a shallow groove at the waist of the surface structure. The vIRF4 peptide (in light gray) is positioned on the groove in a belttype arrangement around the waist.

FIG. 8. Structural comparison between the peptide-free (in yellow, PDB accession code 2F1W) and vIRF4-bound (in magenta) TRAF domain (in gray). No significant conformational differences are observed between the two structures except in the C-terminal region.

FIG. 9. Typical isothermal titration calorimetric measurements of the competitive binding of vIRF4 with TRAF domain against cellular substrates. Each peptide (MDM2¹³⁷⁻¹⁵², p53³⁵⁰⁻³⁶⁴, and p53³⁵⁵⁻³⁶⁹) was first titrated into the HAUSP TRAF domain, resulting in association constants of 9.1×10⁴ M⁻¹, 6.5×10⁴ M⁻¹, and 6.7×10⁴ M⁻¹, respectively. When vIRF4²⁰²⁻²¹⁶ was subsequently titrated against HAUSP cellular substrates as a competitor, the association constant of each titration was markedly increased to 10.9×10⁶ M⁻¹, 44.2×10⁶ M⁻¹, and 35.8×10⁶ M⁻¹, respectively, indicating a considerably tighter interaction between HAUSP TRAF domain and vIRF4 compared to its cellular substrates, MDM2 and p53. In each panel, the raw data are displayed in the upper FIG., and the integrated injection heats are displayed in the lower panel. Each competitive titration is indicated in the panel.

FIG. 10a-b. The HAUSP TRAF domain HAUSP⁶²⁻²⁰⁵ preferentially forms a stable complex with vIRF4¹⁵³⁻²¹⁶ in the presence of excess MDM2¹³⁷⁻¹⁵². (a) HAUSP⁶²⁻²⁰⁵ was reacted in the presence of a 5-fold excess amount of MDM2¹³⁷⁻¹⁵² and subjected to size exclusion chromatography. MDM2¹³⁷⁻¹⁵² was too small to be detected by peptide PAGE analysis using Pepti-Gel™ (Elpis Biotech. Inc., Korea), a polyacrylamide gel system used to separate small peptides (MW 2-30 kDa). It should be noted that ITC experiments revealed an interaction between HAUSP⁶²⁻²⁰⁵ and MDM2¹³⁷⁻¹⁵² (Kd=11.06 μM). (b) vIRF4¹⁵³⁻²¹⁶ was added to the HAUSP⁶²⁻²⁰⁵ and MDM2¹³⁷⁻¹⁵² reaction solution and subjected to size exclusion chromatography. vIRF4¹⁵³⁻²¹⁶ formed a stable complex with the HAUSP TRAF domain even in the presence of a 5-fold molar excess of MDM2 peptide. M, molecular size marker; R, reaction solution.

FIG. 11. The side chain assignments of the vIRF4¹⁵³⁻²⁵⁶ tryptophans. The 2D ¹H-¹⁵N HSQC spectra of vIRF4¹⁵³⁻²⁵⁶ and its mutants vIRF4 (W204A)¹⁵³⁻²⁵⁶ and vIRF4 (W232)¹⁵³⁻²⁵⁶ are superimposed and are shown in black, red and blue contours, respectively. The assigned tryptophan residues are denoted. NMR measurements were performed with 0.1 mM ¹⁵N-labeled protein in 50 mM HEPES (pH 6.5) containing 10% D₂O at 25° C. ¹H-¹⁵N HSQC spectra were measured on a Bruker 900 MHz NMR spectrometer. All NMR spectra were processed with Topspin 2.1 and analyzed with SPARKY 3.1 program.

FIG. 12. The superimposed ¹H-¹⁵N HSQC spectra of ¹⁵N-uniformly labeled vIRF4¹⁵³⁻²⁵⁶ (black) and ¹⁵N-Trp selectively labeled vIRF4¹⁵³⁻²⁵⁶ (red). The selected region near the Trp232 backbone signal is magnified, and its signal is denoted. Selective isotope (¹⁵N) labeling of tryptophan was performed for the tryptophan backbone assignment using an E. coli-based cell-free synthesis system. NMR measurements were performed with 0.1 mM ¹⁵N-labeled protein in 50 mM HEPES (pH 6.5) containing 10% D₂O at 25° C. using a Bruker 900 MHz NMR spectrometer. All NMR spectra were processed with Topspin 2.1 and analyzed with SPARKY 3.1 program.

FIG. 13 shows an illustrative computing device suitable for use with the present disclosure.

### DETAILED DESCRIPTION

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; and Mayer and Walker eds. (1987) Immunochemical Methods in Cell and Molecular Biology (Academic Press, London).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

### DEFINITIONS

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “purified” refers to a composition being substantially free from contaminants. With respect to polynucleotides and polypeptides, purified intends the composition being substantially free from contamination from polynucleotides or polypeptides with different sequences. In certain embodiments, it also refers to polynucleotides and polypeptides substantially free from cell debris or cell culture media.

The term “recombinant” refers to a form of artificial DNA that is created by combining two or more sequences that would not normally occur in their natural environment. A recombinant protein is a protein that is derived from recombinant DNA.

The term “binding” or “binds” as used herein are meant to include interactions between molecules that may be covalent or non-covalent which, in one embodiment, can be detected using, for example, a hybridization assay. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment,” as used herein, also refers to a peptide chain.

The phrase “biologically equivalent polypeptide” or “biologically equivalent peptide fragment” refers to protein, polynucleotide, or peptide fragment which hybridizes to the exemplified polynucleotide or peptide fragment under stringent conditions and which exhibit similar biological activity in vivo, e.g., approximately 100%, or alternatively, over 90% or alternatively over 85% or alternatively over 70%, as compared to the standard or control biological activity. Additional embodiments within the scope of this invention are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

As understood by those of skill in the art, a “retro-inverso” refers to an isomer of a linear peptide in which the direction of the sequence is reversed (“retro”) and the chirality of each amino acid residue is inverted (“inverso”). Compared to the parent peptide, a helical retro-inverso peptide can substantially retain the original spatial conformation of the side chains but has reversed peptide bonds, resulting in a retro-inverso isomer with a topology that closely resembles the parent peptide, since all peptide backbone hydrogen bond interactions are involved in maintaining the helical structure. See Jameson et al., (1994) Nature 368:744-746 (1994) and Brady et al. (1994) Nature 368:692-693. The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into an D retro-inverso peptide by synthesizing a reverse of the sequence for the corresponding native L-amino acid sequence.

The term “polynucleotide” refers to a polymeric form of nucleotides of any length, either deoxyribonucleotides or ribonucleotides or analogs thereof polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Biologically equivalent polynucleotides are those having the specified percent homology and encoding a polypeptide having the same or similar biological activity.

The term “non-contiguous” refers to the presence of an intervening peptide, nucleotide, polypeptide or polynucleotide between a specified region and/or sequence. For example, two polypeptide sequences are non-contiguous because the two sequences are separated by a polypeptide sequences that is not homologous to either of the two sequences. Non-limiting intervening sequences are comprised of at least a single amino acid or nucleotide.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product such as RNA or a polypeptide or protein.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

Various proteins are also disclosed herein with their GenBank Accession Numbers for their human proteins and coding sequences. However, the proteins are not limited to human-derived proteins having the amino acid sequences represented by the disclosed GenBank Accession numbers, but may have an amino acid sequence derived from other animals, particularly, a warm-blooded animal (e.g., rat, guinea pig, mouse, chicken, rabbit, pig, sheep, cow, monkey, etc.).

As used herein, “interferon regulatory factors” or “IRFs” refer to proteins which regulate transcription of interferons. IRFs can play a critical role in antiviral defense, immune response, cell growth regulation and apoptosis. Non-limiting examples of cellular IRF genes include human IRF-1, IRF-2, IRF-3, IRF-4/Pip/ICSAT, IRF-5, IRF-6, IRF-7, ICSBP/IRF-8 and ISGF3γ/p48/IRF-9, as well as virus-encoded analogues of cellular IRF. These factors share significant homology in the N-terminal 115 amino acids, which contains the DNA-binding domain and is characterized by five tryptophan repeats.

As used herein, the term “vIRF-4 interferon regulatory factor” or “vIRF4” refers to a protein having an amino acid sequence substantially identical to any of the representative vIRF4 sequences of GenBank Accession No. YP_{—}001129412.

As used herein, a “vIRF4 peptide” or “vIRF4 peptide fragment” refers to a peptide fragment of the vIRF4 protein, or a peptide that is at least about 70%, or alternatively at least about 75%, or about 80%, or about 85%, or about 90%, or about 95%, or about 98%, or about 99% identical to a peptide fragment of the vIRF4 protein.

As used herein, the term “Herpes virus-associated ubiquitin-specific protease”, “HAUSP”, “HAUSP Deubiquitinase”, or “ubiquitin specific peptidase 7” refers to a protein having an amino acid sequence substantially identical to any of the representative HAUSP sequences of GenBank Accession Nos. NP_{—}003461.2 (human), NP_{—}001003918.2 (mouse) and NP_{—}001019961.1 (rat). Suitable cDNA encoding HAUSP are provided at GenBank Accession Nos. NM_{—}003470.2 (human), NM_{—}001003918.2 (mouse) and NM_{—}001024790.1 (rat).

As used herein, the term “HAUSP activity” refers to any biological activity associated with the full length native HAUSP protein. In one embodiment, the activity of HAUSP refers to destabilization of p53. In suitable embodiments, the HAUSP activity is equivalent to the activity of a protein having an amino acid sequence represented by GenBank Accession No. NP_{—}003461.2, NP_{—}001003918.2 and NP_{—}001019961.1. Increasing or decreasing HAUSP activity, in one embodiment, refers to increasing or decreasing the expression of the HAUSP mRNA or protein and in another embodiment, refers to increasing or decreasing HAUSP's capability to destabilize p53. Measurement of transcriptional activity can be performed using any known method, such as immunohistochemistry, reporter assay or RT-PCR, which can also be used to determine whether the activity of HAUSP is increased or decreased. Measurement of HAUSP's capability to destabilize p53 can be measured by protein assays measuring the expression of the p53 protein or a tumor cell's ability to arrest cell cycle, a function of the p53 protein.

As used herein, the term “p53” refers to a protein having an amino acid sequence substantially identical to any of the representative p53 sequences of GenBank Accession Nos. NP_{—}000537.3 (human), NP_{—}035770.2 (mouse) and NP_{—}112251.2 (rat). Suitable cDNA encoding HAUSP are provided at GenBank Accession Nos. NM_{—}000546.4 (human), NM_{—}011640.3 (mouse) and NM_{—}030989.3 (rat).

As used herein, the term “p53 activity” refers to any biological activity associated with the full length native p53 protein. In one embodiment, the activity of p53 refers to the transcription regulation of a gene regulated by p53. In suitable embodiments, the p53 activity is equivalent to the activity of a protein having an amino acid sequence represented by GenBank Accession No. NP_{—}000537.3 (human), NP_{—}035770.2 (mouse) and NP_{—}112251.2 (rat). Increasing or decreasing p53 activity, in one embodiment, refers to increasing or decreasing the expression of the p53 mRNA or protein and in another embodiment, refers to decreasing or increasing p53's degradation. Measurement of transcriptional activity can be performed using any known method, such as immunohistochemistry, reporter assay or RT-PCR, which can also be used to determine whether the activity of p53 is increased or decreased. Measurement of p53's capability to regulate gene transcription can be measured by protein assays measuring the expression of proteins regulated by p53 or a tumor cell's ability to arrest cell cycle.

As used herein, the term “MDM2 p53 binding protein homolog” or “MDM2” refers to a protein having an amino acid sequence substantially identical to any of the representative MDM2 sequences of GenBank Accession Nos. NP_{—}002383.2 (human), NP_{—}034916.1 (mouse) and NP_{—}001101569.1 (rat). Suitable cDNA encoding HAUSP are provided at GenBank Accession Nos. NM_{—}002392.3 (human), NM_{—}010786.3 (mouse) and NM_{—}001108099.1 (rat).

As used herein, the term “MDM2 activity” refers to any biological activity associated with the full length native MDM2 protein. In one embodiment, the activity of MDM2 refers to the inactivation of p53. In suitable embodiments, the MDM2 activity is equivalent to the activity of a protein having an amino acid sequence represented by GenBank Accession No. NP_{—}002383.2 (human), NP_{—}034916.1 (mouse) and NP_{—}001101569.1 (rat). Increasing or decreasing MDM2 activity, in one embodiment, refers to increasing or decreasing the expression of the MDM2 mRNA or protein and in another embodiment, refers to increasing or decreasing of p53's inactivation. Measurement of transcriptional activity can be performed using any known method, such as immunohistochemistry, reporter assay or RT-PCR, which can also be used to determine whether the activity of MDM2 is increased or decreased. Measurement of MDM2's capability to inactivate p53 can be measured by protein assays measuring the expression of p53 or a tumor cell's ability to arrest cell cycle.

Increasing or decreasing of a gene's activity, in some embodiments, refers to at least about 10% increase or decrease, or alternatively at least about 20%, or about 30%, or about 40%, or about 50%, or about 60%, or about 70%, or about 80%, or about 90%, or about 95%, or about 98%, or about 99% of the gene's activity.

“Short interfering RNA” (siRNA) refers to sequence-specific or gene specific suppression of gene expression (protein synthesis) that is mediated by double-stranded RNA molecules, generally, from about 10 to about 30 nucleotides long that are capable of mediating RNA interference (RNAi). As used herein, the term siRNA includes short hairpin RNAs (shRNAs).

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced there from.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge.

A “gene delivery vehicle” is defined as any molecule that can carry inserted polynucleotides into a host cell. Examples of gene delivery vehicles are liposomes, micelles, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide of this invention can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

The terms “culture” or “culturing” refer to the in vitro propagation of cells or organisms on or in media of various kinds. It is understood that the descendants of a cell grown in culture may not be completely identical (i.e., morphologically, genetically, or phenotypically) to the parent cell.

A “composition” is intended to mean a combination of active polypeptide, polynucleotide or antibody and another compound or composition, inert (e.g. a detectable label) or active (e.g. a gene delivery vehicle) alone or in combination with a carrier which can in one embodiment be a simple carrier like saline or pharmaceutically acceptable or a solid support as defined below.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

A “subject,” “individual” or “patient” is used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

“Cell,” “host cell” or “recombinant host cell” are terms used interchangeably herein. It is understood that such terms refer not only to the particular subject cell but to the progeny or potential progeny of such a cell. The cells can be of any one or more of the type murine, rat, rabbit, simian, bovine, ovine, porcine, canine, feline, equine, and primate, particularly human. Because certain modifications may occur in succeeding generations due to either mutation or environmental influences, such progeny may not, in fact, be identical to the parent cell, but are still included within the scope of the term as used herein.

The terms “disease” and “disorder” are used inclusively and any disease that may be associated with cancer or apoptosis. As used herein, “cancer” may refer both to precancerous cells as well as cancerous cells of a tumor such as a solid tumor.

“Treating,” “treatment,” or “ameliorating” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to a disease. A patient may also be referred to being “at risk of suffering” from a disease. This patient has not yet developed characteristic disease pathology, however are know to be predisposed to the disease due to family history, being genetically predispose to developing the disease, or diagnosed with a disease or disorder that predisposes them to developing the disease to be treated.

### DESCRIPTIVE EMBODIMENTS

### Compositions

One embodiment of the present disclosure provides a purified, isolated or recombinant vIRF4 peptide fragment, wherein the fragment comprises, or alternatively consists essentially of, or yet alternatively consists of, an amino acid sequence: vIRF4 aa 153-256; vIRF4 aa 608-758; vIRF4 aa 202-208; vIRF4 aa 211-216; vIRF4 aa 202-216 (vif1); vIRF4 aa 209-216; vIRF4 aa 153-216; or vIRF4 aa 217-236; and vIRF4 aa 220-236 (vif2), or a biological equivalent of each thereof.

The amino acid sequence of vIRF4 is provided in GenBank accession number: YP_{—}001129412.1 and reproduced below.

| Amino acid sequence of vIRF4 (SEQ ID NO: 3): | |
|---|---|
| 1 | MPKAGGSEWA TLWIIDALEN NKFPYFSWFD RNNLLFAAPA PLPAGSDIPP GWYSVYHAFD |
| 61 | EECDRVYGPS PVVGQTVYGR FGRLLRGTRR AVVRNDLRYS DTFGGSYVVW QLVRTPFKNC |
| 121 | TYCYGAAYGP EKLQRFIQCL LSPPMQTTAT RRSDTREQSY EEAGAAAPAP PKAPSGLRGR |
| 181 | PRKSNRYYNV GDITTEQKAA CSVWIPVNEG ASTSGMGSSG TRQVTQASSF TWRVPGDPPA |
| 241 | PSTLTGPSDP HSSGAGLPGT APPKPQHETR LAGTVSGVSG VAQTPGDTGQ LAPPMRDGSR |
| 301 | LPSTSPWIPA CFPWGDLPVT GWWPQGASGL PEKVHPPTTG QFDPLSPRWT YTGIPSSQLN |
| 361 | PAAPSWIPPH AQAGTFVGEF SQGAPLAPQG LLPQSGQCAS AWLPRRETGA EGACGASTEG |
| 421 | RAPQGAASER VYPFEPQPPS APAPGYAKPS CYNWSPLAEP PATRPIRAPV WHPPVGHAVV |
| 481 | PEVRTPLWIP WSSGGAPNQG LSHTQGGASA TPSAGAPPTP EVAERQEPSS SGIPYVCQGD |
| 541 | NMATGYRRVT TSSGALEVEI IDLTGDSDTP STTVASTPLP VSGPRVFQPT VLYSAPEPAV |
| 601 | NPEVSHLPTE LERRECVCPG SGERPRVPLV STYAGDRYAV GGYGPEQSLV PPPLGLPLTL |
| 661 | SNLQGEDICT WEEGLGNILS ELQEEPSSST RQATDRRRPR SRSPHGRRTP VSHSGPEKPP |
| 721 | SKMFFDPPDS QRVSFVVEIF VYGNLRGTLR REGDAGEAML CSWPVGDTLG HLCQSFVPEL |
| 781 | LRIPRLTVPS PEQMEILNRV FEGLGHGFPI FCSMSGIYSR NATQVEGWWF GNPNSRYERI |
| 841 | LRSFSPRVPQ QLFNTARYLA TTAAIPQTPL SVNPVTCGTV FFGASPASTE NFQNVPLTVK |
| 901 | IFIGSIWDSL H |

In another embodiment, the present disclosure provides a purified, isolated or recombinant vIRF4 peptide comprising two non-contiguous vIRF4 peptide fragments described above.

Another embodiment of the present disclosure provides a purified, isolated or recombinant retro-inverso peptide of any of the above vIRF4 peptides or peptide fragments.

In yet another embodiment, the present disclosure provides an isolated polypeptide consisting essentially of (A) SEQ ID NO: 1 or an equivalent thereof and/or (B) SEQ ID NO: 2 or an equivalent thereof.

SEQ ID NO: 1 corresponds to amino acids 202-216 of vIRF4 (²⁰²SVWIPVNEGASTSGM²¹⁶). It is shown that the upstream region 202-208 is important for the binding activity of this peptide. An equivalent of SEQ ID NO: 1, therefore, includes a sequence that shares the same 202-208 sequence with SEQ ID NO: 1 while having at least about 70%, or about 75%, or about 80%, or about 85%, or about 90%, or about 95%, or about 98% sequence identity with SEQ ID NO: 1 overall.

SEQ ID NO: 2 corresponds to amino acids 220-236 of vIRF4 (²²⁰TRQVTQASSFTWRVPG²³⁶). It is shown that W²³² and nearby amino acids are involved in binding to the HAUSP catalytic domain and thus are important for the binding activity of this peptide. An equivalent of SEQ ID NO: 2, therefore, includes a sequence that maintains W²³² while having at least about 70%, or about 75%, or about 80%, or about 85%, or about 90%, or about 95%, or about 98% sequence identity with SEQ ID NO: 2 overall.

In one aspect, the polypeptide consists essentially of SEQ ID NO: 1 and/or SEQ ID NO: 2. In another aspect, the polypeptide consists essentially of (A) SEQ ID NO: 1 or an equivalent thereof. In yet another aspect, the polypeptide consists essentially of (B) SEQ ID NO: 2 or an equivalent thereof.

In another aspect, the polypeptide of claim 1, wherein the polypeptide consists essentially of (A) SEQ ID NO: 1 of an equivalent thereof and (B) SEQ ID NO: 2 or an equivalent thereof. Such a polypeptide can comprise a peptide linker between (A) and (B).

A “linker” or “peptide linker” refers to a peptide sequence linked to a polypeptide sequence at both ends of the linker peptide sequence. In one aspect, the linker is from about 1 to about 50 amino acid residues long or alternatively 1 to about 45, about 1 to about 40, about 1 to about 35, about 1 to about 30, about 1 to about 25, about 1 to about 20, about 1 to about 15, about 1 to about 10, about 1 to about 9, about 1 to about 8, about 1 to about 7, about 1 to about 6, about 1 to about 5, about 2 to about 40, about 2 to about 30, about 2 to about 25, about 2 to about 20, about 2 to about 15, about 2 to about 10, about 2 to about 9, about 2 to about 8, about 2 to about 7, about 2 to about 6, about 2 to about 5, about 3 to about 40, about 3 to about 30, about 3 to about 20, about 3 to about 15, about 3 to about 10, about 3 to about 9, about 3 to about 8, about 3 to about 7, about 3 to about 5, about 4 to about 40, about 4 to about 30, about 4 to about 20, about 4 to about 10, about 4 to about 8, about 4 to about 6, about 5 to about 40, about 5 to about 30, about 5 to about 20, about or 5 to about 10 amino acid residues long. In a particular aspect, the linker is from about 1 to about 20 amino acid residues long. In another particular aspect, the linker is from about 3 to 10 amino acid residues long.

Any of the above peptides or polypeptides can further comprise a cell penetrating peptide (CPP).

Cell penetrating peptides, (CPPs) or cell penetrating domains, as used herein, refer to short peptides that facilitate cellular uptake of various molecular cargos (from small chemical molecules to nanosize particles and large fragments of DNA). A “cargo”, such as a protein, is associated with the peptides either through chemical linkage via covalent bonds or through non-covalent interactions. The function of the CPPs are to deliver the cargo into cells, a process that commonly occurs through endocytosis with the cargo delivered to the endosomes of living mammalian cells. CPPs typically have an amino acid composition containing either a high relative abundance of positively charged amino acids such as lysine or arginine, or have sequences that contain an alternating pattern of polar/charged amino acids and non-polar, hydrophobic amino acids. It was previously reported that the human immunodeficiency virus transactivator of transcription (HIV-TAT) protein can be delivered to cells using a CPP.

A CPP employed in accordance with one aspect of the invention may include 3 to 35 amino acids, preferably 5 to 25 amino acids, more preferably 10 to 25 amino acids, or even more preferably 15 to 25 amino acids.

A CPP may also be chemically modified, such as prenylated near the C-terminus of the CPP. Prenylation is a post-translation modification resulting in the addition of a 15 (farneysyl) or 20 (geranylgeranyl) carbon isoprenoid chain on the peptide. A chemically modified CPP can be even shorter and still possess the cell penetrating property. Accordingly, a CPP, pursuant to another aspect of the invention, is a chemically modified CPP with 2 to 35 amino acids, preferably 5 to 25 amino acids, more preferably 10 to 25 amino acids, or even more preferably 15 to 25 amino acids.

A CPP suitable for carrying out one aspect of the invention may include at least one basic amino acid such as arginine, lysine and histidine. In another aspect, the CPP may include more, such as 2, 3, 4, 5, 6, 7, 8, 9, 10, or more such basic amino acids, or alternatively about 5%, 10%, 15%, 20%, 25%, 30%, 40%, 50% of the amino acids are basic amino acids. In one embodiment, the CPP contains at least two consecutive basic amino acids, or alternatively at least three, or at least five consecutive basic amino acids. In a particular aspect, the CPP includes at least two, three, four, or five consecutive arginine. In a further aspect, the CPP includes more arginine than lysine or histidine, or preferably includes more arginine than lysine and histidine combined.

CPPs may include acidic amino acids but the number of acidic amino acids should be smaller than the number of basic amino acids. In one embodiment, the CPP includes at most one acidic amino acid. In a preferred embodiment, the CPP does not include acidic amino acid. In a particular embodiment, a suitable CPP is the HIV-TAT peptide.

CPPs can be linked to a protein recombinantly, covalently or non-covalently. A recombinant protein having a CPP peptide can be prepared in bacteria, such as E. coli, a mammalian cell such as a human HEK293 cell, or any cell suitable for protein expression. Covalent and non-covalent methods have also been developed to form CPP/protein complexes. A CPP, Pep-1, has been shown to form a protein complex and proven effective for delivery (Kameyama et al. (2006) Bioconjugate Chem. 17:597-602).

CPPs also include cationic conjugates which also may be used to facilitate delivery of the proteins into the cells or tissue of interest. Cationic conjugates may include a plurality of residues including amines, guanidines, amidines, N-containing heterocycles, or combinations thereof. In related embodiments, the cationic conjugate may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. The cationic conjugate also may be an oligomer including an oligopeptide, oligoamide, cationically functionalized oligoether, cationically functionalized oligosaccharide, oligoamine, oligoethyleneimine, and the like, as well as combinations thereof. The oligomers may be oligopeptides where amino acid residues of the oligopeptide are capable of forming positive charges. The oligopeptides may contain 5 to 25 amino acids; preferably 5 to 15 amino acids; more preferably 5 to 10 cationic amino acids or other cationic subunits.

Recombinant proteins anchoring CPP to the proteins can be generated to be used for delivery to cells or tissue.

In further embodiments, the present disclosure also provides polynucleotides encoding the peptides of the present disclosure, antibodies that specifically bind to the peptides of the present disclosure, and compositions comprising the peptides or polynucleotides of the present disclosure. A composition is also provided, comprising a first polypeptide consisting essentially of SEQ ID NO: 1 or an equivalent thereof and a second polypeptide consisting essentially of SEQ ID NO: 2 or an equivalent thereof.

Polypeptides comprising the amino acid sequences of the disclosure can be prepared by expressing polynucleotides encoding the polypeptide sequences of this disclosure in an appropriate host cell. This can be accomplished by methods of recombinant DNA technology known to those skilled in the art. Accordingly, this disclosure also provides methods for recombinantly producing the polypeptides of this disclosure in a eukaryotic or prokaryotic host cells, as well as the isolated host cells used to produce the proteins. The proteins and polypeptides of this disclosure also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this disclosure also provides a process for chemically synthesizing the proteins of this disclosure by providing the sequence of the protein and reagents, such as amino acids and enzymes and linking together the amino acids in the proper orientation and linear sequence.

It is known to those skilled in the art that modifications can be made to any peptide to provide it with altered properties. Polypeptides of the disclosure can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

In a further embodiment, subunits of polypeptides that confer useful chemical and structural properties will be chosen. For example, peptides comprising D-amino acids may be resistant to L-amino acid-specific proteases in vivo. Modified compounds with D-amino acids may be synthesized with the amino acids aligned in reverse order to produce the peptides of the disclosure as retro-inverso peptides. In addition, the present disclosure envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂NH—R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide ligands with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present disclosure provides a method to produce a constrained peptide that incorporates random sequences at all other positions.

Non-classical amino acids may be incorporated in the peptides of the disclosure in order to introduce particular conformational motifs, examples of which include without limitation: 1,2,3,4-tetrahydroisoquinoline-3-carboxylate (Kazrnierski et al. (1991) J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski & Hruby (1991) Tetrahedron Lett. 32(41):5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1989) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and (Dharanipragada et al. (1992) Acta. Crystallogr. C. 48:1239-1241).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Lett. 29:5057-5060); α-helix inducing analogs (Kemp et al. (1988) Tetrahedron Lett. 29:4935-4938); α-turn inducing analogs (Kemp et al. (1989) J. Org. Chem. 54:109:115); analogs provided by the following references: Nagai & Sato (1985) Tetrahedron Lett. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Clones et al. (1988) Tetrahedron Lett. 29:3853-3856); tetrazole (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobicity, size, or charge. Methods used to determine the appropriate amino acid to be substituted and for which amino acid are know to one of skill in the art. Non-limiting examples include empirical substitution models as described by Dahoff et al. (1978) In Atlas of Protein Sequence and Structure Vol. 5 suppl. 2 (ed. M. O. Dayhoff), pp. 345-352. National Biomedical Research Foundation, Washington D.C.; PAM matrices including Dayhoff matrices (Dahoff et al. (1978), supra, or JTT matrices as described by Jones et al. (1992) Comput. Appl. Biosci. 8:275-282 and Gonnet et al. (1992) Science 256:1443-1145; the empirical model described by Adach & Hasegawa (1996) J. Mol. Evol. 42:459-468; the block substitution matrices (BLOSUM) as described by Henikoff & Henikoff (1992) Proc. Natl. Acad. Sci. USA 89:1-1; Poisson models as described by Nei (1987) Molecular Evolutionary Genetics. Columbia University Press, New York.; and the Maximum Likelihood (ML) Method as described by Müller et al. (2002) Mol. Biol. Evol. 19:8-13.

In another aspect, any of the above compositions further comprises a carrier. The carrier can be a solid phase carrier, a gel, an aqueous liquid carrier, a paste, a liposome, a micelle, albumin, polyethylene glycol, a pharmaceutically acceptable polymer, or a pharmaceutically acceptable carrier, such a phosphate buffered saline.

The compositions of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Compositions may optionally contain stabilizers, pH modifiers, surfactants, bioavailability modifiers and combinations of these.

Compositions may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension compositions may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension compositions.

The compositions of this disclosure are formulated for pharmaceutical administration to a mammal, preferably a human being. Such compositions of the disclosure may be administered in a variety of ways, preferably topically or by injection.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

In addition to dosage forms described above, pharmaceutically acceptable excipients and carriers and dosage forms are generally known to those skilled in the art and are included in the disclosure. It should be understood that a specific dosage and treatment regimen for any particular subject will depend upon a variety of factors, including the activity of the specific antidote employed, the age, body weight, general health, sex and diet, renal and hepatic function of the subject, and the time of administration, rate of excretion, drug combination, judgment of the treating physician or veterinarian and severity of the particular disease being treated.

### Polypeptide Conjugates

Another aspect of the disclosure provides a peptide conjugate comprising, or alternatively consisting essentially of, or alternatively consisting of, a carrier covalently or non-covalently linked to an isolated polypeptide of the disclosure. In some embodiments, the carrier comprises a liposome, or alternatively a micelle, or alternatively a pharmaceutically acceptable polymer, or a pharmaceutically acceptable carrier.

The polypeptides and polypeptide conjugates of the disclosure can be used in a variety of formulations, which may vary depending on the intended use. For example, one or more can be covalently or non-covalently linked (complexed) to various other molecules, the nature of which may vary depending on the particular purpose. For example, a peptide of the disclosure can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. A peptide can be conjugated to a fatty acid, for introduction into a liposome, see U.S. Pat. No. 5,837,249. A peptide of the disclosure can be complexed covalently or non-covalently with a solid support, a variety of which are known in the art and described herein. An antigenic peptide epitope of the disclosure can be associated with an antigen-presenting matrix such as an MHC complex with or without co-stimulatory molecules.

Examples of protein carriers include, but are not limited to, superantigens, serum albumin, tetanus toxoid, ovalbumin, thyroglobulin, myoglobulin, and immunoglobulin.

Peptide-protein carrier polymers may be formed using conventional cross-linking agents such as carbodimides. Examples of carbodimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

Examples of other suitable cross-linking agents are cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound may be used. Also included are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homo-bifunctional cross-linking agents include the bifunctional N-hydroxysuccinimide esters dithiobis(succinimidylpropionate), disuccinimidyl suberate, and disuccinimidyl tartrate; the bifunctional imido-esters dimethyl adipimidate, dimethyl pimelimidate, and dimethyl suberimidate; the bifunctional sulfhydryl-reactive crosslinkers 1,4-di-[3′-(2′-pyridyldithio) propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

Examples of common hetero-bifunctional cross-linking agents that may be used to effect the conjugation of proteins to peptides include, but are not limited to, SMCC (succinimidyl-4-(N-maleimidomethyl)cyclohexane-1-carboxylate), MBS (m-maleimidobenzoyl-N-hydroxysuccinimide ester), SIAB (N-succinimidyl(4-iodoacteyl)aminobenzoate), SMPB (succinimidyl-4-(p-maleimidophenyl)butyrate), GMBS (N-(γ-maleimidobutyryloxy)succinimide ester), MPBH (4-(4-N-maleimidopohenyl) butyric acid hydrazide), M2C2H (4-(N-maleimidomethyl) cyclohexane-1-carboxyl-hydrazide), SMPT (succinimidyloxycarbonyl-α-methyl-α-(2-pyridyldithio)toluene), and SPDP (N-succinimidyl 3-(2-pyridyldithio)propionate).

Cross-linking may be accomplished by coupling a carbonyl group to an amine group or to a hydrazide group by reductive amination.

The polypeptides or the compositions of the disclosure also may be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking cross-linked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. (See Wilchek (1988) Anal. Biochem. 171:1-32). Peptides can be modified to possess biotin groups using common biotinylation reagents such as the N-hydroxysuccinimidyl ester of D-biotin (NHS-biotin) which reacts with available amine groups on the protein. Biotinylated peptides then can be incubated with avidin or streptavidin to create large complexes. The molecular mass of such polymers can be regulated through careful control of the molar ratio of biotinylated peptide to avidin or streptavidin.

Also provided by this application are the peptides and polypeptides described herein conjugated to a label, e.g., a tag (His-tag), label e.g., a fluorescent or bioluminescent label, for use in the diagnostic methods. For example, detectably labeled peptides and polypeptides can be bound to a column and used for the detection and purification of antibodies. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in Haugland, Richard P. (1996) Molecular Probes Handbook.

The polypeptides or the compositions of the disclosure also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete Adjuvant, Freund's Incomplete Adjuvant and mineral salts.

### Isolated Polynucleotides, Host Cells and Compositions

Yet another aspect of the disclosure provides an isolated polynucleotide encoding for an isolated polypeptide, an antibody, or a biologically active fragment of the antibody of the disclosure. Also provided is a DNA construct comprising an expression vector and a polynucleotide. In one aspect of the DNA construct, the vector is a plasmid vector, a yeast artificial chromosome, or a viral vector. In one aspect, the vector of the DNA construct comprises a protein tag. Protein tags can be selected from a GST-tag, a myc-tag, or a FLAG-tag provided in expression constructs commercially available from, e.g., Invitrogen, Carlbad, Calif.

Another aspect of the disclosure provides an isolated host cell transformed with a polynucleotide or a DNA construct of the disclosure. The isolated host cells can be a prokaryotic or a eukaryotic cell. Yet another aspect of the disclosure provides an isolated transformed host cell expressing an isolated polypeptide, an antibody or a biologically active fragment of the antibody of the disclosure. The isolated host cells can be a prokaryotic or a eukaryotic cell.

Also provided are polynucleotides encoding substantially homologous and biologically equivalent polypeptides to the inventive polypeptides and polypeptide complexes. Substantially homologous and biologically equivalent intends those having varying degrees of homology, such as at least 80%, or alternatively, at least 85%, or alternatively at least 90%, or alternatively, at least 95%, or alternatively at least 98% homologous as defined above or those which hybridize under stringent condition to the polynucleotide or its complement and which encode polypeptides having the biological activity as described herein. It should be understood although not always explicitly stated that embodiments to substantially homologous polypeptides and polynucleotides are intended for each aspect of this disclosure, e.g., polypeptides, polynucleotides and antibodies.

The polynucleotides of this disclosure can be replicated using conventional recombinant techniques. Alternatively, the polynucleotides can be replicated using PCR technology. PCR is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein. Yet further, one of skill in the art can use the sequences provided herein and a commercial DNA synthesizer to replicate the DNA. Accordingly, this disclosure also provides a process for obtaining the polynucleotides of this disclosure by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can operatively link the polynucleotides to regulatory sequences for their expression in a host cell, described below. The polynucleotides and regulatory sequences are inserted into the host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

Also provided are host cells comprising one or more of the polypeptides or polynucleotides of this disclosure. In one aspect, the polypeptides are expressed and can be isolated from the host cells. In another aspect, the polypeptides are expressed and secreted. In yet another aspect, the polypeptides are expressed and present on the cell surface (extracellularly). Suitable cells containing the inventive polypeptides include prokaryotic and eukaryotic cells, which include, but are not limited to bacterial cells, algae cells, yeast cells, insect cells, plant cells, animal cells, mammalian cells, murine cells, rat cells, sheep cells, simian cells and human cells. A non-limiting example of algae cells is red alga Griffithsia sp. from which Griffithsin was isolated (Toshiyuki et al. (2005) J. Biol. Chem. 280(10):9345-53). A non-limiting example of plant cells is a Nicotiana benthamiana leaf cell from which Griffithsin can be produced in a large scale (O'Keefe (2009) Proc. Nat. Acad. Sci. USA 106(15):6099-6104). Examples of bacterial cells include Escherichia coli (Giomarelli et al. (2006), supra), Salmonella enteric, Streptococcus gordonii and lactobacillus (Liu et al. (2007) Cellular Microbiology 9:120-130; Rao et al. (2005) PNAS 102:11993-11998; Chang et al. (2003) PNAS 100(20):11672-11677; Liu et al. (2006) Antimicrob. Agents & Chemotherapy 50(10):3250-3259). The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line CHO, BHK-21; the murine cell lines designated NIH3T3, NSO, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

### Antibody Compositions

The disclosure, in another aspect, provides an antibody that binds an isolated polypeptide of the disclosure. The antibody can be a polyclonal antibody, a monoclonal antibody, a chimeric antibody, a humanized antibody or a derivative or fragment thereof as defined below. In one aspect, the antibody is detectably labeled or further comprises a detectable label conjugated to it.

Also provided is a composition comprising the antibody and a carrier. Further provided is a biologically active fragment of the antibody, or a composition comprising the antibody fragment. Suitable carriers are defined supra.

Further provided is an antibody-peptide complex comprising, or alternatively consisting essentially of, or yet alternatively consisting of, the antibody and a polypeptide specifically bound to the antibody. In one aspect, the polypeptide is the polypeptide against which the antibody is raised.

This disclosure also provides an antibody capable of specifically forming a complex with a protein or polypeptide of this disclosure, which are useful in the therapeutic methods of this disclosure. The term “antibody” includes polyclonal antibodies and monoclonal antibodies, antibody fragments, as well as derivatives thereof (described above). The antibodies include, but are not limited to mouse, rat, and rabbit or human antibodies. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. The antibodies are also useful to identify and purify therapeutic polypeptides.

### Methods of the Disclosure

Yet another embodiment of the present disclosure provides a method of increasing or inducing apoptosis in a cell, comprising contacting the cell with an effective amount of one or more of any of the above peptides, polynucleotides or compositions, thereby increasing or inducing apoptosis in the cell. Apoptosis of a cell can be measured by commercially available kits. In one aspect, the method leads to about 5%, or alternatively about 10%, or about 20%, or about 30%, or about 40, or about 50%, or about 60%, or about 70%, or about 80%, or about 90%, or about 95% increase of apoptosis.

Also provided is a method of increasing p53 activity in a cell with functional p53, comprising contacting the cell with an effective amount of one or more of any of the above peptides, polynucleotides or compositions, thereby increasing p53 activity in the cell. p53 activity in a cell can be measured, for example, by the cell's capability to arrest cell cycle in response to DNA damage. In one aspect, the method leads to about 5%, or alternatively about 10%, or about 20%, or about 30%, or about 40, or about 50%, or about 60%, or about 70%, or about 80%, or about 90%, or about 95% increase of p53 activity.

Further provided is a method of increasing MDM2 activity in a cell, comprising contacting the cell with an effective amount of one or more of any of the above peptides, polynucleotides or compositions, thereby increasing MDM2 activity in the cell. MDM2 activity in a cell can be measured, for example, by the cell's capability to inactivate p53. In one aspect, the method leads to about 5%, or alternatively about 10%, or about 20%, or about 30%, or about 40, or about 50%, or about 60%, or about 70%, or about 80%, or about 90%, or about 95% increase of MDM2 activity.

Still further, provided is a method of decreasing HAUSP activity in a cell, comprising contacting the cell with an effective amount of one or more of any of the above peptides, polynucleotides or compositions, thereby decreasing HAUSP activity in the cell. HAUSP activity in a cell can be measured, for example, by the cell's capability to destabilize p53. In one aspect, the method leads to about 5%, or alternatively about 10%, or about 20%, or about 30%, or about 40, or about 50%, or about 60%, or about 70%, or about 80%, or about 90%, or about 95% decrease of HAUSP activity.

In yet another embodiment, the present disclosure provides a method of inhibiting enzyme substrate interaction between p53 and MDM2 in a cell, comprising contacting the cell with an effective amount of one or more of any of the above peptides, polynucleotides or compositions, thereby inhibiting enzyme substrate interaction between p53 and MDM2 in the cell.

Provided also is a method of competitively blocking substrate binding of the TRAF domain of HAUSP in a cell, comprising contacting the cell with an effective amount of one or more of any of the above peptides, polynucleotides or compositions, thereby competitively blocking substrate binding of the TRAF domain of HAUSP in the cell.

Still further provided is a method for suppressing deubiquitination activity of HAUSP in a cell comprising contacting the cell with an effective amount of one or more of any of the above peptides, polynucleotides or compositions, thereby suppressing deubiquitination activity of HAUSP in a cell.

In one embodiment, the present disclosure provides a method of inhibiting the growth of a cancer cell, comprising contacting the cell with an effective amount of one or more of any of the above peptides, polynucleotides or compositions, thereby inhibiting the growth of the cancer cell. The compositions are useful in treating cancer, e.g., through regulation of p53 activity in a cancer cell. The compositions can be combined with another anticancer agent for use on the methods disclosed herein.

Another embodiment provides a method for tuning p53-mediated anti-tumor activity in a cell with functional p53, comprising contacting the cell with an effective amount of one or more of any of the above peptides, polynucleotides or compositions, thereby tuning p53-mediated anti-tumor activity in a cell.

A method is also provided, for stabilizing a p53 protein in a cell, comprising contacting the cell with any of the above peptides, polypeptide or compositions, thereby stabilizing the p53 protein in the cell.

Yet also provided is a method for inhibiting cell growth, promoting cell cycle arrest, promoting apoptosis or promoting cell death, comprising contacting the cell with any of the above peptides, polypeptide or compositions, thereby inhibiting cell growth, promoting cell cycle arrest, promoting apoptosis or promoting cell death of the cell.

In any of the above methods, the contacting can be in vitro or in vivo. In another embodiment, the cell can be a tumor cell. In some aspects, the cell comprises a wild-type p53.

Method for treating cancer in a subject are also provided, comprising administering to the subject an effective amount of any of the above peptides, polynucleotides or compositions, thereby treating cancer in the subject.

In one aspect, the cancer is an adenocarcinoma, a leukemia, a lymphoma, a melanoma, a myeloma, a sarcoma or a teratocarcinoma. In another aspect, the cancer is in one or more of adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid or uterus. In another aspect, the method further comprises administering to the subject a second chemotherapeutic agent.

Route of administration for the methods can be any methods disclosed herein, including but not limited to injection, parenteral administration, inhalation, or topical application.

The present disclosure also provides a screen for a possible therapeutic agent that is useful in any of the above methods, comprising contacting the agent with the catalytic domain of HAUSP (206-560) and comparing the physical interaction of the therapeutic agent to the HAUSP catalytic domain to the interaction of an isolated or purified vIRF4 peptide fragment to the HAUSP catalytic domain, wherein an interaction that is substantially similar or greater than the interaction of vIRF4 peptide interaction identifies the agent as a possible therapeutic agent.

### Kits

An aspect of the disclosure provides a kit for use in inhibiting cell growth, promoting cell cycle arrest, promoting apoptosis or promoting cell death, comprising, or alternatively consisting essentially of, or alternatively consisting of, an isolated polypeptide of the disclosure, and instructions to use.

Kits may further comprise suitable packaging and/or instructions for use of the compositions. The compositions can be in a dry or lyophilized form, in a solution, particularly a sterile solution, or in a gel or cream. The kit may contain a device for administration or for dispensing the compositions, including, but not limited to, syringe, pepitte, transdermal patch and/or microneedle.

The kits may include other therapeutic compounds for use in conjunction with the compounds described herein. These compounds can be provided in a separate form or mixed with the compounds of the present disclosure.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the disclosure, kits for treating a subject who suffers from or is susceptible to the conditions described herein are provided, comprising a container comprising a dosage amount of a composition as disclosed herein, and instructions for use. The container can be any of those known in the art and appropriate for storage and delivery.

Kits may also be provided that contain sufficient dosages of the effective composition or compound to provide effective treatment for a subject for an extended period, such as a week, 2 weeks, 3, weeks, 4 weeks, 6 weeks, or 8 weeks or more.

### Three-Dimensional Structures and Sequences

The present disclosure demonstrates that vif1 and vif2 interact with HAUSP at its TRAF domain and catalytic domain respectively and inhibits HAUSP's activity. Computer-aided methods are thus provided for determining or designing an agent that interacts with HAUSP at one or more such binding amino acid sites or domains, thereby identifying an agent that interacts with HAUSP. Such an agent is also a potential agent that binds HAUSP and thus inhibits the activity of HAUSP. Therefore, the present disclosure provides methods to identify HAUSP inhibitors. Interaction between an agent and a protein refers to the existence of a short distance between an atom of the agent and an atom of the protein, which short distance results in electrical forces between then, either attractive or repulsive.

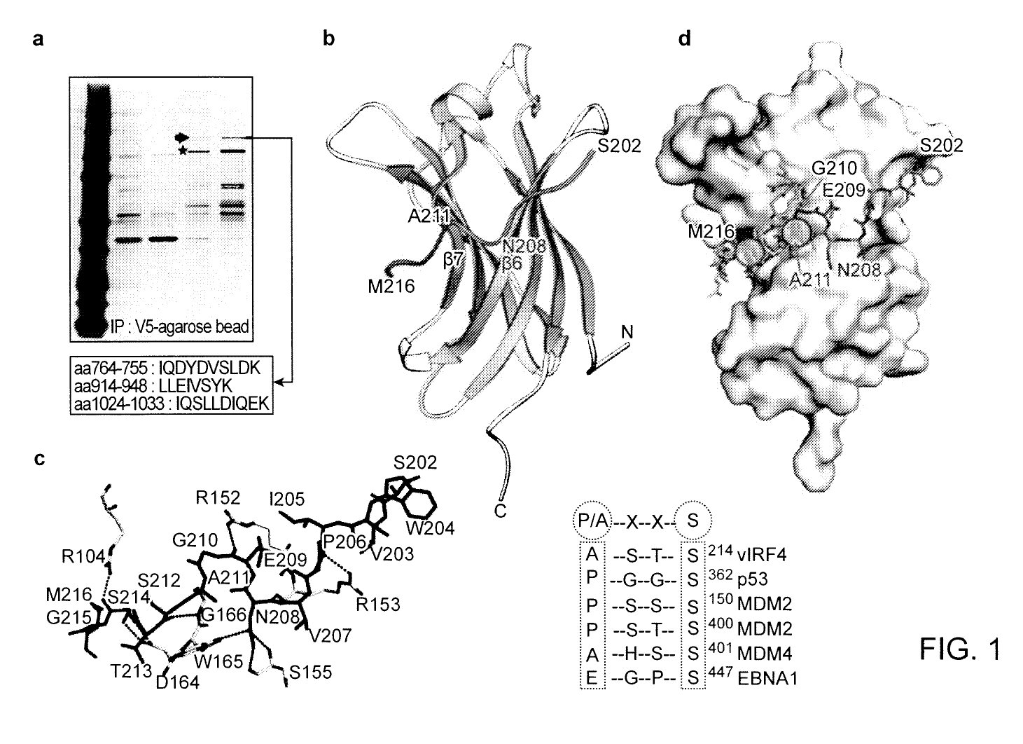

As shown in FIG. 1c, the amino acids in the HAUSP TRAF responsible for interacting with vIRF4 include R104, R152, R153, S155, D164, W165 or G166. The amino acids with HAUSP's catalytic domain include C223, D481 or H464. Additionally, N218, N226, D295, D482 or H456 within the catalytic domain are also involved in the binding of a substrate to the catalytic domain. The catalytic domain of HAUSP is known in the art and has been discussed in Hu, M. et al. (2006) PLoS Biol. 4: e27.

The locations of the amino acids of HAUSP refer to those in human HAUSP, the sequence of which is provided in SEQ ID NO: 4. It would be readily appreciated by one of skill in the art that for a different HAUSP sequence, either a human variant, or HAUSP sequence from a different species, the corresponding locations of these amino acids can be readily obtained by methods known in the art including, for example, sequence alignment. Accordingly, in the present disclosure, a HAUSP sequence encompasses the human HAUSP sequence represented by SEQ ID NO. 4 and HAUSP variants and HAUSP sequences from other species.

The amino acid sequence of vIRF4 is provided in GenBank accession number: YP_{—}001129412.1 and reproduced supra. The amino acid sequence of HAUSP is provided in GenBank accession number: NP_{—}003461.2 and reproduced below.

| Amino acid sequence of HAUSP (SEQ ID NO: 4): | |
|---|---|
| 1 | mnhqqqqqqq kageqqlsep edmemeagdt ddppritqnp vingnvalsd ghntaeedme |
| 61 | ddtswrseat fqftverfsr lsesvlsppc fvrnlpwkim vmprfypdrp hqksvgfflq |
| 121 | cnaesdstsw schaqavlki inyrddeksf srrishlffh kendwgfsnf mawsevtdpe |
| 181 | kgfidddkvt fevfvqadap hgvawdskkh tgyvglknqg atcymnsllq tlfftnqlrk |
| 241 | avymmptegd dssksvplal grvfyelghs dkpvgtkklt ksfgwetlds fmghdvqelc |
| 301 | rvlldnvenk mkgtcvegti pklfrgkmvs yiqckevdyr sdrredyydi qlsikgkkni |
| 361 | fesfvdyvav eqldgdnkyd agehglqeae kgvkfltlpp vlhlqlmrfm ydpqtdqnik |
| 421 | indrfefpeq lpldeflqkt dpkdpanyil havlvhsgdn hgghyvvyln pkgdgkwckf |
| 481 | dddvvsrctk eeaiehnygg hdddlsvrhc tnaymlvyir esklsevlqa vtdhdipqql |
| 541 | verlqeekri eaqkrkerqe ahlymqvqiv aedqfcghqg ndmydeekvk ytvfkvlkns |
| 601 | slaefvqsls qtmgfpqdqi rlwpmqarsn gtkrpamldn eadgnktmie lsdnenpwti |
| 661 | fletvdpela asgatlpkfd kdhdvmlflk mydpktrsln ycghiytpis ckirdllpvm |
| 721 | cdragfiqdt slilyeevkp niteriqdyd vsldkaldel mdgdiivfqk ddpendnsel |
| 781 | ptakeyfrdl yhrvdvifcd ktipndpgfv vtlsnrmnyf qvaktvaqrl ntdpmllqff |
| 841 | ksqgyrdgpg nplrhnyegt lrdllqffkp rqpkklyyqq lkmkitdfen rrsfkciwln |
| 901 | sqfreeeitl ypdkhgcvrd lleeckkave lgekasgklr lleivsykii gvhqedelle |
| 961 | clspatsrtf rieeipldqv didkenemlv tvahfhkevf gtfgipfllr ihqgehfrev |
| 1021 | mkriqslldi qekefekfkf aivmmgrhqy inedeyevnl kdfepqpgnm shprpwlgld |
| 1081 | hfnkapkrsr ytylekaiki hn |

vif1 (SEQ ID NO: 1) corresponds to amino acids 202-216 of vIRF4 (²⁰²SVWIPVNEGASTSGM²¹⁶). vif2 (SEQ ID NO: 2) corresponds to amino acids 220-236 of vIRF4 (²²⁰TRQVTQASSFTWRVPG²³⁶).

Table 3 shows the X, Y and Z atomic coordinates for the vIRF4-HAUSP TRAF domain complex. The table includes 3 chains (A, B, and W) in the coordinates. A, B, and W are coordinates for HAUSP TRAF domain (aa63-205), vIRF4 (aa202-216) which corresponds to vif1, and water molecules (1-238), respectively.

The coordinates of Table 3 provide a measure of atomic location in Angstroms. The coordinates are a relative set of positions that define a shape in three dimensions. It is understood by one of skill in the art that an entirely different set of coordinates having a different origin or axes could define a similar or the same structure. Further, variation of the relative atomic positions of the atoms of the structure so that the root mean square deviation of the conserved residue backbone atoms (i.e. the nitrogen-carbon-carbon backbone atoms of the protein amino acid residues) is less than 2.0 Å, or alternatively less than 1.0 Å or alternatively less than 0.5 Å, when superimposed on the coordinates for the residue backbone atoms, would result in a structure that is substantially identical to the structure in Table 3 in terms of both its structural characteristics and potency for structure-based drug design of HAUSP inhibitors. In the same vein, changing the number and/or positions of the water molecules would not generally affect the structure-based design.

### In Silico Drug Design and Methods of Using the Designed Drug

The present invention provides the three-dimensional structure of the vIRF4-HAUSP TRAF domain complex and the binding positions on HAUSP TRAF domain and the catalytic domain for vIRF4. Accordingly, the disclosure permits the use of virtual design techniques, also known as computer-aided, in silico design or modeling, to design, select, and synthesize agents capable of interacting with, binding to, or inhibiting HAUSP. In turn, the candidate agents may be effective in the treatment of a disease such as cancer. Thus, the present disclosure also provides agents identified or designed by the in silico methods.

Methods of in silico molecule or drug designs are well known in the art, see generally Kapetanovic (2008) Chem Biol. Interact., 171(2):165-76. Briefly, the atomic coordinates of the three-dimensional structure are input into a computer so that images of the structure and various parameters are shown on the display. The design typically involves positioning a three-dimensional structure to the three-dimensional structure of the target molecule. The positioning can be controlled by the user with assistance from a computer's graphic interface, and can be further guided by a computer algorithm looking for potential good matches. Positioning also involves moving either or both of the three-dimensional structures around at any dimension.

Then, the resultant data are input into a virtual compound library. Since a virtual compound library is contained in a virtual screening software such as DOCK-4 (Kuntz, UCSF), the above-described data may be input into such a software. Candidate drugs may be searched for, using a three-dimensional structure database of virtual or non-virtual drug candidate compounds, such as MDDR (Prous Science, Spain).

A candidate agent is found to be able to bind to HAUSP if a desired interaction between the candidate agent and HAUSP is found. The interaction can be quantitative, e.g, strength of interaction and/or number of interaction sites, or qualitative, e.g., interaction or lack of interaction. The output of the method, accordingly, can be quantitative or qualitative. In one aspect, therefore, the present disclosure also provides a method for identifying an agent that does not bind HAUSP.

The potential inhibitory or binding effect (i.e., interaction or association) of a compound may be analyzed prior to its actual synthesis and testing by the use of computer modeling techniques. If the theoretical structure of the given compound suggests insufficient interaction and association between it and HAUSP, synthesis and testing of the compound may be obviated. However, if computer modeling indicates a strong interaction, the molecule may then be synthesized and tested for its ability to bind to or inhibit HAUSP using various methods such as in vitro or in vivo experiments. Methods of testing an agent's ability to bind HAUSP, inhibit HAUSP, inhibit cell growth, promote cell cycle arrest, promote apoptosis or promote cell death are known in the art. In this manner, synthesis of inoperative compounds may be avoided.

One skilled in the art may use any of several methods to screen chemical or biological entities or fragments for their ability to associate with HAUSP and more particularly with the specific binding sites. Selected fragments or chemical entities may then be positioned in a variety of orientations, or docked, within an individual binding site of HAUSP as defined supra. Docking may be accomplished using software such as QUANTA, SYBYL, followed by energy minimization and molecular dynamics with standard molecular mechanics forcefields, such as CHARMM and AMBER.

Commercial computer programs are also available for in silico design. Examples include, without limitation, GRID (Oxford University, Oxford, UK), MCSS (Molecular Simulations, Burlington, Mass.), AUTODOCK (Scripps Research Institute, La Jolla, Calif.), DOCK (University of California, San Francisco, Calif.), GLIDE (Schrodinger Inc.), FlexX (Tripos Inc.) and GOLD (Cambridge Crystallographic Data Centre).

Once a compound has been designed or selected by the above methods, the efficiency with which that compound may bind to HAUSP may be tested and optimized by computational evaluation. For example, an effective HAUSP inhibitor may preferably demonstrate a relatively small difference in energy between its bound and free states (i.e., a small deformation energy of binding). Thus, the most efficient HAUSP inhibitor should preferably be designed with deformation energy of binding of not greater than about 10 kcal/mole, preferably, not greater than 7 kcal/mole. HAUSP inhibitors may interact with HAUSP in more than one conformation that is similar in overall binding energy. In those cases, the deformation energy of binding is taken to be the difference between the energy of the free agent and the average energy of the conformations observed when the agent binds to HAUSP.

A compound designed or selected, as binding to HAUSP may be further computationally optimized so that in its bound state it would preferably lack repulsive electrostatic interaction with the target protein. Such non-complementary (e.g., electrostatic) interactions include repulsive charge-charge, dipole-dipole, and charge-dipole interactions. Specifically, the sum of all electrostatic interactions between the agent and HAUSP when the compound is bound to HAUSP, preferably make a neutral or favorable contribution to the enthalpy of binding.

Computer softwares are also available in the art to evaluate compound deformation energy and electrostatic interaction. Examples include, without limitation, Gaussian 92 [Gaussian, Inc., Pittsburgh, Pa.]; AMBER [University of California at San Francisco]; QUANTA/CHARMM [Molecular Simulations, Inc., Burlington, Mass.]; and Insight II/Discover [Biosysm Technologies Inc., San Diego, Calif.].

Once an HAUSP-binding agent has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to HAUSP by the same computer methods described in detail, above.

This disclosure allows one skilled in the art to study the binding of a candidate agent to HAUSP by exposing either individual agents or mixtures of agents (such as may be obtained from combinatorial libraries) into HAUSP crystals or, alternatively, by co-crystallization of the substances of interest with HAUSP, using methods known to those of ordinary skill in the art, and the crystallization conditions based on those described in the following examples. Acquisition and analysis of X-ray diffraction data from these crystals can be then performed using standard methods. If agents bind to HAUSP then positive difference electron density will be observed in the Fourier maps calculated using the X-ray diffraction intensities and phases obtained from the HAUSP models presented in Table 3. Models of the chemical entities can than be built into the electron density using standard methods and the resulting structures can be refined against the X-ray diffraction data, providing experimental data describing the interaction of the molecules of interest with HAUSP. Those skilled in the art can use these models to design HAUSP inhibitors based either on purely structural data or on combination of structural data with enzyme-activity based structure-activity relationship and in silico drug design.

The present disclosure also provides methods of using the identified agents. In one embodiment, the present disclosure provides a method of increasing or inducing apoptosis in a cell, comprising contacting the cell with an effective amount of one or more of any of the above identified agents, thereby increasing or inducing apoptosis in the cell. Apoptosis of a cell can be measured by commercially available kits. In one aspect, the method leads to about 5%, or alternatively about 10%, or about 20%, or about 30%, or about 40, or about 50%, or about 60%, or about 70%, or about 80%, or about 90%, or about 95% increase of apoptosis.

Also provided is a method of increasing p53 activity in a cell with functional p53, comprising contacting the cell with an effective amount of one or more of any of the above identified agents, thereby increasing p53 activity in the cell. p53 activity in a cell can be measured, for example, by the cell's capability to arrest cell cycle in response to DNA damage. In one aspect, the method leads to about 5%, or alternatively about 10%, or about 20%, or about 30%, or about 40, or about 50%, or about 60%, or about 70%, or about 80%, or about 90%, or about 95% increase of p53 activity.

Further provided is a method of increasing MDM2 activity in a cell, comprising contacting the cell with an effective amount of one or more of any of the above identified agents, thereby increasing MDM2 activity in the cell. MDM2 activity in a cell can be measured, for example, by the cell's capability to inactivate p53. In one aspect, the method leads to about 5%, or alternatively about 10%, or about 20%, or about 30%, or about 40, or about 50%, or about 60%, or about 70%, or about 80%, or about 90%, or about 95% increase of MDM2 activity.

Still further, provided is a method of decreasing HAUSP activity in a cell, comprising contacting the cell with an effective amount of one or more of any of the above identified agents, thereby decreasing HAUSP activity in the cell. HAUSP activity in a cell can be measured, for example, by the cell's capability to destabilize p53. In one aspect, the method leads to about 5%, or alternatively about 10%, or about 20%, or about 30%, or about 40, or about 50%, or about 60%, or about 70%, or about 80%, or about 90%, or about 95% decrease of HAUSP activity.

In yet another embodiment, the present disclosure provides a method of inhibiting enzyme substrate interaction between p53 and MDM2 in a cell, comprising contacting the cell with an effective amount of one or more of any of the above identified agents, thereby inhibiting enzyme substrate interaction between p53 and MDM2 in the cell.

Provided also is a method of competitively blocking substrate binding of the TRAF domain of HAUSP in a cell, comprising contacting the cell with an effective amount of one or more of any of the above identified agents, thereby competitively blocking substrate binding of the TRAF domain of HAUSP in the cell.

Still further provided is a method for suppressing deubiquitination activity of HAUSP in a cell comprising contacting the cell with an effective amount of one or more of any of the above identified agents, thereby suppressing deubiquitination activity of HAUSP in a cell.

In one embodiment, the present disclosure provides a method of inhibiting the growth of a cancer cell, comprising contacting the cell with an effective amount of one or more of any of the above identified agents, thereby inhibiting the growth of the cancer cell.

Another embodiment provides a method for tuning p53-mediated anti-tumor activity in a cell with functional p53, comprising contacting the cell with an effective amount of one or more of any of the above identified agents, thereby tuning p53-mediated anti-tumor activity in a cell.

A method is also provided, for stabilizing a p53 protein in a cell, comprising contacting the cell with any of the above identified agents, thereby stabilizing the p53 protein in the cell.

Yet also provided is a method for inhibiting cell growth, promoting cell cycle arrest, promoting apoptosis or promoting cell death, comprising contacting the cell with any of the above identified agents, thereby inhibiting cell growth, promoting cell cycle arrest, promoting apoptosis or promoting cell death of the cell.

In any of the above methods, the contacting can be in vitro or in vivo. In another embodiment, the cell can be a tumor cell. In some aspects, the cell comprises a wild-type p53.

Method for treating cancer in a subject are also provided, comprising administering to the subject an effective amount of any of the above identified agents, thereby treating cancer in the subject.

In one aspect, the cancer is an adenocarcinoma, a leukemia, a lymphoma, a melanoma, a myeloma, a sarcoma or a teratocarcinoma. In another aspect, the cancer is in one or more of adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid or uterus. In another aspect, the method further comprises administering to the subject a second chemotherapeutic agent.

Route of administration for the methods can be any methods disclosed herein, including but not limited to injection or topical application.

### Computing Devices of the Present Technology

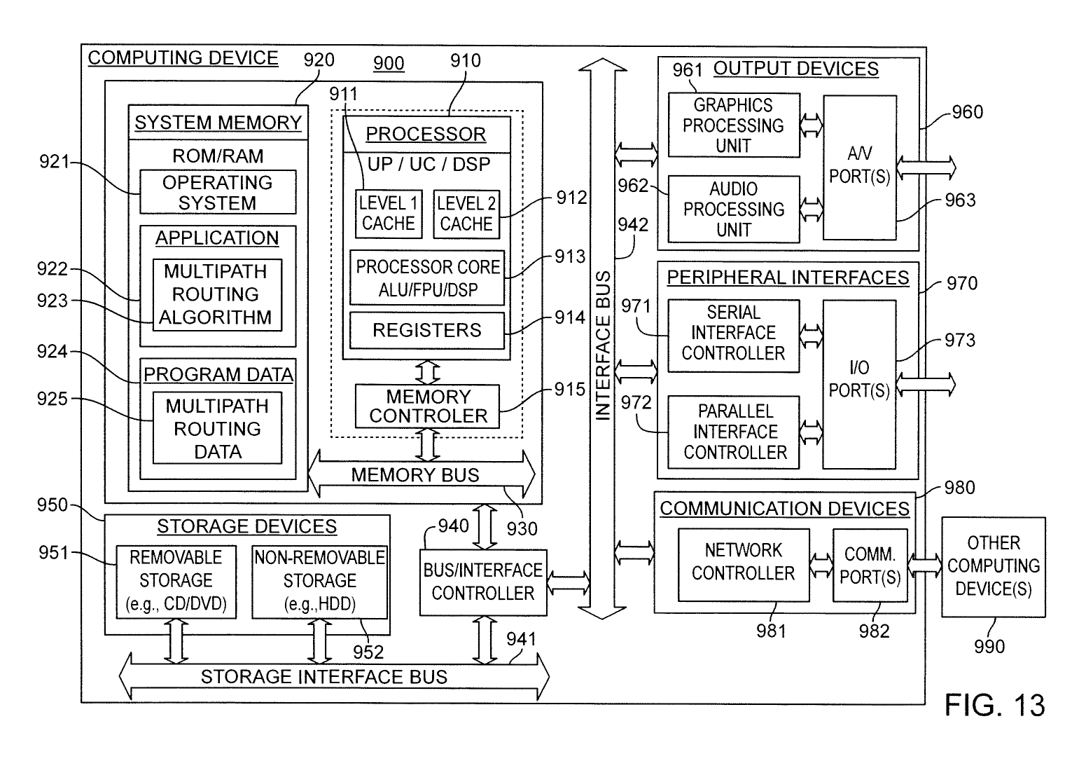

FIG. 13 is a block diagram illustrating an example computing device 900 that is arranged for identifying an agent suitable for binding or inhibiting the activity of HAUSP in accordance with the present disclosure. In a very basic configuration 901, computing device 900 typically includes one or more processors 910 and system memory 920. A memory bus 930 may be used for communicating between the processor 910 and the system memory 920.

Depending on the desired configuration, processor 910 may be of any type including but not limited to a microprocessor (μP), a microcontroller (μC), a digital signal processor (DSP), or any combination thereof. Processor 910 may include one more levels of caching, such as a level one cache 911 and a level two cache 912, a processor core 913, and registers 914. An example processor core 913 may include an arithmetic logic unit (ALU), a floating point unit (FPU), a digital signal processing core (DSP Core), or any combination thereof. An example memory controller 915 may also be used with the processor 910, or in some implementations the memory controller 915 may be an internal part of the processor 910.

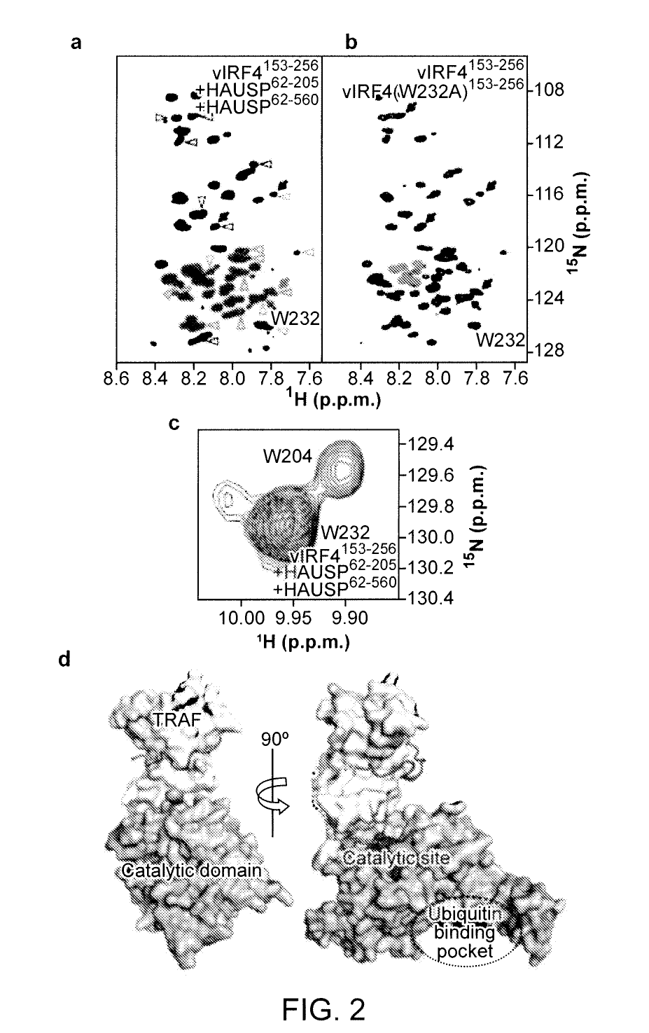

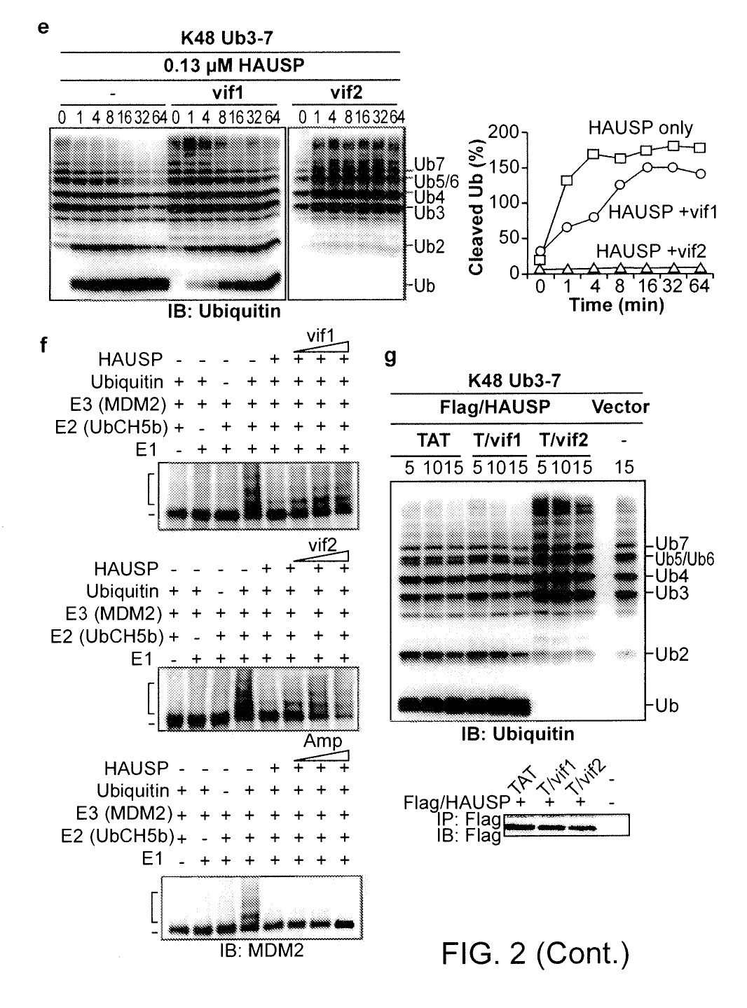

Depending on the desired configuration, the system memory 920 may be of any type including but not limited to volatile memory (such as RAM), non-volatile memory (such as ROM, flash memory, etc.) or any combination thereof. System memory 920 may include an operating system 921, one or more applications 922, and program data 924. Application 922 may include a method 923 for constructing a three-dimensional structure based on X, Y and Z atomic coordinates. Program Data 924 may include atomic coordinates 925 that may be useful for identifying an agent suitable for binding or inhibiting the activity of HAUSP as will be further described below. In some embodiments, application 922 may be arranged to operate with program data 924 on an operating system 921 such that identification of an agent suitable for inhibiting HAUSP activity may be provided as described herein. This described basic configuration is illustrated in FIG. 2 by those components within dashed line 901.

Computing device 900 may have additional features or functionality, and additional interfaces to facilitate communications between the basic configuration 901 and any required devices and interfaces. For example, a bus/interface controller 940 may be used to facilitate communications between the basic configuration 901 and one or more data storage devices 950 via a storage interface bus 941. The data storage devices 950 may be removable storage devices 951, non-removable storage devices 952, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 920, removable storage 951 and non-removable storage 952 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 900. Any such computer storage media may be part of device 900.

Computing device 900 may also include an interface bus 942 for facilitating communication from various interface devices (e.g., output interfaces, peripheral interfaces, and communication interfaces) to the basic configuration 901 via the bus/interface controller 940. Example output devices 960 include a graphics processing unit 961 and an audio processing unit 962, which may be configured to communicate to various external devices such as a display or speakers via one or more A/V ports 963. Example peripheral interfaces 970 include a serial interface controller 971 or a parallel interface controller 972, which may be configured to communicate with external devices such as input devices (e.g., keyboard, mouse, pen, voice input device, touch input device, etc.) or other peripheral devices (e.g., printer, scanner, etc.) via one or more I/O ports 973. An example communication device 980 includes a network controller 981, which may be arranged to facilitate communications with one or more other computing devices 990 over a network communication link via one or more communication ports 982.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 900 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 900 may also be implemented as a personal computer including both laptop computer and non-laptop computer configurations.

### EXAMPLES

The disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

### Example 1

This example shows that two short peptides, vif1 and vif2, derived from Kaposi's-sarcoma-associated-herpesvirus vIRF4 as potent and selective HAUSP antagonists. Thus, these virus-derived-short peptides represent biologically active HAUSP antagonists, potentially leading to a paradigm shift in p53-targeted anti-cancer therapy.

### Materials and Methods

### Cell Culture and Transfection Reagents.

293T cells were cultured in Dulbecco's modified Eagle's medium (DMEM) supplemented with 10% fetal bovine serum (FBS) and 1% penicillin-streptomycin (Gibco-BRL). PEL cell line BC-1 is derived from a HIV-positive patient and coinfected with KSHV and EBV. BC3, VG1, and BCBL-1 cell lines are negative for HIV and infected by only KSHV. PEL cell lines and KSHV-negative control cells (BJAB) were cultured in RPMI 1640 medium supplemented with 10% FBS and 1% penicillin-streptomycin. The prostatic human tumor cell lines LnCap, PC3, and DU145 were kept in culture in RPMI 1640 medium supplemented with 10% FBS and 1% penicillin-streptomycin. The human osteosarcoma cell line SJSA-1 was maintained in RPMI 1640 medium supplemented with 10% FBS and 1% penicillin-streptomycin. The MCF7 human breast cancer cell line was grown in DMEM, supplemented with 10% FBS, 2 mM L-glutamine (Gibco-BRL), and 1% penicillin-streptomycin. The BCBL-1 luciferase (BCBL-1-Luc) cell line was maintained in RPMI 1640 medium supplemented with 10% FBS, 1% penicillin-streptomycin, and 100 μg/ml Hygromycin. Transient transfections were performed with calcium phosphate (Clontech), according to the manufacturer's instructions.

### Plasmid Construction.

All constructs for transient and stable expression in mammalian cells were derived from the pcDNA4N5·His, pEF-IRES-Puro, pEBG-GST, or pCMV-3×Flag expression vectors. DNA fragments corresponding to the coding sequence of the KSHV-vIRF4 and human HAUSP gene were amplified from template DNA by polymerase chain reaction (PCR) and subcloned into pcDNA4N5.His at EcoRI and NotI, into pEF-IRES-Puro at EcoRI and XbaI, into pCMV-3×Flag at the BamHI and NotI, or into pEBG-GST at the BamHI and NotI restriction sites. Several vIRF4 mutants were generated via PCR and have been described in reference 1. The genes corresponding to vIRF4¹⁵³⁻²⁵⁶, vIRF4¹⁵³⁻²¹⁶, HAUSP⁶²⁻²⁰⁵, and HAUSP⁶²⁻⁵⁶⁰ were fused to a hexahistidine-tag2 and expressed in E. coli BL21 (DE3) RIPL cells (Stratagene, Inc.). The deletion mutants vIRF4^{153-256/Δ202-216} and vIRF4^{153-256/Δ202-216/Δ237-256} were generated using the QuikChange protocol (Stratagene, Inc.). For NMR experiments, Trp²⁰⁴ and Trp²³² of vIRF4¹⁵³⁻²⁵⁶ were mutated to alanine All constructs were sequenced using an ABI PRISM 377 automatic DNA sequencer to verify 100% correspondence with the original sequence.

### Yeast Two-Hybrid Screen.

Yeast transformation with the KSHV library was performed using a method described in reference 3. Library screening and recovery of plasmids were performed according to the manufacturer's instructions (Clontech).

### Immunoblotting and Immunoprecipitation.

For immunoblotting, polypeptides were resolved by SDS-PAGE and transferred onto a PVDF membrane (Bio-Rad). The membranes were blocked using 5% non-fat milk, then probed with the indicated antibodies. The primary antibodies were purchased from the following sources: p53 (D0-1), MDM2 (SMP14), p21 (187), and Ubiquitin (P4D1) antibodies from Santa Cruz Biotechnology; HAUSP antibody from Calbiochem; β-Tubulin, Flag (M2), and GST antibody from Sigma; Au antibody from Covance; V5 antibody from Bethyl Laboratories, Inc. Immunodetection was achieved using a chemiluminescence reagent (Denville Scientific) and a Fuji Phosphor Imager (BAS-1500; Fuji Film Co., Tokyo, Japan). For immunoprecipitation, cells were harvested, then lysed in a 1% NP40 lysis buffer supplemented with complete protease inhibitor cocktail (Roche). After pre-clearing using protein A/G agarose beads (Amersham Biosciences) for 1 h at 4° C., whole-cell lysates (WCL) were used for immunoprecipitation with the indicated antibodies. Generally, 1-4 μg of a commercial antibody was added to 1 ml of the cell lysate and incubated at 4° C. for 3 h. After adding the protein A/G agarose beads, incubation was continued for an additional 2 h. Immunoprecipitates were extensively washed using an 1% NP40 lysis buffer and eluted by boiling for 5 min in an 2×SDS-PAGE loading buffer (SIGMA). For GST pulldowns, cells were collected and lysed in the NP40 buffer supplemented with completed protease inhibitor cocktail. Post-centrifugation supernatants were precleared with protein A/G bead for 1 h at 4° C. Pre-cleared lysates were mixed with 50% slurry of glutathione-conjugated Sepharose beads (Amersham Biosciences) and the binding reaction incubated for 1 h at 4° C. Precipitates were then washed extensively with a lysis buffer. Protein bound to glutathione beads were eluted by boiling them with an SDS loading buffer for 5 min.

### Protein Purification.

Protein expression was induced by 0.5 mM IPTG at 18° C. for 18 h. Recombinant proteins were purified as described in reference 4. HAUSP domain proteins were treated with recombinant TEV protease to remove the hexahistidine-tag. Purified proteins were dialyzed against 100 mM NaCl and 20 mM Tris-HCl (pH 7.5). For NMR, E. coli cells harboring an expression plasmid for vIRF4153-256 and its mutants were grown in M9 minimal media enriched with 15NH₄Cl as the sole nitrogen source (99% 15N; Cambridge Isotope Laboratories, Inc.). Selective isotope (15N) labeling of tryptophan was performed for the tryptophan backbone assignments using an E. coli-based cell-free synthesis system⁵.

All ¹⁵N-labeled proteins were expressed and purified as described for the native proteins. Purified 15N-labeled proteins were dialyzed against 50 mM HEPES (pH 6.5) as a final step.

### Isothermal Titration Calorimetry (ITC)

Purified proteins and synthesized peptides (Peptron Inc., Deajeon, Korea) were reconstituted in 150 mM NaCl and 10 mM HEPES (pH 7.0). The calorimetric assays were performed using a VP-ITC system (MicroCal Inc., Northampton, Mass.). Samples were degassed by vacuum aspiration for 15 min prior to loading. All experiments were carried out with a stirring speed of 300 rpm at 20° C., and the thermal power was recorded every 10 s. Data were analyzed using the ORIGIN software package (version 7.0) supplied with the instrument. The amino acid sequences of the peptides used in the ITC experiments were as follows: vIRF4²⁰²⁻²¹⁶, SVWIPVNEGASTSGM; vIRF4²⁰⁹⁻²¹⁶, EGASTSGM; EBNA1⁴³⁵⁻⁴⁴⁹, EQGPADDPGEGPSTG; MDM2¹³⁷⁻¹⁵², LVQELQEEKPSSSHL; p53³⁵⁰⁻³⁶⁴, LKDAQAGKEPGGSRA; p53³⁵⁵⁻³⁶⁹, AGKEPGGSRAHSSHL. Each set of ITC experiments was repeated two or three times.

### Crystallization, Data Collection, and Structure Determination

Crystallization trials were carried out using in situ proteolysis technique. Trypsin-treated protein complexes was used immediately for crystallization trials. Crystals were grown for one week under conditions of 5% PEG 3350 and 0.2 M magnesium formate (pH 5.9) in the alternate reservoir containing a 1.5 M NaCl solution. Crystals were transferred to a cryoprotectant solution containing 30% PEG 3350 and 0.2 M magnesium formate (pH 5.9), incubated for 2 h, and then retrieved and placed immediately in a −173° C. nitrogen gas stream. X-ray diffraction data were collected at 1.6 Å resolution on beamline 4A at the Pohang Accelerator Laboratory (Pohang, Korea). All data were processed using the HKL2000 program suite (Ref 7). Crystal of the protein complex belongs to space group P3221. There is one complex in the asymmetric unit with a packing density of ˜2.26 Å3/Da, corresponding to an estimated solvent content of approximately 45.72%. The crystal structure was determined by molecular replacement using the MOLREP program (Kim, Y. et al. (2010) J. Biol. Chem 285: 14020-14030) using the HAUSP TRAF domain structure (PDB accession code 2F1W) as a search model. The initial model was used as a guide to build the remainder of the protein manually into electron density maps with the program COOT. The refinement was performed with REFMAC5. The refinement included the translation-liberation-screw procedure. The final refined model resulted in Rfree and Rcryst values of 0.174 and 0.158, respectively. The model contains 143 amino acids of the HAUSP TRAF domain, 15 residues of vIRF4, and 238 water molecules, and satisfies the quality criteria limits of the program PROCHECK. The crystallographic data statistics are summarized in Table 2. The atomic coordinates and structure factor amplitudes of the protein complex have been deposited in the Protein Data Bank (PDB)12 under the accession code 2XXN and are further provided in Table 3.

### NMR Spectroscopy

¹H-¹⁵N HSQC spectra of vIRF4¹⁵³⁻²⁵⁶ and its mutants were measured on a Bruker 900 MHz NMR spectrometer at the Korea Basic Science Institute (Ochang, Korea). NMR measurements were performed with 0.1 mM 15N-labeled protein in 50 mM HEPES (pH 6.5) containing 10% D₂O at 25° C. All NMR spectra were processed with Topspin 2.1 and analyzed with SPARKY 3.1 program.

Purification of vIRF4-HAUSP Complexes and Flag Elution

For vIRF4-HAUSP complex elutions, 293T cells were transfected as described above with the indicated constructs. After 48 h, cells were collected, lysed in 1% NP40 lysis buffer and lysates were incubated with anti-Flag M2 affinity gel (SIGMA). After binding of FLAG-tagged proteins, beads were washed three times with 1% NP40 lysis buffer and eluted in DUB buffer (25 mM Tris/HCl (pH 7.5), 50 mM NaCl, 10 mM MgCl2, 2 mM DTT, and 2 mM ATP) containing with 0.4 mg/ml Flag (M2)-peptide for 30 min at RT. Flag-peptide was removed and elutes were used for in vitro DUB assay.

### In Vitro Ubiquitination Assay

MDM2 auto-ubiquitination assays were performed in 10 μl reaction volumes with the following components as indicated: 100 μM ubiquitin (Boston Biochem), 100 nM E1 (Boston Biochem), 5 μM Ubc5b (E2, Boston Biochem), 600 μM MDM2 (E3, Boston Biochem), and ubiquitination reaction buffer (40 mM Tris-HCl (pH 7.6), 5 mM MgCl₂, 2 mM ATP, 2 mM DTT). Reactions were incubated at 37° C. for 3 h and prepared for immunoblot analysis as indicated.

### In Vitro De-Ubiquitination (DUB) Assay

All DUB reactions were continuous. Either ubiquitinated MDM2 or poly—ubiquitin chain (K48 Ub3-7, or K63 Ub3-7, Boston Biochem) were used as a substrate. In vitro DUB activity was assayed by incubating the substrate with purified HAUSP (USP7, Boston Biochem) in DUB buffer for the indicated time at 37° C. The reaction was terminated by the addition of an equal volume of 2×SDS—PAGE sample buffer. Proteins were resolved on 15% SDS—PAGE and blotted with anti—ubiquitin antibody.

### Cell Proliferation and Viability Assays

4×10⁵ cells were treated with the peptides for the indicated periods of time and their viability was measured by trypan blue (Gibco) exclusion, followed by analyses using the Backman Coulter Z2 particle count and size analyzer (BC Z2 CS Analyzer). A minimum of 100 cells per sample using triplicate samples was counted per condition per experiment.

### Cell Cycle and Apoptosis Assay

The proportion of cells at the S phase was determined by measuring incorporation of BrdU and 7-amino-actionomycin D (ADD) into DNA. Cells were grown at a density of 5×10⁶ cells/ml indicated periods with the treatment of either peptide or Nutlin-3a. The cells were pulse-labeled with 10 μM BrdU for 30 min. Cells were permeabilized, fixed and BrdU-, 7AAD-stained using the BrdU Flow Kit (BD Pharmingen) according to manufacturer's instructions. Stained cells were analyzed by flow cytometry to determine the cell-cycle distribution on a FACS Scan (BD FACSCanto™ II). Apoptosis was measured by dual-labeling with the Annexin V-FITC Apoptosis Detection kit I (BD biosciences-Pharmingen) according to the manufacturer's instruction and analyzed by Flow Jo software.

### In Vivo Bioimaging

Female NOD/SCID mice (4-6 weeks old) were purchased from Jackson Laboratory and maintained under specific pathogen free conditions in a temperature and humidity controlled environment. 5×10⁶ BCBL-1-Luc cells were injected intraperitoneally and treatment commenced after tumors were established. Mice were injected intraperitoneally with D-luciferin (50 μl; 75 mg/kg body weight) and were exposed for 1 min beginning 12 min after injection of D-luciferin to generate a bioluminescent image using an IVIS imaging system (Xenogen). D-luciferin firefly potassium salt was purchased from Xenogen and data were analysed with Igor Pro image analysis software (WaveMetrics). A region of interest (ROI) was manually selected over signal intensity and the area of the ROI was manually selected over signal intensity with the area of the ROI kept constant. Data are presented as average radiance (photons/s⁻¹. cm⁻¹sr⁻¹ [steradian] within the ROI. Finally, the mice were humanely killed by CO₂ inhalation immediately after the development of PEL, as defined by a weight gain of greater than 10% total body mass within a 1-week period.

### Results

Ubiquitin-specific-protease HAUSP plays pivotal roles in the stability of p53 tumor suppressor and its negative regulator MDM2 (Ref. 1-5), raising HAUSP as a potential therapeutic target for tuning p53-mediated anti-tumor activity. Here, this example reports the discovery of two short peptides, vif1 and vif2, derived from Kaposi's-sarcoma-associated-herpesvirus vIRF4 as potent and selective HAUSP antagonists. Co-crystal structural analysis of HAUSP-vIRF4 complex reveals a belt-type interaction, resulting in a bilateral inhibition of HAUSP activity. First, the vIRF4 15-amino-acid-sequence vif1 peptide binds the TRAF domain of HAUSP with a high affinity, competitively blocking substrate binding. Second, the vIRF4 17-amino-acid-sequence vif2 peptide broadly binds the TRAF and catalytic domains of HAUSP, robustly suppressing its deubiquitination activity. Consequently, peptide treatments comprehensively blocked HAUSP activity, leading to p53-dependent cell-cycle-arrest, apoptosis, and tumor regression in culture and xenografted mouse model. Thus, these virus-derived-short peptides represent biologically active HAUSP antagonists, potentially leading to a paradigm shift in p53-targeted anti-cancer therapy.

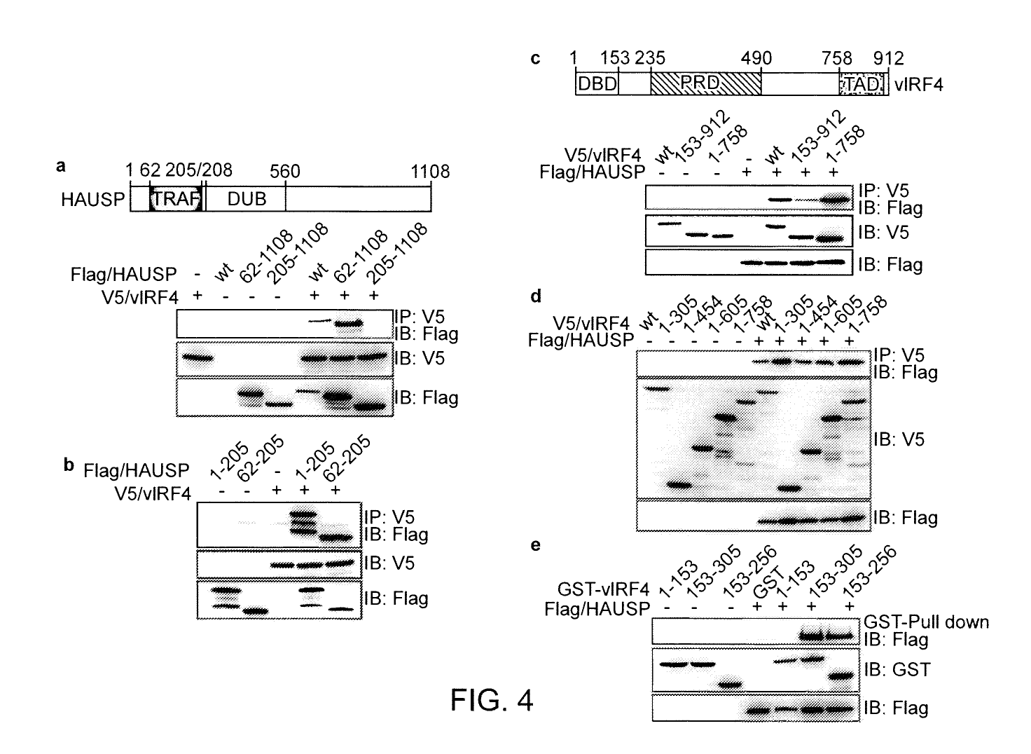

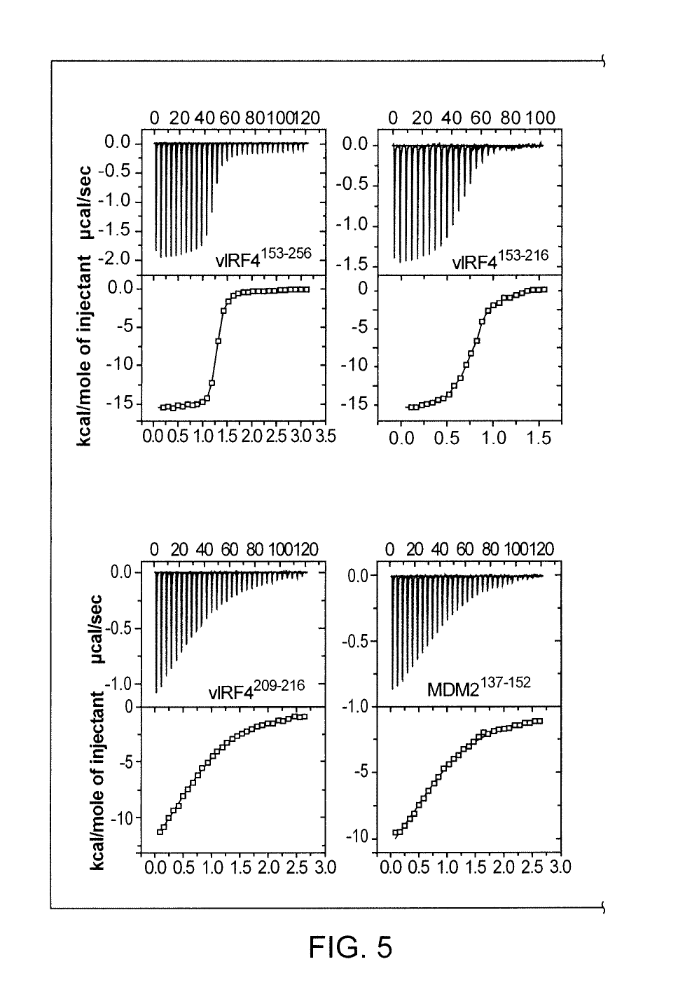

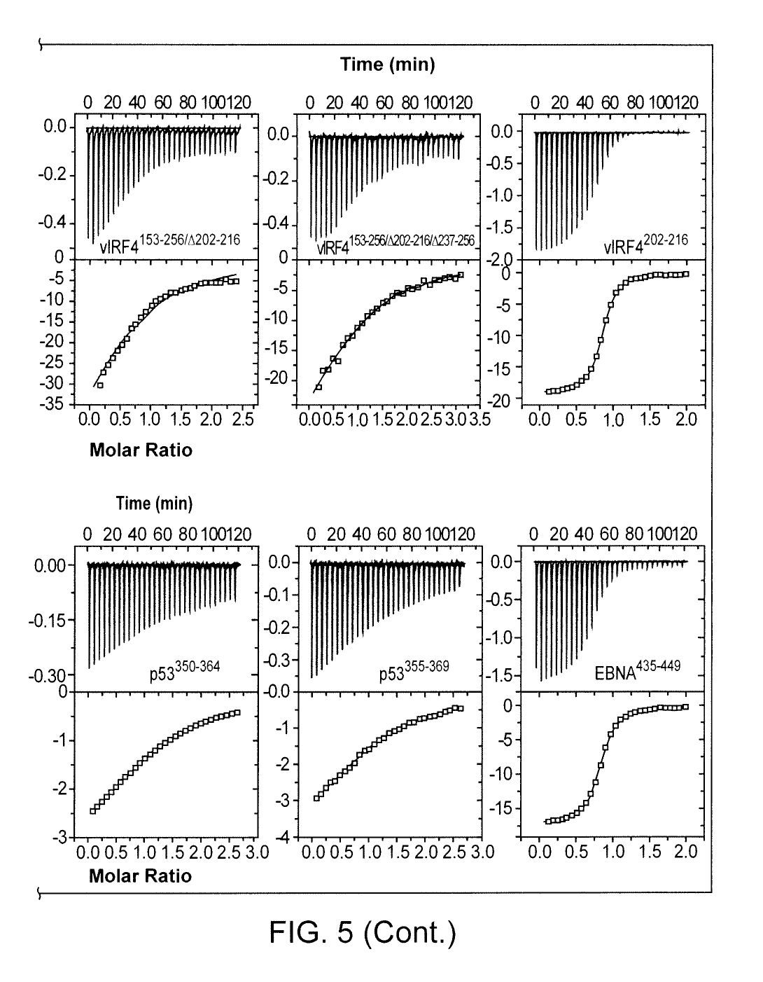

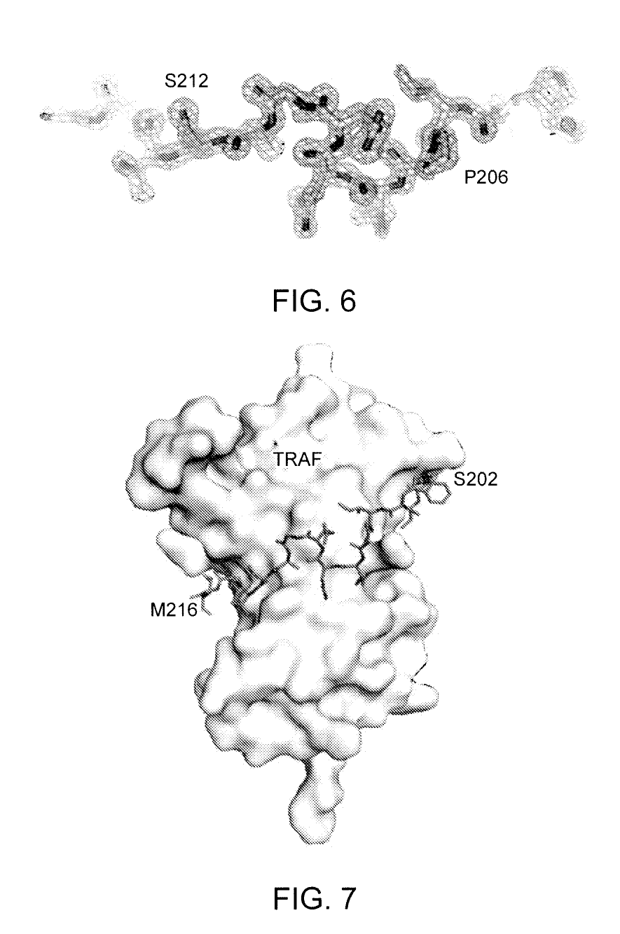

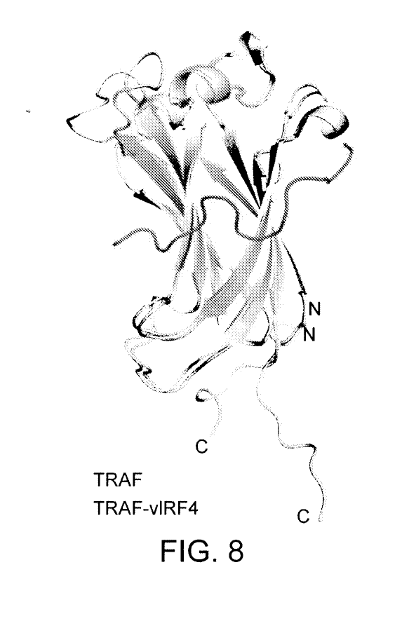

HAUSP has been shown to interact with a number of herpesviral proteins including ICP0 of HSV (α-herpesvirus) and EBNA-1 of Epstein-Barr virus (EBV, γ-1 herpesvirus). Hence, to study the HAUSP interaction network in KSHV (γ-2 herpesvirus), this study performed a yeast-two hybrid screen with a KSHV library (Ref 6) and Mass Spectrometry analysis. Both studies independently discovered a novel interaction between HAUSP and vIRF4 (FIG. 1a). Detailed binding assays indicate that the HAUSP TRAF domain (HAUSP⁶²⁻²⁰⁵) specifically interacts with the vIRF4 aa 153-256 region (vIRF4¹⁵³⁻²⁵⁶) (FIG. 4). Isothermal titration calorimetry (ITC) assay also revealed a robust interaction between vIRF4 and HAUSP with a dissociation constant (K_{d}=76 nM) of HAUSP⁶²⁻²⁰⁵ and vIRF4¹⁵³⁻²⁵⁶, remarkably higher than those (K_{d}=0.5-15 μM) reported for other HAUSP TRAF domain binding substrates (Ref 1, 7-9) (Table 1a and FIG. 5). To gain further insight into the molecular basis of the HAUSP-vIRF4 interaction, the HAUSP⁶²⁻²⁰⁵-vIRF4¹⁵³⁻²⁵⁶ complex was crystallized using an in situ proteolysis technique (Ref. 10). The three-dimensional structure of this crystallized complex was determined by the molecular replacement method using the HAUSP TRAF domain (PDB accession code 2F1W) as a search model, and refined to 1.6 Å resolution (FIG. 1b). All residues of HAUSP⁶²⁻²⁰⁵, with the exception of Asp⁶², are included in the final model, whereas only 15 residues (Ser²⁰² to Met²¹⁶) of vIRF4 are visible in the electron density map (FIG. 6). The overall structure of the HAUSP TRAF domain comprises a typical eight-stranded anti-parallel β-sandwich fold (FIG. 1b) that forms a shallow groove at the waist of the surface structure (FIGS. 1d and 7). No significant conformational changes were observed between the peptide-free (PDB accession code 2F1W) and vIRF4-bound TRAF domain except that the C-terminal region of TRAF domain was less extended upon vIRF4-binding than in a free form (FIG. 8).

TABLE 1a
Thermodynamic parameters of the interactions between the HAUSP
TRAF domain and the vIRF4 protein derivatives or other peptides.

| | | Kₐ | K_{d} | ΔH | TΔS | ΔG |
|---|---|---|---|---|---|---|
| | n | (10⁶M⁻¹) | (μM) | (kcal mol⁻¹) | (kcal mol⁻¹) | (kcal mol⁻¹) |
| vIRF4¹⁵³⁻²⁵⁶ | 1.13 ± 0.18 | 13.20 ± 0.85 | 0.076 ± 0.01 | −17.69 ± 1.17 | −10.05 | −7.64 |
| vIRF4¹⁵³⁻²¹⁶ | 0.82 ± 0.09 | 1.84 ± 0.10 | 0.54 ± 0.03 | −15.76 ± 0.18 | −7.35 | −8.41 |
| vIRF4^{153-256/Δ202-216} | 0.65 ± 0.08 | 0.29 ± 0.05 | 3.45 ± 0.02 | −62.09 ± 9.56 | −54.79 | −7.30 |
| vIRF4^{153-256/Δ202-216/Δ237-256} | 0.89 ± 0.07 | 0.25 ± 0.03 | 4.03 ± 0.03 | −41.35 ± 3.99 | −33.99 | −7.36 |
| vIRF4²⁰²⁻²¹⁶ | 0.84 ± 0.01 | 2.59 ± 0.05 | 0.39 ± 0.01 | −19.22 ± 0.05 | −10.63 | −8.60 |
| vIRF4²⁰⁹⁻²¹⁶ | 0.81 ± 0.01 | 0.10 ± 0.01 | 9.57 ± 0.19 | −17.06 ± 0.21 | −10.31 | −6.74 |
| MDM2¹³⁷⁻¹⁵² | 0.84 ± 0.06 | 0.09 ± 0.00 | 11.06 ± 1.40 | −16.48 ± 1.68 | −9.82 | −6.66 |
| p53³⁵⁰⁻³⁶⁴ | 1.15 ± 0.02 | 0.06 ± 0.00 | 15.46 ± 0.60 | −3.86 ± 0.09 | 2.59 | −6.45 |
| p53³⁵⁵⁻³⁶⁹ | 1.07 ± 0.01 | 0.07 ± 0.00 | 15.07 ± 1.17 | −4.69 ± 0.18 | 1.19 | −5.87 |
| EBNA1⁴³⁵⁻⁴⁴⁹ | 0.81 ± 0.01 | 2.10 ± 0.15 | 0.48 ± 0.04 | −17.17 ± 0.13 | −8.69 | −8.48 |

TABLE 1b
Thermodynamic parameters of competitive binding of vIRF4 with TRAF
domain against cellular substrates.

| | | K_{obs} | K_{d} | ΔH_{obs} | Kₐ | ΔHₐ |
|---|---|---|---|---|---|---|
| Titration | n | (10⁶M⁻¹) | (μM) | (kcal mol⁻¹) | (10⁶M⁻¹) | (kcal mol⁻¹) |
| vIRF4²⁰²⁻²¹⁶ to | 0.703 ± 0.001 | 10.875 ± 1.308 | 0.092 | −25.870 ± 0.240 | 0.091 | −16.480 |
| MDM2¹³⁷⁻¹⁵²- | | | | | | |
| TRAF | | | | | | |
| vIRF4²⁰²⁻²¹⁶ to | 0.779 ± 0.007 | 44.200 ± 4.200 | 0.023 | −19.070 ± 0.194 | 0.065 | −3.863 |
| p53³⁵⁰⁻³⁶⁴-TRAF | | | | | | |
| vIRF4²⁰²⁻²¹⁶ to | 0.761 ± 0.008 | 35.800 ± 3.400 | 0.028 | −19.150 ± 0.203 | 0.067 | −4.688 |
| p53³⁵⁵⁻³⁶⁹-TRAF | | | | | | |

Unlike previous studies that used synthetic peptides of 4-7 amino acids in length in complex with the HAUSP TRAF domain (Ref 1, 7-9), an in situ proteolysis treatment of the HAUSP⁶²⁻²⁰⁵-vIRF4¹⁵³⁻²⁵⁶ protein complex yielded a crystal structure with a remarkably longer 15-residue vIRF4 peptide consisting of an upstream (Ser²⁰² to Asn²⁰⁸) and downstream (Ala²¹¹ to Met²¹⁶) region positioned on the groove in a belt-type arrangement around the waist (FIGS. 1b and d and FIG. 7). The downstream region contains the well-conserved four-residue consensus sequence P/A××S (where x indicates any amino acid) binding motif shared by p53, MDM2, MDM4, and EBNA1 peptides, which binds to the same substrate-recognition site of the TRAF domain through conserved contacts (FIG. 1d). The equivalent motif of vIRF4 consists of Ala²¹¹, Ser²¹², Thr²¹³ and Ser²¹⁴, and engages in extensive polar and nonpolar interactions with one side of the TRAF β-sheet, particularly the β7 strand (FIGS. 1b and c). The methyl group of Ala²¹¹ participates in hydrophobic interactions with the side chains of TRAF Ile¹⁵⁴, Trp¹⁶⁵, and Phe¹⁶⁷. Of note, the vIRF4 Ser²¹² makes decisive contacts with the TRAF Gly¹⁶⁶ through backbone-backbone interactions, while the vIRF4 Thr²¹³ methyl group participates in van der Waals interactions with the aliphatic portions of the side chains of TRAF Glu¹⁶² and Trp¹⁶⁵ (FIG. 1c). The backbone amide of Ser²¹⁴, the most highly conserved residue among all HAUSP TRAF binding substrates, is hydrogen-bonded with the TRAF Aps¹⁶⁴ side chain carboxyl group and Arg¹⁰⁴ side chain amino group (FIGS. 1c and d). These interaction patterns are similar to those reported for other peptides as seen in References 1 and 7-9. In addition to the consensus residues of the 4-amino acid motif, the backbones of the two C-terminal vIRF4 peptide residues, Gly²¹⁵ and Met²¹⁶, participate in water molecule-mediated hydrogen bonding with the TRAF Lys¹⁶¹ backbone and Asp¹⁵⁴ side chain, respectively.

TABLE 2
Crystallographic data collection and refinement statistics

| | |
|---|---|
| Dataset | HAUSP⁶²⁻²⁰⁵-vIRF4¹⁵³⁻²⁵⁶ complex |
| Beamline (PAL) | 4A(MXW) |
| Wavelength | 0.9999 |
| Space group | P3₂21 |
| Cell dimensions (Å) | |
| A | 72.46 |
| B | 72.46 |
| C | 53.84 |
| Resolution (Å) | 1.60(1.66-1.60) |
| No. of total reflections | 476,332 |
| No. of unique reflections | 21,908 |
| Redundancy | 21.7(21.8) |
| Completeness (%) | 100(100) |
| R_{sym} (%)^{a} | 6.1(26.6) |
| I/Ω(I) | 65.2(11.9) |
| Refinement | |
| Resolution (Å) | 30.00-1.60 |
| Reflections in work/test sets | 20,747/1,118 |
| R_{cryst}/R_{free} (%)^{b,c} | 15.8/17.4 |
| R.m.s. deviations | |
| Bond lengths (Å) | 0.008 |
| Bond angles (°) | 1.131 |
| Model composition | 158 residues |
| | 238 waters |
| Geometry | |
| Most favored regions (%) | 88.5 |
| Additional allowed regions | 11.5 |
| (%) | |
| PDB accession code | 2XXN |

The numbers in parentheses describe the relevant value for the highest resolution shell.
^{a}R_{sym}= Σ|Iᵢ − <I>|/ΣI where Iᵢ is the intensity of the i-th observation and <I> is the mean intensity of the reflections.
^{b}R_{cryst}= Σ||F_{obs}| − |F_{calc}||/Σ|F_{obs}| where F_{calc} and F_{obs} are the calculated and observed structure factor amplitude, respectively.
^{c}R_{free}= Σ||F_{obs}| − |F_{calc}||/Σ|F_{obs}| where all reflections belong to a test set of randomly selected data.

TABLE 3
X, Y and Z atomic coordinates of vIRF4-HAUSP TRAF domain complex

| | | |
|---|---|---|
| HEADER | ---- | XX-XXX-9- XXXX |
| COMPND | --- | |

| | | | |
|---|---|---|---|
| REMARK | 3 | | |
| REMARK | 3 | REFINEMENT. | |
| REMARK | 3 | PROGRAM: | REFMAC 5.2.0019 |
| REMARK | 3 | AUTHORS: | MURSHUDOV, VAGIN, DODSON |
| REMARK | 3 | | |

| REMARK | 3 | REFINEMENT TARGET: MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS): | 1.60 |
| REMARK | 3 | RESOLUTION RANGE LOW | (ANGSTROMS): | 30.00 |
| REMARK | 3 | DATA CUTOFF | (SIGMA(F)): | NONE |

| | | | | |
|---|---|---|---|---|
| REMARK | 3 | COMPLETENESS FOR RANGE | (%): | 99.99 |

| | | | |
|---|---|---|---|
| REMARK | 3 | NUMBER OF REFLECTIONS: | 20747 |
| REMARK | 3 | | |

| REMARK | 3 | FIT TO DATA USED IN REFINEMENT. | | |
|---|---|---|---|---|
| REMARK | 3 | CROSS-VALIDATION METHOD: | | THROUGHOUT |
| REMARK | 3 | FREE R VALUE TEST SET SELECTION: | | RANDOM |
| REMARK | 3 | R VALUE | (WORKING + TEST SET): | 0.15861 |
| REMARK | 3 | R VALUE | (WORKING SET): | 0.15777 |
| REMARK | 3 | FREE R VALUE: | | 0.17426 |

| | | | | |
|---|---|---|---|---|
| REMARK | 3 | FREE R VALUE TEST SET SIZE | (%): | 5.1 |

| | | | |
|---|---|---|---|
| REMARK | 3 | FREE R VALUE TEST SET COUNT: | 1118 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED: | | 20 |
| REMARK | 3 | BIN RESOLUTION RANGE HIGH: | | 1.601 |
| REMARK | 3 | BIN RESOLUTION RANGE LOW: | | 1.643 |
| REMARK | 3 | REFLECTION IN BIN | (WORKING SET): | 1539 |
| REMARK | 3 | BIN COMPLETENESS | (WORKING + TEST) (%): | 100.00 |
| REMARK | 3 | BIN R VALUE | (WORKING SET): | 0.143 |

| | | | |
|---|---|---|---|
| REMARK | 3 | BIN FREE R VALUE SET COUNT: | 72 |
| REMARK | 3 | BIN FREE R VALUE: | 0.216 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS: | 1562 |
| REMARK | 3 | | |
| REMARK | 3 | B VALUES. | |

| | | | | |
|---|---|---|---|---|
| REMARK | 3 | FROM WILSON PLOT | (A**2): | NULL |
| REMARK | 3 | MEAN B VALUE | (OVERALL, A**2): | 14.858 |

| REMARK | 3 | OVERALL ANISOTROPIC B VALUE. | |
|---|---|---|---|
| REMARK | 3 | B11 (A**2): | 0.50 |
| REMARK | 3 | B22 (A**2): | 0.50 |
| REMARK | 3 | B33 (A**2): | −0.75 |
| REMARK | 3 | B12 (A**2): | 0.25 |
| REMARK | 3 | B13 (A**2): | 0.00 |
| REMARK | 3 | B23 (A**2): | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A): | 0.124 |
| REMARK | 3 | ESU BASED ON FREE R VALUE | (A): | 0.078 |
| REMARK | 3 | ESU BASED ON MAXIMUM LIKELIHOOD | (A): | 0.045 |
| REMARK | 3 | ESU FOR B VALUES BASED ON MAXIMUM LIKELIHOOD | (A**2): | 2.754 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC: | 0.963 |
| REMARK | 3 | CORRELATION COEFFICIENT FO-FC FREE: | 0.956 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 1369; | 0.008; | 0.022 |

| | | | | | | |
|---|---|---|---|---|---|---|
| REMARK | 3 | BOND ANGLES REFINED ATOMS | (DEGREES): | 1865; | 1.131; | 1.902 |
| REMARK | 3 | TORSION ANGLES, PERIOD 1 | (DEGREES): | 164; | 6.325; | 5.000 |
| REMARK | 3 | TORSION ANGLES, PERIOD 2 | (DEGREES): | 69; | 35.456; | 23.188 |
| REMARK | 3 | TORSION ANGLES, PERIOD 3 | (DEGREES): | 213; | 11.676; | 15.000 |
| REMARK | 3 | TORSION ANGLES, PERIOD 4 | (DEGREES): | 9; | 10.770; | 15.000 |
| REMARK | 3 | CHIRAL-CENTER RESTRAINTS | (A**3): | 190; | 0.081; | 0.200 |

| | | | | | | |
|---|---|---|---|---|---|---|
| REMARK | 3 | GENERAL PLANES REFINED ATOMS | (A): | 1086; | 0.003; | 0.020 |
| REMARK | 3 | NON-BONDED CONTACTS REFINED ATOMS | (A): | 607; | 0.191; | 0.200 |
| REMARK | 3 | NON-BONDED TORSION REFINED ATOMS | (A): | 943; | 0.305; | 0.200 |
| REMARK | 3 | H-BOND (X...Y) REFINED ATOMS | (A): | 157; | 0.122; | 0.200 |
| REMARK | 3 | SYMMETRY VDW REFINED ATOMS | (A): | 49; | 0.158; | 0.200 |
| REMARK | 3 | SYMMETRY H-BOND REFINED ATOMS | (A): | 49; | 0.140; | 0.200 |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2): | 841; | 1.376; | 3.000 |
| REMARK | 3 | MAIN-CHAIN ANGLE REFINED ATOMS | (A**2): | 1332; | 1.912; | 5.000 |
| REMARK | 3 | SIDE-CHAIN BOND REFINED ATOMS | (A**2): | 623; | 2.367; | 8.000 |
| REMARK | 3 | SIDE-CHAIN ANGLE REFINED ATOMS | (A**2): | 533; | 3.334; | 11.000 |
| REMARK | 3 | | | | | |
| REMARK | 3 | ANISOTROPIC THERMAL FACTOR RESTRAINTS. | | COUNT | RMS | WEIGHT |
| REMARK | 3 | RIGID-BOND RESTRAINTS | (A**2): | 1464; | 1.300; | 3.000 |
| REMARK | 3 | SPHERICITY; FREE ATOMS | (A**2): | 240; | 1.820; | 3.000 |
| REMARK | 3 | SPHERICITY; BONDED ATOMS | (A**2): | 1322; | 1.466; | 3.000 |
| REMARK | 3 | | | | | |
| REMARK | 3 | NCS RESTRAINTS STATISTICS | | | | |

| | | |
|---|---|---|
| REMARK | 3 | NUMBER OF NCS GROUPS: NULL |
| REMARK | 3 | |
| REMARK | 3 | |
| REMARK | 3 | TLS DETAILS |
| REMARK | 3 | NUMBER OF TLS GROUPS: NULL |
| REMARK | 3 | |
| REMARK | 3 | |
| REMARK | 3 | BULK SOLVENT MODELLING. |
| REMARK | 3 | METHOD USED: MASK |
| REMARK | 3 | PARAMETERS FOR MASK CALCULATION |
| REMARK | 3 | VDW PROBE RADIUS: 1.20 |
| REMARK | 3 | ION PROBE RADIUS: 0.80 |
| REMARK | 3 | SHRINKAGE RADIUS: 0.80 |
| REMARK | 3 | |
| REMARK | 3 | OTHER REFINEMENT REMARKS: NULL |
| REMARK | 3 | |

| REMARK | [No title given] | | | | |
|---|---|---|---|---|---|
| CRYST1 | 72.462 | 72.462 | 53.840 | 90.00 90.00 | 120.00 P 32 2 1 |

| | | | | |
|---|---|---|---|---|
| SCALE1 | 0.013800 | 0.007968 | 0.000000 | 0.00000 |
| SCALE2 | 0.000000 | 0.015935 | 0.000000 | 0.00000 |
| SCALE3 | 0.000000 | 0.000000 | 0.018574 | 0.00000 |

| | | | | | | | | | | | | | |
|---|---|---|---|---|---|---|---|---|---|---|---|---|---|
| ATOM | 1 | N | THR | A | 63 | −18.464 | −20.926 | −15.505 | 1.00 | 26.89 | | A | N |
| ANISOU | 1 | N | THR | A | 63 | 3347 | 3414 | 3456 | 12 | 1 | −7 | A | N |
| ATOM | 2 | CA | THR | A | 63 | −19.723 | −21.698 | −15.733 | 1.00 | 26.28 | | A | C |
| ANISOU | 2 | CA | THR | A | 63 | 3254 | 3376 | 3357 | 62 | 29 | −37 | A | C |
| ATOM | 3 | CB | THR | A | 63 | −19.603 | −23.148 | −15.213 | 1.00 | 27.79 | | A | C |
| ANISOU | 3 | CB | THR | A | 63 | 3559 | 3469 | 3530 | 43 | 43 | 12 | A | C |
| ATOM | 4 | OG1 | THR | A | 63 | −20.766 | −23.886 | −15.594 | 1.00 | 30.77 | | A | O |
| ANISOU | 4 | OG1 | THR | A | 63 | 3816 | 3922 | 3954 | −27 | −106 | −35 | A | O |
| ATOM | 5 | CG2 | THR | A | 63 | −19.443 | −23.189 | −13.686 | 1.00 | 29.37 | | A | C |
| ANISOU | 5 | CG2 | THR | A | 63 | 3771 | 3752 | 3637 | −4 | −23 | 23 | A | C |
| ATOM | 6 | C | THR | A | 63 | −20.942 | −21.006 | −15.109 | 1.00 | 24.04 | | A | C |
| ANISOU | 6 | C | THR | A | 63 | 2958 | 3099 | 3077 | 8 | −40 | −15 | A | C |
| ATOM | 7 | O | THR | A | 63 | −20.816 | −20.304 | −14.105 | 1.00 | 24.81 | | A | O |
| ANISOU | 7 | O | THR | A | 63 | 3010 | 3233 | 3182 | 39 | −17 | −61 | A | O |
| ATOM | 8 | N | SER | A | 64 | −22.115 | −21.216 | −15.707 | 1.00 | 21.22 | | A | N |
| ANISOU | 8 | N | SER | A | 64 | 2646 | 2721 | 2695 | 69 | 67 | −34 | A | N |
| ATOM | 9 | CA | SER | A | 64 | −23.348 | −20.556 | −15.255 | 1.00 | 18.31 | | A | C |
| ANISOU | 9 | CA | SER | A | 64 | 2348 | 2372 | 2236 | −13 | 0 | 1 | A | C |
| ATOM | 10 | CB | SER | A | 64 | −24.462 | −20.687 | −16.301 | 1.00 | 18.45 | | A | C |
| ANISOU | 10 | CB | SER | A | 64 | 2230 | 2447 | 2332 | −14 | −3 | −10 | A | C |
| ATOM | 11 | OG | SER | A | 64 | −24.178 | −19.908 | −17.446 | 1.00 | 19.58 | | A | O |
| ANISOU | 11 | OG | SER | A | 64 | 2498 | 2447 | 2496 | 44 | −1 | 113 | A | O |
| ATOM | 12 | C | SER | A | 64 | −23.862 | −21.061 | −13.915 | 1.00 | 16.22 | | A | C |
| ANISOU | 12 | C | SER | A | 64 | 2015 | 2041 | 2107 | −10 | 15 | −54 | A | C |
| ATOM | 13 | O | SER | A | 64 | −24.464 | −20.299 | −13.163 | 1.00 | 14.38 | | A | O |
| ANISOU | 13 | O | SER | A | 64 | 1682 | 1823 | 1957 | −81 | 20 | −62 | A | O |
| ATOM | 14 | N | TRP | A | 65 | −23.615 | −22.337 | −13.623 | 1.00 | 15.42 | | A | N |
| ANISOU | 14 | N | TRP | A | 65 | 1936 | 1985 | 1938 | −40 | −4 | −35 | A | N |
| ATOM | 15 | CA | TRP | A | 65 | −24.185 | −22.985 | −12.440 | 1.00 | 15.65 | | A | C |
| ANISOU | 15 | CA | TRP | A | 65 | 2000 | 1980 | 1966 | −46 | −17 | −7 | A | C |
| ATOM | 16 | CB | TRP | A | 65 | −24.426 | −24.483 | −12.694 | 1.00 | 17.87 | | A | C |
| ANISOU | 16 | CB | TRP | A | 65 | 2377 | 2139 | 2273 | −63 | 3 | −108 | A | C |
| ATOM | 17 | CG | TRP | A | 65 | −23.196 | −25.245 | −13.113 | 1.00 | 20.62 | | A | C |
| ANISOU | 17 | CG | TRP | A | 65 | 2570 | 2614 | 2649 | 10 | 56 | −10 | A | C |
| ATOM | 18 | CD1 | TRP | A | 65 | −22.767 | −25.461 | −14.390 | 1.00 | 22.14 | | A | C |
| ANISOU | 18 | CD1 | TRP | A | 65 | 2818 | 2863 | 2730 | 23 | 21 | −57 | A | C |
| ATOM | 19 | NE1 | TRP | A | 65 | −21.608 | −26.201 | −14.384 | 1.00 | 23.49 | | A | N |
| ANISOU | 19 | NE1 | TRP | A | 65 | 2935 | 3029 | 2960 | 75 | −30 | −22 | A | N |
| ATOM | 20 | CE2 | TRP | A | 65 | −21.269 | −26.487 | −13.087 | 1.00 | 22.75 | | A | C |
| ANISOU | 20 | CE2 | TRP | A | 65 | 2839 | 2965 | 2840 | 144 | 25 | 33 | A | C |
| ATOM | 21 | CD2 | TRP | A | 65 | −22.251 | −25.901 | −12.255 | 1.00 | 22.32 | | A | C |
| ANISOU | 21 | CD2 | TRP | A | 65 | 2816 | 2905 | 2761 | 68 | −1 | −8 | A | C |
| ATOM | 22 | CE3 | TRP | A | 65 | −22.135 | −26.050 | −10.865 | 1.00 | 22.09 | | A | C |
| ANISOU | 22 | CE3 | TRP | A | 65 | 2745 | 2885 | 2765 | 97 | −18 | 37 | A | C |
| ATOM | 23 | CZ3 | TRP | A | 65 | −21.055 | −26.772 | −10.356 | 1.00 | 23.36 | | A | C |
| ANISOU | 23 | CZ3 | TRP | A | 65 | 2884 | 3077 | 2916 | 125 | −5 | 7 | A | C |
| ATOM | 24 | CH2 | TRP | A | 65 | −20.094 | −27.340 | −11.213 | 1.00 | 23.04 | | A | C |
| ANISOU | 24 | CH2 | TRP | A | 65 | 2859 | 3038 | 2856 | 119 | −16 | 32 | A | C |
| ATOM | 25 | CZ2 | TRP | A | 65 | −20.184 | −27.209 | −12.577 | 1.00 | 23.30 | | A | C |
| ANISOU | 25 | CZ2 | TRP | A | 65 | 2879 | 3110 | 2865 | 96 | −6 | 26 | A | C |
| ATOM | 26 | C | TRP | A | 65 | −23.374 | −22.783 | −11.160 | 1.00 | 14.29 | | A | C |
| ANISOU | 26 | C | TRP | A | 65 | 1809 | 1784 | 1838 | −34 | 8 | 13 | A | C |
| ATOM | 27 | O | TRP | A | 65 | −23.796 | −23.223 | −10.082 | 1.00 | 14.84 | | A | O |
| ANISOU | 27 | O | TRP | A | 65 | 1917 | 1857 | 1863 | −40 | −27 | −64 | A | O |
| ATOM | 28 | N | ARG | A | 66 | −22.231 | −22.100 | −11.277 | 1.00 | 12.75 | | A | N |
| ANISOU | 28 | N | ARG | A | 66 | 1661 | 1614 | 1571 | 25 | −82 | −22 | A | N |
| ATOM | 29 | CA | ARG | A | 66 | −21.365 | −21.812 | −10.125 | 1.00 | 11.91 | | A | C |
| ANISOU | 29 | CA | ARG | A | 66 | 1562 | 1479 | 1486 | 3 | −19 | −30 | A | C |
| ATOM | 30 | CB | ARG | A | 66 | −20.234 | −20.858 | −10.514 | 1.00 | 11.45 | | A | C |
| ANISOU | 30 | CB | ARG | A | 66 | 1481 | 1480 | 1390 | 23 | −34 | −22 | A | C |
| ATOM | 31 | CG | ARG | A | 66 | −20.712 | −19.481 | −10.934 | 1.00 | 11.33 | | A | C |
| ANISOU | 31 | CG | ARG | A | 66 | 1392 | 1411 | 1501 | −46 | 18 | −17 | A | C |
| ATOM | 32 | CD | ARG | A | 66 | −19.600 | −18.671 | −11.547 | 1.00 | 12.10 | | A | C |
| ANISOU | 32 | CD | ARG | A | 66 | 1581 | 1501 | 1514 | −19 | 66 | 106 | A | C |
| ATOM | 33 | NE | ARG | A | 66 | −20.140 | −17.505 | −12.237 | 1.00 | 11.70 | | A | N |
| ANISOU | 33 | NE | ARG | A | 66 | 1523 | 1477 | 1447 | 100 | −3 | −18 | A | N |
| ATOM | 34 | CZ | ARG | A | 66 | −19.399 | −16.560 | −12.803 | 1.00 | 14.43 | | A | C |
| ANISOU | 34 | CZ | ARG | A | 66 | 1915 | 1804 | 1764 | −77 | 29 | 101 | A | C |
| ATOM | 35 | NH1 | ARG | A | 66 | −19.985 | −15.538 | −13.411 | 1.00 | 14.13 | | A | N |
| ANISOU | 35 | NH1 | ARG | A | 66 | 1714 | 1732 | 1922 | 95 | 32 | −11 | A | N |
| ATOM | 36 | NH2 | ARG | A | 66 | −18.075 | −16.639 | −12.768 | 1.00 | 18.07 | | A | N |
| ANISOU | 36 | NH2 | ARG | A | 66 | 2062 | 2431 | 2373 | 48 | −19 | 61 | A | N |
| ATOM | 37 | C | ARG | A | 66 | −22.162 | −21.229 | −8.962 | 1.00 | 12.13 | | A | C |
| ANISOU | 37 | C | ARG | A | 66 | 1558 | 1529 | 1520 | 35 | −9 | −26 | A | C |
| ATOM | 38 | O | ARG | A | 66 | −23.109 | −20.463 | −9.166 | 1.00 | 13.42 | | A | O |
| ANISOU | 38 | O | ARG | A | 66 | 1666 | 1790 | 1643 | 75 | −94 | −30 | A | O |
| ATOM | 39 | N | SER | A | 67 | −21.767 | −21.593 | −7.746 | 1.00 | 11.67 | | A | N |
| ANISOU | 39 | N | SER | A | 67 | 1535 | 1433 | 1466 | −13 | 3 | −48 | A | N |
| ATOM | 40 | CA | SER | A | 67 | −22.494 | −21.188 | −6.549 | 1.00 | 11.04 | | A | C |
| ANISOU | 40 | CA | SER | A | 67 | 1447 | 1319 | 1429 | −22 | −5 | −57 | A | C |
| ATOM | 41 | CB | SER | A | 67 | −22.182 | −22.132 | −5.384 | 1.00 | 12.14 | | A | C |
| ANISOU | 41 | CB | SER | A | 67 | 1649 | 1446 | 1516 | −78 | 0 | 37 | A | C |
| ATOM | 42 | OG | SER | A | 67 | −20.813 | −22.069 | −5.029 | 1.00 | 15.70 | | A | O |
| ANISOU | 42 | OG | SER | A | 67 | 1870 | 2016 | 2079 | 61 | −10 | −9 | A | O |
| ATOM | 43 | C | SER | A | 67 | −22.202 | −19.748 | −6.144 | 1.00 | 10.63 | | A | C |
| ANISOU | 43 | C | SER | A | 67 | 1307 | 1287 | 1446 | −41 | 35 | −45 | A | C |
| ATOM | 44 | O | SER | A | 67 | −22.981 | −19.143 | −5.411 | 1.00 | 11.07 | | A | O |
| ANISOU | 44 | O | SER | A | 67 | 1387 | 1330 | 1488 | −32 | 92 | −61 | A | O |
| ATOM | 45 | N | GLU | A | 68 | −21.080 | −19.205 | −6.609 | 1.00 | 10.01 | | A | N |
| ANISOU | 45 | N | GLU | A | 68 | 1365 | 1137 | 1302 | −41 | 98 | −91 | A | N |
| ATOM | 46 | CA | GLU | A | 68 | −20.685 | −17.851 | −6.219 | 1.00 | 10.57 | | A | C |
| ANISOU | 46 | CA | GLU | A | 68 | 1367 | 1266 | 1382 | −68 | 82 | −12 | A | C |
| ATOM | 47 | CB | GLU | A | 68 | −19.967 | −17.850 | −4.865 | 1.00 | 11.60 | | A | C |
| ANISOU | 47 | CB | GLU | A | 68 | 1480 | 1435 | 1492 | 15 | 25 | −13 | A | C |
| ATOM | 48 | CG | GLU | A | 68 | −18.644 | −18.609 | −4.836 | 1.00 | 12.93 | | A | C |
| ANISOU | 48 | CG | GLU | A | 68 | 1510 | 1672 | 1732 | 84 | −6 | −5 | A | C |
| ATOM | 49 | CD | GLU | A | 68 | −18.017 | −18.649 | −3.455 | 1.00 | 14.87 | | A | C |
| ANISOU | 49 | CD | GLU | A | 68 | 2016 | 1806 | 1829 | 44 | −81 | −31 | A | C |
| ATOM | 50 | OE1 | GLU | A | 68 | −18.716 | −19.004 | −2.478 | 1.00 | 16.77 | | A | O |
| ANISOU | 50 | OE1 | GLU | A | 68 | 2085 | 2171 | 2116 | 19 | 49 | −23 | A | O |
| ATOM | 51 | OE2 | GLU | A | 68 | −16.814 | −18.335 | −3.350 | 1.00 | 18.03 | | A | O |
| ANISOU | 51 | OE2 | GLU | A | 68 | 2081 | 2323 | 2445 | −50 | −99 | −19 | A | O |
| ATOM | 52 | C | GLU | A | 68 | −19.827 | −17.175 | −7.275 | 1.00 | 10.24 | | A | C |
| ANISOU | 52 | C | GLU | A | 68 | 1341 | 1226 | 1324 | −27 | 65 | 17 | A | C |
| ATOM | 53 | O | GLU | A | 68 | −19.202 | −17.844 | −8.099 | 1.00 | 10.96 | | A | O |
| ANISOU | 53 | O | GLU | A | 68 | 1472 | 1323 | 1371 | −32 | 108 | −41 | A | O |
| ATOM | 54 | N | ALA | A | 69 | −19.814 | −15.845 | −7.240 | 1.00 | 9.63 | | A | N |
| ANISOU | 54 | N | ALA | A | 69 | 1253 | 1223 | 1183 | −54 | 68 | 10 | A | N |
| ATOM | 55 | CA | ALA | A | 69 | −18.995 | −15.044 | −8.141 | 1.00 | 9.82 | | A | C |
| ANISOU | 55 | CA | ALA | A | 69 | 1263 | 1256 | 1211 | −51 | 17 | 21 | A | C |
| ATOM | 56 | CB | ALA | A | 69 | −19.629 | −14.966 | −9.526 | 1.00 | 11.13 | | A | C |
| ANISOU | 56 | CB | ALA | A | 69 | 1464 | 1469 | 1295 | −16 | −70 | 25 | A | C |
| ATOM | 57 | C | ALA | A | 69 | −18.809 | −13.651 | −7.582 | 1.00 | 9.58 | | A | C |
| ANISOU | 57 | C | ALA | A | 69 | 1220 | 1230 | 1189 | 43 | 9 | 31 | A | C |
| ATOM | 58 | O | ALA | A | 69 | −19.622 | −13.170 | −6.791 | 1.00 | 9.70 | | A | O |
| ANISOU | 58 | O | ALA | A | 69 | 1284 | 1278 | 1124 | 41 | 52 | 51 | A | O |
| ATOM | 59 | N | THR | A | 70 | −17.724 | −13.016 | −7.998 | 1.00 | 10.14 | | A | N |
| ANISOU | 59 | N | THR | A | 70 | 1336 | 1260 | 1257 | −51 | 17 | 39 | A | N |
| ATOM | 60 | CA | THR | A | 70 | −17.475 | −11.623 | −7.692 | 1.00 | 10.28 | | A | C |
| ANISOU | 60 | CA | THR | A | 70 | 1312 | 1290 | 1305 | 38 | 65 | −5 | A | C |
| ATOM | 61 | CB | THR | A | 70 | −16.168 | −11.451 | −6.895 | 1.00 | 11.52 | | A | C |
| ANISOU | 61 | CB | THR | A | 70 | 1458 | 1525 | 1393 | 20 | 2 | −15 | A | C |
| ATOM | 62 | OG1 | THR | A | 70 | −16.253 | −12.203 | −5.678 | 1.00 | 13.25 | | A | O |
| ANISOU | 62 | OG1 | THR | A | 70 | 1740 | 1737 | 1558 | 103 | 3 | 112 | A | O |
| ATOM | 63 | CG2 | THR | A | 70 | −15.924 | −9.994 | −6.565 | 1.00 | 12.01 | | A | C |
| ANISOU | 63 | CG2 | THR | A | 70 | 1534 | 1578 | 1451 | −40 | −68 | −16 | A | C |
| ATOM | 64 | C | THR | A | 70 | −17.386 | −10.898 | −9.021 | 1.00 | 10.88 | | A | C |
| ANISOU | 64 | C | THR | A | 70 | 1436 | 1337 | 1361 | 53 | 20 | 8 | A | C |
| ATOM | 65 | O | THR | A | 70 | −16.662 | −11.330 | −9.921 | 1.00 | 11.45 | | A | O |
| ANISOU | 65 | O | THR | A | 70 | 1587 | 1413 | 1351 | 57 | 220 | 50 | A | O |
| ATOM | 66 | N | PHE | A | 71 | −18.152 | −9.821 | −9.164 | 1.00 | 9.84 | | A | N |
| ANISOU | 66 | N | PHE | A | 71 | 1334 | 1212 | 1191 | 26 | 14 | 18 | A | N |
| ATOM | 67 | CA | PHE | A | 71 | −18.051 | −8.996 | −10.367 | 1.00 | 9.82 | | A | C |
| ANISOU | 67 | CA | PHE | A | 71 | 1340 | 1175 | 1215 | −13 | −4 | 11 | A | C |
| ATOM | 68 | CB | PHE | A | 71 | −19.067 | −9.419 | −11.453 | 1.00 | 10.67 | | A | C |
| ANISOU | 68 | CB | PHE | A | 71 | 1420 | 1396 | 1240 | 26 | −24 | −21 | A | C |
| ATOM | 69 | CG | PHE | A | 71 | −20.513 | −9.161 | −11.108 | 1.00 | 10.86 | | A | C |
| ANISOU | 69 | CG | PHE | A | 71 | 1478 | 1371 | 1278 | −31 | −49 | −91 | A | C |
| ATOM | 70 | CD1 | PHE | A | 71 | −21.208 | −8.123 | −11.725 | 1.00 | 11.50 | | A | C |
| ANISOU | 70 | CD1 | PHE | A | 71 | 1501 | 1460 | 1408 | −12 | −38 | −83 | A | C |
| ATOM | 71 | CE1 | PHE | A | 71 | −22.552 | −7.887 | −11.439 | 1.00 | 12.15 | | A | C |
| ANISOU | 71 | CE1 | PHE | A | 71 | 1589 | 1679 | 1348 | 143 | −16 | −18 | A | C |
| ATOM | 72 | CZ | PHE | A | 71 | −23.224 | −8.703 | −10.530 | 1.00 | 13.50 | | A | C |
| ANISOU | 72 | CZ | PHE | A | 71 | 1581 | 1655 | 1894 | 33 | 78 | 20 | A | C |
| ATOM | 73 | CE2 | PHE | A | 71 | −22.547 | −9.751 | −9.908 | 1.00 | 13.14 | | A | C |
| ANISOU | 73 | CE2 | PHE | A | 71 | 1546 | 1708 | 1737 | 19 | 15 | 59 | A | C |
| ATOM | 74 | CD2 | PHE | A | 71 | −21.194 | −9.982 | −10.211 | 1.00 | 12.77 | | A | C |
| ANISOU | 74 | CD2 | PHE | A | 71 | 1530 | 1642 | 1680 | −17 | 97 | 72 | A | C |
| ATOM | 75 | C | PHE | A | 71 | −18.133 | −7.516 | −10.030 | 1.00 | 9.72 | | A | C |
| ANISOU | 75 | C | PHE | A | 71 | 1271 | 1187 | 1234 | −42 | 67 | −50 | A | C |
| ATOM | 76 | O | PHE | A | 71 | −18.745 | −7.136 | −9.028 | 1.00 | 9.21 | | A | O |
| ANISOU | 76 | O | PHE | A | 71 | 1273 | 1114 | 1113 | −111 | 72 | −57 | A | O |
| ATOM | 77 | N | GLN | A | 72 | −17.484 | −6.699 | −10.859 | 1.00 | 9.52 | | A | N |
| ANISOU | 77 | N | GLN | A | 72 | 1368 | 1089 | 1160 | 1 | 34 | 36 | A | N |
| ATOM | 78 | CA | GLN | A | 72 | −17.462 | −5.252 | −10.672 | 1.00 | 10.17 | | A | C |
| ANISOU | 78 | CA | GLN | A | 72 | 1313 | 1205 | 1346 | −43 | −14 | 34 | A | C |
| ATOM | 79 | CB | GLN | A | 72 | −16.025 | −4.724 | −10.685 | 1.00 | 10.42 | | A | C |
| ANISOU | 79 | CB | GLN | A | 72 | 1257 | 1274 | 1427 | −33 | −25 | 23 | A | C |
| ATOM | 80 | CG | GLN | A | 72 | −15.152 | −5.289 | −9.574 | 1.00 | 11.96 | | A | C |
| ANISOU | 80 | CG | GLN | A | 72 | 1539 | 1542 | 1462 | 36 | −118 | −17 | A | C |
| ATOM | 81 | CD | GLN | A | 72 | −13.849 | −4.532 | −9.396 | 1.00 | 14.73 | | A | C |
| ANISOU | 81 | CD | GLN | A | 72 | 1690 | 1852 | 2053 | −55 | 28 | −18 | A | C |
| ATOM | 82 | OE1 | GLN | A | 72 | −13.333 | −3.910 | −10.330 | 1.00 | 16.24 | | A | O |
| ANISOU | 82 | OE1 | GLN | A | 72 | 2081 | 2127 | 1963 | 78 | −14 | 25 | A | O |
| ATOM | 83 | NE2 | GLN | A | 72 | −13.307 | −4.582 | −8.186 | 1.00 | 17.78 | | A | N |
| ANISOU | 83 | NE2 | GLN | A | 72 | 2314 | 2302 | 2139 | 39 | −94 | −27 | A | N |
| ATOM | 84 | C | GLN | A | 72 | −18.276 | −4.536 | −11.739 | 1.00 | 10.02 | | A | C |
| ANISOU | 84 | C | GLN | A | 72 | 1325 | 1214 | 1268 | −21 | 8 | 20 | A | C |
| ATOM | 85 | O | GLN | A | 72 | −18.533 | −5.079 | −12.815 | 1.00 | 10.87 | | A | O |
| ANISOU | 85 | O | GLN | A | 72 | 1587 | 1185 | 1359 | −72 | −71 | −3 | A | O |
| ATOM | 86 | N | PHE | A | 73 | −18.674 | −3.309 | −11.422 | 1.00 | 9.55 | | A | N |
| ANISOU | 86 | N | PHE | A | 73 | 1277 | 1173 | 1179 | −28 | 4 | 31 | A | N |
| ATOM | 87 | CA | PHE | A | 73 | −19.400 | −2.453 | −12.343 | 1.00 | 10.16 | | A | C |
| ANISOU | 87 | CA | PHE | A | 73 | 1363 | 1184 | 1312 | −25 | 29 | 42 | A | C |
| ATOM | 88 | CB | PHE | A | 73 | −20.906 | −2.580 | −12.102 | 1.00 | 11.41 | | A | C |
| ANISOU | 88 | CB | PHE | A | 73 | 1415 | 1421 | 1501 | −39 | −8 | −8 | A | C |
| ATOM | 89 | CG | PHE | A | 73 | −21.749 | −1.693 | −12.978 | 1.00 | 13.61 | | A | C |
| ANISOU | 89 | CG | PHE | A | 73 | 1623 | 1579 | 1968 | −77 | −43 | 110 | A | C |
| ATOM | 90 | CD1 | PHE | A | 73 | −21.536 | −1.639 | −14.353 | 1.00 | 16.84 | | A | C |
| ANISOU | 90 | CD1 | PHE | A | 73 | 2158 | 2126 | 2116 | −30 | −101 | 27 | A | C |
| ATOM | 91 | CE1 | PHE | A | 73 | −22.320 | −0.825 | −15.166 | 1.00 | 17.86 | | A | C |
| ANISOU | 91 | CE1 | PHE | A | 73 | 2223 | 2239 | 2324 | 53 | −104 | −14 | A | C |
| ATOM | 92 | CZ | PHE | A | 73 | −23.341 | −0.072 | −14.599 | 1.00 | 17.48 | | A | C |
| ANISOU | 92 | CZ | PHE | A | 73 | 2091 | 2150 | 2401 | 62 | −115 | 34 | A | C |
| ATOM | 93 | CE2 | PHE | A | 73 | −23.573 | −0.125 | −13.232 | 1.00 | 16.60 | | A | C |
| ANISOU | 93 | CE2 | PHE | A | 73 | 2083 | 1928 | 2298 | −12 | −150 | 59 | A | C |
| ATOM | 94 | CD2 | PHE | A | 73 | −22.775 | −0.938 | −12.423 | 1.00 | 16.59 | | A | C |
| ANISOU | 94 | CD2 | PHE | A | 73 | 2051 | 1931 | 2323 | 21 | −13 | 35 | A | C |
| ATOM | 95 | C | PHE | A | 73 | −18.921 | −1.021 | −12.125 | 1.00 | 10.43 | | A | C |
| ANISOU | 95 | C | PHE | A | 73 | 1359 | 1266 | 1337 | −50 | 37 | −12 | A | C |
| ATOM | 96 | O | PHE | A | 73 | −19.100 | −0.448 | −11.045 | 1.00 | 10.43 | | A | O |
| ANISOU | 96 | O | PHE | A | 73 | 1364 | 1283 | 1316 | −5 | 2 | 7 | A | O |
| ATOM | 97 | N | THR | A | 74 | −18.291 | −0.460 | −13.154 | 1.00 | 10.48 | | A | N |
| ANISOU | 97 | N | THR | A | 74 | 1373 | 1279 | 1329 | −54 | 3 | 53 | A | N |
| ATOM | 98 | CA | THR | A | 74 | −17.791 | 0.908 | −13.089 | 1.00 | 10.00 | | A | C |
| ANISOU | 98 | CA | THR | A | 74 | 1270 | 1232 | 1296 | 8 | −1 | 29 | A | C |
| ATOM | 99 | CB | THR | A | 74 | −16.405 | 1.045 | −13.757 | 1.00 | 10.88 | | A | C |
| ANISOU | 99 | CB | THR | A | 74 | 1338 | 1390 | 1407 | −10 | 51 | 67 | A | C |
| ATOM | 100 | OG1 | THR | A | 74 | −15.465 | 0.209 | −13.071 | 1.00 | 12.56 | | A | O |
| ANISOU | 100 | OG1 | THR | A | 74 | 1554 | 1624 | 1595 | 120 | −42 | 49 | A | O |
| ATOM | 101 | CG2 | THR | A | 74 | −15.911 | 2.491 | −13.702 | 1.00 | 12.17 | | A | C |
| ANISOU | 101 | CG2 | THR | A | 74 | 1583 | 1450 | 1590 | −36 | 0 | 41 | A | C |
| ATOM | 102 | C | THR | A | 74 | −18.791 | 1.843 | −13.743 | 1.00 | 10.58 | | A | C |
| ANISOU | 102 | C | THR | A | 74 | 1405 | 1295 | 1320 | 39 | −22 | 4 | A | C |
| ATOM | 103 | O | THR | A | 74 | −19.197 | 1.630 | −14.891 | 1.00 | 12.22 | | A | O |
| ANISOU | 103 | O | THR | A | 74 | 1623 | 1537 | 1484 | 62 | −136 | −67 | A | O |
| ATOM | 104 | N | VAL | A | 75 | −19.191 | 2.866 | −12.996 | 1.00 | 8.87 | | A | N |
| ANISOU | 104 | N | VAL | A | 75 | 1134 | 1123 | 1113 | 21 | −42 | 27 | A | N |
| ATOM | 105 | CA | VAL | A | 75 | −20.092 | 3.895 | −13.506 | 1.00 | 8.97 | | A | C |
| ANISOU | 105 | CA | VAL | A | 75 | 1139 | 1118 | 1152 | 32 | −8 | 37 | A | C |
| ATOM | 106 | CB | VAL | A | 75 | −21.221 | 4.219 | −12.495 | 1.00 | 9.39 | | A | C |
| ANISOU | 106 | CB | VAL | A | 75 | 1206 | 1161 | 1200 | −14 | −10 | 31 | A | C |
| ATOM | 107 | CG1 | VAL | A | 75 | −22.162 | 5.294 | −13.046 | 1.00 | 10.02 | | A | C |
| ANISOU | 107 | CG1 | VAL | A | 75 | 1191 | 1280 | 1336 | 38 | 17 | 63 | A | C |
| ATOM | 108 | CG2 | VAL | A | 75 | −21.998 | 2.951 | −12.148 | 1.00 | 10.06 | | A | C |
| ANISOU | 108 | CG2 | VAL | A | 75 | 1296 | 1200 | 1326 | −38 | 78 | 98 | A | C |
| ATOM | 109 | C | VAL | A | 75 | −19.282 | 5.145 | −13.839 | 1.00 | 9.25 | | A | C |
| ANISOU | 109 | C | VAL | A | 75 | 1171 | 1192 | 1151 | 10 | 40 | −3 | A | C |
| ATOM | 110 | O | VAL | A | 75 | −18.720 | 5.794 | −12.954 | 1.00 | 9.46 | | A | O |
| ANISOU | 110 | O | VAL | A | 75 | 1160 | 1305 | 1131 | −74 | −11 | 46 | A | O |
| ATOM | 111 | N | GLU | A | 76 | −19.218 | 5.464 | −15.128 | 1.00 | 9.19 | | A | N |
| ANISOU | 111 | N | GLU | A | 76 | 1179 | 1194 | 1117 | 72 | −3 | 34 | A | N |
| ATOM | 112 | CA | GLU | A | 76 | −18.520 | 6.653 | −15.606 | 1.00 | 10.34 | | A | C |
| ANISOU | 112 | CA | GLU | A | 76 | 1307 | 1280 | 1342 | 12 | 57 | 25 | A | C |
| ATOM | 113 | CB | GLU | A | 76 | −17.936 | 6.401 | −17.005 | 1.00 | 11.38 | | A | C |
| ANISOU | 113 | CB | GLU | A | 76 | 1509 | 1434 | 1379 | 62 | 22 | 23 | A | C |
| ATOM | 114 | CG | GLU | A | 76 | −16.971 | 5.205 | −17.017 | 1.00 | 12.98 | | A | C |
| ANISOU | 114 | CG | GLU | A | 76 | 1652 | 1642 | 1639 | 142 | 86 | −12 | A | C |
| ATOM | 115 | CD | GLU | A | 76 | −16.141 | 5.083 | −18.284 | 1.00 | 14.84 | | A | C |
| ANISOU | 115 | CD | GLU | A | 76 | 1880 | 1969 | 1788 | 0 | 131 | −48 | A | C |
| ATOM | 116 | OE1 | GLU | A | 76 | −16.139 | 6.014 | −19.119 | 1.00 | 15.37 | | A | O |
| ANISOU | 116 | OE1 | GLU | A | 76 | 1993 | 1973 | 1875 | 146 | 40 | −8 | A | O |
| ATOM | 117 | OE2 | GLU | A | 76 | −15.472 | 4.035 | −18.428 | 1.00 | 17.42 | | A | O |
| ANISOU | 117 | OE2 | GLU | A | 76 | 2173 | 2144 | 2301 | 120 | 125 | −61 | A | O |
| ATOM | 118 | C | GLU | A | 76 | −19.461 | 7.857 | −15.591 | 1.00 | 9.82 | | A | C |
| ANISOU | 118 | C | GLU | A | 76 | 1220 | 1228 | 1285 | −15 | 8 | 41 | A | C |
| ATOM | 119 | O | GLU | A | 76 | −20.687 | 7.691 | −15.551 | 1.00 | 9.99 | | A | O |
| ANISOU | 119 | O | GLU | A | 76 | 1229 | 1236 | 1330 | 48 | 22 | 52 | A | O |
| ATOM | 120 | N | ARG | A | 77 | −18.883 | 9.061 | −15.613 | 1.00 | 9.86 | | A | N |
| ANISOU | 120 | N | ARG | A | 77 | 1250 | 1222 | 1274 | 28 | −11 | 32 | A | N |
| ATOM | 121 | CA | ARG | A | 77 | −19.646 | 10.310 | −15.493 | 1.00 | 9.91 | | A | C |
| ANISOU | 121 | CA | ARG | A | 77 | 1310 | 1272 | 1184 | 18 | −27 | 44 | A | C |
| ATOM | 122 | CB | ARG | A | 77 | −20.357 | 10.659 | −16.812 | 1.00 | 9.80 | | A | C |
| ANISOU | 122 | CB | ARG | A | 77 | 1227 | 1339 | 1157 | 23 | −22 | −15 | A | C |
| ATOM | 123 | CG | ARG | A | 77 | −19.393 | 10.876 | −17.977 | 1.00 | 9.96 | | A | C |
| ANISOU | 123 | CG | ARG | A | 77 | 1275 | 1350 | 1160 | −49 | 15 | 110 | A | C |
| ATOM | 124 | CD | ARG | A | 77 | −20.129 | 11.137 | −19.290 | 1.00 | 10.02 | | A | C |
| ANISOU | 124 | CD | ARG | A | 77 | 1284 | 1315 | 1207 | −48 | −41 | 39 | A | C |
| ATOM | 125 | NE | ARG | A | 77 | −20.990 | 10.017 | −19.664 | 1.00 | 8.29 | | A | N |
| ANISOU | 125 | NE | ARG | A | 77 | 1145 | 1139 | 864 | 47 | −110 | 109 | A | N |
| ATOM | 126 | CZ | ARG | A | 77 | −20.565 | 8.878 | −20.204 | 1.00 | 8.45 | | A | C |
| ANISOU | 126 | CZ | ARG | A | 77 | 1069 | 1272 | 871 | 67 | 0 | 18 | A | C |
| ATOM | 127 | NH1 | ARG | A | 77 | −19.271 | 8.685 | −20.453 | 1.00 | 10.03 | | A | N |
| ANISOU | 127 | NH1 | ARG | A | 77 | 1119 | 1628 | 1065 | 191 | −24 | 0 | A | N |
| ATOM | 128 | NH2 | ARG | A | 77 | −21.438 | 7.924 | −20.489 | 1.00 | 9.87 | | A | N |
| ANISOU | 128 | NH2 | ARG | A | 77 | 1196 | 1385 | 1168 | 75 | −54 | 0 | A | N |
| ATOM | 129 | C | ARG | A | 77 | −20.621 | 10.241 | −14.316 | 1.00 | 10.02 | | A | C |
| ANISOU | 129 | C | ARG | A | 77 | 1311 | 1293 | 1202 | 8 | −42 | 11 | A | C |
| ATOM | 130 | O | ARG | A | 77 | −21.784 | 10.646 | −14.410 | 1.00 | 10.80 | | A | O |
| ANISOU | 130 | O | ARG | A | 77 | 1351 | 1419 | 1335 | 2 | −25 | −62 | A | O |
| ATOM | 131 | N | PHE | A | 78 | −20.118 | 9.723 | −13.197 | 1.00 | 10.10 | | A | N |
| ANISOU | 131 | N | PHE | A | 78 | 1332 | 1320 | 1187 | 15 | −52 | 74 | A | N |
| ATOM | 132 | CA | PHE | A | 78 | −20.946 | 9.437 | −12.028 | 1.00 | 10.56 | | A | C |
| ANISOU | 132 | CA | PHE | A | 78 | 1374 | 1420 | 1220 | 31 | −56 | 61 | A | C |
| ATOM | 133 | CB | PHE | A | 78 | −20.079 | 8.823 | −10.921 | 1.00 | 10.67 | | A | C |
| ANISOU | 133 | CB | PHE | A | 78 | 1397 | 1433 | 1225 | 60 | −70 | 120 | A | C |
| ATOM | 134 | CG | PHE | A | 78 | −20.864 | 8.320 | −9.746 | 1.00 | 10.80 | | A | C |
| ANISOU | 134 | CG | PHE | A | 78 | 1355 | 1461 | 1289 | 14 | 1 | 11 | A | C |
| ATOM | 135 | CD1 | PHE | A | 78 | −21.455 | 7.063 | −9.778 | 1.00 | 12.26 | | A | C |
| ANISOU | 135 | CD1 | PHE | A | 78 | 1590 | 1525 | 1545 | −30 | 5 | 11 | A | C |
| ATOM | 136 | CE1 | PHE | A | 78 | −22.183 | 6.588 | −8.690 | 1.00 | 12.35 | | A | C |
| ANISOU | 136 | CE1 | PHE | A | 78 | 1544 | 1668 | 1481 | 2 | 0 | 17 | A | C |
| ATOM | 137 | CZ | PHE | A | 78 | −22.323 | 7.376 | −7.553 | 1.00 | 12.16 | | A | C |
| ANISOU | 137 | CZ | PHE | A | 78 | 1489 | 1630 | 1503 | 19 | 6 | 11 | A | C |
| ATOM | 138 | CE2 | PHE | A | 78 | −21.735 | 8.634 | −7.506 | 1.00 | 13.14 | | A | C |
| ANISOU | 138 | CE2 | PHE | A | 78 | 1632 | 1689 | 1671 | −3 | 27 | 62 | A | C |
| ATOM | 139 | CD2 | PHE | A | 78 | −21.007 | 9.103 | −8.601 | 1.00 | 11.38 | | A | C |
| ANISOU | 139 | CD2 | PHE | A | 78 | 1503 | 1424 | 1398 | 1 | −2 | −68 | A | C |
| ATOM | 140 | C | PHE | A | 78 | −21.716 | 10.659 | −11.512 | 1.00 | 11.19 | | A | C |
| ANISOU | 140 | C | PHE | A | 78 | 1443 | 1481 | 1327 | 2 | −17 | 39 | A | C |
| ATOM | 141 | O | PHE | A | 78 | −22.885 | 10.549 | −11.129 | 1.00 | 10.86 | | A | O |
| ANISOU | 141 | O | PHE | A | 78 | 1395 | 1437 | 1294 | 76 | −75 | 63 | A | O |
| ATOM | 142 | N | SER | A | 79 | −21.059 | 11.818 | −11.520 | 1.00 | 12.31 | | A | N |
| ANISOU | 142 | N | SER | A | 79 | 1581 | 1560 | 1535 | −46 | −19 | 14 | A | N |
| ATOM | 143 | CA | SER | A | 79 | −21.664 | 13.067 | −11.051 | 1.00 | 13.55 | | A | C |
| ANISOU | 143 | CA | SER | A | 79 | 1751 | 1647 | 1752 | −20 | 20 | −32 | A | C |
| ATOM | 144 | CB | SER | A | 79 | −20.648 | 14.212 | −11.132 | 1.00 | 14.54 | | A | C |
| ANISOU | 144 | CB | SER | A | 79 | 1846 | 1772 | 1905 | −87 | 30 | −56 | A | C |
| ATOM | 145 | OG | SER | A | 79 | −20.305 | 14.504 | −12.477 | 1.00 | 14.92 | | A | O |
| ANISOU | 145 | OG | SER | A | 79 | 1888 | 1828 | 1954 | −19 | −23 | −58 | A | O |
| ATOM | 146 | C | SER | A | 79 | −22.946 | 13.451 | −11.801 | 1.00 | 13.74 | | A | C |
| ANISOU | 146 | C | SER | A | 79 | 1756 | 1680 | 1785 | −15 | 19 | −17 | A | C |
| ATOM | 147 | O | SER | A | 79 | −23.792 | 14.175 | −11.260 | 1.00 | 14.71 | | A | O |
| ANISOU | 147 | O | SER | A | 79 | 1903 | 1759 | 1927 | −11 | 69 | −68 | A | O |
| ATOM | 148 | N | ARG | A | 80 | −23.086 | 12.962 | −13.034 | 1.00 | 13.31 | | A | N |
| ANISOU | 148 | N | ARG | A | 80 | 1706 | 1682 | 1670 | −24 | 4 | 53 | A | N |
| ATOM | 149 | CA | ARG | A | 80 | −24.244 | 13.287 | −13.877 | 1.00 | 14.00 | | A | C |
| ANISOU | 149 | CA | ARG | A | 80 | 1777 | 1831 | 1710 | −5 | −29 | 40 | A | C |
| ATOM | 150 | CB | ARG | A | 80 | −23.831 | 13.456 | −15.349 | 1.00 | 14.93 | | A | C |
| ANISOU | 150 | CB | ARG | A | 80 | 1921 | 1984 | 1768 | 40 | −17 | −28 | A | C |
| ATOM | 151 | CG | ARG | A | 80 | −22.732 | 14.485 | −15.628 | 1.00 | 17.11 | | A | C |
| ANISOU | 151 | CG | ARG | A | 80 | 2083 | 2261 | 2156 | −71 | 86 | 78 | A | C |
| ATOM | 152 | CD | ARG | A | 80 | −22.936 | 15.833 | −14.927 | 1.00 | 19.20 | | A | C |
| ANISOU | 152 | CD | ARG | A | 80 | 2482 | 2373 | 2439 | −21 | 0 | 10 | A | C |
| ATOM | 153 | NE | ARG | A | 80 | −24.141 | 16.551 | −15.347 | 1.00 | 19.10 | | A | N |
| ANISOU | 153 | NE | ARG | A | 80 | 2486 | 2340 | 2430 | −13 | −39 | 22 | A | N |
| ATOM | 154 | CZ | ARG | A | 80 | −24.203 | 17.420 | −16.357 | 1.00 | 18.53 | | A | C |
| ANISOU | 154 | CZ | ARG | A | 80 | 2436 | 2300 | 2306 | −10 | −36 | −57 | A | C |
| ATOM | 155 | NH1 | ARG | A | 80 | −23.128 | 17.688 | −17.089 | 1.00 | 19.67 | | A | N |
| ANISOU | 155 | NH1 | ARG | A | 80 | 2428 | 2565 | 2480 | −81 | −75 | −116 | A | N |
| ATOM | 156 | NH2 | ARG | A | 80 | −25.353 | 18.020 | −16.641 | 1.00 | 18.48 | | A | N |
| ANISOU | 156 | NH2 | ARG | A | 80 | 2437 | 2334 | 2250 | −10 | −61 | −36 | A | N |
| ATOM | 157 | C | ARG | A | 80 | −25.384 | 12.273 | −13.785 | 1.00 | 13.95 | | A | C |
| ANISOU | 157 | C | ARG | A | 80 | 1807 | 1801 | 1693 | 20 | −24 | 50 | A | C |
| ATOM | 158 | O | ARG | A | 80 | −26.434 | 12.462 | −14.402 | 1.00 | 13.84 | | A | O |
| ANISOU | 158 | O | ARG | A | 80 | 1823 | 1811 | 1626 | 1 | −65 | 55 | A | O |
| ATOM | 159 | N | LEU | A | 81 | −25.188 | 11.199 | −13.022 | 1.00 | 13.49 | | A | N |
| ANISOU | 159 | N | LEU | A | 81 | 1814 | 1730 | 1583 | −11 | 40 | 7 | A | N |
| ATOM | 160 | CA | LEU | A | 81 | −26.218 | 10.170 | −12.898 | 1.00 | 14.01 | | A | C |
| ANISOU | 160 | CA | LEU | A | 81 | 1854 | 1810 | 1659 | −21 | 0 | 21 | A | C |
| ATOM | 161 | CB | LEU | A | 81 | −25.710 | 9.008 | −12.039 | 1.00 | 13.70 | | A | C |
| ANISOU | 161 | CB | LEU | A | 81 | 1843 | 1771 | 1591 | −13 | −30 | 34 | A | C |
| ATOM | 162 | CG | LEU | A | 81 | −26.387 | 7.647 | −12.184 | 1.00 | 14.88 | | A | C |
| ANISOU | 162 | CG | LEU | A | 81 | 2012 | 1849 | 1791 | −1 | −2 | 26 | A | C |
| ATOM | 163 | CD1 | LEU | A | 81 | −26.105 | 7.050 | −13.561 | 1.00 | 17.57 | | A | C |
| ANISOU | 163 | CD1 | LEU | A | 81 | 2410 | 2252 | 2012 | 69 | −16 | −124 | A | C |
| ATOM | 164 | CD2 | LEU | A | 81 | −25.886 | 6.718 | −11.089 | 1.00 | 15.63 | | A | C |
| ANISOU | 164 | CD2 | LEU | A | 81 | 2052 | 1924 | 1962 | 12 | −23 | 106 | A | C |
| ATOM | 165 | C | LEU | A | 81 | −27.504 | 10.760 | −12.312 | 1.00 | 14.38 | | A | C |
| ANISOU | 165 | C | LEU | A | 81 | 1805 | 1845 | 1814 | −1 | −34 | 6 | A | C |
| ATOM | 166 | O | LEU | A | 81 | −27.464 | 11.485 | −11.315 | 1.00 | 15.15 | | A | O |
| ANISOU | 166 | O | LEU | A | 81 | 1899 | 2000 | 1858 | −72 | −19 | −15 | A | O |
| ATOM | 167 | N | SER | A | 82 | −28.636 | 10.455 | −12.944 | 1.00 | 14.49 | | A | N |
| ANISOU | 167 | N | SER | A | 82 | 1834 | 1942 | 1728 | 13 | −17 | 7 | A | N |
| ATOM | 168 | CA | SER | A | 82 | −29.929 | 10.988 | −12.506 | 1.00 | 14.83 | | A | C |
| ANISOU | 168 | CA | SER | A | 82 | 1872 | 1937 | 1827 | 41 | −22 | 10 | A | C |
| ATOM | 169 | CB | SER | A | 82 | −30.504 | 11.928 | −13.564 | 1.00 | 15.32 | | A | C |
| ANISOU | 169 | CB | SER | A | 82 | 2006 | 1954 | 1860 | 7 | −18 | 31 | A | C |
| ATOM | 170 | OG | SER | A | 82 | −30.493 | 11.315 | −14.840 | 1.00 | 15.85 | | A | O |
| ANISOU | 170 | OG | SER | A | 82 | 2115 | 1928 | 1979 | 78 | −43 | −51 | A | O |
| ATOM | 171 | C | SER | A | 82 | −30.939 | 9.894 | −12.186 | 1.00 | 15.63 | | A | C |
| ANISOU | 171 | C | SER | A | 82 | 1982 | 2029 | 1929 | 13 | −17 | 11 | A | C |
| ATOM | 172 | O | SER | A | 82 | −31.923 | 10.132 | −11.482 | 1.00 | 17.33 | | A | O |
| ANISOU | 172 | O | SER | A | 82 | 2051 | 2330 | 2202 | 23 | 45 | −45 | A | O |
| ATOM | 173 | N | GLU | A | 83 | −30.686 | 8.699 | −12.705 | 1.00 | 15.55 | | A | N |
| ANISOU | 173 | N | GLU | A | 83 | 1979 | 2020 | 1908 | 21 | 12 | 34 | A | N |
| ATOM | 174 | CA | GLU | A | 83 | −31.594 | 7.569 | −12.563 | 1.00 | 16.48 | | A | C |
| ANISOU | 174 | CA | GLU | A | 83 | 2105 | 2101 | 2054 | 0 | 25 | −9 | A | C |
| ATOM | 175 | CB | GLU | A | 83 | −32.574 | 7.576 | −13.737 | 1.00 | 17.35 | | A | C |
| ANISOU | 175 | CB | GLU | A | 83 | 2145 | 2188 | 2258 | 121 | −26 | 40 | A | C |
| ATOM | 176 | CG | GLU | A | 83 | −33.666 | 6.534 | −13.728 | 1.00 | 19.95 | | A | C |
| ANISOU | 176 | CG | GLU | A | 83 | 2375 | 2561 | 2645 | 15 | −81 | −1 | A | C |
| ATOM | 177 | CD | GLU | A | 83 | −34.613 | 6.718 | −14.899 | 1.00 | 21.13 | | A | C |
| ANISOU | 177 | CD | GLU | A | 83 | 2667 | 2715 | 2646 | 39 | −116 | −7 | A | C |
| ATOM | 178 | OE1 | GLU | A | 83 | −34.558 | 5.901 | −15.847 | 1.00 | 20.81 | | A | O |
| ANISOU | 178 | OE1 | GLU | A | 83 | 2587 | 2668 | 2652 | 40 | −71 | −48 | A | O |
| ATOM | 179 | OE2 | GLU | A | 83 | −35.389 | 7.703 | −14.880 | 1.00 | 21.64 | | A | O |
| ANISOU | 179 | OE2 | GLU | A | 83 | 2551 | 2650 | 3020 | 103 | 33 | 30 | A | O |
| ATOM | 180 | C | GLU | A | 83 | −30.770 | 6.287 | −12.536 | 1.00 | 15.76 | | A | C |
| ANISOU | 180 | C | GLU | A | 83 | 1986 | 2065 | 1937 | −21 | 27 | 21 | A | C |
| ATOM | 181 | O | GLU | A | 83 | −29.560 | 6.315 | −12.803 | 1.00 | 16.85 | | A | O |
| ANISOU | 181 | O | GLU | A | 83 | 2019 | 2249 | 2135 | 80 | 70 | 77 | A | O |
| ATOM | 182 | N | SER | A | 84 | −31.418 | 5.171 | −12.210 | 1.00 | 14.84 | | A | N |
| ANISOU | 182 | N | SER | A | 84 | 1940 | 1910 | 1788 | 45 | 33 | −58 | A | N |
| ATOM | 183 | CA | SER | A | 84 | −30.746 | 3.879 | −12.129 | 1.00 | 14.50 | | A | C |
| ANISOU | 183 | CA | SER | A | 84 | 1836 | 1959 | 1716 | 67 | 57 | −22 | A | C |
| ATOM | 184 | CB | SER | A | 84 | −31.758 | 2.758 | −11.896 | 1.00 | 16.92 | | A | C |
| ANISOU | 184 | CB | SER | A | 84 | 2128 | 2118 | 2181 | 3 | 17 | 8 | A | C |
| ATOM | 185 | OG | SER | A | 84 | −32.636 | 2.640 | −12.997 | 1.00 | 20.82 | | A | O |
| ANISOU | 185 | OG | SER | A | 84 | 2692 | 2802 | 2416 | −54 | −24 | −51 | A | O |
| ATOM | 186 | C | SER | A | 84 | −29.916 | 3.579 | −13.378 | 1.00 | 13.57 | | A | C |
| ANISOU | 186 | C | SER | A | 84 | 1671 | 1860 | 1626 | 45 | 31 | 17 | A | C |
| ATOM | 187 | O | SER | A | 84 | −30.358 | 3.811 | −14.509 | 1.00 | 15.25 | | A | O |
| ANISOU | 187 | O | SER | A | 84 | 1794 | 2213 | 1787 | 126 | −19 | −29 | A | O |
| ATOM | 188 | N | VAL | A | 85 | −28.707 | 3.085 | −13.145 | 1.00 | 11.51 | | A | N |
| ANISOU | 188 | N | VAL | A | 85 | 1498 | 1533 | 1342 | −5 | 73 | −19 | A | N |
| ATOM | 189 | CA | VAL | A | 85 | −27.867 | 2.506 | −14.187 | 1.00 | 11.01 | | A | C |
| ANISOU | 189 | CA | VAL | A | 85 | 1400 | 1459 | 1323 | −13 | 16 | −8 | A | C |
| ATOM | 190 | CB | VAL | A | 85 | −26.543 | 3.315 | −14.379 | 1.00 | 11.36 | | A | C |
| ANISOU | 190 | CB | VAL | A | 85 | 1458 | 1544 | 1315 | −38 | −10 | 2 | A | C |
| ATOM | 191 | CG1 | VAL | A | 85 | −25.686 | 3.321 | −13.104 | 1.00 | 11.46 | | A | C |
| ANISOU | 191 | CG1 | VAL | A | 85 | 1514 | 1582 | 1260 | −22 | −23 | −23 | A | C |
| ATOM | 192 | CG2 | VAL | A | 85 | −25.746 | 2.800 | −15.575 | 1.00 | 12.06 | | A | C |
| ANISOU | 192 | CG2 | VAL | A | 85 | 1532 | 1614 | 1438 | −14 | 62 | 6 | A | C |
| ATOM | 193 | C | VAL | A | 85 | −27.627 | 1.041 | −13.793 | 1.00 | 10.89 | | A | C |
| ANISOU | 193 | C | VAL | A | 85 | 1433 | 1435 | 1270 | 7 | −37 | 17 | A | C |
| ATOM | 194 | O | VAL | A | 85 | −27.478 | 0.729 | −12.604 | 1.00 | 10.38 | | A | O |
| ANISOU | 194 | O | VAL | A | 85 | 1417 | 1405 | 1122 | −12 | −80 | 32 | A | O |
| ATOM | 195 | N | LEU | A | 86 | −27.627 | 0.148 | −14.782 | 1.00 | 11.92 | | A | N |
| ANISOU | 195 | N | LEU | A | 86 | 1627 | 1565 | 1337 | 27 | −23 | −30 | A | N |
| ATOM | 196 | CA | LEU | A | 86 | −27.548 | −1.293 | −14.540 | 1.00 | 12.57 | | A | C |
| ANISOU | 196 | CA | LEU | A | 86 | 1701 | 1619 | 1456 | −21 | 16 | −31 | A | C |
| ATOM | 197 | CB | LEU | A | 86 | −28.808 | −1.998 | −15.054 | 1.00 | 14.67 | | A | C |
| ANISOU | 197 | CB | LEU | A | 86 | 1834 | 2055 | 1686 | −6 | −124 | −77 | A | C |
| ATOM | 198 | CG | LEU | A | 86 | −30.088 | −2.206 | −14.255 | 1.00 | 18.99 | | A | C |
| ANISOU | 198 | CG | LEU | A | 86 | 2198 | 2666 | 2352 | −55 | 35 | 61 | A | C |
| ATOM | 199 | CD1 | LEU | A | 86 | −31.122 | −2.832 | −15.188 | 1.00 | 19.39 | | A | C |
| ANISOU | 199 | CD1 | LEU | A | 86 | 2507 | 2447 | 2413 | −68 | −44 | −56 | A | C |
| ATOM | 200 | CD2 | LEU | A | 86 | −29.876 | −3.096 | −13.050 | 1.00 | 18.92 | | A | C |
| ANISOU | 200 | CD2 | LEU | A | 86 | 2421 | 2446 | 2323 | −22 | 23 | 15 | A | C |
| ATOM | 201 | C | LEU | A | 86 | −26.369 | −1.910 | −15.263 | 1.00 | 12.04 | | A | C |
| ANISOU | 201 | C | LEU | A | 86 | 1664 | 1454 | 1456 | −14 | −17 | −37 | A | C |
| ATOM | 202 | O | LEU | A | 86 | −26.082 | −1.548 | −16.411 | 1.00 | 11.99 | | A | O |
| ANISOU | 202 | O | LEU | A | 86 | 1861 | 1342 | 1352 | −68 | 22 | −14 | A | O |
| ATOM | 203 | N | SER | A | 87 | −25.714 | −2.865 | −14.606 | 1.00 | 10.94 | | A | N |
| ANISOU | 203 | N | SER | A | 87 | 1506 | 1297 | 1353 | −74 | −4 | −89 | A | N |
| ATOM | 204 | CA | SER | A | 87 | −24.643 | −3.634 | −15.231 | 1.00 | 10.99 | | A | C |
| ANISOU | 204 | CA | SER | A | 87 | 1402 | 1376 | 1396 | −52 | −22 | −18 | A | C |
| ATOM | 205 | CB | SER | A | 87 | −23.838 | −4.381 | −14.162 | 1.00 | 11.35 | | A | C |
| ANISOU | 205 | CB | SER | A | 87 | 1536 | 1324 | 1453 | −77 | 10 | 94 | A | C |
| ATOM | 206 | OG | SER | A | 87 | −24.556 | −5.508 | −13.678 | 1.00 | 10.16 | | A | O |
| ANISOU | 206 | OG | SER | A | 87 | 1421 | 1266 | 1172 | −130 | 97 | 9 | A | O |
| ATOM | 207 | C | SER | A | 87 | −25.222 | −4.649 | −16.218 | 1.00 | 10.28 | | A | C |
| ANISOU | 207 | C | SER | A | 87 | 1338 | 1268 | 1300 | −37 | 13 | 25 | A | C |
| ATOM | 208 | O | SER | A | 87 | −26.416 | −4.958 | −16.161 | 1.00 | 10.16 | | A | O |
| ANISOU | 208 | O | SER | A | 87 | 1337 | 1310 | 1213 | −30 | 5 | −11 | A | O |
| ATOM | 209 | N | PRO | A | 88 | −24.375 | −5.174 | −17.123 | 1.00 | 9.76 | | A | N |
| ANISOU | 209 | N | PRO | A | 88 | 1321 | 1142 | 1246 | −36 | −7 | 56 | A | N |
| ATOM | 210 | CA | PRO | A | 88 | −24.777 | −6.374 | −17.852 | 1.00 | 9.74 | | A | C |
| ANISOU | 210 | CA | PRO | A | 88 | 1381 | 1168 | 1152 | −14 | 51 | 6 | A | C |
| ATOM | 211 | CB | PRO | A | 88 | −23.613 | −6.630 | −18.819 | 1.00 | 10.87 | | A | C |
| ANISOU | 211 | CB | PRO | A | 88 | 1395 | 1378 | 1356 | −3 | 125 | 78 | A | C |
| ATOM | 212 | CG | PRO | A | 88 | −22.721 | −5.456 | −18.730 | 1.00 | 12.34 | | A | C |
| ANISOU | 212 | CG | PRO | A | 88 | 1655 | 1490 | 1544 | −61 | 39 | −99 | A | C |
| ATOM | 213 | CD | PRO | A | 88 | −23.027 | −4.706 | −17.492 | 1.00 | 10.76 | | A | C |
| ANISOU | 213 | CD | PRO | A | 88 | 1308 | 1371 | 1409 | −9 | 13 | 9 | A | C |
| ATOM | 214 | C | PRO | A | 88 | −24.906 | −7.548 | −16.868 | 1.00 | 9.08 | | A | C |
| ANISOU | 214 | C | PRO | A | 88 | 1229 | 1129 | 1093 | 6 | −3 | 20 | A | C |
| ATOM | 215 | O | PRO | A | 88 | −24.340 | −7.489 | −15.767 | 1.00 | 9.18 | | A | O |
| ANISOU | 215 | O | PRO | A | 88 | 1183 | 1147 | 1159 | −5 | −1 | 14 | A | O |
| ATOM | 216 | N | PRO | A | 89 | −25.648 | −8.603 | −17.249 | 1.00 | 8.97 | | A | N |
| ANISOU | 216 | N | PRO | A | 89 | 1227 | 1095 | 1085 | −34 | −16 | 94 | A | N |
| ATOM | 217 | CA | PRO | A | 89 | −25.827 | −9.730 | −16.327 | 1.00 | 8.49 | | A | C |
| ANISOU | 217 | CA | PRO | A | 89 | 1131 | 1045 | 1049 | 10 | −2 | 64 | A | C |
| ATOM | 218 | CB | PRO | A | 89 | −26.858 | −10.607 | −17.038 | 1.00 | 9.49 | | A | C |
| ANISOU | 218 | CB | PRO | A | 89 | 1224 | 1184 | 1199 | −65 | −10 | 21 | A | C |
| ATOM | 219 | CG | PRO | A | 89 | −26.740 | −10.259 | −18.479 | 1.00 | 9.61 | | A | C |
| ANISOU | 219 | CG | PRO | A | 89 | 1369 | 1078 | 1206 | −87 | −83 | 55 | A | C |
| ATOM | 220 | CD | PRO | A | 89 | −26.382 | −8.802 | −18.515 | 1.00 | 9.23 | | A | C |
| ANISOU | 220 | CD | PRO | A | 89 | 1283 | 1043 | 1182 | −18 | −81 | −15 | A | C |
| ATOM | 221 | C | PRO | A | 89 | −24.554 | −10.535 | −16.065 | 1.00 | 9.06 | | A | C |
| ANISOU | 221 | C | PRO | A | 89 | 1134 | 1230 | 1079 | 40 | 16 | 31 | A | C |
| ATOM | 222 | O | PRO | A | 89 | −23.672 | −10.635 | −16.933 | 1.00 | 9.00 | | A | O |
| ANISOU | 222 | O | PRO | A | 89 | 1132 | 1224 | 1062 | 45 | 47 | 104 | A | O |
| ATOM | 223 | N | CYS | A | 90 | −24.480 | −11.090 | −14.861 | 1.00 | 8.66 | | A | N |
| ANISOU | 223 | N | CYS | A | 90 | 1068 | 1138 | 1083 | 48 | 19 | 64 | A | N |
| ATOM | 224 | CA | CYS | A | 90 | −23.442 | −12.031 | −14.473 | 1.00 | 9.10 | | A | C |
| ANISOU | 224 | CA | CYS | A | 90 | 1165 | 1088 | 1204 | 17 | 10 | 0 | A | C |
| ATOM | 225 | CB | CYS | A | 90 | −22.590 | −11.442 | −13.354 | 1.00 | 9.62 | | A | C |
| ANISOU | 225 | CB | CYS | A | 90 | 1201 | 1154 | 1300 | 10 | −29 | 14 | A | C |
| ATOM | 226 | SG | CYS | A | 90 | −21.304 | −12.542 | −12.743 | 1.00 | 12.22 | | A | S |
| ANISOU | 226 | SG | CYS | A | 90 | 1512 | 1486 | 1645 | 172 | −258 | 6 | A | S |
| ATOM | 227 | C | CYS | A | 90 | −24.162 | −13.268 | −13.971 | 1.00 | 8.12 | | A | C |
| ANISOU | 227 | C | CYS | A | 90 | 1077 | 1006 | 1001 | 2 | 23 | 8 | A | C |
| ATOM | 228 | O | CYS | A | 90 | −25.013 | −13.170 | −13.082 | 1.00 | 8.45 | | A | O |
| ANISOU | 228 | O | CYS | A | 90 | 1087 | 1072 | 1050 | 41 | 72 | −13 | A | O |
| ATOM | 229 | N | PHE | A | 91 | −23.832 | −14.430 | −14.534 | 1.00 | 8.30 | | A | N |
| ANISOU | 229 | N | PHE | A | 91 | 1093 | 1056 | 1006 | 30 | 15 | 8 | A | N |
| ATOM | 230 | CA | PHE | A | 91 | −24.534 | −15.666 | −14.173 | 1.00 | 8.82 | | A | C |
| ANISOU | 230 | CA | PHE | A | 91 | 1190 | 1067 | 1096 | −2 | 20 | −36 | A | C |
| ATOM | 231 | CB | PHE | A | 91 | −24.652 | −16.611 | −15.378 | 1.00 | 10.41 | | A | C |
| ANISOU | 231 | CB | PHE | A | 91 | 1395 | 1234 | 1327 | 46 | −51 | −130 | A | C |
| ATOM | 232 | CG | PHE | A | 91 | −25.838 | −16.318 | −16.250 | 1.00 | 10.03 | | A | C |
| ANISOU | 232 | CG | PHE | A | 91 | 1258 | 1340 | 1212 | −11 | 9 | −70 | A | C |
| ATOM | 233 | CD1 | PHE | A | 91 | −25.809 | −15.262 | −17.156 | 1.00 | 11.40 | | A | C |
| ANISOU | 233 | CD1 | PHE | A | 91 | 1491 | 1335 | 1504 | 71 | 49 | −59 | A | C |
| ATOM | 234 | CE1 | PHE | A | 91 | −26.908 | −14.983 | −17.962 | 1.00 | 14.03 | | A | C |
| ANISOU | 234 | CE1 | PHE | A | 91 | 1665 | 1903 | 1762 | −53 | −92 | −73 | A | C |
| ATOM | 235 | CZ | PHE | A | 91 | −28.054 | −15.763 | −17.853 | 1.00 | 12.66 | | A | C |
| ANISOU | 235 | CZ | PHE | A | 91 | 1579 | 1624 | 1607 | 15 | −13 | −2 | A | C |
| ATOM | 236 | CE2 | PHE | A | 91 | −28.094 | −16.819 | −16.952 | 1.00 | 12.50 | | A | C |
| ANISOU | 236 | CE2 | PHE | A | 91 | 1552 | 1762 | 1435 | 39 | −10 | −3 | A | C |
| ATOM | 237 | CD2 | PHE | A | 91 | −26.993 | −17.091 | −16.153 | 1.00 | 11.75 | | A | C |
| ANISOU | 237 | CD2 | PHE | A | 91 | 1413 | 1524 | 1528 | −40 | 2 | −79 | A | C |
| ATOM | 238 | C | PHE | A | 91 | −23.954 | −16.395 | −12.970 | 1.00 | 9.54 | | A | C |
| ANISOU | 238 | C | PHE | A | 91 | 1235 | 1205 | 1185 | −31 | 6 | −16 | A | C |
| ATOM | 239 | O | PHE | A | 91 | −22.753 | −16.681 | −12.913 | 1.00 | 9.84 | | A | O |
| ANISOU | 239 | O | PHE | A | 91 | 1226 | 1189 | 1324 | −80 | −1 | 89 | A | O |
| ATOM | 240 | N | VAL | A | 92 | −24.839 | −16.678 | −12.014 | 1.00 | 9.87 | | A | N |
| ANISOU | 240 | N | VAL | A | 92 | 1299 | 1276 | 1177 | −99 | −14 | 19 | A | N |
| ATOM | 241 | CA | VAL | A | 92 | −24.536 | −17.451 | −10.808 | 1.00 | 9.57 | | A | C |
| ANISOU | 241 | CA | VAL | A | 92 | 1284 | 1122 | 1231 | −46 | −14 | 4 | A | C |
| ATOM | 242 | CB | VAL | A | 92 | −24.266 | −16.529 | −9.583 | 1.00 | 9.74 | | A | C |
| ANISOU | 242 | CB | VAL | A | 92 | 1286 | 1218 | 1198 | −33 | −19 | 17 | A | C |
| ATOM | 243 | CG1 | VAL | A | 92 | −23.947 | −17.342 | −8.327 | 1.00 | 10.73 | | A | C |
| ANISOU | 243 | CG1 | VAL | A | 92 | 1519 | 1349 | 1209 | 21 | −55 | 18 | A | C |
| ATOM | 244 | CG2 | VAL | A | 92 | −23.126 | −15.571 | −9.871 | 1.00 | 11.56 | | A | C |
| ANISOU | 244 | CG2 | VAL | A | 92 | 1462 | 1480 | 1451 | −125 | 62 | 122 | A | C |
| ATOM | 245 | C | VAL | A | 92 | −25.754 | −18.336 | −10.551 | 1.00 | 9.77 | | A | C |
| ANISOU | 245 | C | VAL | A | 92 | 1251 | 1173 | 1290 | −3 | 3 | 50 | A | C |
| ATOM | 246 | O | VAL | A | 92 | −26.895 | −17.862 | −10.610 | 1.00 | 9.36 | | A | O |
| ANISOU | 246 | O | VAL | A | 92 | 1215 | 996 | 1346 | 29 | 69 | −9 | A | O |
| ATOM | 247 | N | ARG | A | 93 | −25.519 | −19.622 | −10.292 | 1.00 | 9.60 | | A | N |
| ANISOU | 247 | N | ARG | A | 93 | 1256 | 1071 | 1320 | −18 | 34 | −35 | A | N |
| ATOM | 248 | CA | ARG | A | 93 | −26.610 | −20.584 | −10.048 | 1.00 | 9.98 | | A | C |
| ANISOU | 248 | CA | ARG | A | 93 | 1246 | 1220 | 1325 | −27 | 34 | −8 | A | C |
| ATOM | 249 | CB | ARG | A | 93 | −27.293 | −20.320 | −8.693 | 1.00 | 9.44 | | A | C |
| ANISOU | 249 | CB | ARG | A | 93 | 1217 | 1073 | 1298 | −24 | 31 | −35 | A | C |
| ATOM | 250 | CG | ARG | A | 93 | −26.422 | −20.598 | −7.476 | 1.00 | 10.65 | | A | C |
| ANISOU | 250 | CG | ARG | A | 93 | 1416 | 1248 | 1384 | 2 | 12 | 55 | A | C |
| ATOM | 251 | CD | ARG | A | 93 | −26.157 | −22.078 | −7.295 | 1.00 | 11.56 | | A | C |
| ANISOU | 251 | CD | ARG | A | 93 | 1492 | 1354 | 1547 | 77 | 23 | 87 | A | C |
| ATOM | 252 | NE | ARG | A | 93 | −25.795 | −22.368 | −5.912 | 1.00 | 12.18 | | A | N |
| ANISOU | 252 | NE | ARG | A | 93 | 1586 | 1532 | 1510 | 47 | 5 | −14 | A | N |
| ATOM | 253 | CZ | ARG | A | 93 | −25.640 | −23.591 | −5.417 | 1.00 | 13.44 | | A | C |
| ANISOU | 253 | CZ | ARG | A | 93 | 1731 | 1618 | 1758 | 56 | −21 | 58 | A | C |
| ATOM | 254 | NH1 | ARG | A | 93 | −25.794 | −24.656 | −6.202 | 1.00 | 14.85 | | A | N |
| ANISOU | 254 | NH1 | ARG | A | 93 | 2023 | 1795 | 1826 | −19 | −13 | −38 | A | N |
| ATOM | 255 | NH2 | ARG | A | 93 | −25.322 | −23.747 | −4.135 | 1.00 | 13.60 | | A | N |
| ANISOU | 255 | NH2 | ARG | A | 93 | 1738 | 1746 | 1685 | −17 | 51 | 32 | A | N |
| ATOM | 256 | C | ARG | A | 93 | −27.640 | −20.592 | −11.180 | 1.00 | 10.60 | | A | C |
| ANISOU | 256 | C | ARG | A | 93 | 1352 | 1246 | 1430 | 26 | −1 | −8 | A | C |
| ATOM | 257 | O | ARG | A | 93 | −28.849 | −20.731 | −10.944 | 1.00 | 11.66 | | A | O |
| ANISOU | 257 | O | ARG | A | 93 | 1378 | 1473 | 1580 | −9 | −23 | 10 | A | O |
| ATOM | 258 | N | ASN | A | 94 | −27.141 | −20.437 | −12.404 | 1.00 | 10.85 | | A | N |
| ANISOU | 258 | N | ASN | A | 94 | 1481 | 1226 | 1416 | 9 | −40 | −5 | A | N |
| ATOM | 259 | CA | ASN | A | 94 | −27.958 | −20.469 | −13.622 | 1.00 | 11.43 | | A | C |
| ANISOU | 259 | CA | ASN | A | 94 | 1449 | 1425 | 1469 | 23 | −58 | −33 | A | C |
| ATOM | 260 | CB | ASN | A | 94 | −28.664 | −21.827 | −13.775 | 1.00 | 12.00 | | A | C |
| ANISOU | 260 | CB | ASN | A | 94 | 1582 | 1404 | 1574 | 5 | −55 | −32 | A | C |
| ATOM | 261 | CG | ASN | A | 94 | −27.708 | −22.952 | −14.152 | 1.00 | 14.57 | | A | C |
| ANISOU | 261 | CG | ASN | A | 94 | 1785 | 1856 | 1895 | 162 | 31 | −18 | A | C |
| ATOM | 262 | OD1 | ASN | A | 94 | −26.654 | −22.719 | −14.741 | 1.00 | 14.37 | | A | O |
| ANISOU | 262 | OD1 | ASN | A | 94 | 1874 | 1656 | 1929 | −1 | −33 | 25 | A | O |
| ATOM | 263 | ND2 | ASN | A | 94 | −28.083 | −24.183 | −13.816 | 1.00 | 19.38 | | A | N |
| ANISOU | 263 | ND2 | ASN | A | 94 | 2339 | 2213 | 2813 | −96 | 68 | 31 | A | N |
| ATOM | 264 | C | ASN | A | 94 | −28.954 | −19.311 | −13.768 | 1.00 | 11.41 | | A | C |
| ANISOU | 264 | C | ASN | A | 94 | 1519 | 1341 | 1477 | 5 | −65 | −65 | A | C |
| ATOM | 265 | O | ASN | A | 94 | −29.873 | −19.381 | −14.589 | 1.00 | 12.02 | | A | O |
| ANISOU | 265 | O | ASN | A | 94 | 1496 | 1431 | 1639 | −45 | −85 | −95 | A | O |
| ATOM | 266 | N | LEU | A | 95 | −28.758 | −18.249 | −12.979 | 1.00 | 10.07 | | A | N |
| ANISOU | 266 | N | LEU | A | 95 | 1312 | 1208 | 1306 | 8 | −46 | −55 | A | N |
| ATOM | 267 | CA | LEU | A | 95 | −29.573 | −17.036 | −13.079 | 1.00 | 9.67 | | A | C |
| ANISOU | 267 | CA | LEU | A | 95 | 1267 | 1188 | 1219 | −18 | 46 | 26 | A | C |
| ATOM | 268 | CB | LEU | A | 95 | −30.400 | −16.817 | −11.804 | 1.00 | 9.09 | | A | C |
| ANISOU | 268 | CB | LEU | A | 95 | 1188 | 1126 | 1139 | 54 | 41 | −26 | A | C |
| ATOM | 269 | CG | LEU | A | 95 | −31.550 | −17.794 | −11.523 | 1.00 | 10.36 | | A | C |
| ANISOU | 269 | CG | LEU | A | 95 | 1364 | 1351 | 1222 | −132 | 48 | 7 | A | C |
| ATOM | 270 | CD1 | LEU | A | 95 | −32.174 | −17.491 | −10.170 | 1.00 | 11.04 | | A | C |
| ANISOU | 270 | CD1 | LEU | A | 95 | 1471 | 1408 | 1314 | 80 | 140 | −47 | A | C |
| ATOM | 271 | CD2 | LEU | A | 95 | −32.619 | −17.757 | −12.612 | 1.00 | 11.47 | | A | C |
| ANISOU | 271 | CD2 | LEU | A | 95 | 1414 | 1447 | 1497 | 30 | −98 | 174 | A | C |
| ATOM | 272 | C | LEU | A | 95 | −28.705 | −15.806 | −13.324 | 1.00 | 9.14 | | A | C |
| ANISOU | 272 | C | LEU | A | 95 | 1137 | 1231 | 1105 | −31 | 33 | 24 | A | C |
| ATOM | 273 | O | LEU | A | 95 | −27.547 | −15.771 | −12.891 | 1.00 | 9.94 | | A | O |
| ANISOU | 273 | O | LEU | A | 95 | 1197 | 1402 | 1179 | −11 | −38 | 72 | A | O |
| ATOM | 274 | N | PRO | A | 96 | −29.258 | −14.794 | −14.018 | 1.00 | 8.42 | | A | N |
| ANISOU | 274 | N | PRO | A | 96 | 1071 | 1082 | 1045 | −16 | −7 | −49 | A | N |
| ATOM | 275 | CA | PRO | A | 96 | −28.524 | −13.546 | −14.232 | 1.00 | 8.93 | | A | C |
| ANISOU | 275 | CA | PRO | A | 96 | 1192 | 1170 | 1031 | −41 | −25 | −13 | A | C |
| ATOM | 276 | CB | PRO | A | 96 | −29.201 | −12.959 | −15.469 | 1.00 | 9.01 | | A | C |
| ANISOU | 276 | CB | PRO | A | 96 | 1054 | 1283 | 1086 | −7 | −58 | 41 | A | C |
| ATOM | 277 | CG | PRO | A | 96 | −30.627 | −13.448 | −15.385 | 1.00 | 8.38 | | A | C |
| ANISOU | 277 | CG | PRO | A | 96 | 1043 | 1095 | 1046 | −40 | 3 | 16 | A | C |
| ATOM | 278 | CD | PRO | A | 96 | −30.583 | −14.782 | −14.678 | 1.00 | 8.95 | | A | C |
| ANISOU | 278 | CD | PRO | A | 96 | 1117 | 1141 | 1141 | 28 | −6 | 16 | A | C |
| ATOM | 279 | C | PRO | A | 96 | −28.649 | −12.576 | −13.056 | 1.00 | 8.76 | | A | C |
| ANISOU | 279 | C | PRO | A | 96 | 1119 | 1139 | 1069 | 7 | 10 | −25 | A | C |
| ATOM | 280 | O | PRO | A | 96 | −29.748 | −12.373 | −12.532 | 1.00 | 9.95 | | A | O |
| ANISOU | 280 | O | PRO | A | 96 | 1133 | 1397 | 1251 | −6 | 52 | −71 | A | O |
| ATOM | 281 | N | TRP | A | 97 | −27.521 | −11.981 | −12.672 | 1.00 | 7.82 | | A | N |
| ANISOU | 281 | N | TRP | A | 97 | 1077 | 983 | 913 | −33 | 7 | −7 | A | N |
| ATOM | 282 | CA | TRP | A | 97 | −27.455 | −11.007 | −11.586 | 1.00 | 7.32 | | A | C |
| ANISOU | 282 | CA | TRP | A | 97 | 1017 | 839 | 925 | −13 | −57 | 22 | A | C |
| ATOM | 283 | CB | TRP | A | 97 | −26.579 | −11.528 | −10.436 | 1.00 | 8.04 | | A | C |
| ANISOU | 283 | CB | TRP | A | 97 | 1024 | 990 | 1039 | −33 | −60 | 41 | A | C |
| ATOM | 284 | CG | TRP | A | 97 | −27.054 | −12.854 | −9.896 | 1.00 | 8.39 | | A | C |
| ANISOU | 284 | CG | TRP | A | 97 | 1182 | 956 | 1050 | 14 | 35 | 108 | A | C |
| ATOM | 285 | CD1 | TRP | A | 97 | −26.851 | −14.083 | −10.459 | 1.00 | 9.20 | | A | C |
| ANISOU | 285 | CD1 | TRP | A | 97 | 1345 | 1038 | 1114 | −106 | 27 | 22 | A | C |
| ATOM | 286 | NE1 | TRP | A | 97 | −27.447 | −15.063 | −9.693 | 1.00 | 8.57 | | A | N |
| ANISOU | 286 | NE1 | TRP | A | 97 | 1147 | 1069 | 1041 | −79 | 51 | 112 | A | N |
| ATOM | 287 | CE2 | TRP | A | 97 | −28.043 | −14.475 | −8.610 | 1.00 | 8.37 | | A | C |
| ANISOU | 287 | CE2 | TRP | A | 97 | 1082 | 994 | 1105 | −60 | 71 | 28 | A | C |
| ATOM | 288 | CD2 | TRP | A | 97 | −27.822 | −13.080 | −8.704 | 1.00 | 8.09 | | A | C |
| ANISOU | 288 | CD2 | TRP | A | 97 | 1134 | 952 | 986 | 37 | −48 | 19 | A | C |
| ATOM | 289 | CE3 | TRP | A | 97 | −28.338 | −12.242 | −7.703 | 1.00 | 9.38 | | A | C |
| ANISOU | 289 | CE3 | TRP | A | 97 | 1193 | 1244 | 1127 | 71 | 85 | 84 | A | C |
| ATOM | 290 | CZ3 | TRP | A | 97 | −29.049 | −12.818 | −6.654 | 1.00 | 9.85 | | A | C |
| ANISOU | 290 | CZ3 | TRP | A | 97 | 1390 | 1262 | 1090 | 9 | 55 | 8 | A | C |
| ATOM | 291 | CH2 | TRP | A | 97 | −29.252 | −14.207 | −6.592 | 1.00 | 9.05 | | A | C |
| ANISOU | 291 | CH2 | TRP | A | 97 | 1129 | 1191 | 1119 | 15 | 93 | 17 | A | C |
| ATOM | 292 | CZ2 | TRP | A | 97 | −28.759 | −15.050 | −7.557 | 1.00 | 8.61 | | A | C |
| ANISOU | 292 | CZ2 | TRP | A | 97 | 1127 | 1126 | 1019 | −60 | 35 | 48 | A | C |
| ATOM | 293 | C | TRP | A | 97 | −26.878 | −9.705 | −12.123 | 1.00 | 6.97 | | A | C |
| ANISOU | 293 | C | TRP | A | 97 | 881 | 884 | 885 | −12 | 22 | −2 | A | C |
| ATOM | 294 | O | TRP | A | 97 | −25.985 | −9.720 | −12.964 | 1.00 | 7.97 | | A | O |
| ANISOU | 294 | O | TRP | A | 97 | 1076 | 1013 | 941 | −29 | 81 | −100 | A | O |
| ATOM | 295 | N | LYS | A | 98 | −27.400 | −8.584 | −11.637 | 1.00 | 7.05 | | A | N |
| ANISOU | 295 | N | LYS | A | 98 | 941 | 838 | 900 | 17 | 10 | −44 | A | N |
| ATOM | 296 | CA | LYS | A | 98 | −26.926 | −7.272 | −12.052 | 1.00 | 7.40 | | A | C |
| ANISOU | 296 | CA | LYS | A | 98 | 949 | 927 | 937 | 37 | 2 | −6 | A | C |
| ATOM | 297 | CB | LYS | A | 98 | −27.930 | −6.606 | −12.994 | 1.00 | 7.63 | | A | C |
| ANISOU | 297 | CB | LYS | A | 98 | 975 | 1007 | 918 | 41 | −62 | −17 | A | C |
| ATOM | 298 | CG | LYS | A | 98 | −28.139 | −7.321 | −14.327 | 1.00 | 8.28 | | A | C |
| ANISOU | 298 | CG | LYS | A | 98 | 1134 | 1131 | 880 | 104 | −48 | −42 | A | C |
| ATOM | 299 | CD | LYS | A | 98 | −29.200 | −6.599 | −15.148 | 1.00 | 8.27 | | A | C |
| ANISOU | 299 | CD | LYS | A | 98 | 1223 | 840 | 1080 | 34 | −170 | 50 | A | C |
| ATOM | 300 | CE | LYS | A | 98 | −29.497 | −7.328 | −16.450 | 1.00 | 10.05 | | A | C |
| ANISOU | 300 | CE | LYS | A | 98 | 1505 | 1224 | 1089 | 40 | 13 | −39 | A | C |
| ATOM | 301 | NZ | LYS | A | 98 | −30.547 | −6.617 | −17.239 | 1.00 | 11.71 | | A | N |
| ANISOU | 301 | NZ | LYS | A | 98 | 1571 | 1472 | 1405 | −26 | −85 | 36 | A | N |
| ATOM | 302 | C | LYS | A | 98 | −26.715 | −6.368 | −10.855 | 1.00 | 8.33 | | A | C |
| ANISOU | 302 | C | LYS | A | 98 | 1087 | 1033 | 1046 | −25 | 48 | −24 | A | C |
| ATOM | 303 | O | LYS | A | 98 | −27.384 | −6.512 | −9.827 | 1.00 | 8.66 | | A | O |
| ANISOU | 303 | O | LYS | A | 98 | 1240 | 1042 | 1010 | −1 | 138 | 39 | A | O |
| ATOM | 304 | N | ILE | A | 99 | −25.787 | −5.428 | −11.004 | 1.00 | 8.09 | | A | N |
| ANISOU | 304 | N | ILE | A | 99 | 1088 | 942 | 1044 | −84 | 24 | −47 | A | N |
| ATOM | 305 | CA | ILE | A | 99 | −25.645 | −4.327 | −10.056 | 1.00 | 8.52 | | A | C |
| ANISOU | 305 | CA | ILE | A | 99 | 1150 | 1030 | 1058 | −2 | −40 | −67 | A | C |
| ATOM | 306 | CB | ILE | A | 99 | −24.171 | −3.912 | −9.898 | 1.00 | 7.98 | | A | C |
| ANISOU | 306 | CB | ILE | A | 99 | 1040 | 1049 | 942 | 13 | 28 | 11 | A | C |
| ATOM | 307 | CG1 | ILE | A | 99 | −23.401 | −5.004 | −9.150 | 1.00 | 9.10 | | A | C |
| ANISOU | 307 | CG1 | ILE | A | 99 | 1219 | 1197 | 1042 | 83 | 57 | 73 | A | C |
| ATOM | 308 | CD1 | ILE | A | 99 | −21.902 | −4.885 | −9.255 | 1.00 | 9.94 | | A | C |
| ANISOU | 308 | CD1 | ILE | A | 99 | 1127 | 1329 | 1320 | −8 | 11 | 54 | A | C |
| ATOM | 309 | CG2 | ILE | A | 99 | −24.045 | −2.559 | −9.185 | 1.00 | 10.85 | | A | C |
| ANISOU | 309 | CG2 | ILE | A | 99 | 1566 | 1175 | 1380 | −41 | −53 | −48 | A | C |
| ATOM | 310 | C | ILE | A | 99 | −26.478 | −3.152 | −10.547 | 1.00 | 9.09 | | A | C |
| ANISOU | 310 | C | ILE | A | 99 | 1227 | 1093 | 1134 | −53 | −4 | 7 | A | C |
| ATOM | 311 | O | ILE | A | 99 | −26.423 | −2.796 | −11.731 | 1.00 | 9.17 | | A | O |
| ANISOU | 311 | O | ILE | A | 99 | 1273 | 1074 | 1136 | −45 | 36 | 14 | A | O |
| ATOM | 312 | N | MET | A | 100 | −27.264 | −2.579 | −9.635 | 1.00 | 9.65 | | A | N |
| ANISOU | 312 | N | MET | A | 100 | 1316 | 1241 | 1110 | −106 | 61 | −79 | A | N |
| ATOM | 313 | CA | MET | A | 100 | −28.069 | −1.397 | −9.915 | 1.00 | 11.20 | | A | C |
| ANISOU | 313 | CA | MET | A | 100 | 1569 | 1321 | 1367 | −69 | 43 | −59 | A | C |
| ATOM | 314 | CB | MET | A | 100 | −29.566 | −1.685 | −9.752 | 1.00 | 12.58 | | A | C |
| ANISOU | 314 | CB | MET | A | 100 | 1727 | 1562 | 1491 | −142 | 9 | −55 | A | C |
| ATOM | 315 | CG | MET | A | 100 | −30.447 | −0.537 | −10.232 | 1.00 | 16.94 | | A | C |
| ANISOU | 315 | CG | MET | A | 100 | 2187 | 2119 | 2132 | 148 | 21 | 33 | A | C |
| ATOM | 316 | SD | MET | A | 100 | −32.202 | −0.922 | −10.377 | 1.00 | 24.38 | | A | S |
| ANISOU | 316 | SD | MET | A | 100 | 2696 | 3323 | 3244 | −32 | −113 | 23 | A | S |
| ATOM | 317 | CE | MET | A | 100 | −32.523 | −1.506 | −8.720 | 1.00 | 24.89 | | A | C |
| ANISOU | 317 | CE | MET | A | 100 | 3112 | 3340 | 3006 | −28 | 4 | 24 | A | C |
| ATOM | 318 | C | MET | A | 100 | −27.653 | −0.266 | −8.985 | 1.00 | 9.85 | | A | C |
| ANISOU | 318 | C | MET | A | 100 | 1430 | 1143 | 1169 | −71 | 9 | 19 | A | C |
| ATOM | 319 | O | MET | A | 100 | −27.589 | −0.446 | −7.770 | 1.00 | 10.15 | | A | O |
| ANISOU | 319 | O | MET | A | 100 | 1566 | 1095 | 1195 | −39 | 0 | 46 | A | O |
| ATOM | 320 | N | VAL | A | 101 | −27.366 | 0.895 | −9.570 | 1.00 | 8.59 | | A | N |
| ANISOU | 320 | N | VAL | A | 101 | 1153 | 1141 | 969 | −64 | 22 | 37 | A | N |
| ATOM | 321 | CA | VAL | A | 101 | −26.928 | 2.065 | −8.822 | 1.00 | 9.06 | | A | C |
| ANISOU | 321 | CA | VAL | A | 101 | 1248 | 1070 | 1124 | 3 | −7 | 33 | A | C |
| ATOM | 322 | CB | VAL | A | 101 | −25.433 | 2.368 | −9.109 | 1.00 | 9.23 | | A | C |
| ANISOU | 322 | CB | VAL | A | 101 | 1134 | 1170 | 1203 | −53 | −81 | 1 | A | C |
| ATOM | 323 | CG1 | VAL | A | 101 | −24.998 | 3.703 | −8.501 | 1.00 | 11.39 | | A | C |
| ANISOU | 323 | CG1 | VAL | A | 101 | 1556 | 1232 | 1538 | −7 | −102 | −31 | A | C |
| ATOM | 324 | CG2 | VAL | A | 101 | −24.550 | 1.241 | −8.580 | 1.00 | 11.48 | | A | C |
| ANISOU | 324 | CG2 | VAL | A | 101 | 1455 | 1304 | 1604 | 120 | −61 | 29 | A | C |
| ATOM | 325 | C | VAL | A | 101 | −27.787 | 3.262 | −9.221 | 1.00 | 9.48 | | A | C |
| ANISOU | 325 | C | VAL | A | 101 | 1265 | 1162 | 1174 | 15 | −25 | −2 | A | C |
| ATOM | 326 | O | VAL | A | 101 | −28.082 | 3.445 | −10.400 | 1.00 | 10.46 | | A | O |
| ANISOU | 326 | O | VAL | A | 101 | 1535 | 1246 | 1192 | 23 | −54 | 139 | A | O |
| ATOM | 327 | N | MET | A | 102 | −28.190 | 4.067 | −8.242 | 1.00 | 9.57 | | A | N |
| ANISOU | 327 | N | MET | A | 102 | 1376 | 1130 | 1132 | 8 | −40 | −8 | A | N |
| ATOM | 328 | CA | MET | A | 102 | −29.042 | 5.230 | −8.518 | 1.00 | 11.64 | | A | C |
| ANISOU | 328 | CA | MET | A | 102 | 1637 | 1388 | 1398 | 114 | −26 | −70 | A | C |
| ATOM | 329 | CB | MET | A | 102 | −30.518 | 4.813 | −8.586 | 1.00 | 13.39 | | A | C |
| ANISOU | 329 | CB | MET | A | 102 | 1772 | 1643 | 1673 | 26 | −50 | −56 | A | C |
| ATOM | 330 | CG | MET | A | 102 | −31.111 | 4.349 | −7.249 | 1.00 | 16.67 | | A | C |
| ANISOU | 330 | CG | MET | A | 102 | 2134 | 2299 | 1900 | 14 | 99 | −133 | A | C |
| ATOM | 331 | SD | MET | A | 102 | −32.685 | 3.498 | −7.425 | 1.00 | 22.19 | | A | S |
| ANISOU | 331 | SD | MET | A | 102 | 2552 | 2876 | 3004 | −131 | 326 | −36 | A | S |
| ATOM | 332 | CE | MET | A | 102 | −32.116 | 1.846 | −7.781 | 1.00 | 22.70 | | A | C |
| ANISOU | 332 | CE | MET | A | 102 | 2921 | 2849 | 2854 | 13 | 39 | −66 | A | C |
| ATOM | 333 | C | MET | A | 102 | −28.858 | 6.294 | −7.446 | 1.00 | 10.76 | | A | C |
| ANISOU | 333 | C | MET | A | 102 | 1497 | 1261 | 1329 | 60 | −27 | 8 | A | C |
| ATOM | 334 | O | MET | A | 102 | −28.578 | 5.959 | −6.301 | 1.00 | 11.31 | | A | O |
| ANISOU | 334 | O | MET | A | 102 | 1727 | 1340 | 1230 | 72 | −61 | 13 | A | O |
| ATOM | 335 | N | PRO | A | 103 | −29.019 | 7.580 | −7.808 | 1.00 | 9.24 | | A | N |
| ANISOU | 335 | N | PRO | A | 103 | 1308 | 1145 | 1056 | 88 | 1 | 7 | A | N |
| ATOM | 336 | CA | PRO | A | 103 | −29.138 | 8.581 | −6.744 | 1.00 | 10.20 | | A | C |
| ANISOU | 336 | CA | PRO | A | 103 | 1460 | 1172 | 1245 | 17 | −28 | 1 | A | C |
| ATOM | 337 | CB | PRO | A | 103 | −28.972 | 9.913 | −7.482 | 1.00 | 10.28 | | A | C |
| ANISOU | 337 | CB | PRO | A | 103 | 1484 | 1224 | 1197 | 2 | −19 | 33 | A | C |
| ATOM | 338 | CG | PRO | A | 103 | −29.371 | 9.617 | −8.903 | 1.00 | 10.32 | | A | C |
| ANISOU | 338 | CG | PRO | A | 103 | 1522 | 1234 | 1165 | 102 | −59 | −31 | A | C |
| ATOM | 339 | CD | PRO | A | 103 | −29.112 | 8.165 | −9.159 | 1.00 | 9.86 | | A | C |
| ANISOU | 339 | CD | PRO | A | 103 | 1392 | 1233 | 1122 | 130 | 47 | 59 | A | C |
| ATOM | 340 | C | PRO | A | 103 | −30.519 | 8.475 | −6.102 | 1.00 | 10.89 | | A | C |
| ANISOU | 340 | C | PRO | A | 103 | 1455 | 1346 | 1337 | 12 | −13 | −20 | A | C |
| ATOM | 341 | O | PRO | A | 103 | −31.498 | 8.142 | −6.786 | 1.00 | 12.15 | | A | O |
| ANISOU | 341 | O | PRO | A | 103 | 1591 | 1641 | 1384 | −77 | 12 | −104 | A | O |
| ATOM | 342 | N | ARG | A | 104 | −30.591 | 8.723 | −4.795 | 1.00 | 10.97 | | A | N |
| ANISOU | 342 | N | ARG | A | 104 | 1490 | 1370 | 1310 | −9 | −44 | 33 | A | N |
| ATOM | 343 | CA | ARG | A | 104 | −31.847 | 8.638 | −4.053 | 1.00 | 11.90 | | A | C |
| ANISOU | 343 | CA | ARG | A | 104 | 1551 | 1461 | 1509 | 21 | −27 | −31 | A | C |
| ATOM | 344 | CB | ARG | A | 104 | −31.901 | 7.365 | −3.199 | 1.00 | 13.49 | | A | C |
| ANISOU | 344 | CB | ARG | A | 104 | 1795 | 1657 | 1673 | 27 | 1 | 73 | A | C |
| ATOM | 345 | CG | ARG | A | 104 | −31.609 | 6.050 | −3.914 | 1.00 | 19.10 | | A | C |
| ANISOU | 345 | CG | ARG | A | 104 | 2642 | 2201 | 2416 | 112 | 107 | −175 | A | C |
| ATOM | 346 | CD | ARG | A | 104 | −32.871 | 5.363 | −4.390 | 1.00 | 24.82 | | A | C |
| ANISOU | 346 | CD | ARG | A | 104 | 3004 | 3173 | 3253 | −109 | −64 | −98 | A | C |
| ATOM | 347 | NE | ARG | A | 104 | −33.795 | 5.030 | −3.306 | 1.00 | 26.71 | | A | N |
| ANISOU | 347 | NE | ARG | A | 104 | 3448 | 3329 | 3371 | −71 | 94 | 33 | A | N |
| ATOM | 348 | CZ | ARG | A | 104 | −34.706 | 4.060 | −3.370 | 1.00 | 26.55 | | A | C |
| ANISOU | 348 | CZ | ARG | A | 104 | 3367 | 3389 | 3333 | −26 | −35 | −21 | A | C |
| ATOM | 349 | NH1 | ARG | A | 104 | −34.808 | 3.307 | −4.456 | 1.00 | 25.25 | | A | N |
| ANISOU | 349 | NH1 | ARG | A | 104 | 3206 | 3024 | 3363 | −29 | 44 | 34 | A | N |
| ATOM | 350 | NH2 | ARG | A | 104 | −35.509 | 3.838 | −2.340 | 1.00 | 28.14 | | A | N |
| ANISOU | 350 | NH2 | ARG | A | 104 | 3534 | 3571 | 3588 | −90 | 64 | 80 | A | N |
| ATOM | 351 | C | ARG | A | 104 | −31.949 | 9.811 | −3.107 | 1.00 | 12.05 | | A | C |
| ANISOU | 351 | C | ARG | A | 104 | 1530 | 1534 | 1515 | 26 | −23 | −47 | A | C |
| ATOM | 352 | O | ARG | A | 104 | −30.956 | 10.211 | −2.510 | 1.00 | 10.96 | | A | O |
| ANISOU | 352 | O | ARG | A | 104 | 1387 | 1409 | 1368 | 118 | −59 | −114 | A | O |
| ATOM | 353 | N | PHE | A | 105 | −33.150 | 10.355 | −2.954 | 1.00 | 13.20 | | A | N |
| ANISOU | 353 | N | PHE | A | 105 | 1593 | 1743 | 1679 | 23 | −23 | −52 | A | N |
| ATOM | 354 | CA | PHE | A | 105 | −33.405 | 11.249 | −1.829 | 1.00 | 14.40 | | A | C |
| ANISOU | 354 | CA | PHE | A | 105 | 1791 | 1835 | 1844 | 26 | 17 | −68 | A | C |
| ATOM | 355 | CB | PHE | A | 105 | −32.916 | 12.686 | −2.096 | 1.00 | 15.16 | | A | C |
| ANISOU | 355 | CB | PHE | A | 105 | 1896 | 1907 | 1958 | −10 | −57 | −7 | A | C |
| ATOM | 356 | CG | PHE | A | 105 | −33.878 | 13.539 | −2.881 | 1.00 | 15.62 | | A | C |
| ANISOU | 356 | CG | PHE | A | 105 | 1947 | 1978 | 2011 | 34 | 4 | 22 | A | C |
| ATOM | 357 | CD1 | PHE | A | 105 | −34.742 | 14.421 | −2.226 | 1.00 | 15.73 | | A | C |
| ANISOU | 357 | CD1 | PHE | A | 105 | 1924 | 1991 | 2063 | 41 | 9 | 30 | A | C |
| ATOM | 358 | CE1 | PHE | A | 105 | −35.634 | 15.223 | −2.949 | 1.00 | 14.77 | | A | C |
| ANISOU | 358 | CE1 | PHE | A | 105 | 1858 | 1859 | 1894 | −38 | 63 | 58 | A | C |
| ATOM | 359 | CZ | PHE | A | 105 | −35.654 | 15.150 | −4.337 | 1.00 | 15.16 | | A | C |
| ANISOU | 359 | CZ | PHE | A | 105 | 1995 | 1827 | 1938 | −10 | −67 | −34 | A | C |
| ATOM | 360 | CE2 | PHE | A | 105 | −34.788 | 14.281 | −5.006 | 1.00 | 14.60 | | A | C |
| ANISOU | 360 | CE2 | PHE | A | 105 | 1748 | 1808 | 1991 | −46 | 6 | 75 | A | C |
| ATOM | 361 | CD2 | PHE | A | 105 | −33.905 | 13.479 | −4.274 | 1.00 | 14.93 | | A | C |
| ANISOU | 361 | CD2 | PHE | A | 105 | 1773 | 1980 | 1920 | −40 | −70 | 55 | A | C |
| ATOM | 362 | C | PHE | A | 105 | −34.862 | 11.209 | −1.416 | 1.00 | 15.21 | | A | C |
| ANISOU | 362 | C | PHE | A | 105 | 1840 | 1955 | 1986 | 25 | 6 | −56 | A | C |
| ATOM | 363 | O | PHE | A | 105 | −35.718 | 10.723 | −2.158 | 1.00 | 14.08 | | A | O |
| ANISOU | 363 | O | PHE | A | 105 | 1640 | 1795 | 1913 | 92 | 61 | −135 | A | O |
| ATOM | 364 | N | TYR | A | 106 | −35.120 | 11.716 | −0.216 | 1.00 | 16.61 | | A | N |
| ANISOU | 364 | N | TYR | A | 106 | 2060 | 2186 | 2064 | 10 | −32 | −72 | A | N |
| ATOM | 365 | CA | TYR | A | 106 | −36.456 | 11.747 | 0.344 | 1.00 | 18.54 | | A | C |
| ANISOU | 365 | CA | TYR | A | 106 | 2259 | 2384 | 2403 | 24 | 35 | −67 | A | C |
| ATOM | 366 | CB | TYR | A | 106 | −36.519 | 10.873 | 1.602 | 1.00 | 22.35 | | A | C |
| ANISOU | 366 | CB | TYR | A | 106 | 2780 | 3001 | 2710 | −41 | 43 | 133 | A | C |
| ATOM | 367 | CG | TYR | A | 106 | −36.264 | 9.409 | 1.297 | 1.00 | 28.28 | | A | C |
| ANISOU | 367 | CG | TYR | A | 106 | 3724 | 3485 | 3538 | 48 | 23 | −4 | A | C |
| ATOM | 368 | CD1 | TYR | A | 106 | −37.323 | 8.539 | 1.026 | 1.00 | 30.61 | | A | C |
| ANISOU | 368 | CD1 | TYR | A | 106 | 3834 | 3858 | 3939 | −57 | −31 | 0 | A | C |
| ATOM | 369 | CE1 | TYR | A | 106 | −37.097 | 7.194 | 0.728 | 1.00 | 31.66 | | A | C |
| ANISOU | 369 | CE1 | TYR | A | 106 | 3965 | 3944 | 4120 | 11 | 0 | −21 | A | C |
| ATOM | 370 | CZ | TYR | A | 106 | −35.797 | 6.710 | 0.697 | 1.00 | 31.30 | | A | C |
| ANISOU | 370 | CZ | TYR | A | 106 | 4051 | 3824 | 4019 | 58 | 11 | −48 | A | C |
| ATOM | 371 | OH | TYR | A | 106 | −35.565 | 5.383 | 0.404 | 1.00 | 30.17 | | A | O |
| ANISOU | 371 | OH | TYR | A | 106 | 3931 | 3673 | 3861 | 32 | 24 | −67 | A | O |
| ATOM | 372 | CE2 | TYR | A | 106 | −34.727 | 7.556 | 0.956 | 1.00 | 31.63 | | A | C |
| ANISOU | 372 | CE2 | TYR | A | 106 | 4027 | 3914 | 4078 | 12 | −6 | −26 | A | C |
| ATOM | 373 | CD2 | TYR | A | 106 | −34.965 | 8.900 | 1.253 | 1.00 | 30.02 | | A | C |
| ANISOU | 373 | CD2 | TYR | A | 106 | 3712 | 3872 | 3822 | 18 | 6 | 32 | A | C |
| ATOM | 374 | C | TYR | A | 106 | −36.817 | 13.199 | 0.625 | 1.00 | 16.36 | | A | C |
| ANISOU | 374 | C | TYR | A | 106 | 1999 | 2145 | 2073 | −7 | −22 | −34 | A | C |
| ATOM | 375 | O | TYR | A | 106 | −35.946 | 13.991 | 0.992 | 1.00 | 14.66 | | A | O |
| ANISOU | 375 | O | TYR | A | 106 | 1783 | 1864 | 1924 | 77 | 3 | −39 | A | O |
| ATOM | 376 | N | PRO | A | 107 | −38.093 | 13.564 | 0.414 | 1.00 | 15.44 | | A | N |
| ANISOU | 376 | N | PRO | A | 107 | 1952 | 1974 | 1941 | −11 | −10 | −47 | A | N |
| ATOM | 377 | CA | PRO | A | 107 | −38.500 | 14.965 | 0.527 | 1.00 | 14.63 | | A | C |
| ANISOU | 377 | CA | PRO | A | 107 | 1847 | 1881 | 1831 | 6 | −32 | −49 | A | C |
| ATOM | 378 | CB | PRO | A | 107 | −40.009 | 14.932 | 0.243 | 1.00 | 15.54 | | A | C |
| ANISOU | 378 | CB | PRO | A | 107 | 1876 | 2052 | 1976 | 2 | −36 | −14 | A | C |
| ATOM | 379 | CG | PRO | A | 107 | −40.413 | 13.509 | 0.359 | 1.00 | 17.09 | | A | C |
| ANISOU | 379 | CG | PRO | A | 107 | 2085 | 2127 | 2283 | 13 | −14 | −20 | A | C |
| ATOM | 380 | CD | PRO | A | 107 | −39.217 | 12.687 | 0.034 | 1.00 | 15.97 | | A | C |
| ANISOU | 380 | CD | PRO | A | 107 | 1989 | 2031 | 2048 | −36 | −44 | −41 | A | C |
| ATOM | 381 | C | PRO | A | 107 | −38.222 | 15.601 | 1.892 | 1.00 | 12.92 | | A | C |
| ANISOU | 381 | C | PRO | A | 107 | 1604 | 1662 | 1642 | 14 | 13 | 10 | A | C |
| ATOM | 382 | O | PRO | A | 107 | −38.020 | 16.813 | 1.963 | 1.00 | 12.19 | | A | O |
| ANISOU | 382 | O | PRO | A | 107 | 1462 | 1599 | 1569 | 22 | 44 | −31 | A | O |
| ATOM | 383 | N | ASP | A | 108 | −38.196 | 14.784 | 2.942 | 1.00 | 12.69 | | A | N |
| ANISOU | 383 | N | ASP | A | 108 | 1548 | 1728 | 1545 | −41 | 34 | −33 | A | N |
| ATOM | 384 | CA | ASP | A | 108 | −37.968 | 15.281 | 4.305 | 1.00 | 12.74 | | A | C |
| ANISOU | 384 | CA | ASP | A | 108 | 1558 | 1689 | 1594 | −61 | −24 | −24 | A | C |
| ATOM | 385 | CB | ASP | A | 108 | −38.678 | 14.399 | 5.347 | 1.00 | 15.04 | | A | C |
| ANISOU | 385 | CB | ASP | A | 108 | 2020 | 2061 | 1634 | −63 | 78 | 2 | A | C |
| ATOM | 386 | CG | ASP | A | 108 | −40.189 | 14.347 | 5.148 | 1.00 | 22.22 | | A | C |
| ANISOU | 386 | CG | ASP | A | 108 | 2566 | 3028 | 2847 | −7 | −98 | −93 | A | C |
| ATOM | 387 | OD1 | ASP | A | 108 | −40.773 | 15.314 | 4.638 | 1.00 | 24.65 | | A | O |
| ANISOU | 387 | OD1 | ASP | A | 108 | 3157 | 3037 | 3171 | 119 | −20 | 65 | A | O |
| ATOM | 388 | OD2 | ASP | A | 108 | −40.806 | 13.326 | 5.518 | 1.00 | 26.59 | | A | O |
| ANISOU | 388 | OD2 | ASP | A | 108 | 3411 | 3273 | 3419 | −201 | 119 | 141 | A | O |
| ATOM | 389 | C | ASP | A | 108 | −36.492 | 15.407 | 4.668 | 1.00 | 11.23 | | A | C |
| ANISOU | 389 | C | ASP | A | 108 | 1435 | 1496 | 1336 | −53 | 37 | −12 | A | C |
| ATOM | 390 | O | ASP | A | 108 | −36.175 | 15.871 | 5.728 | 1.00 | 10.52 | | A | O |
| ANISOU | 390 | O | ASP | A | 108 | 1368 | 1289 | 1339 | −144 | 42 | 71 | A | O |
| ATOM | 391 | N | ARG | A | 109 | −35.611 | 14.987 | 3.766 | 1.00 | 10.39 | | A | N |
| ANISOU | 391 | N | ARG | A | 109 | 1264 | 1331 | 1353 | −50 | −23 | 8 | A | N |
| ATOM | 392 | CA | ARG | A | 109 | −34.159 | 15.112 | 3.943 | 1.00 | 9.64 | | A | C |
| ANISOU | 392 | CA | ARG | A | 109 | 1206 | 1245 | 1210 | −29 | 51 | 60 | A | C |
| ATOM | 393 | CB | ARG | A | 109 | −33.543 | 13.824 | 4.524 | 1.00 | 10.51 | | A | C |
| ANISOU | 393 | CB | ARG | A | 109 | 1354 | 1345 | 1296 | −4 | 49 | 53 | A | C |
| ATOM | 394 | CG | ARG | A | 109 | −34.026 | 13.422 | 5.924 | 1.00 | 11.90 | | A | C |
| ANISOU | 394 | CG | ARG | A | 109 | 1612 | 1489 | 1420 | −75 | 96 | 108 | A | C |
| ATOM | 395 | CD | ARG | A | 109 | −33.442 | 14.300 | 7.030 | 1.00 | 14.72 | | A | C |
| ANISOU | 395 | CD | ARG | A | 109 | 1927 | 1865 | 1801 | −99 | 36 | −42 | A | C |
| ATOM | 396 | NE | ARG | A | 109 | −33.697 | 13.731 | 8.355 | 1.00 | 17.00 | | A | N |
| ANISOU | 396 | NE | ARG | A | 109 | 2358 | 2197 | 1906 | −71 | −30 | 59 | A | N |
| ATOM | 397 | CZ | ARG | A | 109 | −34.846 | 13.831 | 9.018 | 1.00 | 18.38 | | A | C |
| ANISOU | 397 | CZ | ARG | A | 109 | 2323 | 2332 | 2329 | −18 | 2 | −31 | A | C |
| ATOM | 398 | NH1 | ARG | A | 109 | −35.880 | 14.485 | 8.494 | 1.00 | 18.80 | | A | N |
| ANISOU | 398 | NH1 | ARG | A | 109 | 2549 | 2225 | 2369 | 7 | −64 | −22 | A | N |
| ATOM | 399 | NH2 | ARG | A | 109 | −34.964 | 13.268 | 10.216 | 1.00 | 18.61 | | A | N |
| ANISOU | 399 | NH2 | ARG | A | 109 | 2471 | 2239 | 2362 | −90 | 5 | 4 | A | N |
| ATOM | 400 | C | ARG | A | 109 | −33.496 | 15.467 | 2.604 | 1.00 | 8.90 | | A | C |
| ANISOU | 400 | C | ARG | A | 109 | 1125 | 1135 | 1122 | 19 | 28 | 12 | A | C |
| ATOM | 401 | O | ARG | A | 109 | −32.674 | 14.697 | 2.089 | 1.00 | 9.27 | | A | O |
| ANISOU | 401 | O | ARG | A | 109 | 1128 | 1121 | 1272 | −35 | 14 | −5 | A | O |
| ATOM | 402 | N | PRO | A | 110 | −33.841 | 16.644 | 2.037 | 1.00 | 8.12 | | A | N |
| ANISOU | 402 | N | PRO | A | 110 | 1030 | 1068 | 986 | −1 | 13 | 22 | A | N |
| ATOM | 403 | CA | PRO | A | 110 | −33.367 | 16.980 | 0.690 | 1.00 | 8.27 | | A | C |
| ANISOU | 403 | CA | PRO | A | 110 | 1046 | 1104 | 992 | −11 | 55 | 12 | A | C |
| ATOM | 404 | CB | PRO | A | 110 | −34.154 | 18.247 | 0.350 | 1.00 | 8.05 | | A | C |
| ANISOU | 404 | CB | PRO | A | 110 | 1087 | 1002 | 968 | −38 | −11 | −15 | A | C |
| ATOM | 405 | CG | PRO | A | 110 | −34.428 | 18.878 | 1.680 | 1.00 | 8.05 | | A | C |
| ANISOU | 405 | CG | PRO | A | 110 | 1136 | 1012 | 912 | 0 | 16 | 7 | A | C |
| ATOM | 406 | CD | PRO | A | 110 | −34.677 | 17.720 | 2.605 | 1.00 | 7.89 | | A | C |
| ANISOU | 406 | CD | PRO | A | 110 | 1063 | 1006 | 930 | −18 | 18 | 10 | A | C |
| ATOM | 407 | C | PRO | A | 110 | −31.865 | 17.247 | 0.610 | 1.00 | 9.26 | | A | C |
| ANISOU | 407 | C | PRO | A | 110 | 1127 | 1297 | 1095 | −38 | −13 | 4 | A | C |
| ATOM | 408 | O | PRO | A | 110 | −31.315 | 17.333 | −0.490 | 1.00 | 10.16 | | A | O |
| ANISOU | 408 | O | PRO | A | 110 | 1224 | 1471 | 1167 | −83 | 95 | −33 | A | O |
| ATOM | 409 | N | HIS | A | 111 | −31.223 | 17.371 | 1.768 | 1.00 | 8.41 | | A | N |
| ANISOU | 409 | N | HIS | A | 111 | 1078 | 1112 | 1005 | −81 | −7 | −28 | A | N |
| ATOM | 410 | CA | HIS | A | 111 | −29.788 | 17.621 | 1.866 | 1.00 | 8.87 | | A | C |
| ANISOU | 410 | CA | HIS | A | 111 | 1113 | 1144 | 1113 | −28 | −3 | −23 | A | C |
| ATOM | 411 | CB | HIS | A | 111 | −29.526 | 18.557 | 3.043 | 1.00 | 8.32 | | A | C |
| ANISOU | 411 | CB | HIS | A | 111 | 1119 | 1049 | 994 | −8 | −35 | 6 | A | C |
| ATOM | 412 | CG | HIS | A | 111 | −29.997 | 18.012 | 4.356 | 1.00 | 8.35 | | A | C |
| ANISOU | 412 | CG | HIS | A | 111 | 1094 | 1060 | 1020 | −23 | 34 | −26 | A | C |
| ATOM | 413 | ND1 | HIS | A | 111 | −31.324 | 17.739 | 4.614 | 1.00 | 8.65 | | A | N |
| ANISOU | 413 | ND1 | HIS | A | 111 | 1109 | 1088 | 1090 | 28 | 15 | −30 | A | N |
| ATOM | 414 | CE1 | HIS | A | 111 | −31.442 | 17.266 | 5.842 | 1.00 | 10.18 | | A | C |
| ANISOU | 414 | CE1 | HIS | A | 111 | 1290 | 1406 | 1171 | 47 | 25 | 10 | A | C |
| ATOM | 415 | NE2 | HIS | A | 111 | −30.242 | 17.228 | 6.392 | 1.00 | 8.78 | | A | N |
| ANISOU | 415 | NE2 | HIS | A | 111 | 1153 | 1053 | 1131 | 14 | 48 | −97 | A | N |
| ATOM | 416 | CD2 | HIS | A | 111 | −29.320 | 17.690 | 5.484 | 1.00 | 8.92 | | A | C |
| ANISOU | 416 | CD2 | HIS | A | 111 | 1186 | 1109 | 1093 | −24 | −2 | 6 | A | C |
| ATOM | 417 | C | HIS | A | 111 | −28.975 | 16.337 | 2.053 | 1.00 | 9.30 | | A | C |
| ANISOU | 417 | C | HIS | A | 111 | 1175 | 1158 | 1200 | −16 | −5 | −16 | A | C |
| ATOM | 418 | O | HIS | A | 111 | −27.743 | 16.387 | 2.198 | 1.00 | 9.77 | | A | O |
| ANISOU | 418 | O | HIS | A | 111 | 1126 | 1232 | 1355 | 40 | 9 | −8 | A | O |
| ATOM | 419 | N | GLN | A | 112 | −29.656 | 15.192 | 2.061 | 1.00 | 9.61 | | A | N |
| ANISOU | 419 | N | GLN | A | 112 | 1257 | 1143 | 1253 | 23 | 19 | −2 | A | N |
| ATOM | 420 | CA | GLN | A | 112 | −28.989 | 13.920 | 2.347 | 1.00 | 9.93 | | A | C |
| ANISOU | 420 | CA | GLN | A | 112 | 1266 | 1211 | 1296 | 18 | 3 | −23 | A | C |
| ATOM | 421 | CB | GLN | A | 112 | −29.508 | 13.320 | 3.659 | 1.00 | 10.03 | | A | C |
| ANISOU | 421 | CB | GLN | A | 112 | 1301 | 1215 | 1296 | 58 | −33 | 18 | A | C |
| ATOM | 422 | CG | GLN | A | 112 | −29.125 | 14.135 | 4.896 | 1.00 | 11.18 | | A | C |
| ANISOU | 422 | CG | GLN | A | 112 | 1485 | 1424 | 1337 | 26 | −25 | −85 | A | C |
| ATOM | 423 | CD | GLN | A | 112 | −29.639 | 13.532 | 6.192 | 1.00 | 11.74 | | A | C |
| ANISOU | 423 | CD | GLN | A | 112 | 1547 | 1433 | 1481 | 57 | −25 | 71 | A | C |
| ATOM | 424 | OE1 | GLN | A | 112 | −30.627 | 12.798 | 6.204 | 1.00 | 14.14 | | A | O |
| ANISOU | 424 | OE1 | GLN | A | 112 | 1685 | 1916 | 1771 | −79 | −167 | 134 | A | O |
| ATOM | 425 | NE2 | GLN | A | 112 | −28.970 | 13.848 | 7.297 | 1.00 | 12.35 | | A | N |
| ANISOU | 425 | NE2 | GLN | A | 112 | 1650 | 1564 | 1479 | 66 | −16 | −65 | A | N |
| ATOM | 426 | C | GLN | A | 112 | −29.091 | 12.912 | 1.203 | 1.00 | 9.55 | | A | C |
| ANISOU | 426 | C | GLN | A | 112 | 1192 | 1204 | 1231 | 64 | 50 | −3 | A | C |
| ATOM | 427 | O | GLN | A | 112 | −29.205 | 11.703 | 1.441 | 1.00 | 10.58 | | A | O |
| ANISOU | 427 | O | GLN | A | 112 | 1335 | 1256 | 1428 | 9 | 21 | −55 | A | O |
| ATOM | 428 | N | LYS | A | 113 | −29.025 | 13.411 | −0.033 | 1.00 | 8.33 | | A | N |
| ANISOU | 428 | N | LYS | A | 113 | 1125 | 996 | 1045 | 92 | 15 | −73 | A | N |
| ATOM | 429 | CA | LYS | A | 113 | −28.998 | 12.547 | −1.215 | 1.00 | 8.24 | | A | C |
| ANISOU | 429 | CA | LYS | A | 113 | 1167 | 999 | 965 | 69 | 16 | −1 | A | C |
| ATOM | 430 | CB | LYS | A | 113 | −28.677 | 13.361 | −2.472 | 1.00 | 9.35 | | A | C |
| ANISOU | 430 | CB | LYS | A | 113 | 1330 | 1177 | 1047 | 65 | 56 | 51 | A | C |
| ATOM | 431 | CG | LYS | A | 113 | −28.789 | 12.575 | −3.771 | 1.00 | 11.95 | | A | C |
| ANISOU | 431 | CG | LYS | A | 113 | 1673 | 1462 | 1407 | 8 | −27 | −110 | A | C |
| ATOM | 432 | CD | LYS | A | 113 | −28.822 | 13.501 | −4.965 | 1.00 | 15.87 | | A | C |
| ANISOU | 432 | CD | LYS | A | 113 | 2276 | 2014 | 1740 | 53 | 80 | 113 | A | C |
| ATOM | 433 | CE | LYS | A | 113 | −27.480 | 14.126 | −5.221 | 1.00 | 18.60 | | A | C |
| ANISOU | 433 | CE | LYS | A | 113 | 2284 | 2438 | 2347 | −9 | −23 | 10 | A | C |
| ATOM | 434 | NZ | LYS | A | 113 | −27.481 | 14.830 | −6.539 | 1.00 | 22.06 | | A | N |
| ANISOU | 434 | NZ | LYS | A | 113 | 2808 | 2934 | 2641 | 31 | −27 | 272 | A | N |
| ATOM | 435 | C | LYS | A | 113 | −27.974 | 11.428 | −1.039 | 1.00 | 7.90 | | A | C |
| ANISOU | 435 | C | LYS | A | 113 | 1096 | 947 | 957 | 6 | 0 | −15 | A | C |
| ATOM | 436 | O | LYS | A | 113 | −26.863 | 11.661 | −0.562 | 1.00 | 8.43 | | A | O |
| ANISOU | 436 | O | LYS | A | 113 | 1074 | 991 | 1139 | 44 | 20 | 1 | A | O |
| ATOM | 437 | N | SER | A | 114 | −28.359 | 10.216 | −1.423 | 1.00 | 7.25 | | A | N |
| ANISOU | 437 | N | SER | A | 114 | 1043 | 835 | 877 | 72 | −12 | 10 | A | N |
| ATOM | 438 | CA | SER | A | 114 | −27.510 | 9.054 | −1.212 | 1.00 | 7.39 | | A | C |
| ANISOU | 438 | CA | SER | A | 114 | 1008 | 869 | 932 | 71 | 3 | −24 | A | C |
| ATOM | 439 | CB | SER | A | 114 | −28.177 | 8.102 | −0.215 | 1.00 | 7.69 | | A | C |
| ANISOU | 439 | CB | SER | A | 114 | 899 | 954 | 1069 | 70 | 7 | 62 | A | C |
| ATOM | 440 | OG | SER | A | 114 | −29.419 | 7.628 | −0.714 | 1.00 | 9.45 | | A | O |
| ANISOU | 440 | OG | SER | A | 114 | 1315 | 1082 | 1192 | −70 | −53 | −10 | A | O |
| ATOM | 441 | C | SER | A | 114 | −27.243 | 8.307 | −2.505 | 1.00 | 8.43 | | A | C |
| ANISOU | 441 | C | SER | A | 114 | 1112 | 1067 | 1025 | 54 | −19 | −41 | A | C |
| ATOM | 442 | O | SER | A | 114 | −27.944 | 8.490 | −3.503 | 1.00 | 8.99 | | A | O |
| ANISOU | 442 | O | SER | A | 114 | 1174 | 1226 | 1015 | 42 | −1 | −49 | A | O |
| ATOM | 443 | N | VAL | A | 115 | −26.212 | 7.471 | −2.478 | 1.00 | 9.32 | | A | N |
| ANISOU | 443 | N | VAL | A | 115 | 1227 | 1114 | 1200 | 72 | 38 | −131 | A | N |
| ATOM | 444 | CA | VAL | A | 115 | −26.060 | 6.441 | −3.494 | 1.00 | 9.13 | | A | C |
| ANISOU | 444 | CA | VAL | A | 115 | 1251 | 1082 | 1136 | 27 | 29 | −122 | A | C |
| ATOM | 445 | CB | VAL | A | 115 | −24.592 | 5.986 | −3.652 | 1.00 | 11.28 | | A | C |
| ANISOU | 445 | CB | VAL | A | 115 | 1351 | 1405 | 1529 | 36 | 88 | −177 | A | C |
| ATOM | 446 | CG1 | VAL | A | 115 | −24.470 | 4.977 | −4.796 | 1.00 | 11.92 | | A | C |
| ANISOU | 446 | CG1 | VAL | A | 115 | 1643 | 1312 | 1575 | −44 | 46 | −198 | A | C |
| ATOM | 447 | CG2 | VAL | A | 115 | −23.683 | 7.174 | −3.902 | 1.00 | 13.15 | | A | C |
| ANISOU | 447 | CG2 | VAL | A | 115 | 1686 | 1582 | 1729 | −14 | 152 | −12 | A | C |
| ATOM | 448 | C | VAL | A | 115 | −26.908 | 5.247 | −3.071 | 1.00 | 8.94 | | A | C |
| ANISOU | 448 | C | VAL | A | 115 | 1173 | 1149 | 1073 | 31 | −18 | −24 | A | C |
| ATOM | 449 | O | VAL | A | 115 | −26.730 | 4.715 | −1.969 | 1.00 | 8.72 | | A | O |
| ANISOU | 449 | O | VAL | A | 115 | 1203 | 1054 | 1057 | 33 | −20 | 25 | A | O |
| ATOM | 450 | N | GLY | A | 116 | −27.849 | 4.862 | −3.931 | 1.00 | 8.41 | | A | N |
| ANISOU | 450 | N | GLY | A | 116 | 1193 | 997 | 1004 | −70 | 11 | −56 | A | N |
| ATOM | 451 | CA | GLY | A | 116 | −28.585 | 3.610 | −3.779 | 1.00 | 8.13 | | A | C |
| ANISOU | 451 | CA | GLY | A | 116 | 1086 | 980 | 1023 | −12 | −21 | −15 | A | C |
| ATOM | 452 | C | GLY | A | 116 | −27.838 | 2.537 | −4.546 | 1.00 | 8.70 | | A | C |
| ANISOU | 452 | C | GLY | A | 116 | 1194 | 998 | 1113 | 23 | 11 | 30 | A | C |
| ATOM | 453 | O | GLY | A | 116 | −27.450 | 2.745 | −5.701 | 1.00 | 9.35 | | A | O |
| ANISOU | 453 | O | GLY | A | 116 | 1445 | 997 | 1110 | 84 | 41 | −41 | A | O |
| ATOM | 454 | N | PHE | A | 117 | −27.631 | 1.394 | −3.898 | 1.00 | 7.90 | | A | N |
| ANISOU | 454 | N | PHE | A | 117 | 1105 | 879 | 1019 | −2 | 1 | 0 | A | N |
| ATOM | 455 | CA | PHE | A | 117 | −26.783 | 0.331 | −4.425 | 1.00 | 7.93 | | A | C |
| ANISOU | 455 | CA | PHE | A | 117 | 1025 | 938 | 1049 | −23 | 1 | 8 | A | C |
| ATOM | 456 | CB | PHE | A | 117 | −25.422 | 0.441 | −3.717 | 1.00 | 7.80 | | A | C |
| ANISOU | 456 | CB | PHE | A | 117 | 921 | 974 | 1068 | 18 | −39 | 1 | A | C |
| ATOM | 457 | CG | PHE | A | 117 | −24.386 | −0.596 | −4.100 | 1.00 | 7.30 | | A | C |
| ANISOU | 457 | CG | PHE | A | 117 | 965 | 939 | 871 | −6 | 16 | 31 | A | C |
| ATOM | 458 | CD1 | PHE | A | 117 | −24.557 | −1.493 | −5.158 | 1.00 | 7.81 | | A | C |
| ANISOU | 458 | CD1 | PHE | A | 117 | 944 | 1018 | 1007 | 6 | 160 | −15 | A | C |
| ATOM | 459 | CE1 | PHE | A | 117 | −23.558 | −2.431 | −5.470 | 1.00 | 8.26 | | A | C |
| ANISOU | 459 | CE1 | PHE | A | 117 | 980 | 1123 | 1034 | 63 | −39 | −34 | A | C |
| ATOM | 460 | CZ | PHE | A | 117 | −22.368 | −2.448 | −4.739 | 1.00 | 8.35 | | A | C |
| ANISOU | 460 | CZ | PHE | A | 117 | 1079 | 1120 | 974 | −6 | 0 | −26 | A | C |
| ATOM | 461 | CE2 | PHE | A | 117 | −22.183 | −1.541 | −3.697 | 1.00 | 6.67 | | A | C |
| ANISOU | 461 | CE2 | PHE | A | 117 | 803 | 865 | 868 | 39 | 34 | 85 | A | C |
| ATOM | 462 | CD2 | PHE | A | 117 | −23.186 | −0.627 | −3.387 | 1.00 | 7.00 | | A | C |
| ANISOU | 462 | CD2 | PHE | A | 117 | 857 | 848 | 955 | −59 | 58 | 9 | A | C |
| ATOM | 463 | C | PHE | A | 117 | −27.507 | −0.994 | −4.165 | 1.00 | 7.59 | | A | C |
| ANISOU | 463 | C | PHE | A | 117 | 951 | 989 | 943 | −46 | 43 | −21 | A | C |
| ATOM | 464 | O | PHE | A | 117 | −27.716 | −1.381 | −3.016 | 1.00 | 7.57 | | A | O |
| ANISOU | 464 | O | PHE | A | 117 | 960 | 965 | 951 | 74 | 13 | 32 | A | O |
| ATOM | 465 | N | PHE | A | 118 | −27.924 | −1.661 | −5.245 | 1.00 | 7.99 | | A | N |
| ANISOU | 465 | N | PHE | A | 118 | 1085 | 930 | 1019 | −30 | −16 | −58 | A | N |
| ATOM | 466 | CA | PHE | A | 118 | −28.728 | −2.885 | −5.143 | 1.00 | 7.86 | | A | C |
| ANISOU | 466 | CA | PHE | A | 118 | 950 | 1007 | 1031 | −54 | −17 | −69 | A | C |
| ATOM | 467 | CB | PHE | A | 118 | −30.182 | −2.606 | −5.561 | 1.00 | 8.59 | | A | C |
| ANISOU | 467 | CB | PHE | A | 118 | 1018 | 1020 | 1227 | −36 | 17 | −34 | A | C |
| ATOM | 468 | CG | PHE | A | 118 | −30.857 | −1.569 | −4.718 | 1.00 | 7.12 | | A | C |
| ANISOU | 468 | CG | PHE | A | 118 | 888 | 781 | 1037 | 32 | 40 | 52 | A | C |
| ATOM | 469 | CD1 | PHE | A | 118 | −30.735 | −0.212 | −5.026 | 1.00 | 7.98 | | A | C |
| ANISOU | 469 | CD1 | PHE | A | 118 | 1028 | 887 | 1117 | −48 | 65 | 101 | A | C |
| ATOM | 470 | CE1 | PHE | A | 118 | −31.341 | 0.752 | −4.230 | 1.00 | 8.39 | | A | C |
| ANISOU | 470 | CE1 | PHE | A | 118 | 987 | 1015 | 1186 | 29 | 16 | 21 | A | C |
| ATOM | 471 | CZ | PHE | A | 118 | −32.081 | 0.370 | −3.111 | 1.00 | 10.08 | | A | C |
| ANISOU | 471 | CZ | PHE | A | 118 | 1255 | 1284 | 1291 | 15 | 68 | 17 | A | C |
| ATOM | 472 | CE2 | PHE | A | 118 | −32.209 | −0.973 | −2.793 | 1.00 | 8.43 | | A | C |
| ANISOU | 472 | CE2 | PHE | A | 118 | 1011 | 1139 | 1054 | 7 | 6 | −91 | A | C |
| ATOM | 473 | CD2 | PHE | A | 118 | −31.597 | −1.939 | −3.599 | 1.00 | 8.40 | | A | C |
| ANISOU | 473 | CD2 | PHE | A | 118 | 1087 | 1068 | 1035 | 15 | 100 | −44 | A | C |
| ATOM | 474 | C | PHE | A | 118 | −28.176 | −4.031 | −5.982 | 1.00 | 7.99 | | A | C |
| ANISOU | 474 | C | PHE | A | 118 | 1042 | 973 | 1019 | −31 | 24 | −4 | A | C |
| ATOM | 475 | O | PHE | A | 118 | −27.535 | −3.811 | −7.017 | 1.00 | 8.77 | | A | O |
| ANISOU | 475 | O | PHE | A | 118 | 1212 | 1024 | 1097 | −65 | 185 | 4 | A | O |
| ATOM | 476 | N | LEU | A | 119 | −28.435 | −5.252 | −5.522 | 1.00 | 7.27 | | A | N |
| ANISOU | 476 | N | LEU | A | 119 | 934 | 904 | 924 | −10 | −55 | −42 | A | N |
| ATOM | 477 | CA | LEU | A | 119 | −28.218 | −6.454 | −6.312 | 1.00 | 7.57 | | A | C |
| ANISOU | 477 | CA | LEU | A | 119 | 948 | 953 | 975 | 25 | −32 | −6 | A | C |
| ATOM | 478 | CB | LEU | A | 119 | −27.613 | −7.560 | −5.443 | 1.00 | 7.52 | | A | C |
| ANISOU | 478 | CB | LEU | A | 119 | 1038 | 795 | 1023 | 23 | −15 | −10 | A | C |
| ATOM | 479 | CG | LEU | A | 119 | −27.380 | −8.937 | −6.092 | 1.00 | 6.77 | | A | C |
| ANISOU | 479 | CG | LEU | A | 119 | 1053 | 725 | 796 | 23 | 37 | 21 | A | C |
| ATOM | 480 | CD1 | LEU | A | 119 | −26.347 | −8.872 | −7.221 | 1.00 | 7.86 | | A | C |
| ANISOU | 480 | CD1 | LEU | A | 119 | 972 | 1215 | 799 | 39 | −14 | −47 | A | C |
| ATOM | 481 | CD2 | LEU | A | 119 | −26.928 | −9.927 | −5.022 | 1.00 | 7.98 | | A | C |
| ANISOU | 481 | CD2 | LEU | A | 119 | 1123 | 1030 | 879 | 75 | −79 | 140 | A | C |
| ATOM | 482 | C | LEU | A | 119 | −29.566 | −6.889 | −6.867 | 1.00 | 8.38 | | A | C |
| ANISOU | 482 | C | LEU | A | 119 | 1053 | 1142 | 990 | −34 | 18 | −4 | A | C |
| ATOM | 483 | O | LEU | A | 119 | −30.542 | −6.972 | −6.124 | 1.00 | 7.41 | | A | O |
| ANISOU | 483 | O | LEU | A | 119 | 856 | 1138 | 823 | −13 | −12 | −8 | A | O |
| ATOM | 484 | N | GLN | A | 120 | −29.611 | −7.136 | −8.176 | 1.00 | 8.11 | | A | N |
| ANISOU | 484 | N | GLN | A | 120 | 1086 | 1049 | 946 | −46 | −47 | −79 | A | N |
| ATOM | 485 | CA | GLN | A | 120 | −30.853 | −7.456 | −8.875 | 1.00 | 7.90 | | A | C |
| ANISOU | 485 | CA | GLN | A | 120 | 1058 | 1025 | 917 | −2 | −17 | 22 | A | C |
| ATOM | 486 | CB | GLN | A | 120 | −31.148 | −6.388 | −9.933 | 1.00 | 7.36 | | A | C |
| ANISOU | 486 | CB | GLN | A | 120 | 970 | 983 | 842 | 61 | −34 | −14 | A | C |
| ATOM | 487 | CG | GLN | A | 120 | −32.498 | −6.533 | −10.607 | 1.00 | 8.64 | | A | C |
| ANISOU | 487 | CG | GLN | A | 120 | 1100 | 1257 | 926 | −5 | −30 | 71 | A | C |
| ATOM | 488 | CD | GLN | A | 120 | −32.597 | −5.702 | −11.876 | 1.00 | 8.87 | | A | C |
| ANISOU | 488 | CD | GLN | A | 120 | 1116 | 1258 | 996 | −34 | 10 | 62 | A | C |
| ATOM | 489 | OE1 | GLN | A | 120 | −33.305 | −4.691 | −11.921 | 1.00 | 13.04 | | A | O |
| ANISOU | 489 | OE1 | GLN | A | 120 | 1748 | 1573 | 1632 | 113 | −51 | −25 | A | O |
| ATOM | 490 | NE2 | GLN | A | 120 | −31.893 | −6.130 | −12.918 | 1.00 | 7.19 | | A | N |
| ANISOU | 490 | NE2 | GLN | A | 120 | 995 | 875 | 860 | −51 | −19 | 8 | A | N |
| ATOM | 491 | C | GLN | A | 120 | −30.761 | −8.833 | −9.521 | 1.00 | 8.65 | | A | C |
| ANISOU | 491 | C | GLN | A | 120 | 1082 | 1116 | 1089 | −14 | 2 | −28 | A | C |
| ATOM | 492 | O | GLN | A | 120 | −29.817 | −9.110 | −10.259 | 1.00 | 10.12 | | A | O |
| ANISOU | 492 | O | GLN | A | 120 | 1245 | 1265 | 1335 | −35 | 144 | −175 | A | O |
| ATOM | 493 | N | CYS | A | 121 | −31.749 | −9.683 | −9.248 | 1.00 | 8.28 | | A | N |
| ANISOU | 493 | N | CYS | A | 121 | 1140 | 1016 | 989 | −55 | 21 | −18 | A | N |
| ATOM | 494 | CA | CYS | A | 121 | −31.752 | −11.057 | −9.741 | 1.00 | 8.61 | | A | C |
| ANISOU | 494 | CA | CYS | A | 121 | 1184 | 1031 | 1057 | −11 | −49 | 11 | A | C |
| ATOM | 495 | CB | CYS | A | 121 | −31.891 | −12.042 | −8.580 | 1.00 | 8.32 | | A | C |
| ANISOU | 495 | CB | CYS | A | 121 | 1131 | 1050 | 980 | −37 | −20 | 41 | A | C |
| ATOM | 496 | SG | CYS | A | 121 | −31.857 | −13.781 | −9.084 | 1.00 | 10.85 | | A | S |
| ANISOU | 496 | SG | CYS | A | 121 | 1381 | 1224 | 1517 | 50 | −10 | 1 | A | S |
| ATOM | 497 | C | CYS | A | 121 | −32.887 | −11.305 | −10.716 | 1.00 | 7.94 | | A | C |
| ANISOU | 497 | C | CYS | A | 121 | 1043 | 962 | 1011 | 54 | −30 | 6 | A | C |
| ATOM | 498 | O | CYS | A | 121 | −34.056 | −11.087 | −10.382 | 1.00 | 8.48 | | A | O |
| ANISOU | 498 | O | CYS | A | 121 | 1078 | 1150 | 994 | 4 | 43 | −12 | A | O |
| ATOM | 499 | N | ASN | A | 122 | −32.534 | −11.758 | −11.919 | 1.00 | 8.29 | | A | N |
| ANISOU | 499 | N | ASN | A | 122 | 1086 | 1052 | 1012 | −3 | −72 | −19 | A | N |
| ATOM | 500 | CA | ASN | A | 122 | −33.509 | −12.297 | −12.874 | 1.00 | 8.55 | | A | C |
| ANISOU | 500 | CA | ASN | A | 122 | 1075 | 1107 | 1066 | −50 | −61 | −3 | A | C |
| ATOM | 501 | CB | ASN | A | 122 | −34.060 | −13.632 | −12.333 | 1.00 | 8.59 | | A | C |
| ANISOU | 501 | CB | ASN | A | 122 | 1147 | 1046 | 1069 | −96 | −5 | −69 | A | C |
| ATOM | 502 | CG | ASN | A | 122 | −34.610 | −14.539 | −13.425 | 1.00 | 9.19 | | A | C |
| ANISOU | 502 | CG | ASN | A | 122 | 1184 | 1162 | 1144 | −114 | −66 | −51 | A | C |
| ATOM | 503 | OD1 | ASN | A | 122 | −34.084 | −14.586 | −14.536 | 1.00 | 10.82 | | A | O |
| ANISOU | 503 | OD1 | ASN | A | 122 | 1469 | 1363 | 1280 | −87 | 6 | −122 | A | O |
| ATOM | 504 | ND2 | ASN | A | 122 | −35.662 | −15.287 | −13.096 | 1.00 | 9.27 | | A | N |
| ANISOU | 504 | ND2 | ASN | A | 122 | 1085 | 1267 | 1172 | −57 | 8 | −54 | A | N |
| ATOM | 505 | C | ASN | A | 122 | −34.659 | −11.335 | −13.177 | 1.00 | 9.69 | | A | C |
| ANISOU | 505 | C | ASN | A | 122 | 1233 | 1178 | 1271 | 14 | 17 | −26 | A | C |
| ATOM | 506 | O | ASN | A | 122 | −35.794 | −11.769 | −13.395 | 1.00 | 10.78 | | A | O |
| ANISOU | 506 | O | ASN | A | 122 | 1255 | 1348 | 1492 | −30 | −36 | 54 | A | O |
| ATOM | 507 | N | ALA | A | 123 | −34.371 | −10.031 | −13.198 | 1.00 | 10.30 | | A | N |
| ANISOU | 507 | N | ALA | A | 123 | 1400 | 1280 | 1233 | −52 | 2 | −6 | A | N |
| ATOM | 508 | CA | ALA | A | 123 | −35.427 | −9.032 | −13.383 | 1.00 | 11.45 | | A | C |
| ANISOU | 508 | CA | ALA | A | 123 | 1503 | 1404 | 1442 | −3 | −45 | −42 | A | C |
| ATOM | 509 | CB | ALA | A | 123 | −34.970 | −7.651 | −12.932 | 1.00 | 11.66 | | A | C |
| ANISOU | 509 | CB | ALA | A | 123 | 1574 | 1414 | 1443 | −14 | −23 | −9 | A | C |
| ATOM | 510 | C | ALA | A | 123 | −35.976 | −8.977 | −14.808 | 1.00 | 11.72 | | A | C |
| ANISOU | 510 | C | ALA | A | 123 | 1551 | 1490 | 1413 | 8 | −26 | −12 | A | C |
| ATOM | 511 | O | ALA | A | 123 | −37.053 | −8.416 | −15.029 | 1.00 | 13.82 | | A | O |
| ANISOU | 511 | O | ALA | A | 123 | 1637 | 1858 | 1756 | −4 | −22 | −2 | A | O |
| ATOM | 512 | N | GLU | A | 124 | −35.254 | −9.565 | −15.764 | 1.00 | 11.75 | | A | N |
| ANISOU | 512 | N | GLU | A | 124 | 1454 | 1576 | 1433 | −71 | −10 | 20 | A | N |
| ATOM | 513 | CA | GLU | A | 124 | −35.724 | −9.616 | −17.154 | 1.00 | 11.81 | | A | C |
| ANISOU | 513 | CA | GLU | A | 124 | 1464 | 1546 | 1478 | −63 | −25 | −2 | A | C |
| ATOM | 514 | CB | GLU | A | 124 | −34.586 | −9.976 | −18.116 | 1.00 | 11.87 | | A | C |
| ANISOU | 514 | CB | GLU | A | 124 | 1534 | 1582 | 1393 | −116 | 0 | −3 | A | C |
| ATOM | 515 | CG | GLU | A | 124 | −33.535 | −8.873 | −18.271 | 1.00 | 13.63 | | A | C |
| ANISOU | 515 | CG | GLU | A | 124 | 1654 | 1667 | 1856 | −115 | 40 | 21 | A | C |
| ATOM | 516 | CD | GLU | A | 124 | −32.423 | −9.233 | −19.239 | 1.00 | 14.46 | | A | C |
| ANISOU | 516 | CD | GLU | A | 124 | 1780 | 1894 | 1822 | −29 | 15 | 24 | A | C |
| ATOM | 517 | OE1 | GLU | A | 124 | −32.664 | −10.045 | −20.161 | 1.00 | 15.69 | | A | O |
| ANISOU | 517 | OE1 | GLU | A | 124 | 1971 | 1925 | 2066 | −24 | −21 | −74 | A | O |
| ATOM | 518 | OE2 | GLU | A | 124 | −31.307 | −8.681 | −19.092 | 1.00 | 14.00 | | A | O |
| ANISOU | 518 | OE2 | GLU | A | 124 | 1689 | 1838 | 1793 | −97 | 26 | −60 | A | O |
| ATOM | 519 | C | GLU | A | 124 | −36.875 | −10.604 | −17.294 | 1.00 | 11.80 | | A | C |
| ANISOU | 519 | C | GLU | A | 124 | 1497 | 1543 | 1442 | −82 | −39 | 75 | A | C |
| ATOM | 520 | O | GLU | A | 124 | −37.580 | −10.607 | −18.307 | 1.00 | 12.58 | | A | O |
| ANISOU | 520 | O | GLU | A | 124 | 1579 | 1762 | 1437 | −103 | −56 | 122 | A | O |
| ATOM | 521 | N | SER | A | 125 | −37.062 | −11.437 | −16.270 | 1.00 | 10.53 | | A | N |
| ANISOU | 521 | N | SER | A | 125 | 1309 | 1375 | 1316 | −86 | −20 | 71 | A | N |
| ATOM | 522 | CA | SER | A | 125 | −38.156 | −12.402 | −16.244 | 1.00 | 10.74 | | A | C |
| ANISOU | 522 | CA | SER | A | 125 | 1313 | 1357 | 1410 | −49 | 4 | 34 | A | C |
| ATOM | 523 | CB | SER | A | 125 | −37.721 | −13.689 | −15.534 | 1.00 | 10.67 | | A | C |
| ANISOU | 523 | CB | SER | A | 125 | 1316 | 1304 | 1436 | −91 | −39 | 67 | A | C |
| ATOM | 524 | OG | SER | A | 125 | −38.794 | −14.620 | −15.455 | 1.00 | 9.96 | | A | O |
| ANISOU | 524 | OG | SER | A | 125 | 1176 | 1434 | 1174 | −41 | 25 | 147 | A | O |
| ATOM | 525 | C | SER | A | 125 | −39.359 | −11.820 | −15.522 | 1.00 | 11.17 | | A | C |
| ANISOU | 525 | C | SER | A | 125 | 1390 | 1425 | 1428 | −34 | 26 | −9 | A | C |
| ATOM | 526 | O | SER | A | 125 | −39.216 | −11.141 | −14.501 | 1.00 | 12.69 | | A | O |
| ANISOU | 526 | O | SER | A | 125 | 1558 | 1764 | 1498 | −142 | −3 | −56 | A | O |
| ATOM | 527 | N | ASP | A | 126 | −40.544 | −12.100 | −16.053 | 1.00 | 10.83 | | A | N |
| ANISOU | 527 | N | ASP | A | 126 | 1339 | 1449 | 1328 | −22 | 41 | −4 | A | N |
| ATOM | 528 | CA | ASP | A | 126 | −41.788 | −11.776 | −15.364 | 1.00 | 11.66 | | A | C |
| ANISOU | 528 | CA | ASP | A | 126 | 1454 | 1511 | 1467 | −1 | 54 | −27 | A | C |
| ATOM | 529 | CB | ASP | A | 126 | −42.768 | −11.080 | −16.312 | 1.00 | 13.44 | | A | C |
| ANISOU | 529 | CB | ASP | A | 126 | 1654 | 1775 | 1676 | 82 | 19 | −12 | A | C |
| ATOM | 530 | CG | ASP | A | 126 | −42.342 | −9.663 | −16.644 | 1.00 | 19.39 | | A | C |
| ANISOU | 530 | CG | ASP | A | 126 | 2548 | 2133 | 2685 | −66 | −3 | 59 | A | C |
| ATOM | 531 | OD1 | ASP | A | 126 | −42.262 | −8.831 | −15.717 | 1.00 | 23.47 | | A | O |
| ANISOU | 531 | OD1 | ASP | A | 126 | 3270 | 2749 | 2898 | −96 | 39 | −119 | A | O |
| ATOM | 532 | OD2 | ASP | A | 126 | −42.089 | −9.380 | −17.830 | 1.00 | 24.10 | | A | O |
| ANISOU | 532 | OD2 | ASP | A | 126 | 3347 | 2847 | 2964 | −25 | 139 | 49 | A | O |
| ATOM | 533 | C | ASP | A | 126 | −42.415 | −13.016 | −14.723 | 1.00 | 10.58 | | A | C |
| ANISOU | 533 | C | ASP | A | 126 | 1305 | 1333 | 1383 | 31 | 27 | −55 | A | C |
| ATOM | 534 | O | ASP | A | 126 | −43.560 | −12.972 | −14.263 | 1.00 | 11.39 | | A | O |
| ANISOU | 534 | O | ASP | A | 126 | 1282 | 1509 | 1536 | 14 | −18 | 0 | A | O |
| ATOM | 535 | N | SER | A | 127 | −41.648 | −14.109 | −14.669 | 1.00 | 8.97 | | A | N |
| ANISOU | 535 | N | SER | A | 127 | 1094 | 1181 | 1132 | −66 | −16 | 16 | A | N |
| ATOM | 536 | CA | SER | A | 127 | −42.116 | −15.358 | −14.066 | 1.00 | 8.07 | | A | C |
| ANISOU | 536 | CA | SER | A | 127 | 1063 | 1042 | 962 | −12 | −15 | −36 | A | C |
| ATOM | 537 | CB | SER | A | 127 | −41.129 | −16.497 | −14.309 | 1.00 | 9.22 | | A | C |
| ANISOU | 537 | CB | SER | A | 127 | 1233 | 1077 | 1195 | 17 | −8 | 44 | A | C |
| ATOM | 538 | OG | SER | A | 127 | −41.496 | −17.635 | −13.540 | 1.00 | 9.95 | | A | O |
| ANISOU | 538 | OG | SER | A | 127 | 1339 | 1284 | 1156 | −101 | 87 | 117 | A | O |
| ATOM | 539 | C | SER | A | 127 | −42.363 | −15.230 | −12.569 | 1.00 | 8.50 | | A | C |
| ANISOU | 539 | C | SER | A | 127 | 1054 | 1169 | 1005 | −81 | 7 | −11 | A | C |
| ATOM | 540 | O | SER | A | 127 | −41.522 | −14.704 | −11.825 | 1.00 | 8.68 | | A | O |
| ANISOU | 540 | O | SER | A | 127 | 1048 | 1359 | 892 | −56 | −25 | 22 | A | O |
| ATOM | 541 | N | THR | A | 128 | −43.514 | −15.734 | −12.135 | 1.00 | 8.35 | | A | N |
| ANISOU | 541 | N | THR | A | 128 | 1072 | 1104 | 996 | 7 | 20 | 0 | A | N |
| ATOM | 542 | CA | THR | A | 128 | −43.860 | −15.743 | −10.722 | 1.00 | 8.77 | | A | C |
| ANISOU | 542 | CA | THR | A | 128 | 1078 | 1178 | 1078 | −22 | 8 | −11 | A | C |
| ATOM | 543 | CB | THR | A | 128 | −45.379 | −15.515 | −10.512 | 1.00 | 9.07 | | A | C |
| ANISOU | 543 | CB | THR | A | 128 | 1101 | 1194 | 1153 | −17 | 14 | 43 | A | C |
| ATOM | 544 | OG1 | THR | A | 128 | −46.128 | −16.401 | −11.356 | 1.00 | 8.30 | | A | O |
| ANISOU | 544 | OG1 | THR | A | 128 | 1124 | 1133 | 896 | 72 | 22 | 85 | A | O |
| ATOM | 545 | CG2 | THR | A | 128 | −45.756 | −14.069 | −10.826 | 1.00 | 10.24 | | A | C |
| ANISOU | 545 | CG2 | THR | A | 128 | 1368 | 1272 | 1251 | 102 | −12 | −12 | A | C |
| ATOM | 546 | C | THR | A | 128 | −43.426 | −17.026 | −10.013 | 1.00 | 8.99 | | A | C |
| ANISOU | 546 | C | THR | A | 128 | 1046 | 1184 | 1184 | −32 | −6 | −23 | A | C |
| ATOM | 547 | O | THR | A | 128 | −43.651 | −17.175 | −8.811 | 1.00 | 9.62 | | A | O |
| ANISOU | 547 | O | THR | A | 128 | 1199 | 1327 | 1129 | −35 | −34 | −33 | A | O |
| ATOM | 548 | N | ASER | A | 129 | −42.790 | −17.932 | −10.754 | 0.50 | 8.60 | | A | N |
| ANISOU | 548 | N | ASER | A | 129 | 988 | 1141 | 1138 | −43 | −19 | −22 | A | N |
| ATOM | 549 | N | BSER | A | 129 | −42.801 | −17.944 | −10.750 | 0.50 | 9.02 | | A | N |
| ANISOU | 549 | N | BSER | A | 129 | 1048 | 1191 | 1188 | −40 | −16 | −33 | A | N |
| ATOM | 550 | CA | ASER | A | 129 | −42.421 | −19.248 | −10.233 | 0.50 | 8.87 | | A | C |
| ANISOU | 550 | CA | ASER | A | 129 | 1050 | 1156 | 1166 | −35 | −4 | 7 | A | C |
| ATOM | 551 | CA | BSER | A | 129 | −42.425 | −19.244 | −10.191 | 0.50 | 9.70 | | A | C |
| ANISOU | 551 | CA | BSER | A | 129 | 1167 | 1253 | 1265 | −28 | −11 | 16 | A | C |
| ATOM | 552 | CB | ASER | A | 129 | −42.546 | −20.304 | −11.333 | 0.50 | 8.98 | | A | C |
| ANISOU | 552 | CB | ASER | A | 129 | 1069 | 1174 | 1169 | −25 | 17 | −21 | A | C |
| ATOM | 553 | CB | BSER | A | 129 | −42.547 | −20.348 | −11.247 | 0.50 | 10.49 | | A | C |
| ANISOU | 553 | CB | BSER | A | 129 | 1325 | 1311 | 1351 | −13 | −27 | −36 | A | C |
| ATOM | 554 | OG | ASER | A | 129 | −43.878 | −20.388 | −11.810 | 0.50 | 8.95 | | A | O |
| ANISOU | 554 | OG | ASER | A | 129 | 1021 | 1134 | 1244 | −10 | −37 | −14 | A | O |
| ATOM | 555 | OG | BSER | A | 129 | −41.544 | −20.236 | −12.240 | 0.50 | 12.38 | | A | O |
| ANISOU | 555 | OG | BSER | A | 129 | 1514 | 1635 | 1555 | −3 | 28 | 21 | A | O |
| ATOM | 556 | C | ASER | A | 129 | −41.028 | −19.328 | −9.607 | 0.50 | 9.04 | | A | C |
| ANISOU | 556 | C | ASER | A | 129 | 1100 | 1199 | 1136 | −26 | −4 | 5 | A | C |
| ATOM | 557 | C | BSER | A | 129 | −41.034 | −19.289 | −9.558 | 0.50 | 9.51 | | A | C |
| ANISOU | 557 | C | BSER | A | 129 | 1172 | 1246 | 1194 | −37 | 1 | 4 | A | C |
| ATOM | 558 | O | ASER | A | 129 | −40.800 | −20.137 | −8.706 | 0.50 | 9.31 | | A | O |
| ANISOU | 558 | O | ASER | A | 129 | 1157 | 1292 | 1089 | 0 | 28 | 22 | A | O |
| ATOM | 559 | O | BSER | A | 129 | −40.815 | −20.038 | −8.604 | 0.50 | 10.03 | | A | O |
| ANISOU | 559 | O | BSER | A | 129 | 1260 | 1367 | 1185 | −14 | 6 | 27 | A | O |
| ATOM | 560 | N | TRP | A | 130 | −40.098 | −18.509 | −10.093 | 1.00 | 8.84 | | A | N |
| ANISOU | 560 | N | TRP | A | 130 | 1046 | 1216 | 1095 | −45 | −26 | −38 | A | N |
| ATOM | 561 | CA | TRP | A | 130 | −38.701 | −18.598 | −9.649 | 1.00 | 8.35 | | A | C |
| ANISOU | 561 | CA | TRP | A | 130 | 991 | 1172 | 1009 | −37 | 10 | −7 | A | C |
| ATOM | 562 | CB | TRP | A | 130 | −37.747 | −17.976 | −10.682 | 1.00 | 8.36 | | A | C |
| ANISOU | 562 | CB | TRP | A | 130 | 1034 | 1089 | 1054 | −50 | 25 | 43 | A | C |
| ATOM | 563 | CG | TRP | A | 130 | −37.778 | −16.479 | −10.695 | 1.00 | 7.48 | | A | C |
| ANISOU | 563 | CG | TRP | A | 130 | 867 | 1024 | 950 | −52 | −42 | −41 | A | C |
| ATOM | 564 | CD1 | TRP | A | 130 | −38.628 | −15.681 | −11.410 | 1.00 | 8.54 | | A | C |
| ANISOU | 564 | CD1 | TRP | A | 130 | 1066 | 1094 | 1084 | 38 | −22 | 8 | A | C |
| ATOM | 565 | NE1 | TRP | A | 130 | −38.361 | −14.355 | −11.161 | 1.00 | 8.31 | | A | N |
| ANISOU | 565 | NE1 | TRP | A | 130 | 925 | 1130 | 1102 | −120 | −19 | 21 | A | N |
| ATOM | 566 | CE2 | TRP | A | 130 | −37.326 | −14.273 | −10.261 | 1.00 | 7.83 | | A | C |
| ANISOU | 566 | CE2 | TRP | A | 130 | 1014 | 1003 | 957 | 20 | −75 | −29 | A | C |
| ATOM | 567 | CD2 | TRP | A | 130 | −36.927 | −15.594 | −9.950 | 1.00 | 7.27 | | A | C |
| ANISOU | 567 | CD2 | TRP | A | 130 | 891 | 995 | 877 | −119 | −1 | 27 | A | C |
| ATOM | 568 | CE3 | TRP | A | 130 | −35.872 | −15.791 | −9.046 | 1.00 | 7.85 | | A | C |
| ANISOU | 568 | CE3 | TRP | A | 130 | 1009 | 1003 | 971 | −47 | 10 | 104 | A | C |
| ATOM | 569 | CZ3 | TRP | A | 130 | −35.246 | −14.673 | −8.493 | 1.00 | 8.02 | | A | C |
| ANISOU | 569 | CZ3 | TRP | A | 130 | 1089 | 984 | 974 | −10 | −7 | −7 | A | C |
| ATOM | 570 | CH2 | TRP | A | 130 | −35.664 | −13.367 | −8.828 | 1.00 | 8.48 | | A | C |
| ANISOU | 570 | CH2 | TRP | A | 130 | 1055 | 1114 | 1054 | −17 | −53 | 50 | A | C |
| ATOM | 571 | CZ2 | TRP | A | 130 | −36.694 | −13.148 | −9.711 | 1.00 | 8.52 | | A | C |
| ANISOU | 571 | CZ2 | TRP | A | 130 | 1038 | 1170 | 1031 | −58 | −44 | 18 | A | C |
| ATOM | 572 | C | TRP | A | 130 | −38.459 | −17.980 | −8.270 | 1.00 | 8.45 | | A | C |
| ANISOU | 572 | C | TRP | A | 130 | 1057 | 1137 | 1015 | −24 | 3 | 20 | A | C |
| ATOM | 573 | O | TRP | A | 130 | −39.125 | −17.018 | −7.871 | 1.00 | 8.94 | | A | O |
| ANISOU | 573 | O | TRP | A | 130 | 1114 | 1210 | 1072 | 12 | −16 | 43 | A | O |
| ATOM | 574 | N | SER | A | 131 | −37.495 | −18.547 | −7.554 | 1.00 | 8.60 | | A | N |
| ANISOU | 574 | N | SER | A | 131 | 1087 | 1203 | 976 | −90 | −32 | 19 | A | N |
| ATOM | 575 | CA | SER | A | 131 | −36.959 | −17.911 | −6.357 | 1.00 | 8.21 | | A | C |
| ANISOU | 575 | CA | SER | A | 131 | 1009 | 1142 | 970 | −39 | −22 | −15 | A | C |
| ATOM | 576 | CB | SER | A | 131 | −37.801 | −18.247 | −5.118 | 1.00 | 8.81 | | A | C |
| ANISOU | 576 | CB | SER | A | 131 | 1179 | 1216 | 952 | 16 | 43 | −38 | A | C |
| ATOM | 577 | OG | SER | A | 131 | −37.711 | −19.622 | −4.798 | 1.00 | 10.00 | | A | O |
| ANISOU | 577 | OG | SER | A | 131 | 1276 | 1265 | 1257 | −59 | 47 | 52 | A | O |
| ATOM | 578 | C | SER | A | 131 | −35.510 | −18.336 | −6.160 | 1.00 | 8.09 | | A | C |
| ANISOU | 578 | C | SER | A | 131 | 995 | 1035 | 1042 | −27 | −21 | −3 | A | C |
| ATOM | 579 | O | SER | A | 131 | −35.093 | −19.407 | −6.614 | 1.00 | 8.79 | | A | O |
| ANISOU | 579 | O | SER | A | 131 | 1048 | 1136 | 1155 | −17 | −18 | −67 | A | O |
| ATOM | 580 | N | CYS | A | 132 | −34.736 | −17.490 | −5.492 | 1.00 | 7.88 | | A | N |
| ANISOU | 580 | N | CYS | A | 132 | 1007 | 969 | 1019 | −13 | −26 | −29 | A | N |
| ATOM | 581 | CA | CYS | A | 132 | −33.342 | −17.815 | −5.214 | 1.00 | 7.36 | | A | C |
| ANISOU | 581 | CA | CYS | A | 132 | 920 | 931 | 947 | −41 | −20 | −5 | A | C |
| ATOM | 582 | CB | CYS | A | 132 | −32.465 | −17.476 | −6.427 | 1.00 | 7.98 | | A | C |
| ANISOU | 582 | CB | CYS | A | 132 | 1049 | 1017 | 966 | 14 | 5 | −11 | A | C |
| ATOM | 583 | SG | CYS | A | 132 | −30.736 | −17.921 | −6.231 | 1.00 | 10.78 | | A | S |
| ANISOU | 583 | SG | CYS | A | 132 | 1190 | 1504 | 1403 | 12 | 65 | 43 | A | S |
| ATOM | 584 | C | CYS | A | 132 | −32.879 | −17.041 | −3.989 | 1.00 | 8.42 | | A | C |
| ANISOU | 584 | C | CYS | A | 132 | 1091 | 1042 | 1067 | −37 | −30 | −5 | A | C |
| ATOM | 585 | O | CYS | A | 132 | −32.920 | −15.812 | −3.988 | 1.00 | 8.81 | | A | O |
| ANISOU | 585 | O | CYS | A | 132 | 1186 | 961 | 1199 | −44 | −37 | −28 | A | O |
| ATOM | 586 | N | HIS | A | 133 | −32.464 | −17.759 | −2.947 | 1.00 | 8.66 | | A | N |
| ANISOU | 586 | N | HIS | A | 133 | 1105 | 1199 | 986 | −40 | −58 | −46 | A | N |
| ATOM | 587 | CA | HIS | A | 133 | −31.915 | −17.109 | −1.757 | 1.00 | 8.71 | | A | C |
| ANISOU | 587 | CA | HIS | A | 133 | 1145 | 1100 | 1064 | −43 | −36 | −15 | A | C |
| ATOM | 588 | CB | HIS | A | 133 | −32.153 | −17.957 | −0.499 | 1.00 | 9.67 | | A | C |
| ANISOU | 588 | CB | HIS | A | 133 | 1356 | 1188 | 1129 | 32 | −13 | 29 | A | C |
| ATOM | 589 | CG | HIS | A | 133 | −32.348 | −17.140 | 0.745 | 1.00 | 11.11 | | A | C |
| ANISOU | 589 | CG | HIS | A | 133 | 1656 | 1305 | 1261 | 50 | 0 | 49 | A | C |
| ATOM | 590 | ND1 | HIS | A | 133 | −31.321 | −16.454 | 1.354 | 1.00 | 12.16 | | A | N |
| ANISOU | 590 | ND1 | HIS | A | 133 | 1615 | 1536 | 1471 | 1 | 4 | 202 | A | N |
| ATOM | 591 | CE1 | HIS | A | 133 | −31.783 | −15.817 | 2.414 | 1.00 | 12.03 | | A | C |
| ANISOU | 591 | CE1 | HIS | A | 133 | 1532 | 1505 | 1532 | 40 | 16 | 110 | A | C |
| ATOM | 592 | NE2 | HIS | A | 133 | −33.077 | −16.073 | 2.521 | 1.00 | 14.12 | | A | N |
| ANISOU | 592 | NE2 | HIS | A | 133 | 1707 | 1785 | 1872 | −78 | −82 | −7 | A | N |
| ATOM | 593 | CD2 | HIS | A | 133 | −33.454 | −16.899 | 1.490 | 1.00 | 13.39 | | A | C |
| ANISOU | 593 | CD2 | HIS | A | 133 | 1739 | 1678 | 1672 | 108 | −6 | −3 | A | C |
| ATOM | 594 | C | HIS | A | 133 | −30.425 | −16.855 | −1.976 | 1.00 | 9.12 | | A | C |
| ANISOU | 594 | C | HIS | A | 133 | 1165 | 1120 | 1180 | −35 | −20 | −37 | A | C |
| ATOM | 595 | O | HIS | A | 133 | −29.694 | −17.744 | −2.409 | 1.00 | 10.56 | | A | O |
| ANISOU | 595 | O | HIS | A | 133 | 1299 | 1259 | 1456 | −7 | −29 | −174 | A | O |
| ATOM | 596 | N | ALA | A | 134 | −29.974 | −15.634 | −1.707 | 1.00 | 7.59 | | A | N |
| ANISOU | 596 | N | ALA | A | 134 | 849 | 994 | 1040 | −46 | −9 | −69 | A | N |
| ATOM | 597 | CA | ALA | A | 134 | −28.542 | −15.337 | −1.829 | 1.00 | 7.45 | | A | C |
| ANISOU | 597 | CA | ALA | A | 134 | 913 | 946 | 973 | −37 | 41 | −43 | A | C |
| ATOM | 598 | CB | ALA | A | 134 | −28.240 | −14.723 | −3.186 | 1.00 | 8.49 | | A | C |
| ANISOU | 598 | CB | ALA | A | 134 | 1075 | 1127 | 1022 | −13 | 88 | 46 | A | C |
| ATOM | 599 | C | ALA | A | 134 | −28.017 | −14.443 | −0.714 | 1.00 | 7.55 | | A | C |
| ANISOU | 599 | C | ALA | A | 134 | 981 | 962 | 925 | 44 | −14 | −11 | A | C |
| ATOM | 600 | O | ALA | A | 134 | −28.754 | −13.602 | −0.188 | 1.00 | 7.77 | | A | O |
| ANISOU | 600 | O | ALA | A | 134 | 973 | 1019 | 959 | 9 | −64 | −148 | A | O |
| ATOM | 601 | N | GLN | A | 135 | −26.747 | −14.669 | −0.367 | 1.00 | 7.28 | | A | N |
| ANISOU | 601 | N | GLN | A | 135 | 920 | 878 | 969 | −103 | −43 | −19 | A | N |
| ATOM | 602 | CA | GLN | A | 135 | −25.924 | −13.764 | 0.435 | 1.00 | 7.54 | | A | C |
| ANISOU | 602 | CA | GLN | A | 135 | 946 | 945 | 973 | −44 | −36 | 10 | A | C |
| ATOM | 603 | CB | GLN | A | 135 | −24.893 | −14.555 | 1.251 | 1.00 | 8.34 | | A | C |
| ANISOU | 603 | CB | GLN | A | 135 | 1029 | 1059 | 1079 | −16 | −2 | 108 | A | C |
| ATOM | 604 | CG | GLN | A | 135 | −25.450 | −15.697 | 2.082 | 1.00 | 8.06 | | A | C |
| ANISOU | 604 | CG | GLN | A | 135 | 1103 | 948 | 1011 | −53 | −16 | −1 | A | C |
| ATOM | 605 | CD | GLN | A | 135 | −26.329 | −15.216 | 3.217 | 1.00 | 8.59 | | A | C |
| ANISOU | 605 | CD | GLN | A | 135 | 1089 | 1100 | 1074 | 17 | 23 | 37 | A | C |
| ATOM | 606 | OE1 | GLN | A | 135 | −27.532 | −15.470 | 3.237 | 1.00 | 9.30 | | A | O |
| ANISOU | 606 | OE1 | GLN | A | 135 | 1247 | 1044 | 1241 | −125 | −130 | −34 | A | O |
| ATOM | 607 | NE2 | GLN | A | 135 | −25.732 | −14.503 | 4.165 | 1.00 | 9.92 | | A | N |
| ANISOU | 607 | NE2 | GLN | A | 135 | 1455 | 1095 | 1219 | −33 | −72 | −90 | A | N |
| ATOM | 608 | C | GLN | A | 135 | −25.155 | −12.879 | −0.531 | 1.00 | 7.46 | | A | C |
| ANISOU | 608 | C | GLN | A | 135 | 980 | 920 | 936 | −47 | −11 | −18 | A | C |
| ATOM | 609 | O | GLN | A | 135 | −24.818 | −13.313 | −1.631 | 1.00 | 9.19 | | A | O |
| ANISOU | 609 | O | GLN | A | 135 | 1315 | 1044 | 1134 | 1 | 67 | −81 | A | O |
| ATOM | 610 | N | ALA | A | 136 | −24.853 | −11.648 | −0.130 | 1.00 | 7.15 | | A | N |
| ANISOU | 610 | N | ALA | A | 136 | 982 | 800 | 936 | −47 | 47 | 37 | A | N |
| ATOM | 611 | CA | ALA | A | 136 | −23.932 | −10.834 | −0.926 | 1.00 | 7.19 | | A | C |
| ANISOU | 611 | CA | ALA | A | 136 | 964 | 893 | 874 | −50 | −3 | 48 | A | C |
| ATOM | 612 | CB | ALA | A | 136 | −24.649 | −10.178 | −2.116 | 1.00 | 7.20 | | A | C |
| ANISOU | 612 | CB | ALA | A | 136 | 906 | 975 | 854 | −60 | −46 | 99 | A | C |
| ATOM | 613 | C | ALA | A | 136 | −23.197 | −9.786 | −0.113 | 1.00 | 7.48 | | A | C |
| ANISOU | 613 | C | ALA | A | 136 | 964 | 906 | 973 | −23 | 12 | −2 | A | C |
| ATOM | 614 | O | ALA | A | 136 | −23.715 | −9.270 | 0.876 | 1.00 | 8.26 | | A | O |
| ANISOU | 614 | O | ALA | A | 136 | 1092 | 1093 | 955 | 9 | 67 | −9 | A | O |
| ATOM | 615 | N | VAL | A | 137 | −21.983 | −9.481 | −0.553 | 1.00 | 6.86 | | A | N |
| ANISOU | 615 | N | VAL | A | 137 | 893 | 855 | 858 | −15 | 66 | 2 | A | N |
| ATOM | 616 | CA | VAL | A | 137 | −21.244 | −8.335 | −0.054 | 1.00 | 7.09 | | A | C |
| ANISOU | 616 | CA | VAL | A | 137 | 889 | 912 | 891 | −47 | 16 | 57 | A | C |
| ATOM | 617 | CB | VAL | A | 137 | −19.767 | −8.687 | 0.255 | 1.00 | 8.52 | | A | C |
| ANISOU | 617 | CB | VAL | A | 137 | 996 | 1125 | 1117 | 21 | 37 | 10 | A | C |
| ATOM | 618 | CG1 | VAL | A | 137 | −19.040 | −7.471 | 0.802 | 1.00 | 8.96 | | A | C |
| ANISOU | 618 | CG1 | VAL | A | 137 | 989 | 1129 | 1286 | 63 | −58 | 24 | A | C |
| ATOM | 619 | CG2 | VAL | A | 137 | −19.686 | −9.847 | 1.245 | 1.00 | 9.79 | | A | C |
| ANISOU | 619 | CG2 | VAL | A | 137 | 1253 | 1257 | 1210 | 36 | −79 | 25 | A | C |
| ATOM | 620 | C | VAL | A | 137 | −21.309 | −7.256 | −1.132 | 1.00 | 7.60 | | A | C |
| ANISOU | 620 | C | VAL | A | 137 | 991 | 976 | 921 | 18 | −2 | 27 | A | C |
| ATOM | 621 | O | VAL | A | 137 | −20.846 | −7.465 | −2.260 | 1.00 | 8.81 | | A | O |
| ANISOU | 621 | O | VAL | A | 137 | 1244 | 1095 | 1007 | −24 | 54 | 22 | A | O |
| ATOM | 622 | N | LEU | A | 138 | −21.885 | −6.122 | −0.777 | 1.00 | 7.11 | | A | N |
| ANISOU | 622 | N | LEU | A | 138 | 941 | 868 | 892 | −57 | −41 | 35 | A | N |
| ATOM | 623 | CA | LEU | A | 138 | −21.980 | −4.966 | −1.639 | 1.00 | 7.21 | | A | C |
| ANISOU | 623 | CA | LEU | A | 138 | 926 | 872 | 942 | 39 | −5 | 33 | A | C |
| ATOM | 624 | CB | LEU | A | 138 | −23.367 | −4.332 | −1.535 | 1.00 | 7.73 | | A | C |
| ANISOU | 624 | CB | LEU | A | 138 | 858 | 1062 | 1017 | −70 | −65 | −43 | A | C |
| ATOM | 625 | CG | LEU | A | 138 | −24.444 | −4.880 | −2.470 | 1.00 | 7.29 | | A | C |
| ANISOU | 625 | CG | LEU | A | 138 | 868 | 978 | 924 | −1 | −81 | 0 | A | C |
| ATOM | 626 | CD1 | LEU | A | 138 | −24.637 | −6.356 | −2.194 | 1.00 | 10.19 | | A | C |
| ANISOU | 626 | CD1 | LEU | A | 138 | 1352 | 1216 | 1304 | 61 | −7 | −41 | A | C |
| ATOM | 627 | CD2 | LEU | A | 138 | −25.750 | −4.149 | −2.279 | 1.00 | 8.92 | | A | C |
| ANISOU | 627 | CD2 | LEU | A | 138 | 1101 | 1112 | 1176 | 60 | −25 | −10 | A | C |
| ATOM | 628 | C | LEU | A | 138 | −20.887 | −3.960 | −1.253 | 1.00 | 7.98 | | A | C |
| ANISOU | 628 | C | LEU | A | 138 | 1018 | 996 | 1017 | −24 | 32 | −51 | A | C |
| ATOM | 629 | O | LEU | A | 138 | −20.891 | −3.452 | −0.184 | 1.00 | 8.00 | | A | O |
| ANISOU | 629 | O | LEU | A | 138 | 1081 | 1131 | 829 | −11 | −10 | −28 | A | O |
| ATOM | 630 | N | LYS | A | 139 | −19.962 | −3.727 | −2.180 | 1.00 | 8.32 | | A | N |
| ANISOU | 630 | N | LYS | A | 139 | 1115 | 1054 | 993 | −24 | 49 | −10 | A | N |
| ATOM | 631 | CA | LYS | A | 139 | −18.766 | −2.963 | −1.906 | 1.00 | 8.61 | | A | C |
| ANISOU | 631 | CA | LYS | A | 139 | 1125 | 1065 | 1083 | −48 | 9 | −15 | A | C |
| ATOM | 632 | CB | LYS | A | 139 | −17.553 | −3.895 | −1.976 | 1.00 | 8.79 | | A | C |
| ANISOU | 632 | CB | LYS | A | 139 | 1204 | 1075 | 1061 | −11 | 66 | −15 | A | C |
| ATOM | 633 | CG | LYS | A | 139 | −16.213 | −3.289 | −1.618 | 1.00 | 10.07 | | A | C |
| ANISOU | 633 | CG | LYS | A | 139 | 1246 | 1266 | 1315 | −1 | −1 | −52 | A | C |
| ATOM | 634 | CD | LYS | A | 139 | −15.100 | −4.276 | −1.884 | 1.00 | 14.75 | | A | C |
| ANISOU | 634 | CD | LYS | A | 139 | 1675 | 1873 | 2055 | 199 | 40 | −153 | A | C |
| ATOM | 635 | CE | LYS | A | 139 | −15.112 | −5.328 | −0.866 | 1.00 | 20.39 | | A | C |
| ANISOU | 635 | CE | LYS | A | 139 | 2645 | 2594 | 2507 | −101 | −52 | 151 | A | C |
| ATOM | 636 | NZ | LYS | A | 139 | −14.258 | −6.510 | −1.192 | 1.00 | 26.36 | | A | N |
| ANISOU | 636 | NZ | LYS | A | 139 | 3328 | 3271 | 3418 | 177 | 10 | −27 | A | N |
| ATOM | 637 | C | LYS | A | 139 | −18.576 | −1.818 | −2.908 | 1.00 | 8.91 | | A | C |
| ANISOU | 637 | C | LYS | A | 139 | 1186 | 1086 | 1114 | −48 | −10 | −42 | A | C |
| ATOM | 638 | O | LYS | A | 139 | −18.818 | −2.009 | −4.049 | 1.00 | 9.28 | | A | O |
| ANISOU | 638 | O | LYS | A | 139 | 1377 | 1076 | 1073 | −83 | 36 | −27 | A | O |
| ATOM | 639 | N | AILE | A | 140 | −18.202 | −0.638 | −2.384 | 0.50 | 8.33 | | A | N |
| ANISOU | 639 | N | AILE | A | 140 | 1066 | 1037 | 1063 | −36 | 9 | −9 | A | N |
| ATOM | 640 | N | BILE | A | 140 | −18.171 | −0.659 | −2.369 | 0.50 | 9.02 | | A | N |
| ANISOU | 640 | N | BILE | A | 140 | 1160 | 1122 | 1147 | −43 | 2 | −30 | A | N |
| ATOM | 641 | CA | AILE | A | 140 | −17.676 | 0.417 | −3.252 | 0.50 | 8.55 | | A | C |
| ANISOU | 641 | CA | AILE | A | 140 | 1085 | 1035 | 1128 | 0 | −13 | −7 | A | C |
| ATOM | 642 | CA | BILE | A | 140 | −17.678 | 0.428 | −3.205 | 0.50 | 9.80 | | A | C |
| ANISOU | 642 | CA | BILE | A | 140 | 1243 | 1213 | 1266 | −20 | −7 | −2 | A | C |
| ATOM | 643 | CB | AILE | A | 140 | −18.287 | 1.818 | −2.977 | 0.50 | 8.53 | | A | C |
| ANISOU | 643 | CB | AILE | A | 140 | 1043 | 1068 | 1129 | 29 | −34 | −22 | A | C |
| ATOM | 644 | CB | BILE | A | 140 | −18.363 | 1.785 | −2.863 | 0.50 | 10.23 | | A | C |
| ANISOU | 644 | CB | BILE | A | 140 | 1305 | 1272 | 1309 | 16 | −13 | −15 | A | C |
| ATOM | 645 | CG1 | AILE | A | 140 | −19.805 | 1.795 | −3.173 | 0.50 | 7.58 | | A | C |
| ANISOU | 645 | CG1 | AILE | A | 140 | 949 | 861 | 1069 | 20 | −25 | −10 | A | C |
| ATOM | 646 | CG1 | BILE | A | 140 | −17.909 | 2.883 | −3.828 | 0.50 | 10.77 | | A | C |
| ANISOU | 646 | CG1 | BILE | A | 140 | 1307 | 1367 | 1420 | 6 | 0 | 32 | A | C |
| ATOM | 647 | CD1 | AILE | A | 140 | −20.494 | 3.156 | −2.978 | 0.50 | 7.51 | | A | C |
| ANISOU | 647 | CD1 | AILE | A | 140 | 975 | 813 | 1067 | 24 | −35 | −16 | A | C |
| ATOM | 648 | CD1 | BILE | A | 140 | −18.637 | 4.189 | −3.642 | 0.50 | 10.60 | | A | C |
| ANISOU | 648 | CD1 | BILE | A | 140 | 1411 | 1254 | 1364 | −42 | 8 | 8 | A | C |
| ATOM | 649 | CG2 | AILE | A | 140 | −17.658 | 2.865 | −3.898 | 0.50 | 10.28 | | A | C |
| ANISOU | 649 | CG2 | AILE | A | 140 | 1298 | 1297 | 1312 | −10 | 8 | 60 | A | C |
| ATOM | 650 | CG2 | BILE | A | 140 | −18.146 | 2.175 | −1.391 | 0.50 | 10.58 | | A | C |
| ANISOU | 650 | CG2 | BILE | A | 140 | 1322 | 1366 | 1333 | 18 | −47 | −49 | A | C |
| ATOM | 651 | C | AILE | A | 140 | −16.166 | 0.454 | −3.066 | 0.50 | 9.45 | | A | C |
| ANISOU | 651 | C | AILE | A | 140 | 1154 | 1202 | 1233 | 2 | −8 | −6 | A | C |
| ATOM | 652 | C | BILE | A | 140 | −16.154 | 0.483 | −3.058 | 0.50 | 10.21 | | A | C |
| ANISOU | 652 | C | BILE | A | 140 | 1274 | 1303 | 1301 | −1 | 10 | −7 | A | C |
| ATOM | 653 | O | AILE | A | 140 | −15.670 | 0.553 | −1.943 | 0.50 | 10.07 | | A | O |
| ANISOU | 653 | O | AILE | A | 140 | 1264 | 1349 | 1212 | −16 | −19 | −36 | A | O |
| ATOM | 654 | O | BILE | A | 140 | −15.634 | 0.603 | −1.947 | 0.50 | 10.83 | | A | O |
| ANISOU | 654 | O | BILE | A | 140 | 1373 | 1446 | 1297 | −16 | −9 | −33 | A | O |
| ATOM | 655 | N | ILE | A | 141 | −15.432 | 0.390 | −4.140 | 1.00 | 10.09 | | A | N |
| ANISOU | 655 | N | ILE | A | 141 | 1236 | 1274 | 1325 | −40 | 31 | −31 | A | N |
| ATOM | 656 | CA | ILE | A | 141 | −13.986 | 0.298 | −4.150 | 1.00 | 11.31 | | A | C |
| ANISOU | 656 | CA | ILE | A | 141 | 1337 | 1493 | 1469 | −10 | −26 | 0 | A | C |
| ATOM | 657 | CB | ILE | A | 141 | −13.474 | −0.261 | −5.486 | 1.00 | 12.21 | | A | C |
| ANISOU | 657 | CB | ILE | A | 141 | 1483 | 1557 | 1600 | −26 | 26 | −52 | A | C |
| ATOM | 658 | CG1 | ILE | A | 141 | −14.131 | −1.604 | −5.814 | 1.00 | 13.49 | | A | C |
| ANISOU | 658 | CG1 | ILE | A | 141 | 1648 | 1734 | 1742 | −71 | −10 | −32 | A | C |
| ATOM | 659 | CD1 | ILE | A | 141 | −13.737 | −2.703 | −4.963 | 1.00 | 15.27 | | A | C |
| ANISOU | 659 | CD1 | ILE | A | 141 | 1941 | 1879 | 1983 | 17 | −17 | −1 | A | C |
| ATOM | 660 | CG2 | ILE | A | 141 | −11.957 | −0.372 | −5.499 | 1.00 | 14.29 | | A | C |
| ANISOU | 660 | CG2 | ILE | A | 141 | 1610 | 1951 | 1869 | 12 | −7 | −28 | A | C |
| ATOM | 661 | C | ILE | A | 141 | −13.373 | 1.676 | −3.916 | 1.00 | 12.83 | | A | C |
| ANISOU | 661 | C | ILE | A | 141 | 1641 | 1575 | 1657 | −46 | −36 | −9 | A | C |
| ATOM | 662 | O | ILE | A | 141 | −13.717 | 2.638 | −4.559 | 1.00 | 13.68 | | A | O |
| ANISOU | 662 | O | ILE | A | 141 | 1782 | 1687 | 1727 | −69 | −114 | 46 | A | O |
| ATOM | 663 | N | ASN | A | 142 | −12.471 | 1.744 | −2.956 | 1.00 | 14.16 | | A | N |
| ANISOU | 663 | N | ASN | A | 142 | 1763 | 1842 | 1774 | −19 | −75 | 44 | A | N |
| ATOM | 664 | CA | ASN | A | 142 | −11.670 | 2.941 | −2.777 | 1.00 | 16.00 | | A | C |
| ANISOU | 664 | CA | ASN | A | 142 | 2057 | 1975 | 2049 | −59 | −38 | −5 | A | C |
| ATOM | 665 | CB | ASN | A | 142 | −11.263 | 3.105 | −1.310 | 1.00 | 16.09 | | A | C |
| ANISOU | 665 | CB | ASN | A | 142 | 2049 | 2063 | 2003 | −11 | −23 | 38 | A | C |
| ATOM | 666 | CG | ASN | A | 142 | −10.795 | 4.513 | −0.984 | 1.00 | 17.33 | | A | C |
| ANISOU | 666 | CG | ASN | A | 142 | 2239 | 2182 | 2165 | −26 | −17 | 2 | A | C |
| ATOM | 667 | OD1 | ASN | A | 142 | −9.649 | 4.874 | −1.246 | 1.00 | 19.37 | | A | O |
| ANISOU | 667 | OD1 | ASN | A | 142 | 2284 | 2590 | 2486 | −113 | 22 | 38 | A | O |
| ATOM | 668 | ND2 | ASN | A | 142 | −11.680 | 5.309 | −0.397 | 1.00 | 17.60 | | A | N |
| ANISOU | 668 | ND2 | ASN | A | 142 | 2228 | 2274 | 2185 | −7 | −51 | 6 | A | N |
| ATOM | 669 | C | ASN | A | 142 | −10.456 | 2.789 | −3.684 | 1.00 | 18.32 | | A | C |
| ANISOU | 669 | C | ASN | A | 142 | 2255 | 2338 | 2369 | −15 | 26 | −6 | A | C |
| ATOM | 670 | O | ASN | A | 142 | −9.644 | 1.878 | −3.496 | 1.00 | 18.15 | | A | O |
| ANISOU | 670 | O | ASN | A | 142 | 2153 | 2323 | 2422 | −57 | 86 | 51 | A | O |
| ATOM | 671 | N | TYR | A | 143 | −10.363 | 3.651 | −4.695 | 1.00 | 21.17 | | A | N |
| ANISOU | 671 | N | TYR | A | 143 | 2728 | 2649 | 2668 | −36 | −32 | 58 | A | N |
| ATOM | 672 | CA | TYR | A | 143 | −9.307 | 3.535 | −5.699 | 1.00 | 24.71 | | A | C |
| ANISOU | 672 | CA | TYR | A | 143 | 3073 | 3205 | 3112 | 0 | 59 | −14 | A | C |
| ATOM | 673 | CB | TYR | A | 143 | −9.673 | 4.304 | −6.980 | 1.00 | 26.93 | | A | C |
| ANISOU | 673 | CB | TYR | A | 143 | 3369 | 3582 | 3281 | 46 | −4 | 51 | A | C |
| ATOM | 674 | CG | TYR | A | 143 | −9.139 | 5.717 | −7.046 | 1.00 | 30.45 | | A | C |
| ANISOU | 674 | CG | TYR | A | 143 | 3917 | 3786 | 3866 | −5 | −51 | 27 | A | C |
| ATOM | 675 | CD1 | TYR | A | 143 | −7.960 | 6.005 | −7.735 | 1.00 | 31.76 | | A | C |
| ANISOU | 675 | CD1 | TYR | A | 143 | 3984 | 4037 | 4046 | 42 | 30 | 1 | A | C |
| ATOM | 676 | CE1 | TYR | A | 143 | −7.458 | 7.302 | −7.794 | 1.00 | 32.72 | | A | C |
| ANISOU | 676 | CE1 | TYR | A | 143 | 4164 | 4083 | 4187 | 0 | 8 | 4 | A | C |
| ATOM | 677 | CZ | TYR | A | 143 | −8.141 | 8.328 | −7.160 | 1.00 | 32.35 | | A | C |
| ANISOU | 677 | CZ | TYR | A | 143 | 4123 | 4024 | 4145 | −46 | −20 | −1 | A | C |
| ATOM | 678 | OH | TYR | A | 143 | −7.654 | 9.613 | −7.219 | 1.00 | 32.61 | | A | O |
| ANISOU | 678 | OH | TYR | A | 143 | 4132 | 4048 | 4210 | −99 | 23 | −7 | A | O |
| ATOM | 679 | CE2 | TYR | A | 143 | −9.316 | 8.069 | −6.469 | 1.00 | 32.44 | | A | C |
| ANISOU | 679 | CE2 | TYR | A | 143 | 4150 | 4079 | 4097 | −16 | 10 | 14 | A | C |
| ATOM | 680 | CD2 | TYR | A | 143 | −9.809 | 6.768 | −6.417 | 1.00 | 31.86 | | A | C |
| ANISOU | 680 | CD2 | TYR | A | 143 | 4027 | 4064 | 4013 | 55 | 37 | −15 | A | C |
| ATOM | 681 | C | TYR | A | 143 | −7.937 | 3.954 | −5.155 | 1.00 | 25.52 | | A | C |
| ANISOU | 681 | C | TYR | A | 143 | 3166 | 3267 | 3264 | −35 | 16 | 24 | A | C |
| ATOM | 682 | O | TYR | A | 143 | −6.910 | 3.454 | −5.614 | 1.00 | 25.74 | | A | O |
| ANISOU | 682 | O | TYR | A | 143 | 3238 | 3224 | 3318 | 2 | 51 | 21 | A | O |
| ATOM | 683 | N | ARG | A | 144 | −7.929 | 4.862 | −4.178 | 1.00 | 26.54 | | A | N |
| ANISOU | 683 | N | ARG | A | 144 | 3337 | 3386 | 3362 | −35 | 35 | −13 | A | N |
| ATOM | 684 | CA | ARG | A | 144 | −6.677 | 5.348 | −3.582 | 1.00 | 28.03 | | A | C |
| ANISOU | 684 | CA | ARG | A | 144 | 3475 | 3600 | 3575 | −48 | −5 | −19 | A | C |
| ATOM | 685 | CB | ARG | A | 144 | −6.883 | 6.694 | −2.879 | 1.00 | 28.74 | | A | C |
| ANISOU | 685 | CB | ARG | A | 144 | 3551 | 3683 | 3684 | −32 | 15 | −76 | A | C |
| ATOM | 686 | CG | ARG | A | 144 | −6.975 | 7.870 | −3.828 | 1.00 | 32.40 | | A | C |
| ANISOU | 686 | CG | ARG | A | 144 | 4117 | 4121 | 4073 | 24 | −10 | 136 | A | C |
| ATOM | 687 | CD | ARG | A | 144 | −6.626 | 9.180 | −3.144 | 1.00 | 36.69 | | A | C |
| ANISOU | 687 | CD | ARG | A | 144 | 4709 | 4547 | 4684 | −37 | −26 | −139 | A | C |
| ATOM | 688 | NE | ARG | A | 144 | −6.673 | 10.301 | −4.082 | 1.00 | 39.42 | | A | N |
| ANISOU | 688 | NE | ARG | A | 144 | 5109 | 4916 | 4954 | −4 | −15 | 55 | A | N |
| ATOM | 689 | CZ | ARG | A | 144 | −5.662 | 10.686 | −4.859 | 1.00 | 40.47 | | A | C |
| ANISOU | 689 | CZ | ARG | A | 144 | 5121 | 5142 | 5113 | −20 | 23 | 0 | A | C |
| ATOM | 690 | NH1 | ARG | A | 144 | −4.499 | 10.047 | −4.825 | 1.00 | 41.15 | | A | N |
| ANISOU | 690 | NH1 | ARG | A | 144 | 5203 | 5208 | 5223 | 22 | 9 | 0 | A | N |
| ATOM | 691 | NH2 | ARG | A | 144 | −5.817 | 11.717 | −5.678 | 1.00 | 40.63 | | A | N |
| ANISOU | 691 | NH2 | ARG | A | 144 | 5146 | 5134 | 5158 | −6 | 12 | 8 | A | N |
| ATOM | 692 | C | ARG | A | 144 | −6.057 | 4.349 | −2.613 | 1.00 | 28.31 | | A | C |
| ANISOU | 692 | C | ARG | A | 144 | 3536 | 3608 | 3612 | −8 | 3 | −6 | A | C |
| ATOM | 693 | O | ARG | A | 144 | −4.837 | 4.185 | −2.579 | 1.00 | 28.35 | | A | O |
| ANISOU | 693 | O | ARG | A | 144 | 3513 | 3633 | 3625 | −55 | 34 | −8 | A | O |
| ATOM | 694 | N | ASP | A | 145 | −6.903 | 3.689 | −1.828 | 1.00 | 28.70 | | A | N |
| ANISOU | 694 | N | ASP | A | 145 | 3583 | 3657 | 3666 | −24 | 25 | −1 | A | N |
| ATOM | 695 | CA | ASP | A | 145 | −6.452 | 2.719 | −0.837 | 1.00 | 29.49 | | A | C |
| ANISOU | 695 | CA | ASP | A | 145 | 3693 | 3769 | 3741 | −4 | 1 | 26 | A | C |
| ATOM | 696 | CB | ASP | A | 145 | −6.292 | 3.399 | 0.532 | 1.00 | 29.90 | | A | C |
| ANISOU | 696 | CB | ASP | A | 145 | 3731 | 3832 | 3797 | −17 | 9 | −14 | A | C |
| ATOM | 697 | CG | ASP | A | 145 | −5.665 | 2.490 | 1.585 | 1.00 | 31.64 | | A | C |
| ANISOU | 697 | CG | ASP | A | 145 | 3937 | 4044 | 4041 | 1 | −23 | 76 | A | C |
| ATOM | 698 | OD1 | ASP | A | 145 | −5.312 | 1.331 | 1.278 | 1.00 | 32.60 | | A | O |
| ANISOU | 698 | OD1 | ASP | A | 145 | 4059 | 4161 | 4168 | 37 | 2 | −5 | A | O |
| ATOM | 699 | OD2 | ASP | A | 145 | −5.524 | 2.951 | 2.739 | 1.00 | 33.81 | | A | O |
| ANISOU | 699 | OD2 | ASP | A | 145 | 4304 | 4346 | 4196 | 16 | −32 | −26 | A | O |
| ATOM | 700 | C | ASP | A | 145 | −7.443 | 1.560 | −0.769 | 1.00 | 29.99 | | A | C |
| ANISOU | 700 | C | ASP | A | 145 | 3772 | 3834 | 3790 | −11 | −15 | 19 | A | C |
| ATOM | 701 | O | ASP | A | 145 | −8.519 | 1.683 | −0.182 | 1.00 | 29.17 | | A | O |
| ANISOU | 701 | O | ASP | A | 145 | 3644 | 3734 | 3706 | −18 | −70 | 35 | A | O |
| ATOM | 702 | N | ASP | A | 146 | −7.059 | 0.436 | −1.374 | 1.00 | 31.04 | | A | N |
| ANISOU | 702 | N | ASP | A | 146 | 3942 | 3926 | 3924 | 31 | −8 | −1 | A | N |
| ATOM | 703 | CA | ASP | A | 146 | −7.897 | −0.766 | −1.450 | 1.00 | 32.43 | | A | C |
| ANISOU | 703 | CA | ASP | A | 146 | 4105 | 4112 | 4106 | −29 | −10 | 10 | A | C |
| ATOM | 704 | CB | ASP | A | 146 | −7.213 | −1.848 | −2.295 | 1.00 | 33.63 | | A | C |
| ANISOU | 704 | CB | ASP | A | 146 | 4333 | 4221 | 4223 | 6 | 27 | −16 | A | C |
| ATOM | 705 | CG | ASP | A | 146 | −5.744 | −2.029 | −1.944 | 1.00 | 36.98 | | A | C |
| ANISOU | 705 | CG | ASP | A | 146 | 4532 | 4828 | 4692 | 33 | −41 | −38 | A | C |
| ATOM | 706 | OD1 | ASP | A | 146 | −4.945 | −1.100 | −2.201 | 1.00 | 38.83 | | A | O |
| ANISOU | 706 | OD1 | ASP | A | 146 | 4891 | 4893 | 4968 | −52 | 8 | 23 | A | O |
| ATOM | 707 | OD2 | ASP | A | 146 | −5.388 | −3.106 | −1.420 | 1.00 | 39.29 | | A | O |
| ANISOU | 707 | OD2 | ASP | A | 146 | 4981 | 4896 | 5050 | 25 | −38 | 66 | A | O |
| ATOM | 708 | C | ASP | A | 146 | −8.291 | −1.334 | −0.083 | 1.00 | 32.23 | | A | C |
| ANISOU | 708 | C | ASP | A | 146 | 4085 | 4100 | 4060 | −18 | −22 | 1 | A | C |
| ATOM | 709 | O | ASP | A | 146 | −9.197 | −2.165 | 0.011 | 1.00 | 33.23 | | A | O |
| ANISOU | 709 | O | ASP | A | 146 | 4212 | 4180 | 4232 | −66 | −44 | 25 | A | O |
| ATOM | 710 | N | GLU | A | 147 | −7.608 | −0.881 | 0.967 | 1.00 | 31.31 | | A | N |
| ANISOU | 710 | N | GLU | A | 147 | 3953 | 3992 | 3953 | 4 | 5 | 23 | A | N |
| ATOM | 711 | CA | GLU | A | 147 | −7.949 | −1.259 | 2.337 | 1.00 | 30.28 | | A | C |
| ANISOU | 711 | CA | GLU | A | 147 | 3800 | 3857 | 3849 | 22 | −5 | −13 | A | C |
| ATOM | 712 | CB | GLU | A | 147 | −6.688 | −1.322 | 3.216 | 1.00 | 30.72 | | A | C |
| ANISOU | 712 | CB | GLU | A | 147 | 3869 | 3912 | 3892 | −17 | −22 | 20 | A | C |
| ATOM | 713 | CG | GLU | A | 147 | −5.731 | −2.459 | 2.843 | 1.00 | 31.95 | | A | C |
| ANISOU | 713 | CG | GLU | A | 147 | 3986 | 4076 | 4079 | 27 | −13 | −18 | A | C |
| ATOM | 714 | CD | GLU | A | 147 | −4.599 | −2.655 | 3.844 | 1.00 | 33.14 | | A | C |
| ANISOU | 714 | CD | GLU | A | 147 | 4125 | 4299 | 4169 | 18 | −26 | 11 | A | C |
| ATOM | 715 | OE1 | GLU | A | 147 | −4.052 | −1.651 | 4.352 | 1.00 | 33.55 | | A | O |
| ANISOU | 715 | OE1 | GLU | A | 147 | 4225 | 4252 | 4271 | −24 | −16 | −29 | A | O |
| ATOM | 716 | OE2 | GLU | A | 147 | −4.246 | −3.826 | 4.111 | 1.00 | 34.62 | | A | O |
| ANISOU | 716 | OE2 | GLU | A | 147 | 4404 | 4319 | 4430 | 49 | 0 | 31 | A | O |
| ATOM | 717 | C | GLU | A | 147 | −9.002 | −0.324 | 2.944 | 1.00 | 28.43 | | A | C |
| ANISOU | 717 | C | GLU | A | 147 | 3607 | 3613 | 3581 | −22 | −39 | 59 | A | C |
| ATOM | 718 | O | GLU | A | 147 | −9.500 | −0.576 | 4.044 | 1.00 | 28.31 | | A | O |
| ANISOU | 718 | O | GLU | A | 147 | 3575 | 3646 | 3537 | 3 | −39 | 47 | A | O |
| ATOM | 719 | N | LYS | A | 148 | −9.346 | 0.741 | 2.215 | 1.00 | 26.25 | | A | N |
| ANISOU | 719 | N | LYS | A | 148 | 3267 | 3391 | 3315 | −7 | −7 | −7 | A | N |
| ATOM | 720 | CA | LYS | A | 148 | −10.344 | 1.718 | 2.665 | 1.00 | 23.89 | | A | C |
| ANISOU | 720 | CA | LYS | A | 148 | 2969 | 3148 | 2959 | −60 | −88 | 24 | A | C |
| ATOM | 721 | CB | LYS | A | 148 | −9.805 | 3.149 | 2.563 | 1.00 | 25.36 | | A | C |
| ANISOU | 721 | CB | LYS | A | 148 | 3228 | 3228 | 3178 | −35 | −11 | 13 | A | C |
| ATOM | 722 | CG | LYS | A | 148 | −8.724 | 3.497 | 3.583 | 1.00 | 28.07 | | A | C |
| ANISOU | 722 | CG | LYS | A | 148 | 3447 | 3745 | 3473 | −91 | −120 | 7 | A | C |
| ATOM | 723 | CD | LYS | A | 148 | −8.972 | 4.851 | 4.262 | 1.00 | 32.08 | | A | C |
| ANISOU | 723 | CD | LYS | A | 148 | 4079 | 4007 | 4104 | 47 | 14 | −117 | A | C |
| ATOM | 724 | CE | LYS | A | 148 | −8.736 | 6.050 | 3.351 | 1.00 | 35.01 | | A | C |
| ANISOU | 724 | CE | LYS | A | 148 | 4556 | 4356 | 4390 | −28 | 45 | 40 | A | C |
| ATOM | 725 | NZ | LYS | A | 148 | −9.949 | 6.443 | 2.570 | 1.00 | 37.34 | | A | N |
| ANISOU | 725 | NZ | LYS | A | 148 | 4662 | 4778 | 4746 | 24 | −31 | 5 | A | N |
| ATOM | 726 | C | LYS | A | 148 | −11.676 | 1.612 | 1.909 | 1.00 | 21.28 | | A | C |
| ANISOU | 726 | C | LYS | A | 148 | 2725 | 2709 | 2650 | −22 | 2 | −4 | A | C |
| ATOM | 727 | O | LYS | A | 148 | −12.519 | 2.508 | 1.997 | 1.00 | 21.11 | | A | O |
| ANISOU | 727 | O | LYS | A | 148 | 2694 | 2754 | 2573 | −34 | −35 | 5 | A | O |
| ATOM | 728 | N | SER | A | 149 | −11.862 | 0.521 | 1.172 | 1.00 | 18.28 | | A | N |
| ANISOU | 728 | N | SER | A | 149 | 2199 | 2515 | 2233 | −14 | −66 | 89 | A | N |
| ATOM | 729 | CA | SER | A | 149 | −13.141 | 0.259 | 0.508 | 1.00 | 15.80 | | A | C |
| ANISOU | 729 | CA | SER | A | 149 | 1967 | 2097 | 1938 | −23 | 55 | 53 | A | C |
| ATOM | 730 | CB | SER | A | 149 | −13.028 | −0.934 | −0.440 | 1.00 | 15.64 | | A | C |
| ANISOU | 730 | CB | SER | A | 149 | 1989 | 2066 | 1889 | −18 | 92 | 77 | A | C |
| ATOM | 731 | OG | SER | A | 149 | −12.049 | −0.688 | −1.439 | 1.00 | 14.78 | | A | O |
| ANISOU | 731 | OG | SER | A | 149 | 1739 | 2074 | 1803 | 59 | 83 | 140 | A | O |
| ATOM | 732 | C | SER | A | 149 | −14.245 | 0.041 | 1.544 | 1.00 | 14.52 | | A | C |
| ANISOU | 732 | C | SER | A | 149 | 1756 | 1947 | 1813 | 4 | −35 | 73 | A | C |
| ATOM | 733 | O | SER | A | 149 | −13.971 | −0.289 | 2.704 | 1.00 | 15.50 | | A | O |
| ANISOU | 733 | O | SER | A | 149 | 1866 | 2188 | 1834 | 13 | −47 | 140 | A | O |
| ATOM | 734 | N | PHE | A | 150 | −15.490 | 0.243 | 1.127 | 1.00 | 11.64 | | A | N |
| ANISOU | 734 | N | PHE | A | 150 | 1501 | 1512 | 1411 | −80 | 30 | 79 | A | N |
| ATOM | 735 | CA | PHE | A | 150 | −16.615 | 0.193 | 2.048 | 1.00 | 11.37 | | A | C |
| ANISOU | 735 | CA | PHE | A | 150 | 1413 | 1416 | 1492 | −94 | −3 | −44 | A | C |
| ATOM | 736 | CB | PHE | A | 150 | −17.286 | 1.564 | 2.146 | 1.00 | 12.04 | | A | C |
| ANISOU | 736 | CB | PHE | A | 150 | 1554 | 1487 | 1535 | −25 | −55 | −28 | A | C |
| ATOM | 737 | CG | PHE | A | 150 | −18.481 | 1.593 | 3.065 | 1.00 | 13.68 | | A | C |
| ANISOU | 737 | CG | PHE | A | 150 | 1729 | 1619 | 1849 | −29 | 67 | −71 | A | C |
| ATOM | 738 | CD1 | PHE | A | 150 | −19.717 | 2.006 | 2.595 | 1.00 | 16.32 | | A | C |
| ANISOU | 738 | CD1 | PHE | A | 150 | 1947 | 2111 | 2141 | 6 | −7 | −47 | A | C |
| ATOM | 739 | CE1 | PHE | A | 150 | −20.828 | 2.037 | 3.438 | 1.00 | 15.88 | | A | C |
| ANISOU | 739 | CE1 | PHE | A | 150 | 1796 | 2101 | 2138 | 0 | 38 | 24 | A | C |
| ATOM | 740 | CZ | PHE | A | 150 | −20.705 | 1.639 | 4.763 | 1.00 | 16.38 | | A | C |
| ANISOU | 740 | CZ | PHE | A | 150 | 1916 | 2139 | 2168 | 23 | 39 | −54 | A | C |
| ATOM | 741 | CE2 | PHE | A | 150 | −19.472 | 1.217 | 5.247 | 1.00 | 17.08 | | A | C |
| ANISOU | 741 | CE2 | PHE | A | 150 | 2058 | 2201 | 2229 | 16 | −17 | 21 | A | C |
| ATOM | 742 | CD2 | PHE | A | 150 | −18.370 | 1.189 | 4.397 | 1.00 | 16.08 | | A | C |
| ANISOU | 742 | CD2 | PHE | A | 150 | 2072 | 2070 | 1969 | −31 | 12 | −34 | A | C |
| ATOM | 743 | C | PHE | A | 150 | −17.622 | −0.841 | 1.589 | 1.00 | 10.87 | | A | C |
| ANISOU | 743 | C | PHE | A | 150 | 1376 | 1397 | 1358 | −92 | −20 | 21 | A | C |
| ATOM | 744 | O | PHE | A | 150 | −18.052 | −0.816 | 0.441 | 1.00 | 10.76 | | A | O |
| ANISOU | 744 | O | PHE | A | 150 | 1367 | 1353 | 1369 | −129 | −5 | −28 | A | O |
| ATOM | 745 | N | SER | A | 151 | −18.004 | −1.738 | 2.492 | 1.00 | 9.88 | | A | N |
| ANISOU | 745 | N | SER | A | 151 | 1220 | 1274 | 1261 | −114 | 6 | −40 | A | N |
| ATOM | 746 | CA | SER | A | 151 | −18.947 | −2.785 | 2.134 | 1.00 | 10.87 | | A | C |
| ANISOU | 746 | CA | SER | A | 151 | 1316 | 1387 | 1427 | −119 | 38 | −27 | A | C |
| ATOM | 747 | CB | SER | A | 151 | −18.217 | −4.079 | 1.790 | 1.00 | 13.13 | | A | C |
| ANISOU | 747 | CB | SER | A | 151 | 1695 | 1554 | 1738 | −29 | 56 | −86 | A | C |
| ATOM | 748 | OG | SER | A | 151 | −17.541 | −4.582 | 2.914 | 1.00 | 16.50 | | A | O |
| ANISOU | 748 | OG | SER | A | 151 | 2134 | 1974 | 2163 | 153 | −121 | −4 | A | O |
| ATOM | 749 | C | SER | A | 151 | −19.982 | −3.035 | 3.210 | 1.00 | 9.39 | | A | C |
| ANISOU | 749 | C | SER | A | 151 | 1129 | 1260 | 1180 | −77 | −37 | −24 | A | C |
| ATOM | 750 | O | SER | A | 151 | −19.743 | −2.786 | 4.398 | 1.00 | 9.62 | | A | O |
| ANISOU | 750 | O | SER | A | 151 | 1142 | 1254 | 1260 | −133 | 9 | −97 | A | O |
| ATOM | 751 | N | ARG | A | 152 | −21.135 | −3.535 | 2.767 | 1.00 | 8.45 | | A | N |
| ANISOU | 751 | N | ARG | A | 152 | 1057 | 1098 | 1054 | −133 | 38 | −14 | A | N |
| ATOM | 752 | CA | ARG | A | 152 | −22.254 | −3.876 | 3.640 | 1.00 | 7.46 | | A | C |
| ANISOU | 752 | CA | ARG | A | 152 | 916 | 950 | 970 | 20 | 25 | −7 | A | C |
| ATOM | 753 | CB | ARG | A | 152 | −23.293 | −2.746 | 3.636 | 1.00 | 7.32 | | A | C |
| ANISOU | 753 | CB | ARG | A | 152 | 947 | 944 | 890 | 42 | −25 | −27 | A | C |
| ATOM | 754 | CG | ARG | A | 152 | −22.759 | −1.427 | 4.199 | 1.00 | 7.25 | | A | C |
| ANISOU | 754 | CG | ARG | A | 152 | 973 | 882 | 901 | 27 | −57 | −15 | A | C |
| ATOM | 755 | CD | ARG | A | 152 | −23.690 | −0.249 | 3.965 | 1.00 | 7.62 | | A | C |
| ANISOU | 755 | CD | ARG | A | 152 | 939 | 915 | 1043 | 38 | 69 | −21 | A | C |
| ATOM | 756 | NE | ARG | A | 152 | −24.907 | −0.319 | 4.770 | 1.00 | 8.37 | | A | N |
| ANISOU | 756 | NE | ARG | A | 152 | 1056 | 1062 | 1064 | 43 | 87 | −23 | A | N |
| ATOM | 757 | CZ | ARG | A | 152 | −25.798 | 0.665 | 4.861 | 1.00 | 7.20 | | A | C |
| ANISOU | 757 | CZ | ARG | A | 152 | 991 | 812 | 934 | −22 | 54 | 61 | A | C |
| ATOM | 758 | NH1 | ARG | A | 152 | −25.597 | 1.813 | 4.214 | 1.00 | 6.94 | | A | N |
| ANISOU | 758 | NH1 | ARG | A | 152 | 1188 | 687 | 760 | −46 | −12 | −42 | A | N |
| ATOM | 759 | NH2 | ARG | A | 152 | −26.881 | 0.509 | 5.611 | 1.00 | 6.93 | | A | N |
| ANISOU | 759 | NH2 | ARG | A | 152 | 915 | 852 | 866 | 23 | 49 | 38 | A | N |
| ATOM | 760 | C | ARG | A | 152 | −22.855 | −5.179 | 3.125 | 1.00 | 7.98 | | A | C |
| ANISOU | 760 | C | ARG | A | 152 | 1049 | 996 | 988 | 30 | 5 | −17 | A | C |
| ATOM | 761 | O | ARG | A | 152 | −22.976 | −5.364 | 1.911 | 1.00 | 8.37 | | A | O |
| ANISOU | 761 | O | ARG | A | 152 | 1158 | 1070 | 952 | −12 | 54 | 8 | A | O |
| ATOM | 762 | N | ARG | A | 153 | −23.222 | −6.071 | 4.047 | 1.00 | 7.77 | | A | N |
| ANISOU | 762 | N | ARG | A | 153 | 929 | 1011 | 1011 | 0 | 51 | −15 | A | N |
| ATOM | 763 | CA | ARG | A | 153 | −23.685 | −7.422 | 3.711 | 1.00 | 7.96 | | A | C |
| ANISOU | 763 | CA | ARG | A | 153 | 1055 | 1003 | 965 | −13 | 5 | −9 | A | C |
| ATOM | 764 | CB | ARG | A | 153 | −23.178 | −8.439 | 4.739 | 1.00 | 9.24 | | A | C |
| ANISOU | 764 | CB | ARG | A | 153 | 1151 | 1143 | 1216 | 18 | −18 | 57 | A | C |
| ATOM | 765 | CG | ARG | A | 153 | −21.682 | −8.706 | 4.706 | 1.00 | 9.60 | | A | C |
| ANISOU | 765 | CG | ARG | A | 153 | 1155 | 1353 | 1140 | 77 | −14 | 85 | A | C |
| ATOM | 766 | CD | ARG | A | 153 | −21.275 | −9.601 | 5.871 | 1.00 | 10.87 | | A | C |
| ANISOU | 766 | CD | ARG | A | 153 | 1438 | 1507 | 1187 | 88 | −102 | 16 | A | C |
| ATOM | 767 | NE | ARG | A | 153 | −21.454 | −8.924 | 7.156 | 1.00 | 10.59 | | A | N |
| ANISOU | 767 | NE | ARG | A | 153 | 1290 | 1450 | 1285 | 104 | 63 | −55 | A | N |
| ATOM | 768 | CZ | ARG | A | 153 | −21.306 | −9.511 | 8.340 | 1.00 | 12.76 | | A | C |
| ANISOU | 768 | CZ | ARG | A | 153 | 1783 | 1579 | 1486 | −7 | −88 | 52 | A | C |
| ATOM | 769 | NH1 | ARG | A | 153 | −20.967 | −10.794 | 8.414 | 1.00 | 13.03 | | A | N |
| ANISOU | 769 | NH1 | ARG | A | 153 | 1796 | 1581 | 1572 | 155 | −16 | −83 | A | N |
| ATOM | 770 | NH2 | ARG | A | 153 | −21.495 | −8.814 | 9.456 | 1.00 | 13.53 | | A | N |
| ANISOU | 770 | NH2 | ARG | A | 153 | 1860 | 1619 | 1660 | 141 | 62 | −54 | A | N |
| ATOM | 771 | C | ARG | A | 153 | −25.201 | −7.525 | 3.662 | 1.00 | 8.40 | | A | C |
| ANISOU | 771 | C | ARG | A | 153 | 1100 | 1062 | 1028 | 3 | 20 | −23 | A | C |
| ATOM | 772 | O | ARG | A | 153 | −25.906 | −6.869 | 4.445 | 1.00 | 8.52 | | A | O |
| ANISOU | 772 | O | ARG | A | 153 | 1170 | 1007 | 1060 | 16 | 39 | −96 | A | O |
| ATOM | 773 | N | ILE | A | 154 | −25.689 | −8.382 | 2.763 | 1.00 | 7.37 | | A | N |
| ANISOU | 773 | N | ILE | A | 154 | 920 | 931 | 950 | −24 | −2 | −3 | A | N |
| ATOM | 774 | CA | ILE | A | 154 | −27.122 | −8.676 | 2.643 | 1.00 | 8.09 | | A | C |
| ANISOU | 774 | CA | ILE | A | 154 | 1028 | 1024 | 1023 | −18 | −78 | −18 | A | C |
| ATOM | 775 | CB | ILE | A | 154 | −27.765 | −8.019 | 1.380 | 1.00 | 8.49 | | A | C |
| ANISOU | 775 | CB | ILE | A | 154 | 1204 | 965 | 1058 | −11 | −10 | 20 | A | C |
| ATOM | 776 | CG1 | ILE | A | 154 | −27.137 | −8.577 | 0.093 | 1.00 | 9.25 | | A | C |
| ANISOU | 776 | CG1 | ILE | A | 154 | 1297 | 1219 | 997 | 32 | 16 | −8 | A | C |
| ATOM | 777 | CD1 | ILE | A | 154 | −27.899 | −8.237 | −1.179 | 1.00 | 9.93 | | A | C |
| ANISOU | 777 | CD1 | ILE | A | 154 | 1391 | 1296 | 1086 | 94 | −61 | −28 | A | C |
| ATOM | 778 | CG2 | ILE | A | 154 | −27.684 | −6.493 | 1.449 | 1.00 | 9.18 | | A | C |
| ANISOU | 778 | CG2 | ILE | A | 154 | 1304 | 996 | 1189 | 87 | −7 | −56 | A | C |
| ATOM | 779 | C | ILE | A | 154 | −27.398 | −10.180 | 2.607 | 1.00 | 8.06 | | A | C |
| ANISOU | 779 | C | ILE | A | 154 | 1018 | 1068 | 977 | −10 | −4 | 10 | A | C |
| ATOM | 780 | O | ILE | A | 154 | −26.501 | −10.985 | 2.346 | 1.00 | 8.34 | | A | O |
| ANISOU | 780 | O | ILE | A | 154 | 1053 | 1057 | 1057 | 47 | −50 | −15 | A | O |
| ATOM | 781 | N | SER | A | 155 | −28.651 | −10.536 | 2.887 | 1.00 | 7.39 | | A | N |
| ANISOU | 781 | N | SER | A | 155 | 898 | 967 | 943 | −59 | −69 | −71 | A | N |
| ATOM | 782 | CA | SER | A | 155 | −29.171 | −11.896 | 2.756 | 1.00 | 7.41 | | A | C |
| ANISOU | 782 | CA | SER | A | 155 | 985 | 888 | 944 | −22 | 19 | 58 | A | C |
| ATOM | 783 | CB | SER | A | 155 | −29.049 | −12.641 | 4.092 | 1.00 | 7.78 | | A | C |
| ANISOU | 783 | CB | SER | A | 155 | 1133 | 924 | 900 | −46 | −54 | 15 | A | C |
| ATOM | 784 | OG | SER | A | 155 | −29.624 | −13.931 | 3.988 | 1.00 | 9.60 | | A | O |
| ANISOU | 784 | OG | SER | A | 155 | 1308 | 1078 | 1260 | −139 | 79 | 67 | A | O |
| ATOM | 785 | C | SER | A | 155 | −30.636 | −11.766 | 2.375 | 1.00 | 7.60 | | A | C |
| ANISOU | 785 | C | SER | A | 155 | 1024 | 950 | 912 | −32 | −68 | −54 | A | C |
| ATOM | 786 | O | SER | A | 155 | −31.384 | −11.068 | 3.058 | 1.00 | 8.97 | | A | O |
| ANISOU | 786 | O | SER | A | 155 | 1037 | 1256 | 1117 | 38 | −22 | −158 | A | O |
| ATOM | 787 | N | HIS | A | 156 | −31.064 | −12.421 | 1.294 | 1.00 | 6.78 | | A | N |
| ANISOU | 787 | N | HIS | A | 156 | 822 | 891 | 863 | −42 | −55 | 11 | A | N |
| ATOM | 788 | CA | HIS | A | 156 | −32.408 | −12.158 | 0.768 | 1.00 | 6.99 | | A | C |
| ANISOU | 788 | CA | HIS | A | 156 | 908 | 830 | 918 | −44 | −77 | −13 | A | C |
| ATOM | 789 | CB | HIS | A | 156 | −32.398 | −10.819 | 0.006 | 1.00 | 7.48 | | A | C |
| ANISOU | 789 | CB | HIS | A | 156 | 959 | 941 | 942 | −5 | −100 | 97 | A | C |
| ATOM | 790 | CG | HIS | A | 156 | −33.685 | −10.473 | −0.685 | 1.00 | 8.74 | | A | C |
| ANISOU | 790 | CG | HIS | A | 156 | 1043 | 1183 | 1096 | −13 | −103 | 3 | A | C |
| ATOM | 791 | ND1 | HIS | A | 156 | −34.843 | −10.157 | −0.005 | 1.00 | 9.94 | | A | N |
| ANISOU | 791 | ND1 | HIS | A | 156 | 1232 | 1228 | 1318 | −39 | −69 | −4 | A | N |
| ATOM | 792 | CE1 | HIS | A | 156 | −35.796 | −9.868 | −0.875 | 1.00 | 9.31 | | A | C |
| ANISOU | 792 | CE1 | HIS | A | 156 | 1224 | 1189 | 1126 | −4 | 66 | 117 | A | C |
| ATOM | 793 | NE2 | HIS | A | 156 | −35.296 | −9.977 | −2.094 | 1.00 | 8.52 | | A | N |
| ANISOU | 793 | NE2 | HIS | A | 156 | 1102 | 1049 | 1088 | 147 | 45 | 10 | A | N |
| ATOM | 794 | CD2 | HIS | A | 156 | −33.975 | −10.346 | −2.003 | 1.00 | 7.82 | | A | C |
| ANISOU | 794 | CD2 | HIS | A | 156 | 990 | 979 | 1004 | −102 | −43 | 73 | A | C |
| ATOM | 795 | C | HIS | A | 156 | −32.914 | −13.282 | −0.124 | 1.00 | 7.26 | | A | C |
| ANISOU | 795 | C | HIS | A | 156 | 938 | 900 | 919 | −24 | −42 | −48 | A | C |
| ATOM | 796 | O | HIS | A | 156 | −32.153 | −13.844 | −0.921 | 1.00 | 7.76 | | A | O |
| ANISOU | 796 | O | HIS | A | 156 | 1001 | 982 | 965 | 55 | −44 | −60 | A | O |
| ATOM | 797 | N | LEU | A | 157 | −34.198 | −13.602 | 0.021 | 1.00 | 7.31 | | A | N |
| ANISOU | 797 | N | LEU | A | 157 | 919 | 887 | 973 | −19 | −29 | −23 | A | N |
| ATOM | 798 | CA | LEU | A | 157 | −34.883 | −14.466 | −0.940 | 1.00 | 7.39 | | A | C |
| ANISOU | 798 | CA | LEU | A | 157 | 910 | 973 | 924 | −66 | −71 | 8 | A | C |
| ATOM | 799 | CB | LEU | A | 157 | −36.075 | −15.194 | −0.304 | 1.00 | 8.06 | | A | C |
| ANISOU | 799 | CB | LEU | A | 157 | 977 | 1112 | 975 | −31 | 17 | 0 | A | C |
| ATOM | 800 | CG | LEU | A | 157 | −36.843 | −16.156 | −1.225 | 1.00 | 8.39 | | A | C |
| ANISOU | 800 | CG | LEU | A | 157 | 962 | 1044 | 1181 | −70 | 63 | −25 | A | C |
| ATOM | 801 | CD1 | LEU | A | 157 | −36.010 | −17.390 | −1.572 | 1.00 | 10.21 | | A | C |
| ANISOU | 801 | CD1 | LEU | A | 157 | 1443 | 1138 | 1298 | 121 | 27 | −15 | A | C |
| ATOM | 802 | CD2 | LEU | A | 157 | −38.161 | −16.573 | −0.582 | 1.00 | 8.51 | | A | C |
| ANISOU | 802 | CD2 | LEU | A | 157 | 966 | 1115 | 1151 | −119 | 83 | 6 | A | C |
| ATOM | 803 | C | LEU | A | 157 | −35.358 | −13.626 | −2.117 | 1.00 | 7.23 | | A | C |
| ANISOU | 803 | C | LEU | A | 157 | 903 | 950 | 895 | −30 | −10 | 21 | A | C |
| ATOM | 804 | O | LEU | A | 157 | −36.324 | −12.862 | −2.001 | 1.00 | 7.46 | | A | O |
| ANISOU | 804 | O | LEU | A | 157 | 1085 | 896 | 853 | −3 | 6 | −15 | A | O |
| ATOM | 805 | N | PHE | A | 158 | −34.673 | −13.770 | −3.248 | 1.00 | 7.89 | | A | N |
| ANISOU | 805 | N | PHE | A | 158 | 1066 | 1031 | 900 | −26 | −33 | 37 | A | N |
| ATOM | 806 | CA | PHE | A | 158 | −35.055 | −13.071 | −4.472 | 1.00 | 7.30 | | A | C |
| ANISOU | 806 | CA | PHE | A | 158 | 919 | 1009 | 846 | −19 | −21 | 14 | A | C |
| ATOM | 807 | CB | PHE | A | 158 | −33.851 | −12.939 | −5.418 | 1.00 | 7.47 | | A | C |
| ANISOU | 807 | CB | PHE | A | 158 | 840 | 1000 | 1000 | −47 | 8 | −34 | A | C |
| ATOM | 808 | CG | PHE | A | 158 | −32.756 | −12.047 | −4.886 | 1.00 | 7.06 | | A | C |
| ANISOU | 808 | CG | PHE | A | 158 | 899 | 838 | 945 | 15 | −54 | −50 | A | C |
| ATOM | 809 | CD1 | PHE | A | 158 | −31.832 | −12.526 | −3.952 | 1.00 | 7.57 | | A | C |
| ANISOU | 809 | CD1 | PHE | A | 158 | 973 | 1018 | 885 | −57 | −73 | −46 | A | C |
| ATOM | 810 | CE1 | PHE | A | 158 | −30.825 | −11.687 | −3.445 | 1.00 | 7.26 | | A | C |
| ANISOU | 810 | CE1 | PHE | A | 158 | 919 | 922 | 919 | −48 | −49 | 54 | A | C |
| ATOM | 811 | CZ | PHE | A | 158 | −30.730 | −10.373 | −3.896 | 1.00 | 7.71 | | A | C |
| ANISOU | 811 | CZ | PHE | A | 158 | 1027 | 920 | 984 | −49 | 36 | −41 | A | C |
| ATOM | 812 | CE2 | PHE | A | 158 | −31.643 | −9.890 | −4.829 | 1.00 | 7.49 | | A | C |
| ANISOU | 812 | CE2 | PHE | A | 158 | 978 | 937 | 930 | −68 | 58 | −33 | A | C |
| ATOM | 813 | CD2 | PHE | A | 158 | −32.650 | −10.726 | −5.318 | 1.00 | 7.76 | | A | C |
| ANISOU | 813 | CD2 | PHE | A | 158 | 950 | 933 | 1065 | −45 | 48 | −19 | A | C |
| ATOM | 814 | C | PHE | A | 158 | −36.201 | −13.808 | −5.154 | 1.00 | 7.67 | | A | C |
| ANISOU | 814 | C | PHE | A | 158 | 978 | 978 | 959 | −14 | 0 | 23 | A | C |
| ATOM | 815 | O | PHE | A | 158 | −36.210 | −15.031 | −5.212 | 1.00 | 7.39 | | A | O |
| ANISOU | 815 | O | PHE | A | 158 | 1024 | 931 | 853 | 40 | −56 | −84 | A | O |
| ATOM | 816 | N | PHE | A | 159 | −37.178 | −13.042 | −5.631 | 1.00 | 7.55 | | A | N |
| ANISOU | 816 | N | PHE | A | 159 | 915 | 1052 | 902 | −4 | −71 | 1 | A | N |
| ATOM | 817 | CA | PHE | A | 159 | −38.284 | −13.554 | −6.448 | 1.00 | 7.48 | | A | C |
| ANISOU | 817 | CA | PHE | A | 159 | 846 | 993 | 1004 | −26 | −57 | 55 | A | C |
| ATOM | 818 | CB | PHE | A | 159 | −39.281 | −14.383 | −5.620 | 1.00 | 7.89 | | A | C |
| ANISOU | 818 | CB | PHE | A | 159 | 951 | 1070 | 976 | −25 | 31 | 47 | A | C |
| ATOM | 819 | CG | PHE | A | 159 | −40.067 | −13.586 | −4.608 | 1.00 | 8.47 | | A | C |
| ANISOU | 819 | CG | PHE | A | 159 | 1121 | 1109 | 988 | −2 | −7 | −4 | A | C |
| ATOM | 820 | CD1 | PHE | A | 159 | −41.380 | −13.217 | −4.875 | 1.00 | 10.47 | | A | C |
| ANISOU | 820 | CD1 | PHE | A | 159 | 1256 | 1407 | 1315 | 62 | 103 | −25 | A | C |
| ATOM | 821 | CE1 | PHE | A | 159 | −42.124 | −12.500 | −3.944 | 1.00 | 11.38 | | A | C |
| ANISOU | 821 | CE1 | PHE | A | 159 | 1547 | 1461 | 1316 | 77 | 67 | −105 | A | C |
| ATOM | 822 | CZ | PHE | A | 159 | −41.553 | −12.140 | −2.731 | 1.00 | 10.24 | | A | C |
| ANISOU | 822 | CZ | PHE | A | 159 | 1443 | 1240 | 1209 | 39 | 58 | −48 | A | C |
| ATOM | 823 | CE2 | PHE | A | 159 | −40.249 | −12.509 | −2.441 | 1.00 | 10.72 | | A | C |
| ANISOU | 823 | CE2 | PHE | A | 159 | 1373 | 1484 | 1215 | −4 | 122 | −19 | A | C |
| ATOM | 824 | CD2 | PHE | A | 159 | −39.507 | −13.234 | −3.380 | 1.00 | 9.25 | | A | C |
| ANISOU | 824 | CD2 | PHE | A | 159 | 1313 | 1067 | 1133 | 3 | 17 | −26 | A | C |
| ATOM | 825 | C | PHE | A | 159 | −38.938 | −12.357 | −7.131 | 1.00 | 8.40 | | A | C |
| ANISOU | 825 | C | PHE | A | 159 | 1054 | 1081 | 1057 | 13 | −5 | 42 | A | C |
| ATOM | 826 | O | PHE | A | 159 | −38.514 | −11.227 | −6.910 | 1.00 | 9.21 | | A | O |
| ANISOU | 826 | O | PHE | A | 159 | 1198 | 1157 | 1144 | 36 | −65 | 47 | A | O |
| ATOM | 827 | N | HIS | A | 160 | −39.958 | −12.603 | −7.949 | 1.00 | 9.79 | | A | N |
| ANISOU | 827 | N | HIS | A | 160 | 1158 | 1306 | 1256 | 25 | −91 | −6 | A | N |
| ATOM | 828 | CA | HIS | A | 160 | −40.540 | −11.554 | −8.802 | 1.00 | 10.74 | | A | C |
| ANISOU | 828 | CA | HIS | A | 160 | 1372 | 1349 | 1358 | −1 | −93 | 34 | A | C |
| ATOM | 829 | CB | HIS | A | 160 | −41.714 | −12.095 | −9.640 | 1.00 | 11.16 | | A | C |
| ANISOU | 829 | CB | HIS | A | 160 | 1375 | 1422 | 1445 | −3 | −148 | −8 | A | C |
| ATOM | 830 | CG | HIS | A | 160 | −42.960 | −12.382 | −8.854 | 1.00 | 12.87 | | A | C |
| ANISOU | 830 | CG | HIS | A | 160 | 1592 | 1613 | 1684 | 30 | 22 | −43 | A | C |
| ATOM | 831 | ND1 | HIS | A | 160 | −43.133 | −13.537 | −8.116 | 1.00 | 13.38 | | A | N |
| ANISOU | 831 | ND1 | HIS | A | 160 | 1717 | 1824 | 1542 | 64 | 80 | −41 | A | N |
| ATOM | 832 | CE1 | HIS | A | 160 | −44.330 | −13.522 | −7.555 | 1.00 | 14.93 | | A | C |
| ANISOU | 832 | CE1 | HIS | A | 160 | 1914 | 1897 | 1861 | −79 | 75 | 42 | A | C |
| ATOM | 833 | NE2 | HIS | A | 160 | −44.941 | −12.404 | −7.904 | 1.00 | 16.71 | | A | N |
| ANISOU | 833 | NE2 | HIS | A | 160 | 1980 | 2205 | 2163 | 26 | 90 | 42 | A | N |
| ATOM | 834 | CD2 | HIS | A | 160 | −44.108 | −11.674 | −8.718 | 1.00 | 13.36 | | A | C |
| ANISOU | 834 | CD2 | HIS | A | 160 | 1650 | 1699 | 1727 | 43 | −67 | −36 | A | C |
| ATOM | 835 | C | HIS | A | 160 | −40.916 | −10.251 | −8.088 | 1.00 | 12.03 | | A | C |
| ANISOU | 835 | C | HIS | A | 160 | 1495 | 1509 | 1568 | 44 | −39 | −13 | A | C |
| ATOM | 836 | O | HIS | A | 160 | −40.606 | −9.164 | −8.592 | 1.00 | 13.34 | | A | O |
| ANISOU | 836 | O | HIS | A | 160 | 1811 | 1605 | 1653 | −31 | 8 | 58 | A | O |
| ATOM | 837 | N | LYS | A | 161 | −41.554 | −10.357 | −6.923 | 1.00 | 12.44 | | A | N |
| ANISOU | 837 | N | LYS | A | 161 | 1502 | 1632 | 1594 | 45 | −19 | 9 | A | N |
| ATOM | 838 | CA | LYS | A | 161 | −42.019 | −9.172 | −6.174 | 1.00 | 13.18 | | A | C |
| ANISOU | 838 | CA | LYS | A | 161 | 1682 | 1640 | 1685 | 20 | −43 | −87 | A | C |
| ATOM | 839 | CB | LYS | A | 161 | −43.061 | −9.554 | −5.116 | 1.00 | 14.68 | | A | C |
| ANISOU | 839 | CB | LYS | A | 161 | 1744 | 1852 | 1980 | 37 | 75 | −27 | A | C |
| ATOM | 840 | CG | LYS | A | 161 | −44.325 | −10.236 | −5.609 | 1.00 | 16.73 | | A | C |
| ANISOU | 840 | CG | LYS | A | 161 | 2081 | 2126 | 2149 | −54 | −45 | −70 | A | C |
| ATOM | 841 | CD | LYS | A | 161 | −45.213 | −10.629 | −4.426 | 1.00 | 18.35 | | A | C |
| ANISOU | 841 | CD | LYS | A | 161 | 2356 | 2353 | 2263 | 52 | 57 | 27 | A | C |
| ATOM | 842 | CE | LYS | A | 161 | −46.378 | −11.507 | −4.858 | 1.00 | 21.33 | | A | C |
| ANISOU | 842 | CE | LYS | A | 161 | 2687 | 2666 | 2753 | −148 | −14 | −44 | A | C |
| ATOM | 843 | NZ | LYS | A | 161 | −47.283 | −11.851 | −3.717 | 1.00 | 23.16 | | A | N |
| ANISOU | 843 | NZ | LYS | A | 161 | 2925 | 2937 | 2937 | −38 | 88 | 37 | A | N |
| ATOM | 844 | C | LYS | A | 161 | −40.887 | −8.432 | −5.458 | 1.00 | 11.78 | | A | C |
| ANISOU | 844 | C | LYS | A | 161 | 1442 | 1447 | 1585 | 34 | −9 | 15 | A | C |
| ATOM | 845 | O | LYS | A | 161 | −41.033 | −7.264 | −5.101 | 1.00 | 12.80 | | A | O |
| ANISOU | 845 | O | LYS | A | 161 | 1514 | 1606 | 1744 | 91 | −101 | −98 | A | O |
| ATOM | 846 | N | GLU | A | 162 | −39.783 | −9.132 | −5.212 | 1.00 | 10.06 | | A | N |
| ANISOU | 846 | N | GLU | A | 162 | 1256 | 1255 | 1312 | −40 | −28 | 7 | A | N |
| ATOM | 847 | CA | GLU | A | 162 | −38.626 | −8.556 | −4.518 | 1.00 | 9.39 | | A | C |
| ANISOU | 847 | CA | GLU | A | 162 | 1145 | 1236 | 1187 | 17 | −13 | −25 | A | C |
| ATOM | 848 | CB | GLU | A | 162 | −38.607 | −8.960 | −3.033 | 1.00 | 9.64 | | A | C |
| ANISOU | 848 | CB | GLU | A | 162 | 1233 | 1211 | 1219 | 2 | 59 | 1 | A | C |
| ATOM | 849 | CG | GLU | A | 162 | −39.862 | −8.534 | −2.251 | 1.00 | 10.48 | | A | C |
| ANISOU | 849 | CG | GLU | A | 162 | 1214 | 1453 | 1314 | 119 | 18 | −9 | A | C |
| ATOM | 850 | CD | GLU | A | 162 | −39.719 | −8.653 | −0.739 | 1.00 | 12.33 | | A | C |
| ANISOU | 850 | CD | GLU | A | 162 | 1647 | 1618 | 1421 | 54 | 44 | 45 | A | C |
| ATOM | 851 | OE1 | GLU | A | 162 | −40.551 | −8.045 | −0.019 | 1.00 | 15.74 | | A | O |
| ANISOU | 851 | OE1 | GLU | A | 162 | 2008 | 2186 | 1788 | 174 | 213 | −55 | A | O |
| ATOM | 852 | OE2 | GLU | A | 162 | −38.800 | −9.353 | −0.264 | 1.00 | 11.73 | | A | O |
| ANISOU | 852 | OE2 | GLU | A | 162 | 1503 | 1625 | 1328 | 36 | 101 | −27 | A | O |
| ATOM | 853 | C | GLU | A | 162 | −37.364 | −9.020 | −5.231 | 1.00 | 9.24 | | A | C |
| ANISOU | 853 | C | GLU | A | 162 | 1181 | 1213 | 1116 | 11 | −55 | −39 | A | C |
| ATOM | 854 | O | GLU | A | 162 | −36.562 | −9.782 | −4.679 | 1.00 | 8.70 | | A | O |
| ANISOU | 854 | O | GLU | A | 162 | 1089 | 1166 | 1050 | 13 | −24 | 46 | A | O |
| ATOM | 855 | N | ASN | A | 163 | −37.210 | −8.579 | −6.477 | 1.00 | 8.31 | | A | N |
| ANISOU | 855 | N | ASN | A | 163 | 1045 | 1098 | 1016 | −88 | 31 | 1 | A | N |
| ATOM | 856 | CA | ASN | A | 163 | −36.124 | −9.073 | −7.321 | 1.00 | 8.08 | | A | C |
| ANISOU | 856 | CA | ASN | A | 163 | 935 | 1096 | 1040 | −14 | −31 | −42 | A | C |
| ATOM | 857 | CB | ASN | A | 163 | −36.556 | −9.246 | −8.792 | 1.00 | 9.33 | | A | C |
| ANISOU | 857 | CB | ASN | A | 163 | 1147 | 1318 | 1080 | −83 | −3 | 23 | A | C |
| ATOM | 858 | CG | ASN | A | 163 | −36.849 | −7.926 | −9.495 | 1.00 | 12.12 | | A | C |
| ANISOU | 858 | CG | ASN | A | 163 | 1543 | 1509 | 1552 | 20 | −42 | 66 | A | C |
| ATOM | 859 | OD1 | ASN | A | 163 | −36.434 | −6.859 | −9.049 | 1.00 | 13.25 | | A | O |
| ANISOU | 859 | OD1 | ASN | A | 163 | 2013 | 1459 | 1562 | 70 | −76 | 109 | A | O |
| ATOM | 860 | ND2 | ASN | A | 163 | −37.557 | −8.006 | −10.626 | 1.00 | 15.83 | | A | N |
| ANISOU | 860 | ND2 | ASN | A | 163 | 1868 | 2337 | 1809 | 2 | −161 | 23 | A | N |
| ATOM | 861 | C | ASN | A | 163 | −34.837 | −8.266 | −7.190 | 1.00 | 7.83 | | A | C |
| ANISOU | 861 | C | ASN | A | 163 | 996 | 902 | 1077 | −33 | 22 | −46 | A | C |
| ATOM | 862 | O | ASN | A | 163 | −33.853 | −8.566 | −7.853 | 1.00 | 8.84 | | A | O |
| ANISOU | 862 | O | ASN | A | 163 | 1121 | 1123 | 1114 | −51 | 71 | −21 | A | O |
| ATOM | 863 | N | ASP | A | 164 | −34.855 | −7.236 | −6.343 | 1.00 | 7.81 | | A | N |
| ANISOU | 863 | N | ASP | A | 164 | 953 | 1035 | 981 | −43 | −45 | −16 | A | N |
| ATOM | 864 | CA | ASP | A | 164 | −33.615 | −6.585 | −5.948 | 1.00 | 8.54 | | A | C |
| ANISOU | 864 | CA | ASP | A | 164 | 1145 | 1078 | 1021 | −87 | 8 | −3 | A | C |
| ATOM | 865 | CB | ASP | A | 164 | −33.358 | −5.282 | −6.736 | 1.00 | 9.17 | | A | C |
| ANISOU | 865 | CB | ASP | A | 164 | 1203 | 1073 | 1207 | −21 | 21 | 91 | A | C |
| ATOM | 866 | CG | ASP | A | 164 | −34.346 | −4.162 | −6.408 | 1.00 | 11.50 | | A | C |
| ANISOU | 866 | CG | ASP | A | 164 | 1560 | 1325 | 1486 | 79 | 49 | −65 | A | C |
| ATOM | 867 | OD1 | ASP | A | 164 | −34.694 | −3.393 | −7.331 | 1.00 | 12.64 | | A | O |
| ANISOU | 867 | OD1 | ASP | A | 164 | 1863 | 1429 | 1512 | −14 | 79 | 49 | A | O |
| ATOM | 868 | OD2 | ASP | A | 164 | −34.760 | −4.025 | −5.243 | 1.00 | 13.39 | | A | O |
| ANISOU | 868 | OD2 | ASP | A | 164 | 2156 | 1493 | 1437 | 231 | 189 | 144 | A | O |
| ATOM | 869 | C | ASP | A | 164 | −33.559 | −6.389 | −4.436 | 1.00 | 8.75 | | A | C |
| ANISOU | 869 | C | ASP | A | 164 | 1120 | 1172 | 1032 | −61 | −46 | −21 | A | C |
| ATOM | 870 | O | ASP | A | 164 | −34.576 | −6.497 | −3.742 | 1.00 | 9.00 | | A | O |
| ANISOU | 870 | O | ASP | A | 164 | 1187 | 1266 | 966 | −17 | −48 | −93 | A | O |
| ATOM | 871 | N | TRP | A | 165 | −32.359 | −6.132 | −3.935 | 1.00 | 8.36 | | A | N |
| ANISOU | 871 | N | TRP | A | 165 | 1085 | 1164 | 928 | −36 | −51 | −15 | A | N |
| ATOM | 872 | CA | TRP | A | 165 | −32.135 | −5.983 | −2.501 | 1.00 | 7.64 | | A | C |
| ANISOU | 872 | CA | TRP | A | 165 | 1048 | 969 | 887 | −58 | −64 | 36 | A | C |
| ATOM | 873 | CB | TRP | A | 165 | −32.115 | −7.352 | −1.820 | 1.00 | 8.47 | | A | C |
| ANISOU | 873 | CB | TRP | A | 165 | 1208 | 1046 | 964 | −24 | −72 | 55 | A | C |
| ATOM | 874 | CG | TRP | A | 165 | −32.205 | −7.304 | −0.320 | 1.00 | 6.98 | | A | C |
| ANISOU | 874 | CG | TRP | A | 165 | 937 | 793 | 923 | 8 | −43 | −15 | A | C |
| ATOM | 875 | CD1 | TRP | A | 165 | −31.184 | −7.483 | 0.565 | 1.00 | 7.74 | | A | C |
| ANISOU | 875 | CD1 | TRP | A | 165 | 1145 | 847 | 950 | −64 | −82 | 20 | A | C |
| ATOM | 876 | NE1 | TRP | A | 165 | −31.650 | −7.393 | 1.854 | 1.00 | 8.31 | | A | N |
| ANISOU | 876 | NE1 | TRP | A | 165 | 1011 | 1136 | 1009 | 54 | 73 | 53 | A | N |
| ATOM | 877 | CE2 | TRP | A | 165 | −33.000 | −7.154 | 1.824 | 1.00 | 8.39 | | A | C |
| ANISOU | 877 | CE2 | TRP | A | 165 | 1086 | 1051 | 1049 | 25 | −60 | 62 | A | C |
| ATOM | 878 | CD2 | TRP | A | 165 | −33.386 | −7.093 | 0.465 | 1.00 | 7.46 | | A | C |
| ANISOU | 878 | CD2 | TRP | A | 165 | 1006 | 955 | 875 | −72 | −8 | −53 | A | C |
| ATOM | 879 | CE3 | TRP | A | 165 | −34.734 | −6.856 | 0.151 | 1.00 | 9.77 | | A | C |
| ANISOU | 879 | CE3 | TRP | A | 165 | 1185 | 1241 | 1288 | 166 | 31 | −23 | A | C |
| ATOM | 880 | CZ3 | TRP | A | 165 | −35.645 | −6.691 | 1.197 | 1.00 | 9.88 | | A | C |
| ANISOU | 880 | CZ3 | TRP | A | 165 | 1200 | 1384 | 1170 | 24 | 26 | 30 | A | C |
| ATOM | 881 | CH2 | TRP | A | 165 | −35.225 | −6.753 | 2.544 | 1.00 | 9.96 | | A | C |
| ANISOU | 881 | CH2 | TRP | A | 165 | 1213 | 1396 | 1174 | 10 | 37 | 61 | A | C |
| ATOM | 882 | CZ2 | TRP | A | 165 | −33.911 | −6.984 | 2.874 | 1.00 | 9.16 | | A | C |
| ANISOU | 882 | CZ2 | TRP | A | 165 | 1222 | 1159 | 1101 | 30 | −28 | 43 | A | C |
| ATOM | 883 | C | TRP | A | 165 | −30.804 | −5.291 | −2.313 | 1.00 | 7.80 | | A | C |
| ANISOU | 883 | C | TRP | A | 165 | 1051 | 994 | 917 | −49 | −30 | −14 | A | C |
| ATOM | 884 | O | TRP | A | 165 | −29.848 | −5.547 | −3.047 | 1.00 | 8.44 | | A | O |
| ANISOU | 884 | O | TRP | A | 165 | 1018 | 1128 | 1062 | −125 | 30 | −52 | A | O |
| ATOM | 885 | N | GLY | A | 166 | −30.745 | −4.410 | −1.327 | 1.00 | 7.18 | | A | N |
| ANISOU | 885 | N | GLY | A | 166 | 994 | 913 | 822 | −67 | 52 | −37 | A | N |
| ATOM | 886 | CA | GLY | A | 166 | −29.551 | −3.613 | −1.115 | 1.00 | 7.20 | | A | C |
| ANISOU | 886 | CA | GLY | A | 166 | 950 | 912 | 874 | −63 | 0 | −125 | A | C |
| ATOM | 887 | C | GLY | A | 166 | −29.861 | −2.432 | −0.236 | 1.00 | 7.50 | | A | C |
| ANISOU | 887 | C | GLY | A | 166 | 991 | 907 | 951 | −11 | 16 | −32 | A | C |
| ATOM | 888 | O | GLY | A | 166 | −30.658 | −2.543 | 0.687 | 1.00 | 8.47 | | A | O |
| ANISOU | 888 | O | GLY | A | 166 | 1080 | 1157 | 982 | −77 | 37 | −75 | A | O |
| ATOM | 889 | N | PHE | A | 167 | −29.240 | −1.295 | −0.539 | 1.00 | 6.71 | | A | N |
| ANISOU | 889 | N | PHE | A | 167 | 995 | 761 | 793 | −73 | −35 | −54 | A | N |
| ATOM | 890 | CA | PHE | A | 167 | −29.288 | −0.149 | 0.359 | 1.00 | 7.12 | | A | C |
| ANISOU | 890 | CA | PHE | A | 167 | 1014 | 775 | 918 | 23 | −26 | −7 | A | C |
| ATOM | 891 | CB | PHE | A | 167 | −27.939 | 0.001 | 1.068 | 1.00 | 7.94 | | A | C |
| ANISOU | 891 | CB | PHE | A | 167 | 1083 | 900 | 1034 | 65 | −88 | 4 | A | C |
| ATOM | 892 | CG | PHE | A | 167 | −27.485 | −1.249 | 1.758 | 1.00 | 7.68 | | A | C |
| ANISOU | 892 | CG | PHE | A | 167 | 1056 | 877 | 985 | 53 | 61 | 64 | A | C |
| ATOM | 893 | CD1 | PHE | A | 167 | −26.656 | −2.158 | 1.102 | 1.00 | 7.19 | | A | C |
| ANISOU | 893 | CD1 | PHE | A | 167 | 852 | 1027 | 851 | 81 | −28 | 91 | A | C |
| ATOM | 894 | CE1 | PHE | A | 167 | −26.247 | −3.325 | 1.735 | 1.00 | 7.20 | | A | C |
| ANISOU | 894 | CE1 | PHE | A | 167 | 982 | 800 | 952 | 43 | 23 | 13 | A | C |
| ATOM | 895 | CZ | PHE | A | 167 | −26.675 | −3.597 | 3.037 | 1.00 | 7.18 | | A | C |
| ANISOU | 895 | CZ | PHE | A | 167 | 849 | 977 | 902 | 29 | −18 | −39 | A | C |
| ATOM | 896 | CE2 | PHE | A | 167 | −27.512 | −2.701 | 3.695 | 1.00 | 7.41 | | A | C |
| ANISOU | 896 | CE2 | PHE | A | 167 | 909 | 924 | 983 | 26 | −4 | −9 | A | C |
| ATOM | 897 | CD2 | PHE | A | 167 | −27.914 | −1.534 | 3.054 | 1.00 | 8.38 | | A | C |
| ANISOU | 897 | CD2 | PHE | A | 167 | 1180 | 1073 | 930 | 4 | −26 | 38 | A | C |
| ATOM | 898 | C | PHE | A | 167 | −29.629 | 1.129 | −0.379 | 1.00 | 7.36 | | A | C |
| ANISOU | 898 | C | PHE | A | 167 | 985 | 930 | 880 | 40 | −25 | 11 | A | C |
| ATOM | 899 | O | PHE | A | 167 | −28.857 | 1.586 | −1.225 | 1.00 | 8.24 | | A | O |
| ANISOU | 899 | O | PHE | A | 167 | 1121 | 996 | 1015 | 58 | 50 | 120 | A | O |
| ATOM | 900 | N | ASER | A | 168 | −30.787 | 1.704 | −0.063 | 0.50 | 6.68 | | A | N |
| ANISOU | 900 | N | ASER | A | 168 | 888 | 861 | 788 | 16 | −15 | 12 | A | N |
| ATOM | 901 | N | BSER | A | 168 | −30.781 | 1.711 | −0.049 | 0.50 | 7.40 | | A | N |
| ANISOU | 901 | N | BSER | A | 168 | 952 | 966 | 893 | 22 | −9 | 3 | A | N |
| ATOM | 902 | CA | ASER | A | 168 | −31.179 | 2.982 | −0.646 | 0.50 | 6.92 | | A | C |
| ANISOU | 902 | CA | ASER | A | 168 | 882 | 871 | 877 | 39 | −34 | 5 | A | C |
| ATOM | 903 | CA | BSER | A | 168 | −31.195 | 2.982 | −0.643 | 0.50 | 8.30 | | A | C |
| ANISOU | 903 | CA | BSER | A | 168 | 1067 | 1029 | 1059 | 36 | −40 | 13 | A | C |
| ATOM | 904 | CB | ASER | A | 168 | −32.611 | 3.349 | −0.252 | 0.50 | 7.14 | | A | C |
| ANISOU | 904 | CB | ASER | A | 168 | 882 | 953 | 877 | −23 | −4 | −4 | A | C |
| ATOM | 905 | CB | BSER | A | 168 | −32.669 | 3.280 | −0.351 | 0.50 | 9.56 | | A | C |
| ANISOU | 905 | CB | BSER | A | 168 | 1124 | 1218 | 1290 | 8 | −7 | 13 | A | C |
| ATOM | 906 | OG | ASER | A | 168 | −33.540 | 2.465 | −0.856 | 0.50 | 7.33 | | A | O |
| ANISOU | 906 | OG | ASER | A | 168 | 771 | 990 | 1025 | −11 | −51 | −46 | A | O |
| ATOM | 907 | OG | BSER | A | 168 | −32.913 | 3.393 | 1.040 | 0.50 | 12.47 | | A | O |
| ANISOU | 907 | OG | BSER | A | 168 | 1603 | 1643 | 1491 | 19 | 61 | −83 | A | O |
| ATOM | 908 | C | ASER | A | 168 | −30.213 | 4.065 | −0.199 | 0.50 | 7.19 | | A | C |
| ANISOU | 908 | C | ASER | A | 168 | 933 | 895 | 902 | 1 | 11 | −10 | A | C |
| ATOM | 909 | C | BSER | A | 168 | −30.317 | 4.122 | −0.151 | 0.50 | 8.12 | | A | C |
| ANISOU | 909 | C | BSER | A | 168 | 1025 | 1052 | 1008 | 3 | −15 | 8 | A | C |
| ATOM | 910 | O | ASER | A | 168 | −29.867 | 4.961 | −0.976 | 0.50 | 7.57 | | A | O |
| ANISOU | 910 | O | ASER | A | 168 | 1087 | 915 | 875 | 12 | −7 | −13 | A | O |
| ATOM | 911 | O | BSER | A | 168 | −30.147 | 5.126 | −0.848 | 0.50 | 8.95 | | A | O |
| ANISOU | 911 | O | BSER | A | 168 | 1196 | 1131 | 1075 | −17 | −71 | 49 | A | O |
| ATOM | 912 | N | ASN | A | 169 | −29.767 | 3.949 | 1.053 | 1.00 | 7.23 | | A | N |
| ANISOU | 912 | N | ASN | A | 169 | 891 | 927 | 930 | −6 | −32 | −15 | A | N |
| ATOM | 913 | CA | ASN | A | 169 | −28.860 | 4.909 | 1.669 | 1.00 | 7.25 | | A | C |
| ANISOU | 913 | CA | ASN | A | 169 | 970 | 890 | 893 | 47 | −1 | −58 | A | C |
| ATOM | 914 | CB | ASN | A | 169 | −29.472 | 5.420 | 2.980 | 1.00 | 7.00 | | A | C |
| ANISOU | 914 | CB | ASN | A | 169 | 932 | 874 | 853 | 3 | −11 | −27 | A | C |
| ATOM | 915 | CG | ASN | A | 169 | −28.781 | 6.666 | 3.513 | 1.00 | 7.63 | | A | C |
| ANISOU | 915 | CG | ASN | A | 169 | 1035 | 862 | 1003 | 2 | 25 | 0 | A | C |
| ATOM | 916 | OD1 | ASN | A | 169 | −27.994 | 7.310 | 2.813 | 1.00 | 8.56 | | A | O |
| ANISOU | 916 | OD1 | ASN | A | 169 | 1094 | 1071 | 1087 | −154 | −33 | −52 | A | O |
| ATOM | 917 | ND2 | ASN | A | 169 | −29.080 | 7.014 | 4.769 | 1.00 | 9.47 | | A | N |
| ANISOU | 917 | ND2 | ASN | A | 169 | 1254 | 1272 | 1074 | 15 | −19 | −80 | A | N |
| ATOM | 918 | C | ASN | A | 169 | −27.518 | 4.221 | 1.921 | 1.00 | 7.22 | | A | C |
| ANISOU | 918 | C | ASN | A | 169 | 904 | 958 | 881 | 6 | 14 | −58 | A | C |
| ATOM | 919 | O | ASN | A | 169 | −27.082 | 4.080 | 3.068 | 1.00 | 7.62 | | A | O |
| ANISOU | 919 | O | ASN | A | 169 | 1022 | 976 | 897 | 53 | 32 | 22 | A | O |
| ATOM | 920 | N | PHE | A | 170 | −26.868 | 3.771 | 0.846 | 1.00 | 7.61 | | A | N |
| ANISOU | 920 | N | PHE | A | 170 | 1038 | 865 | 989 | −13 | 58 | −20 | A | N |
| ATOM | 921 | CA | PHE | A | 170 | −25.617 | 3.024 | 0.990 | 1.00 | 7.31 | | A | C |
| ANISOU | 921 | CA | PHE | A | 170 | 928 | 907 | 941 | −35 | 102 | −48 | A | C |
| ATOM | 922 | CB | PHE | A | 170 | −25.171 | 2.425 | −0.351 | 1.00 | 7.73 | | A | C |
| ANISOU | 922 | CB | PHE | A | 170 | 1087 | 935 | 916 | 25 | 66 | −78 | A | C |
| ATOM | 923 | CG | PHE | A | 170 | −23.955 | 1.548 | −0.236 | 1.00 | 8.16 | | A | C |
| ANISOU | 923 | CG | PHE | A | 170 | 967 | 993 | 1139 | 4 | 11 | −77 | A | C |
| ATOM | 924 | CD1 | PHE | A | 170 | −24.086 | 0.203 | 0.094 | 1.00 | 9.17 | | A | C |
| ANISOU | 924 | CD1 | PHE | A | 170 | 1353 | 1025 | 1107 | 78 | 74 | 43 | A | C |
| ATOM | 925 | CE1 | PHE | A | 170 | −22.965 | −0.619 | 0.210 | 1.00 | 9.76 | | A | C |
| ANISOU | 925 | CE1 | PHE | A | 170 | 1147 | 1311 | 1250 | 0 | −11 | −2 | A | C |
| ATOM | 926 | CZ | PHE | A | 170 | −21.700 | −0.095 | 0.000 | 1.00 | 10.16 | | A | C |
| ANISOU | 926 | CZ | PHE | A | 170 | 1223 | 1435 | 1203 | −2 | 77 | 81 | A | C |
| ATOM | 927 | CE2 | PHE | A | 170 | −21.556 | 1.246 | −0.330 | 1.00 | 12.15 | | A | C |
| ANISOU | 927 | CE2 | PHE | A | 170 | 1403 | 1352 | 1861 | 75 | −10 | −98 | A | C |
| ATOM | 928 | CD2 | PHE | A | 170 | −22.681 | 2.062 | −0.454 | 1.00 | 10.37 | | A | C |
| ANISOU | 928 | CD2 | PHE | A | 170 | 1210 | 1197 | 1535 | −61 | −22 | −99 | A | C |
| ATOM | 929 | C | PHE | A | 170 | −24.511 | 3.903 | 1.579 | 1.00 | 8.09 | | A | C |
| ANISOU | 929 | C | PHE | A | 170 | 1059 | 1044 | 971 | −8 | −25 | −31 | A | C |
| ATOM | 930 | O | PHE | A | 170 | −23.832 | 3.517 | 2.533 | 1.00 | 8.39 | | A | O |
| ANISOU | 930 | O | PHE | A | 170 | 1086 | 1014 | 1086 | 8 | 16 | 2 | A | O |
| ATOM | 931 | N | MET | A | 171 | −24.361 | 5.086 | 0.986 | 1.00 | 8.66 | | A | N |
| ANISOU | 931 | N | MET | A | 171 | 1163 | 1046 | 1082 | −61 | 26 | −11 | A | N |
| ATOM | 932 | CA | MET | A | 171 | −23.431 | 6.119 | 1.424 | 1.00 | 9.27 | | A | C |
| ANISOU | 932 | CA | MET | A | 171 | 1208 | 1061 | 1255 | −52 | 0 | 5 | A | C |
| ATOM | 933 | CB | MET | A | 171 | −22.057 | 5.925 | 0.776 | 1.00 | 11.05 | | A | C |
| ANISOU | 933 | CB | MET | A | 171 | 1384 | 1433 | 1380 | 34 | 36 | 13 | A | C |
| ATOM | 934 | CG | MET | A | 171 | −21.102 | 5.058 | 1.556 | 1.00 | 14.07 | | A | C |
| ANISOU | 934 | CG | MET | A | 171 | 1644 | 1785 | 1917 | 43 | 12 | 89 | A | C |
| ATOM | 935 | SD | MET | A | 171 | −19.456 | 5.076 | 0.817 | 1.00 | 17.43 | | A | S |
| ANISOU | 935 | SD | MET | A | 171 | 1725 | 2416 | 2483 | 29 | 215 | −170 | A | S |
| ATOM | 936 | CE | MET | A | 171 | −18.961 | 6.774 | 1.083 | 1.00 | 15.84 | | A | C |
| ANISOU | 936 | CE | MET | A | 171 | 1812 | 2008 | 2199 | 97 | 97 | 126 | A | C |
| ATOM | 937 | C | MET | A | 171 | −24.004 | 7.453 | 0.971 | 1.00 | 8.25 | | A | C |
| ANISOU | 937 | C | MET | A | 171 | 1073 | 1026 | 1037 | −35 | −18 | −10 | A | C |
| ATOM | 938 | O | MET | A | 171 | −24.790 | 7.501 | 0.019 | 1.00 | 8.60 | | A | O |
| ANISOU | 938 | O | MET | A | 171 | 1082 | 1125 | 1059 | −2 | 1 | −73 | A | O |
| ATOM | 939 | N | ALA | A | 172 | −23.617 | 8.537 | 1.640 | 1.00 | 8.44 | | A | N |
| ANISOU | 939 | N | ALA | A | 172 | 1174 | 963 | 1068 | −36 | −32 | 0 | A | N |
| ATOM | 940 | CA | ALA | A | 172 | −24.043 | 9.866 | 1.206 | 1.00 | 8.28 | | A | C |
| ANISOU | 940 | CA | ALA | A | 172 | 1140 | 938 | 1069 | −26 | −49 | 10 | A | C |
| ATOM | 941 | CB | ALA | A | 172 | −23.642 | 10.925 | 2.230 | 1.00 | 9.59 | | A | C |
| ANISOU | 941 | CB | ALA | A | 172 | 1289 | 1164 | 1190 | −47 | −48 | −62 | A | C |
| ATOM | 942 | C | ALA | A | 172 | −23.435 | 10.183 | −0.159 | 1.00 | 8.86 | | A | C |
| ANISOU | 942 | C | ALA | A | 172 | 1190 | 1041 | 1136 | 0 | −25 | −10 | A | C |
| ATOM | 943 | O | ALA | A | 172 | −22.260 | 9.907 | −0.402 | 1.00 | 9.76 | | A | O |
| ANISOU | 943 | O | ALA | A | 172 | 1288 | 1175 | 1244 | 34 | 80 | −83 | A | O |
| ATOM | 944 | N | TRP | A | 173 | −24.239 | 10.762 | −1.047 | 1.00 | 8.97 | | A | N |
| ANISOU | 944 | N | TRP | A | 173 | 1233 | 1126 | 1049 | −63 | −53 | 48 | A | N |
| ATOM | 945 | CA | TRP | A | 173 | −23.769 | 11.143 | −2.385 | 1.00 | 9.47 | | A | C |
| ANISOU | 945 | CA | TRP | A | 173 | 1218 | 1235 | 1147 | −2 | 20 | 7 | A | C |
| ATOM | 946 | CB | TRP | A | 173 | −24.919 | 11.765 | −3.185 | 1.00 | 10.07 | | A | C |
| ANISOU | 946 | CB | TRP | A | 173 | 1272 | 1311 | 1244 | 38 | 1 | 150 | A | C |
| ATOM | 947 | CG | TRP | A | 173 | −24.613 | 11.966 | −4.649 | 1.00 | 11.50 | | A | C |
| ANISOU | 947 | CG | TRP | A | 173 | 1407 | 1623 | 1340 | 37 | 34 | 46 | A | C |
| ATOM | 948 | CD1 | TRP | A | 173 | −24.080 | 13.084 | −5.228 | 1.00 | 13.44 | | A | C |
| ANISOU | 948 | CD1 | TRP | A | 173 | 1673 | 1803 | 1629 | 0 | 53 | 51 | A | C |
| ATOM | 949 | NE1 | TRP | A | 173 | −23.950 | 12.900 | −6.587 | 1.00 | 14.09 | | A | N |
| ANISOU | 949 | NE1 | TRP | A | 173 | 1877 | 1907 | 1568 | 52 | −57 | 43 | A | N |
| ATOM | 950 | CE2 | TRP | A | 173 | −24.407 | 11.649 | −6.909 | 1.00 | 12.99 | | A | C |
| ANISOU | 950 | CE2 | TRP | A | 173 | 1725 | 1744 | 1467 | 48 | −24 | 115 | A | C |
| ATOM | 951 | CD2 | TRP | A | 173 | −24.837 | 11.031 | −5.708 | 1.00 | 11.02 | | A | C |
| ANISOU | 951 | CD2 | TRP | A | 173 | 1218 | 1580 | 1388 | 84 | 50 | 68 | A | C |
| ATOM | 952 | CE3 | TRP | A | 173 | −25.354 | 9.731 | −5.763 | 1.00 | 12.54 | | A | C |
| ANISOU | 952 | CE3 | TRP | A | 173 | 1530 | 1712 | 1522 | 22 | −55 | −48 | A | C |
| ATOM | 953 | CZ3 | TRP | A | 173 | −25.426 | 9.090 | −7.002 | 1.00 | 14.73 | | A | C |
| ANISOU | 953 | CZ3 | TRP | A | 173 | 1975 | 1931 | 1691 | 43 | 8 | −78 | A | C |
| ATOM | 954 | CH2 | TRP | A | 173 | −24.991 | 9.733 | −8.176 | 1.00 | 14.59 | | A | C |
| ANISOU | 954 | CH2 | TRP | A | 173 | 1865 | 2000 | 1679 | 27 | −58 | −49 | A | C |
| ATOM | 955 | CZ2 | TRP | A | 173 | −24.480 | 11.004 | −8.151 | 1.00 | 13.88 | | A | C |
| ANISOU | 955 | CZ2 | TRP | A | 173 | 1804 | 1900 | 1570 | 51 | 9 | 37 | A | C |
| ATOM | 956 | C | TRP | A | 173 | −22.581 | 12.106 | −2.311 | 1.00 | 10.32 | | A | C |
| ANISOU | 956 | C | TRP | A | 173 | 1315 | 1312 | 1293 | 14 | −7 | 54 | A | C |
| ATOM | 957 | O | TRP | A | 173 | −21.614 | 11.974 | −3.069 | 1.00 | 10.66 | | A | O |
| ANISOU | 957 | O | TRP | A | 173 | 1286 | 1365 | 1400 | 2 | 24 | 38 | A | O |
| ATOM | 958 | N | SER | A | 174 | −22.647 | 13.051 | −1.377 | 1.00 | 11.02 | | A | N |
| ANISOU | 958 | N | SER | A | 174 | 1387 | 1357 | 1444 | −5 | −49 | −36 | A | N |
| ATOM | 959 | CA | SER | A | 174 | −21.580 | 14.043 | −1.214 | 1.00 | 12.25 | | A | C |
| ANISOU | 959 | CA | SER | A | 174 | 1539 | 1477 | 1640 | −56 | 1 | −52 | A | C |
| ATOM | 960 | CB | SER | A | 174 | −21.975 | 15.098 | −0.180 | 1.00 | 13.17 | | A | C |
| ANISOU | 960 | CB | SER | A | 174 | 1699 | 1525 | 1779 | −38 | 15 | −122 | A | C |
| ATOM | 961 | OG | SER | A | 174 | −22.157 | 14.514 | 1.095 | 1.00 | 14.30 | | A | O |
| ANISOU | 961 | OG | SER | A | 174 | 1910 | 1745 | 1777 | 61 | −28 | −132 | A | O |
| ATOM | 962 | C | SER | A | 174 | −20.242 | 13.415 | −0.831 | 1.00 | 12.61 | | A | C |
| ANISOU | 962 | C | SER | A | 174 | 1601 | 1524 | 1665 | −3 | 10 | −3 | A | C |
| ATOM | 963 | O | SER | A | 174 | −19.183 | 13.963 | −1.147 | 1.00 | 14.20 | | A | O |
| ANISOU | 963 | O | SER | A | 174 | 1664 | 1763 | 1968 | −5 | 66 | 1 | A | O |
| ATOM | 964 | N | GLU | A | 175 | −20.291 | 12.275 | −0.156 | 1.00 | 11.97 | | A | N |
| ANISOU | 964 | N | GLU | A | 175 | 1500 | 1496 | 1551 | 16 | −16 | −64 | A | N |
| ATOM | 965 | CA | GLU | A | 175 | −19.064 | 11.603 | 0.245 | 1.00 | 12.97 | | A | C |
| ANISOU | 965 | CA | GLU | A | 175 | 1617 | 1627 | 1684 | 40 | −8 | 8 | A | C |
| ATOM | 966 | CB | GLU | A | 175 | −19.272 | 10.831 | 1.535 | 1.00 | 14.04 | | A | C |
| ANISOU | 966 | CB | GLU | A | 175 | 1684 | 1857 | 1794 | 16 | −19 | 106 | A | C |
| ATOM | 967 | CG | GLU | A | 175 | −19.411 | 11.789 | 2.708 | 1.00 | 20.91 | | A | C |
| ANISOU | 967 | CG | GLU | A | 175 | 2730 | 2534 | 2680 | 134 | −1 | −243 | A | C |
| ATOM | 968 | CD | GLU | A | 175 | −19.833 | 11.109 | 3.973 | 1.00 | 27.31 | | A | C |
| ANISOU | 968 | CD | GLU | A | 175 | 3512 | 3593 | 3272 | −166 | −34 | 113 | A | C |
| ATOM | 969 | OE1 | GLU | A | 175 | −20.424 | 11.770 | 4.874 | 1.00 | 30.56 | | A | O |
| ANISOU | 969 | OE1 | GLU | A | 175 | 3991 | 3756 | 3863 | 65 | 44 | −39 | A | O |
| ATOM | 970 | OE2 | GLU | A | 175 | −19.602 | 9.902 | 4.046 | 1.00 | 30.76 | | A | O |
| ANISOU | 970 | OE2 | GLU | A | 175 | 3890 | 3810 | 3989 | 82 | −21 | −19 | A | O |
| ATOM | 971 | C | GLU | A | 175 | −18.459 | 10.734 | −0.851 | 1.00 | 12.74 | | A | C |
| ANISOU | 971 | C | GLU | A | 175 | 1600 | 1621 | 1619 | −29 | −40 | −16 | A | C |
| ATOM | 972 | O | GLU | A | 175 | −17.279 | 10.754 | −1.063 | 1.00 | 12.98 | | A | O |
| ANISOU | 972 | O | GLU | A | 175 | 1573 | 1707 | 1652 | 19 | −31 | −60 | A | O |
| ATOM | 973 | N | VAL | A | 176 | −19.297 | 9.974 | −1.536 | 1.00 | 11.84 | | A | N |
| ANISOU | 973 | N | VAL | A | 176 | 1533 | 1487 | 1477 | 5 | −68 | 47 | A | N |
| ATOM | 974 | CA | VAL | A | 176 | −18.822 | 9.177 | −2.664 | 1.00 | 12.53 | | A | C |
| ANISOU | 974 | CA | VAL | A | 176 | 1616 | 1569 | 1577 | 6 | 17 | −10 | A | C |
| ATOM | 975 | CB | VAL | A | 176 | −19.952 | 8.317 | −3.266 | 1.00 | 13.95 | | A | C |
| ANISOU | 975 | CB | VAL | A | 176 | 1773 | 1730 | 1796 | −53 | 2 | −47 | A | C |
| ATOM | 976 | CG1 | VAL | A | 176 | −19.462 | 7.559 | −4.495 | 1.00 | 16.72 | | A | C |
| ANISOU | 976 | CG1 | VAL | A | 176 | 2185 | 2165 | 2003 | 1 | 79 | −131 | A | C |
| ATOM | 977 | CG2 | VAL | A | 176 | −20.475 | 7.340 | −2.228 | 1.00 | 16.33 | | A | C |
| ANISOU | 977 | CG2 | VAL | A | 176 | 2182 | 2033 | 1990 | −8 | 85 | 67 | A | C |
| ATOM | 978 | C | VAL | A | 176 | −18.184 | 10.065 | −3.741 | 1.00 | 12.61 | | A | C |
| ANISOU | 978 | C | VAL | A | 176 | 1573 | 1601 | 1616 | 13 | 11 | −2 | A | C |
| ATOM | 979 | O | VAL | A | 176 | −17.164 | 9.697 | −4.327 | 1.00 | 12.11 | | A | O |
| ANISOU | 979 | O | VAL | A | 176 | 1423 | 1636 | 1541 | 30 | 8 | −54 | A | O |
| ATOM | 980 | N | THR | A | 177 | −18.770 | 11.240 | −3.976 | 1.00 | 12.75 | | A | N |
| ANISOU | 980 | N | THR | A | 177 | 1659 | 1566 | 1621 | −14 | 0 | 12 | A | N |
| ATOM | 981 | CA | THR | A | 177 | −18.308 | 12.140 | −5.041 | 1.00 | 12.85 | | A | C |
| ANISOU | 981 | CA | THR | A | 177 | 1647 | 1607 | 1629 | −10 | 30 | −9 | A | C |
| ATOM | 982 | CB | THR | A | 177 | −19.480 | 12.935 | −5.662 | 1.00 | 12.61 | | A | C |
| ANISOU | 982 | CB | THR | A | 177 | 1650 | 1558 | 1582 | −44 | 16 | 36 | A | C |
| ATOM | 983 | OG1 | THR | A | 177 | −20.097 | 13.747 | −4.655 | 1.00 | 13.85 | | A | O |
| ANISOU | 983 | OG1 | THR | A | 177 | 1845 | 1624 | 1793 | −11 | 18 | −62 | A | O |
| ATOM | 984 | CG2 | THR | A | 177 | −20.520 | 11.988 | −6.266 | 1.00 | 13.18 | | A | C |
| ANISOU | 984 | CG2 | THR | A | 177 | 1646 | 1780 | 1583 | −3 | −99 | −43 | A | C |
| ATOM | 985 | C | THR | A | 177 | −17.210 | 13.112 | −4.593 | 1.00 | 13.90 | | A | C |
| ANISOU | 985 | C | THR | A | 177 | 1780 | 1743 | 1758 | −42 | −6 | −14 | A | C |
| ATOM | 986 | O | THR | A | 177 | −16.725 | 13.913 | −5.397 | 1.00 | 14.32 | | A | O |
| ANISOU | 986 | O | THR | A | 177 | 1886 | 1770 | 1785 | −89 | 20 | −5 | A | O |
| ATOM | 987 | N | ASP | A | 178 | −16.824 | 13.044 | −3.319 | 1.00 | 14.23 | | A | N |
| ANISOU | 987 | N | ASP | A | 178 | 1812 | 1804 | 1792 | −60 | −26 | −20 | A | N |
| ATOM | 988 | CA | ASP | A | 178 | −15.753 | 13.895 | −2.784 | 1.00 | 15.67 | | A | C |
| ANISOU | 988 | CA | ASP | A | 178 | 1921 | 2006 | 2028 | −58 | −46 | −35 | A | C |
| ATOM | 989 | CB | ASP | A | 178 | −15.868 | 13.977 | −1.253 | 1.00 | 15.82 | | A | C |
| ANISOU | 989 | CB | ASP | A | 178 | 1937 | 2070 | 2004 | −13 | −35 | −5 | A | C |
| ATOM | 990 | CG | ASP | A | 178 | −14.865 | 14.946 | −0.622 | 1.00 | 17.73 | | A | C |
| ANISOU | 990 | CG | ASP | A | 178 | 2192 | 2248 | 2295 | −93 | −57 | −5 | A | C |
| ATOM | 991 | OD1 | ASP | A | 178 | −13.865 | 15.316 | −1.275 | 1.00 | 17.71 | | A | O |
| ANISOU | 991 | OD1 | ASP | A | 178 | 2094 | 2223 | 2411 | −143 | −23 | −65 | A | O |
| ATOM | 992 | OD2 | ASP | A | 178 | −15.082 | 15.334 | 0.547 | 1.00 | 20.34 | | A | O |
| ANISOU | 992 | OD2 | ASP | A | 178 | 2527 | 2677 | 2525 | −75 | 9 | −113 | A | O |
| ATOM | 993 | C | ASP | A | 178 | −14.394 | 13.326 | −3.196 | 1.00 | 16.33 | | A | C |
| ANISOU | 993 | C | ASP | A | 178 | 2001 | 2093 | 2112 | −33 | −24 | −19 | A | C |
| ATOM | 994 | O | ASP | A | 178 | −14.006 | 12.257 | −2.721 | 1.00 | 15.39 | | A | O |
| ANISOU | 994 | O | ASP | A | 178 | 1832 | 1977 | 2039 | −83 | −27 | 2 | A | O |
| ATOM | 995 | N | PRO | A | 179 | −13.651 | 14.045 | −4.064 | 1.00 | 18.00 | | A | N |
| ANISOU | 995 | N | PRO | A | 179 | 2263 | 2286 | 2289 | −51 | −18 | 35 | A | N |
| ATOM | 996 | CA | PRO | A | 179 | −12.376 | 13.499 | −4.555 | 1.00 | 19.70 | | A | C |
| ANISOU | 996 | CA | PRO | A | 179 | 2450 | 2506 | 2530 | 7 | 39 | −9 | A | C |
| ATOM | 997 | CB | PRO | A | 179 | −11.821 | 14.629 | −5.429 | 1.00 | 20.22 | | A | C |
| ANISOU | 997 | CB | PRO | A | 179 | 2525 | 2579 | 2578 | 7 | 61 | 38 | A | C |
| ATOM | 998 | CG | PRO | A | 179 | −13.017 | 15.429 | −5.819 | 1.00 | 20.36 | | A | C |
| ANISOU | 998 | CG | PRO | A | 179 | 2564 | 2651 | 2519 | 18 | 32 | 73 | A | C |
| ATOM | 999 | CD | PRO | A | 179 | −13.925 | 15.379 | −4.627 | 1.00 | 18.74 | | A | C |
| ANISOU | 999 | CD | PRO | A | 179 | 2310 | 2350 | 2461 | −12 | 5 | 24 | A | C |
| ATOM | 1000 | C | PRO | A | 179 | −11.385 | 13.152 | −3.439 | 1.00 | 20.94 | | A | C |
| ANISOU | 1000 | C | PRO | A | 179 | 2625 | 2685 | 2645 | 6 | −8 | −10 | A | C |
| ATOM | 1001 | O | PRO | A | 179 | −10.547 | 12.263 | −3.613 | 1.00 | 21.83 | | A | O |
| ANISOU | 1001 | O | PRO | A | 179 | 2665 | 2824 | 2806 | 90 | −32 | −92 | A | O |
| ATOM | 1002 | N | GLU | A | 180 | −11.497 | 13.830 | −2.300 | 1.00 | 20.90 | | A | N |
| ANISOU | 1002 | N | GLU | A | 180 | 2650 | 2619 | 2671 | −28 | 2 | −21 | A | N |
| ATOM | 1003 | CA | GLU | A | 180 | −10.583 | 13.604 | −1.178 | 1.00 | 21.62 | | A | C |
| ANISOU | 1003 | CA | GLU | A | 180 | 2721 | 2766 | 2728 | −22 | −18 | 13 | A | C |
| ATOM | 1004 | CB | GLU | A | 180 | −10.626 | 14.782 | −0.199 | 1.00 | 22.97 | | A | C |
| ANISOU | 1004 | CB | GLU | A | 180 | 2827 | 3020 | 2882 | −26 | 10 | −94 | A | C |
| ATOM | 1005 | CG | GLU | A | 180 | −10.666 | 16.150 | −0.872 | 1.00 | 28.76 | | A | C |
| ANISOU | 1005 | CG | GLU | A | 180 | 3734 | 3514 | 3678 | 102 | 69 | 173 | A | C |
| ATOM | 1006 | CD | GLU | A | 180 | −9.829 | 17.179 | −0.147 | 1.00 | 35.43 | | A | C |
| ANISOU | 1006 | CD | GLU | A | 180 | 4581 | 4419 | 4460 | −180 | −130 | −57 | A | C |
| ATOM | 1007 | OE1 | GLU | A | 180 | −10.413 | 18.133 | 0.415 | 1.00 | 38.47 | | A | O |
| ANISOU | 1007 | OE1 | GLU | A | 180 | 4900 | 4832 | 4886 | 62 | 46 | −65 | A | O |
| ATOM | 1008 | OE2 | GLU | A | 180 | −8.585 | 17.030 | −0.137 | 1.00 | 38.92 | | A | O |
| ANISOU | 1008 | OE2 | GLU | A | 180 | 4766 | 5039 | 4982 | 26 | 38 | 4 | A | O |
| ATOM | 1009 | C | GLU | A | 180 | −10.842 | 12.286 | −0.438 | 1.00 | 20.73 | | A | C |
| ANISOU | 1009 | C | GLU | A | 180 | 2539 | 2693 | 2643 | −24 | −27 | −11 | A | C |
| ATOM | 1010 | O | GLU | A | 180 | −9.984 | 11.816 | 0.314 | 1.00 | 20.88 | | A | O |
| ANISOU | 1010 | O | GLU | A | 180 | 2546 | 2763 | 2624 | −27 | −50 | −2 | A | O |
| ATOM | 1011 | N | LYS | A | 181 | −12.013 | 11.688 | −0.658 | 1.00 | 19.55 | | A | N |
| ANISOU | 1011 | N | LYS | A | 181 | 2448 | 2491 | 2491 | −46 | −24 | −22 | A | N |
| ATOM | 1012 | CA | LYS | A | 181 | −12.392 | 10.462 | 0.050 | 1.00 | 19.08 | | A | C |
| ANISOU | 1012 | CA | LYS | A | 181 | 2365 | 2427 | 2459 | 2 | −51 | −9 | A | C |
| ATOM | 1013 | CB | LYS | A | 181 | −13.912 | 10.390 | 0.272 | 1.00 | 20.60 | | A | C |
| ANISOU | 1013 | CB | LYS | A | 181 | 2430 | 2642 | 2755 | −25 | −42 | 31 | A | C |
| ATOM | 1014 | CG | LYS | A | 181 | −14.488 | 11.472 | 1.196 | 1.00 | 23.49 | | A | C |
| ANISOU | 1014 | CG | LYS | A | 181 | 2919 | 2941 | 3067 | 21 | 38 | −46 | A | C |
| ATOM | 1015 | CD | LYS | A | 181 | −13.795 | 11.506 | 2.557 | 1.00 | 26.91 | | A | C |
| ANISOU | 1015 | CD | LYS | A | 181 | 3455 | 3506 | 3262 | 42 | −75 | −26 | A | C |
| ATOM | 1016 | CE | LYS | A | 181 | −14.495 | 12.447 | 3.532 | 1.00 | 29.64 | | A | C |
| ANISOU | 1016 | CE | LYS | A | 181 | 3755 | 3887 | 3619 | 48 | 35 | −146 | A | C |
| ATOM | 1017 | NZ | LYS | A | 181 | −14.370 | 13.879 | 3.144 | 1.00 | 33.29 | | A | N |
| ANISOU | 1017 | NZ | LYS | A | 181 | 4309 | 4086 | 4253 | −10 | −4 | 18 | A | N |
| ATOM | 1018 | C | LYS | A | 181 | −11.873 | 9.180 | −0.608 | 1.00 | 17.68 | | A | C |
| ANISOU | 1018 | C | LYS | A | 181 | 2175 | 2318 | 2225 | −38 | −39 | 7 | A | C |
| ATOM | 1019 | O | LYS | A | 181 | −11.787 | 8.141 | 0.048 | 1.00 | 18.47 | | A | O |
| ANISOU | 1019 | O | LYS | A | 181 | 2349 | 2391 | 2276 | −9 | −34 | 19 | A | O |
| ATOM | 1020 | N | GLY | A | 182 | −11.539 | 9.249 | −1.896 | 1.00 | 15.85 | | A | N |
| ANISOU | 1020 | N | GLY | A | 182 | 1859 | 2038 | 2124 | −30 | −47 | −17 | A | N |
| ATOM | 1021 | CA | GLY | A | 182 | −10.854 | 8.144 | −2.573 | 1.00 | 15.07 | | A | C |
| ANISOU | 1021 | CA | GLY | A | 182 | 1829 | 1964 | 1934 | −60 | −59 | −27 | A | C |
| ATOM | 1022 | C | GLY | A | 182 | −11.703 | 7.139 | −3.334 | 1.00 | 13.88 | | A | C |
| ANISOU | 1022 | C | GLY | A | 182 | 1772 | 1755 | 1745 | −4 | −13 | 43 | A | C |
| ATOM | 1023 | O | GLY | A | 182 | −11.182 | 6.147 | −3.834 | 1.00 | 14.21 | | A | O |
| ANISOU | 1023 | O | GLY | A | 182 | 1785 | 1822 | 1794 | −13 | −41 | 0 | A | O |
| ATOM | 1024 | N | PHE | A | 183 | −13.005 | 7.388 | −3.428 | 1.00 | 13.33 | | A | N |
| ANISOU | 1024 | N | PHE | A | 183 | 1703 | 1692 | 1669 | −64 | −20 | 41 | A | N |
| ATOM | 1025 | CA | PHE | A | 183 | −13.896 | 6.481 | −4.151 | 1.00 | 13.42 | | A | C |
| ANISOU | 1025 | CA | PHE | A | 183 | 1705 | 1716 | 1677 | −54 | −14 | 29 | A | C |
| ATOM | 1026 | CB | PHE | A | 183 | −15.266 | 6.406 | −3.470 | 1.00 | 13.10 | | A | C |
| ANISOU | 1026 | CB | PHE | A | 183 | 1658 | 1700 | 1620 | −32 | −17 | 2 | A | C |
| ATOM | 1027 | CG | PHE | A | 183 | −15.217 | 5.840 | −2.080 | 1.00 | 12.79 | | A | C |
| ANISOU | 1027 | CG | PHE | A | 183 | 1599 | 1640 | 1619 | −66 | −23 | 9 | A | C |
| ATOM | 1028 | CD1 | PHE | A | 183 | −15.356 | 6.671 | −0.973 | 1.00 | 12.94 | | A | C |
| ANISOU | 1028 | CD1 | PHE | A | 183 | 1627 | 1693 | 1595 | −34 | −23 | 1 | A | C |
| ATOM | 1029 | CE1 | PHE | A | 183 | −15.308 | 6.150 | 0.319 | 1.00 | 13.39 | | A | C |
| ANISOU | 1029 | CE1 | PHE | A | 183 | 1676 | 1744 | 1666 | 6 | 39 | 36 | A | C |
| ATOM | 1030 | CZ | PHE | A | 183 | −15.110 | 4.784 | 0.511 | 1.00 | 13.62 | | A | C |
| ANISOU | 1030 | CZ | PHE | A | 183 | 1850 | 1664 | 1661 | −60 | 47 | 36 | A | C |
| ATOM | 1031 | CE2 | PHE | A | 183 | −14.971 | 3.942 | −0.589 | 1.00 | 13.52 | | A | C |
| ANISOU | 1031 | CE2 | PHE | A | 183 | 1751 | 1736 | 1650 | −64 | −4 | 56 | A | C |
| ATOM | 1032 | CD2 | PHE | A | 183 | −15.021 | 4.476 | −1.878 | 1.00 | 13.35 | | A | C |
| ANISOU | 1032 | CD2 | PHE | A | 183 | 1686 | 1697 | 1688 | −24 | −35 | 24 | A | C |
| ATOM | 1033 | C | PHE | A | 183 | −14.056 | 6.857 | −5.620 | 1.00 | 14.18 | | A | C |
| ANISOU | 1033 | C | PHE | A | 183 | 1883 | 1749 | 1757 | −18 | −65 | 43 | A | C |
| ATOM | 1034 | O | PHE | A | 183 | −14.352 | 6.007 | −6.455 | 1.00 | 17.11 | | A | O |
| ANISOU | 1034 | O | PHE | A | 183 | 2511 | 2047 | 1942 | −43 | −141 | −10 | A | O |
| ATOM | 1035 | N | ILE | A | 184 | −13.851 | 8.130 | −5.932 | 1.00 | 12.48 | | A | N |
| ANISOU | 1035 | N | ILE | A | 184 | 1589 | 1633 | 1519 | −27 | −27 | 35 | A | N |
| ATOM | 1036 | CA | ILE | A | 184 | −14.110 | 8.634 | −7.281 | 1.00 | 12.05 | | A | C |
| ANISOU | 1036 | CA | ILE | A | 184 | 1525 | 1560 | 1493 | −39 | −21 | 18 | A | C |
| ATOM | 1037 | CB | ILE | A | 184 | −15.175 | 9.767 | −7.256 | 1.00 | 11.66 | | A | C |
| ANISOU | 1037 | CB | ILE | A | 184 | 1525 | 1503 | 1402 | −33 | −4 | 30 | A | C |
| ATOM | 1038 | CG1 | ILE | A | 184 | −15.708 | 10.056 | −8.665 | 1.00 | 11.51 | | A | C |
| ANISOU | 1038 | CG1 | ILE | A | 184 | 1433 | 1528 | 1412 | 24 | −77 | −20 | A | C |
| ATOM | 1039 | CD1 | ILE | A | 184 | −17.076 | 10.718 | −8.670 | 1.00 | 11.78 | | A | C |
| ANISOU | 1039 | CD1 | ILE | A | 184 | 1503 | 1431 | 1540 | 54 | 0 | 62 | A | C |
| ATOM | 1040 | CG2 | ILE | A | 184 | −14.640 | 11.035 | −6.558 | 1.00 | 13.01 | | A | C |
| ANISOU | 1040 | CG2 | ILE | A | 184 | 1727 | 1599 | 1619 | −41 | −13 | −77 | A | C |
| ATOM | 1041 | C | ILE | A | 184 | −12.811 | 9.075 | −7.956 | 1.00 | 12.65 | | A | C |
| ANISOU | 1041 | C | ILE | A | 184 | 1605 | 1686 | 1517 | −50 | 43 | −1 | A | C |
| ATOM | 1042 | O | ILE | A | 184 | −11.970 | 9.720 | −7.328 | 1.00 | 13.43 | | A | O |
| ANISOU | 1042 | O | ILE | A | 184 | 1666 | 1792 | 1644 | −118 | −68 | −16 | A | O |
| ATOM | 1043 | N | ASP | A | 185 | −12.641 | 8.699 | −9.223 | 1.00 | 12.01 | | A | N |
| ANISOU | 1043 | N | ASP | A | 185 | 1525 | 1574 | 1466 | −10 | −33 | 23 | A | N |
| ATOM | 1044 | CA | ASP | A | 185 | −11.475 | 9.117 | −10.006 | 1.00 | 12.57 | | A | C |
| ANISOU | 1044 | CA | ASP | A | 185 | 1555 | 1610 | 1611 | −39 | 3 | −4 | A | C |
| ATOM | 1045 | CB | ASP | A | 185 | −10.375 | 8.055 | −9.947 | 1.00 | 13.91 | | A | C |
| ANISOU | 1045 | CB | ASP | A | 185 | 1770 | 1746 | 1770 | 43 | −33 | 50 | A | C |
| ATOM | 1046 | CG | ASP | A | 185 | −9.128 | 8.466 | −10.710 | 1.00 | 17.35 | | A | C |
| ANISOU | 1046 | CG | ASP | A | 185 | 1974 | 2326 | 2292 | 40 | 85 | 78 | A | C |
| ATOM | 1047 | OD1 | ASP | A | 185 | −8.411 | 9.375 | −10.242 | 1.00 | 19.88 | | A | O |
| ANISOU | 1047 | OD1 | ASP | A | 185 | 2357 | 2579 | 2619 | −52 | 39 | −36 | A | O |
| ATOM | 1048 | OD2 | ASP | A | 185 | −8.873 | 7.881 | −11.781 | 1.00 | 20.90 | | A | O |
| ANISOU | 1048 | OD2 | ASP | A | 185 | 2586 | 2774 | 2580 | 18 | 70 | −111 | A | O |
| ATOM | 1049 | C | ASP | A | 185 | −11.888 | 9.328 | −11.449 | 1.00 | 13.03 | | A | C |
| ANISOU | 1049 | C | ASP | A | 185 | 1646 | 1663 | 1641 | −46 | 31 | 43 | A | C |
| ATOM | 1050 | O | ASP | A | 185 | −12.507 | 8.445 | −12.044 | 1.00 | 12.35 | | A | O |
| ANISOU | 1050 | O | ASP | A | 185 | 1543 | 1620 | 1530 | −72 | 73 | 16 | A | O |
| ATOM | 1051 | N | ASP | A | 186 | −11.551 | 10.493 | −12.014 | 1.00 | 14.38 | | A | N |
| ANISOU | 1051 | N | ASP | A | 186 | 1770 | 1800 | 1893 | −78 | −5 | 64 | A | N |
| ATOM | 1052 | CA | ASP | A | 186 | −11.929 | 10.816 | −13.397 | 1.00 | 15.06 | | A | C |
| ANISOU | 1052 | CA | ASP | A | 186 | 1858 | 1917 | 1947 | −50 | −4 | 54 | A | C |
| ATOM | 1053 | CB | ASP | A | 186 | −11.142 | 9.914 | −14.371 | 1.00 | 16.64 | | A | C |
| ANISOU | 1053 | CB | ASP | A | 186 | 2027 | 2119 | 2176 | −4 | 46 | −23 | A | C |
| ATOM | 1054 | CG | ASP | A | 186 | −11.304 | 10.313 | −15.835 | 1.00 | 20.66 | | A | C |
| ANISOU | 1054 | CG | ASP | A | 186 | 2607 | 2784 | 2458 | −2 | −31 | 56 | A | C |
| ATOM | 1055 | OD1 | ASP | A | 186 | −11.462 | 11.518 | −16.132 | 1.00 | 23.13 | | A | O |
| ANISOU | 1055 | OD1 | ASP | A | 186 | 2913 | 2864 | 3011 | −16 | 16 | 92 | A | O |
| ATOM | 1056 | OD2 | ASP | A | 186 | −11.256 | 9.403 | −16.694 | 1.00 | 22.17 | | A | O |
| ANISOU | 1056 | OD2 | ASP | A | 186 | 2795 | 2908 | 2721 | 14 | 17 | −57 | A | O |
| ATOM | 1057 | C | ASP | A | 186 | −13.453 | 10.646 | −13.553 | 1.00 | 14.16 | | A | C |
| ANISOU | 1057 | C | ASP | A | 186 | 1778 | 1771 | 1832 | −30 | 9 | 34 | A | C |
| ATOM | 1058 | O | ASP | A | 186 | −13.943 | 10.174 | −14.586 | 1.00 | 14.56 | | A | O |
| ANISOU | 1058 | O | ASP | A | 186 | 1810 | 1879 | 1842 | −83 | 11 | 107 | A | O |
| ATOM | 1059 | N | ASP | A | 187 | −14.177 | 11.023 | −12.492 | 1.00 | 13.07 | | A | N |
| ANISOU | 1059 | N | ASP | A | 187 | 1589 | 1589 | 1789 | −32 | −33 | 48 | A | N |
| ATOM | 1060 | CA | ASP | A | 187 | −15.645 | 10.908 | −12.381 | 1.00 | 12.01 | | A | C |
| ANISOU | 1060 | CA | ASP | A | 187 | 1541 | 1431 | 1593 | −10 | −29 | 51 | A | C |
| ATOM | 1061 | CB | ASP | A | 187 | −16.352 | 11.884 | −13.340 | 1.00 | 12.13 | | A | C |
| ANISOU | 1061 | CB | ASP | A | 187 | 1530 | 1529 | 1549 | −4 | −60 | 64 | A | C |
| ATOM | 1062 | CG | ASP | A | 187 | −17.732 | 12.317 | −12.844 | 1.00 | 12.22 | | A | C |
| ANISOU | 1062 | CG | ASP | A | 187 | 1603 | 1456 | 1584 | −27 | −3 | 30 | A | C |
| ATOM | 1063 | OD1 | ASP | A | 187 | −18.513 | 12.840 | −13.664 | 1.00 | 13.12 | | A | O |
| ANISOU | 1063 | OD1 | ASP | A | 187 | 1607 | 1595 | 1783 | −10 | −98 | 74 | A | O |
| ATOM | 1064 | OD2 | ASP | A | 187 | −18.047 | 12.143 | −11.647 | 1.00 | 12.27 | | A | O |
| ANISOU | 1064 | OD2 | ASP | A | 187 | 1661 | 1475 | 1525 | 41 | −32 | 51 | A | O |
| ATOM | 1065 | C | ASP | A | 187 | −16.159 | 9.474 | −12.564 | 1.00 | 11.53 | | A | C |
| ANISOU | 1065 | C | ASP | A | 187 | 1431 | 1432 | 1519 | −6 | −13 | 26 | A | C |
| ATOM | 1066 | O | ASP | A | 187 | −17.283 | 9.261 | −13.026 | 1.00 | 11.81 | | A | O |
| ANISOU | 1066 | O | ASP | A | 187 | 1448 | 1480 | 1558 | 28 | −87 | 43 | A | O |
| ATOM | 1067 | N | LYS | A | 188 | −15.329 | 8.498 | −12.196 | 1.00 | 10.71 | | A | N |
| ANISOU | 1067 | N | LYS | A | 188 | 1397 | 1294 | 1378 | −39 | 16 | 35 | A | N |
| ATOM | 1068 | CA | LYS | A | 188 | −15.711 | 7.084 | −12.247 | 1.00 | 9.82 | | A | C |
| ANISOU | 1068 | CA | LYS | A | 188 | 1269 | 1264 | 1200 | −45 | 73 | 25 | A | C |
| ATOM | 1069 | CB | LYS | A | 188 | −14.735 | 6.274 | −13.103 | 1.00 | 10.24 | | A | C |
| ANISOU | 1069 | CB | LYS | A | 188 | 1263 | 1322 | 1304 | 65 | 6 | 38 | A | C |
| ATOM | 1070 | CG | LYS | A | 188 | −14.510 | 6.790 | −14.519 | 1.00 | 12.79 | | A | C |
| ANISOU | 1070 | CG | LYS | A | 188 | 1703 | 1755 | 1401 | 23 | −5 | 45 | A | C |
| ATOM | 1071 | CD | LYS | A | 188 | −13.532 | 5.873 | −15.238 | 1.00 | 14.78 | | A | C |
| ANISOU | 1071 | CD | LYS | A | 188 | 1760 | 1969 | 1886 | 68 | 32 | −64 | A | C |
| ATOM | 1072 | CE | LYS | A | 188 | −13.233 | 6.338 | −16.651 | 1.00 | 17.30 | | A | C |
| ANISOU | 1072 | CE | LYS | A | 188 | 2231 | 2283 | 2059 | 1 | 20 | 37 | A | C |
| ATOM | 1073 | NZ | LYS | A | 188 | −12.227 | 5.436 | −17.288 | 1.00 | 18.60 | | A | N |
| ANISOU | 1073 | NZ | LYS | A | 188 | 2343 | 2408 | 2315 | 55 | −4 | −36 | A | N |
| ATOM | 1074 | C | LYS | A | 188 | −15.737 | 6.497 | −10.839 | 1.00 | 10.19 | | A | C |
| ANISOU | 1074 | C | LYS | A | 188 | 1292 | 1329 | 1250 | −57 | −20 | 70 | A | C |
| ATOM | 1075 | O | LYS | A | 188 | −14.832 | 6.748 | −10.036 | 1.00 | 10.21 | | A | O |
| ANISOU | 1075 | O | LYS | A | 188 | 1292 | 1423 | 1163 | −77 | −2 | 129 | A | O |
| ATOM | 1076 | N | VAL | A | 189 | −16.779 | 5.721 | −10.553 | 1.00 | 9.70 | | A | N |
| ANISOU | 1076 | N | VAL | A | 189 | 1280 | 1223 | 1183 | −50 | −25 | 63 | A | N |
| ATOM | 1077 | CA | VAL | A | 189 | −16.910 | 5.010 | −9.283 | 1.00 | 9.75 | | A | C |
| ANISOU | 1077 | CA | VAL | A | 189 | 1275 | 1204 | 1224 | −14 | −2 | 42 | A | C |
| ATOM | 1078 | CB | VAL | A | 189 | −18.102 | 5.541 | −8.441 | 1.00 | 10.67 | | A | C |
| ANISOU | 1078 | CB | VAL | A | 189 | 1392 | 1355 | 1308 | −5 | 10 | 19 | A | C |
| ATOM | 1079 | CG1 | VAL | A | 189 | −18.280 | 4.729 | −7.170 | 1.00 | 9.92 | | A | C |
| ANISOU | 1079 | CG1 | VAL | A | 189 | 1349 | 1266 | 1155 | 10 | 29 | −16 | A | C |
| ATOM | 1080 | CG2 | VAL | A | 189 | −17.910 | 7.011 | −8.100 | 1.00 | 10.70 | | A | C |
| ANISOU | 1080 | CG2 | VAL | A | 189 | 1433 | 1294 | 1337 | 29 | 12 | −31 | A | C |
| ATOM | 1081 | C | VAL | A | 189 | −17.119 | 3.536 | −9.611 | 1.00 | 9.73 | | A | C |
| ANISOU | 1081 | C | VAL | A | 189 | 1224 | 1233 | 1239 | −7 | −15 | 20 | A | C |
| ATOM | 1082 | O | VAL | A | 189 | −17.898 | 3.208 | −10.510 | 1.00 | 10.22 | | A | O |
| ANISOU | 1082 | O | VAL | A | 189 | 1283 | 1321 | 1278 | −47 | −28 | 33 | A | O |
| ATOM | 1083 | N | THR | A | 190 | −16.404 | 2.661 | −8.904 | 1.00 | 8.62 | | A | N |
| ANISOU | 1083 | N | THR | A | 190 | 1001 | 1118 | 1158 | −41 | 52 | 109 | A | N |
| ATOM | 1084 | CA | THR | A | 190 | −16.476 | 1.220 | −9.145 | 1.00 | 10.00 | | A | C |
| ANISOU | 1084 | CA | THR | A | 190 | 1270 | 1261 | 1267 | −15 | 33 | 16 | A | C |
| ATOM | 1085 | CB | THR | A | 190 | −15.078 | 0.599 | −9.403 | 1.00 | 10.37 | | A | C |
| ANISOU | 1085 | CB | THR | A | 190 | 1262 | 1302 | 1376 | −24 | 42 | 41 | A | C |
| ATOM | 1086 | OG1 | THR | A | 190 | −14.460 | 1.259 | −10.518 | 1.00 | 12.91 | | A | O |
| ANISOU | 1086 | OG1 | THR | A | 190 | 1593 | 1725 | 1588 | −49 | 188 | 151 | A | O |
| ATOM | 1087 | CG2 | THR | A | 190 | −15.190 | −0.900 | −9.698 | 1.00 | 11.30 | | A | C |
| ANISOU | 1087 | CG2 | THR | A | 190 | 1503 | 1404 | 1385 | 88 | 48 | −55 | A | C |
| ATOM | 1088 | C | THR | A | 190 | −17.165 | 0.500 | −7.992 | 1.00 | 10.17 | | A | C |
| ANISOU | 1088 | C | THR | A | 190 | 1274 | 1312 | 1280 | −74 | 5 | −5 | A | C |
| ATOM | 1089 | O | THR | A | 190 | −16.771 | 0.636 | −6.823 | 1.00 | 10.42 | | A | O |
| ANISOU | 1089 | O | THR | A | 190 | 1289 | 1407 | 1263 | −49 | 30 | 42 | A | O |
| ATOM | 1090 | N | PHE | A | 191 | −18.192 | −0.264 | −8.351 | 1.00 | 9.37 | | A | N |
| ANISOU | 1090 | N | PHE | A | 191 | 1138 | 1221 | 1201 | −84 | 2 | 80 | A | N |
| ATOM | 1091 | CA | PHE | A | 191 | −18.969 | −1.066 | −7.424 | 1.00 | 9.38 | | A | C |
| ANISOU | 1091 | CA | PHE | A | 191 | 1238 | 1215 | 1110 | −9 | 53 | −6 | A | C |
| ATOM | 1092 | CB | PHE | A | 191 | −20.457 | −0.853 | −7.693 | 1.00 | 9.73 | | A | C |
| ANISOU | 1092 | CB | PHE | A | 191 | 1251 | 1231 | 1214 | 37 | 72 | −58 | A | C |
| ATOM | 1093 | CG | PHE | A | 191 | −20.893 | 0.570 | −7.515 | 1.00 | 9.51 | | A | C |
| ANISOU | 1093 | CG | PHE | A | 191 | 1346 | 1072 | 1197 | 1 | 55 | 31 | A | C |
| ATOM | 1094 | CD1 | PHE | A | 191 | −20.729 | 1.507 | −8.541 | 1.00 | 9.10 | | A | C |
| ANISOU | 1094 | CD1 | PHE | A | 191 | 1077 | 1122 | 1259 | −12 | −33 | 77 | A | C |
| ATOM | 1095 | CE1 | PHE | A | 191 | −21.119 | 2.835 | −8.358 | 1.00 | 10.77 | | A | C |
| ANISOU | 1095 | CE1 | PHE | A | 191 | 1278 | 1342 | 1472 | 63 | 57 | −30 | A | C |
| ATOM | 1096 | CZ | PHE | A | 191 | −21.677 | 3.234 | −7.151 | 1.00 | 11.13 | | A | C |
| ANISOU | 1096 | CZ | PHE | A | 191 | 1399 | 1423 | 1408 | 18 | 36 | 48 | A | C |
| ATOM | 1097 | CE2 | PHE | A | 191 | −21.848 | 2.304 | −6.125 | 1.00 | 11.38 | | A | C |
| ANISOU | 1097 | CE2 | PHE | A | 191 | 1584 | 1258 | 1483 | 33 | 22 | 26 | A | C |
| ATOM | 1098 | CD2 | PHE | A | 191 | −21.451 | 0.983 | −6.311 | 1.00 | 9.78 | | A | C |
| ANISOU | 1098 | CD2 | PHE | A | 191 | 1286 | 1270 | 1161 | 14 | 149 | 76 | A | C |
| ATOM | 1099 | C | PHE | A | 191 | −18.610 | −2.529 | −7.601 | 1.00 | 9.35 | | A | C |
| ANISOU | 1099 | C | PHE | A | 191 | 1200 | 1167 | 1184 | −11 | 74 | 34 | A | C |
| ATOM | 1100 | O | PHE | A | 191 | −18.209 | −2.948 | −8.691 | 1.00 | 9.95 | | A | O |
| ANISOU | 1100 | O | PHE | A | 191 | 1407 | 1210 | 1162 | −31 | 82 | −18 | A | O |
| ATOM | 1101 | N | GLU | A | 192 | −18.751 | −3.307 | −6.533 | 1.00 | 8.73 | | A | N |
| ANISOU | 1101 | N | GLU | A | 192 | 1193 | 1038 | 1085 | 17 | 48 | −3 | A | N |
| ATOM | 1102 | CA | GLU | A | 192 | −18.448 | −4.731 | −6.599 | 1.00 | 9.10 | | A | C |
| ANISOU | 1102 | CA | GLU | A | 192 | 1188 | 1115 | 1155 | 10 | 56 | 47 | A | C |
| ATOM | 1103 | CB | GLU | A | 192 | −17.030 | −4.988 | −6.095 | 1.00 | 9.49 | | A | C |
| ANISOU | 1103 | CB | GLU | A | 192 | 1204 | 1161 | 1242 | 23 | −42 | −9 | A | C |
| ATOM | 1104 | CG | GLU | A | 192 | −16.596 | −6.443 | −6.151 | 1.00 | 10.77 | | A | C |
| ANISOU | 1104 | CG | GLU | A | 192 | 1430 | 1222 | 1440 | 49 | −14 | −55 | A | C |
| ATOM | 1105 | CD | GLU | A | 192 | −15.300 | −6.687 | −5.410 | 1.00 | 14.30 | | A | C |
| ANISOU | 1105 | CD | GLU | A | 192 | 1625 | 1923 | 1885 | 17 | −109 | −24 | A | C |
| ATOM | 1106 | OE1 | GLU | A | 192 | −15.359 | −7.039 | −4.211 | 1.00 | 15.66 | | A | O |
| ANISOU | 1106 | OE1 | GLU | A | 192 | 1851 | 2090 | 2009 | 18 | −28 | 52 | A | O |
| ATOM | 1107 | OE2 | GLU | A | 192 | −14.225 | −6.525 | −6.026 | 1.00 | 15.91 | | A | O |
| ANISOU | 1107 | OE2 | GLU | A | 192 | 1854 | 2079 | 2113 | −5 | 92 | 58 | A | O |
| ATOM | 1108 | C | GLU | A | 192 | −19.444 | −5.521 | −5.768 | 1.00 | 9.57 | | A | C |
| ANISOU | 1108 | C | GLU | A | 192 | 1258 | 1149 | 1229 | −65 | 5 | 16 | A | C |
| ATOM | 1109 | O | GLU | A | 192 | −19.814 | −5.103 | −4.675 | 1.00 | 9.65 | | A | O |
| ANISOU | 1109 | O | GLU | A | 192 | 1385 | 1136 | 1145 | 3 | 65 | 43 | A | O |
| ATOM | 1110 | N | VAL | A | 193 | −19.878 | −6.661 | −6.296 | 1.00 | 8.70 | | A | N |
| ANISOU | 1110 | N | VAL | A | 193 | 1146 | 1018 | 1142 | −26 | 26 | 17 | A | N |
| ATOM | 1111 | CA | VAL | A | 193 | −20.701 | −7.585 | −5.526 | 1.00 | 8.08 | | A | C |
| ANISOU | 1111 | CA | VAL | A | 193 | 1022 | 1016 | 1031 | −20 | 0 | 57 | A | C |
| ATOM | 1112 | CB | VAL | A | 193 | −22.153 | −7.687 | −6.085 | 1.00 | 9.31 | | A | C |
| ANISOU | 1112 | CB | VAL | A | 193 | 1094 | 1216 | 1229 | −31 | −44 | 62 | A | C |
| ATOM | 1113 | CG1 | VAL | A | 193 | −22.904 | −8.835 | −5.432 | 1.00 | 13.05 | | A | C |
| ANISOU | 1113 | CG1 | VAL | A | 193 | 1687 | 1604 | 1668 | −137 | 79 | 146 | A | C |
| ATOM | 1114 | CG2 | VAL | A | 193 | −22.911 | −6.396 | −5.856 | 1.00 | 11.14 | | A | C |
| ANISOU | 1114 | CG2 | VAL | A | 193 | 1412 | 1364 | 1456 | 42 | 23 | 44 | A | C |
| ATOM | 1115 | C | VAL | A | 193 | −20.039 | −8.958 | −5.508 | 1.00 | 8.95 | | A | C |
| ANISOU | 1115 | C | VAL | A | 193 | 1161 | 1090 | 1149 | −53 | 33 | −4 | A | C |
| ATOM | 1116 | O | VAL | A | 193 | −19.600 | −9.452 | −6.544 | 1.00 | 9.30 | | A | O |
| ANISOU | 1116 | O | VAL | A | 193 | 1294 | 1152 | 1088 | −42 | 164 | 33 | A | O |
| ATOM | 1117 | N | PHE | A | 194 | −19.954 | −9.541 | −4.315 | 1.00 | 8.32 | | A | N |
| ANISOU | 1117 | N | PHE | A | 194 | 1106 | 982 | 1074 | −33 | −30 | 63 | A | N |
| ATOM | 1118 | CA | PHE | A | 194 | −19.602 | −10.951 | −4.127 | 1.00 | 8.51 | | A | C |
| ANISOU | 1118 | CA | PHE | A | 194 | 1064 | 1080 | 1089 | 29 | 37 | 6 | A | C |
| ATOM | 1119 | CB | PHE | A | 194 | −18.548 | −11.088 | −3.020 | 1.00 | 9.07 | | A | C |
| ANISOU | 1119 | CB | PHE | A | 194 | 1118 | 1236 | 1092 | 5 | 53 | −1 | A | C |
| ATOM | 1120 | CG | PHE | A | 194 | −18.262 | −12.514 | −2.600 | 1.00 | 10.30 | | A | C |
| ANISOU | 1120 | CG | PHE | A | 194 | 1227 | 1325 | 1360 | −43 | 28 | 20 | A | C |
| ATOM | 1121 | CD1 | PHE | A | 194 | −18.137 | −13.533 | −3.541 | 1.00 | 11.90 | | A | C |
| ANISOU | 1121 | CD1 | PHE | A | 194 | 1598 | 1519 | 1403 | 0 | 37 | −35 | A | C |
| ATOM | 1122 | CE1 | PHE | A | 194 | −17.855 | −14.839 | −3.152 | 1.00 | 13.86 | | A | C |
| ANISOU | 1122 | CE1 | PHE | A | 194 | 1845 | 1646 | 1774 | 22 | −11 | 59 | A | C |
| ATOM | 1123 | CZ | PHE | A | 194 | −17.678 | −15.141 | −1.805 | 1.00 | 14.40 | | A | C |
| ANISOU | 1123 | CZ | PHE | A | 194 | 1931 | 1727 | 1812 | 155 | −89 | 53 | A | C |
| ATOM | 1124 | CE2 | PHE | A | 194 | −17.784 | −14.133 | −0.851 | 1.00 | 14.86 | | A | C |
| ANISOU | 1124 | CE2 | PHE | A | 194 | 2059 | 1688 | 1900 | 84 | −39 | 9 | A | C |
| ATOM | 1125 | CD2 | PHE | A | 194 | −18.070 | −12.822 | −1.254 | 1.00 | 12.85 | | A | C |
| ANISOU | 1125 | CD2 | PHE | A | 194 | 1674 | 1712 | 1497 | 36 | −2 | 126 | A | C |
| ATOM | 1126 | C | PHE | A | 194 | −20.899 | −11.655 | −3.750 | 1.00 | 8.53 | | A | C |
| ANISOU | 1126 | C | PHE | A | 194 | 1124 | 1087 | 1030 | −30 | 12 | −26 | A | C |
| ATOM | 1127 | O | PHE | A | 194 | −21.403 | −11.480 | −2.636 | 1.00 | 8.92 | | A | O |
| ANISOU | 1127 | O | PHE | A | 194 | 1220 | 1149 | 1021 | −70 | 74 | −70 | A | O |
| ATOM | 1128 | N | AVAL | A | 195 | −21.451 | −12.436 | −4.674 | 0.50 | 7.81 | | A | N |
| ANISOU | 1128 | N | AVAL | A | 195 | 988 | 1012 | 966 | −56 | 11 | −13 | A | N |
| ATOM | 1129 | N | BVAL | A | 195 | −21.431 | −12.432 | −4.694 | 0.50 | 8.02 | | A | N |
| ANISOU | 1129 | N | BVAL | A | 195 | 1014 | 1030 | 1002 | −56 | 7 | −17 | A | N |
| ATOM | 1130 | CA | AVAL | A | 195 | −22.746 | −13.076 | −4.448 | 0.50 | 7.71 | | A | C |
| ANISOU | 1130 | CA | AVAL | A | 195 | 984 | 973 | 971 | −5 | 9 | 5 | A | C |
| ATOM | 1131 | CA | BVAL | A | 195 | −22.701 | −13.141 | −4.540 | 0.50 | 8.13 | | A | C |
| ANISOU | 1131 | CA | BVAL | A | 195 | 1026 | 1000 | 1062 | −7 | 13 | 0 | A | C |
| ATOM | 1132 | CB | AVAL | A | 195 | −23.780 | −12.725 | −5.563 | 0.50 | 7.92 | | A | C |
| ANISOU | 1132 | CB | AVAL | A | 195 | 975 | 1074 | 962 | −10 | 49 | 19 | A | C |
| ATOM | 1133 | CB | BVAL | A | 195 | −23.512 | −13.176 | −5.867 | 0.50 | 8.50 | | A | C |
| ANISOU | 1133 | CB | BVAL | A | 195 | 1064 | 1054 | 1111 | 12 | 25 | −1 | A | C |
| ATOM | 1134 | CG1 | AVAL | A | 195 | −23.299 | −13.183 | −6.948 | 0.50 | 6.86 | | A | C |
| ANISOU | 1134 | CG1 | AVAL | A | 195 | 959 | 801 | 845 | −22 | −12 | 38 | A | C |
| ATOM | 1135 | CG1 | BVAL | A | 195 | −24.921 | −13.727 | −5.634 | 0.50 | 7.87 | | A | C |
| ANISOU | 1135 | CG1 | BVAL | A | 195 | 1017 | 971 | 1002 | −26 | −37 | −12 | A | C |
| ATOM | 1136 | CG2 | AVAL | A | 195 | −25.161 | −13.303 | −5.231 | 0.50 | 7.43 | | A | C |
| ANISOU | 1136 | CG2 | AVAL | A | 195 | 883 | 933 | 1007 | −31 | −16 | 38 | A | C |
| ATOM | 1137 | CG2 | BVAL | A | 195 | −23.578 | −11.806 | −6.517 | 0.50 | 9.23 | | A | C |
| ANISOU | 1137 | CG2 | BVAL | A | 195 | 1155 | 1079 | 1273 | −23 | −30 | 26 | A | C |
| ATOM | 1138 | C | AVAL | A | 195 | −22.600 | −14.584 | −4.260 | 0.50 | 7.96 | | A | C |
| ANISOU | 1138 | C | AVAL | A | 195 | 1017 | 1006 | 1003 | −13 | 21 | −14 | A | C |
| ATOM | 1139 | C | BVAL | A | 195 | −22.422 | −14.582 | −4.149 | 0.50 | 8.20 | | A | C |
| ANISOU | 1139 | C | BVAL | A | 195 | 1016 | 1013 | 1085 | −30 | 36 | −17 | A | C |
| ATOM | 1140 | O | AVAL | A | 195 | −21.994 | −15.267 | −5.086 | 0.50 | 7.51 | | A | O |
| ANISOU | 1140 | O | AVAL | A | 195 | 983 | 935 | 936 | 49 | −23 | −58 | A | O |
| ATOM | 1141 | O | BVAL | A | 195 | −21.562 | −15.227 | −4.744 | 0.50 | 7.95 | | A | O |
| ANISOU | 1141 | O | BVAL | A | 195 | 956 | 931 | 1132 | 7 | 8 | −82 | A | O |
| ATOM | 1142 | N | GLN | A | 196 | −23.150 | −15.082 | −3.154 | 1.00 | 7.74 | | A | N |
| ANISOU | 1142 | N | GLN | A | 196 | 1045 | 954 | 942 | −4 | 40 | −7 | A | N |
| ATOM | 1143 | CA | GLN | A | 196 | −23.120 | −16.504 | −2.827 | 1.00 | 8.20 | | A | C |
| ANISOU | 1143 | CA | GLN | A | 196 | 1127 | 1018 | 971 | −51 | 17 | 21 | A | C |
| ATOM | 1144 | CB | GLN | A | 196 | −22.436 | −16.730 | −1.479 | 1.00 | 9.37 | | A | C |
| ANISOU | 1144 | CB | GLN | A | 196 | 1239 | 1178 | 1145 | 22 | −52 | 57 | A | C |
| ATOM | 1145 | CG | GLN | A | 196 | −20.946 | −16.464 | −1.510 | 1.00 | 10.39 | | A | C |
| ANISOU | 1145 | CG | GLN | A | 196 | 1247 | 1253 | 1446 | 17 | −24 | 6 | A | C |
| ATOM | 1146 | CD | GLN | A | 196 | −20.322 | −16.505 | −0.132 | 1.00 | 14.85 | | A | C |
| ANISOU | 1146 | CD | GLN | A | 196 | 1966 | 1983 | 1694 | 42 | −159 | −18 | A | C |
| ATOM | 1147 | OE1 | GLN | A | 196 | −20.594 | −15.649 | 0.712 | 1.00 | 14.69 | | A | O |
| ANISOU | 1147 | OE1 | GLN | A | 196 | 1918 | 1807 | 1856 | 126 | −155 | 20 | A | O |
| ATOM | 1148 | NE2 | GLN | A | 196 | −19.477 | −17.507 | 0.106 | 1.00 | 16.84 | | A | N |
| ANISOU | 1148 | NE2 | GLN | A | 196 | 2104 | 2062 | 2231 | 116 | −64 | 44 | A | N |
| ATOM | 1149 | C | GLN | A | 196 | −24.562 | −16.970 | −2.801 | 1.00 | 8.65 | | A | C |
| ANISOU | 1149 | C | GLN | A | 196 | 1152 | 1049 | 1084 | 14 | −15 | −33 | A | C |
| ATOM | 1150 | O | GLN | A | 196 | −25.274 | −16.756 | −1.819 | 1.00 | 9.10 | | A | O |
| ANISOU | 1150 | O | GLN | A | 196 | 1267 | 1052 | 1137 | −18 | 77 | 61 | A | O |
| ATOM | 1151 | N | ALA | A | 197 | −24.992 | −17.587 | −3.901 | 1.00 | 8.72 | | A | N |
| ANISOU | 1151 | N | ALA | A | 197 | 1181 | 1129 | 1005 | 1 | −13 | −87 | A | N |
| ATOM | 1152 | CA | ALA | A | 197 | −26.397 | −17.906 | −4.110 | 1.00 | 8.35 | | A | C |
| ANISOU | 1152 | CA | ALA | A | 197 | 1120 | 1075 | 979 | −11 | 43 | −44 | A | C |
| ATOM | 1153 | CB | ALA | A | 197 | −26.825 | −17.504 | −5.513 | 1.00 | 9.17 | | A | C |
| ANISOU | 1153 | CB | ALA | A | 197 | 1127 | 1231 | 1125 | −11 | −11 | 60 | A | C |
| ATOM | 1154 | C | ALA | A | 197 | −26.678 | −19.381 | −3.883 | 1.00 | 8.70 | | A | C |
| ANISOU | 1154 | C | ALA | A | 197 | 1166 | 1064 | 1075 | −11 | 21 | 1 | A | C |
| ATOM | 1155 | O | ALA | A | 197 | −25.822 | −20.226 | −4.141 | 1.00 | 10.35 | | A | O |
| ANISOU | 1155 | O | ALA | A | 197 | 1281 | 1252 | 1398 | 78 | 92 | −29 | A | O |
| ATOM | 1156 | N | ASP | A | 198 | −27.877 | −19.678 | −3.389 | 1.00 | 7.87 | | A | N |
| ANISOU | 1156 | N | ASP | A | 198 | 1025 | 937 | 1027 | 36 | 10 | −20 | A | N |
| ATOM | 1157 | CA | ASP | A | 198 | −28.353 | −21.060 | −3.284 | 1.00 | 8.30 | | A | C |
| ANISOU | 1157 | CA | ASP | A | 198 | 1000 | 1066 | 1089 | −39 | 63 | −20 | A | C |
| ATOM | 1158 | CB | ASP | A | 198 | −29.470 | −21.167 | −2.238 | 1.00 | 8.73 | | A | C |
| ANISOU | 1158 | CB | ASP | A | 198 | 1077 | 1221 | 1020 | −28 | 82 | 4 | A | C |
| ATOM | 1159 | CG | ASP | A | 198 | −28.975 | −20.986 | −0.813 | 1.00 | 8.67 | | A | C |
| ANISOU | 1159 | CG | ASP | A | 198 | 1115 | 1088 | 1091 | −45 | 27 | −55 | A | C |
| ATOM | 1160 | OD1 | ASP | A | 198 | −29.844 | −20.916 | 0.085 | 1.00 | 9.19 | | A | O |
| ANISOU | 1160 | OD1 | ASP | A | 198 | 1301 | 1099 | 1093 | 6 | 109 | −119 | A | O |
| ATOM | 1161 | OD2 | ASP | A | 198 | −27.744 | −20.929 | −0.584 | 1.00 | 9.03 | | A | O |
| ANISOU | 1161 | OD2 | ASP | A | 198 | 1244 | 1077 | 1111 | −39 | −59 | −58 | A | O |
| ATOM | 1162 | C | ASP | A | 198 | −28.905 | −21.518 | −4.625 | 1.00 | 8.77 | | A | C |
| ANISOU | 1162 | C | ASP | A | 198 | 1113 | 1121 | 1099 | 27 | 41 | −2 | A | C |
| ATOM | 1163 | O | ASP | A | 198 | −29.184 | −20.699 | −5.505 | 1.00 | 9.95 | | A | O |
| ANISOU | 1163 | O | ASP | A | 198 | 1331 | 1224 | 1224 | 23 | 21 | 59 | A | O |
| ATOM | 1164 | N | ALA | A | 199 | −29.077 | −22.828 | −4.781 | 1.00 | 9.51 | | A | N |
| ANISOU | 1164 | N | ALA | A | 199 | 1222 | 1204 | 1187 | −66 | 12 | −74 | A | N |
| ATOM | 1165 | CA | ALA | A | 199 | −29.779 | −23.356 | −5.949 | 1.00 | 9.57 | | A | C |
| ANISOU | 1165 | CA | ALA | A | 199 | 1145 | 1239 | 1254 | −34 | 1 | −62 | A | C |
| ATOM | 1166 | CB | ALA | A | 199 | −29.860 | −24.864 | −5.879 | 1.00 | 10.93 | | A | C |
| ANISOU | 1166 | CB | ALA | A | 199 | 1367 | 1292 | 1494 | 9 | −5 | −84 | A | C |
| ATOM | 1167 | C | ALA | A | 199 | −31.179 | −22.737 | −6.012 | 1.00 | 9.51 | | A | C |
| ANISOU | 1167 | C | ALA | A | 199 | 1189 | 1174 | 1250 | −62 | −7 | −7 | A | C |
| ATOM | 1168 | O | ALA | A | 199 | −31.881 | −22.691 | −5.003 | 1.00 | 9.65 | | A | O |
| ANISOU | 1168 | O | ALA | A | 199 | 1161 | 1254 | 1250 | 40 | 14 | 9 | A | O |
| ATOM | 1169 | N | PRO | A | 200 | −31.569 | −22.211 | −7.184 | 1.00 | 10.35 | | A | N |
| ANISOU | 1169 | N | PRO | A | 200 | 1242 | 1377 | 1312 | 8 | 12 | −17 | A | N |
| ATOM | 1170 | CA | PRO | A | 200 | −32.902 | −21.629 | −7.294 | 1.00 | 9.92 | | A | C |
| ANISOU | 1170 | CA | PRO | A | 200 | 1233 | 1293 | 1244 | −14 | −17 | 30 | A | C |
| ATOM | 1171 | CB | PRO | A | 200 | −32.834 | −20.873 | −8.620 | 1.00 | 10.89 | | A | C |
| ANISOU | 1171 | CB | PRO | A | 200 | 1447 | 1419 | 1273 | −12 | 15 | 44 | A | C |
| ATOM | 1172 | CG | PRO | A | 200 | −31.866 | −21.666 | −9.429 | 1.00 | 12.00 | | A | C |
| ANISOU | 1172 | CG | PRO | A | 200 | 1388 | 1731 | 1439 | 118 | −11 | 58 | A | C |
| ATOM | 1173 | CD | PRO | A | 200 | −30.813 | −22.093 | −8.445 | 1.00 | 11.26 | | A | C |
| ANISOU | 1173 | CD | PRO | A | 200 | 1404 | 1621 | 1252 | 17 | −10 | 53 | A | C |
| ATOM | 1174 | C | PRO | A | 200 | −33.993 | −22.691 | −7.362 | 1.00 | 10.00 | | A | C |
| ANISOU | 1174 | C | PRO | A | 200 | 1241 | 1238 | 1322 | −19 | 7 | −31 | A | C |
| ATOM | 1175 | O | PRO | A | 200 | −33.712 | −23.865 | −7.638 | 1.00 | 10.98 | | A | O |
| ANISOU | 1175 | O | PRO | A | 200 | 1345 | 1241 | 1587 | −9 | −30 | −99 | A | O |
| ATOM | 1176 | N | HIS | A | 201 | −35.225 | −22.271 | −7.098 | 1.00 | 9.36 | | A | N |
| ANISOU | 1176 | N | HIS | A | 201 | 1178 | 1115 | 1263 | 14 | 10 | −32 | A | N |
| ATOM | 1177 | CA | HIS | A | 201 | −36.400 | −23.091 | −7.362 | 1.00 | 9.51 | | A | C |
| ANISOU | 1177 | CA | HIS | A | 201 | 1202 | 1230 | 1182 | −24 | −3 | 6 | A | C |
| ATOM | 1178 | CB | HIS | A | 201 | −37.246 | −23.252 | −6.098 | 1.00 | 10.70 | | A | C |
| ANISOU | 1178 | CB | HIS | A | 201 | 1438 | 1365 | 1264 | 38 | 92 | 46 | A | C |
| ATOM | 1179 | CG | HIS | A | 201 | −36.640 | −24.161 | −5.078 | 1.00 | 12.53 | | A | C |
| ANISOU | 1179 | CG | HIS | A | 201 | 1496 | 1645 | 1621 | 98 | −52 | 95 | A | C |
| ATOM | 1180 | ND1 | HIS | A | 201 | −36.005 | −23.695 | −3.947 | 1.00 | 15.43 | | A | N |
| ANISOU | 1180 | ND1 | HIS | A | 201 | 2031 | 2050 | 1783 | 35 | 5 | −13 | A | N |
| ATOM | 1181 | CE1 | HIS | A | 201 | −35.575 | −24.721 | −3.233 | 1.00 | 15.94 | | A | C |
| ANISOU | 1181 | CE1 | HIS | A | 201 | 2033 | 2089 | 1933 | 73 | −9 | 62 | A | C |
| ATOM | 1182 | NE2 | HIS | A | 201 | −35.907 | −25.834 | −3.861 | 1.00 | 17.34 | | A | N |
| ANISOU | 1182 | NE2 | HIS | A | 201 | 2195 | 2228 | 2167 | 10 | −121 | 130 | A | N |
| ATOM | 1183 | CD2 | HIS | A | 201 | −36.578 | −25.513 | −5.015 | 1.00 | 14.12 | | A | C |
| ANISOU | 1183 | CD2 | HIS | A | 201 | 1755 | 1750 | 1860 | −33 | 80 | 26 | A | C |
| ATOM | 1184 | C | HIS | A | 201 | −37.236 | −22.445 | −8.461 | 1.00 | 9.30 | | A | C |
| ANISOU | 1184 | C | HIS | A | 201 | 1148 | 1160 | 1225 | 14 | −1 | −12 | A | C |
| ATOM | 1185 | O | HIS | A | 201 | −37.108 | −21.248 | −8.731 | 1.00 | 9.20 | | A | O |
| ANISOU | 1185 | O | HIS | A | 201 | 1162 | 1157 | 1177 | 27 | −72 | −54 | A | O |
| ATOM | 1186 | N | GLY | A | 202 | −38.074 | −23.247 | −9.107 | 1.00 | 9.47 | | A | N |
| ANISOU | 1186 | N | GLY | A | 202 | 1134 | 1276 | 1188 | 12 | 7 | −41 | A | N |
| ATOM | 1187 | CA | GLY | A | 202 | −39.027 | −22.733 | −10.084 | 1.00 | 9.97 | | A | C |
| ANISOU | 1187 | CA | GLY | A | 202 | 1216 | 1353 | 1221 | 0 | −23 | 21 | A | C |
| ATOM | 1188 | C | GLY | A | 202 | −38.414 | −22.254 | −11.386 | 1.00 | 10.98 | | A | C |
| ANISOU | 1188 | C | GLY | A | 202 | 1373 | 1420 | 1377 | −19 | 31 | 32 | A | C |
| ATOM | 1189 | O | GLY | A | 202 | −38.982 | −21.390 | −12.052 | 1.00 | 12.50 | | A | O |
| ANISOU | 1189 | O | GLY | A | 202 | 1622 | 1571 | 1558 | −3 | 3 | 77 | A | O |
| ATOM | 1190 | N | VAL | A | 203 | −37.262 | −22.818 | −11.751 | 1.00 | 11.11 | | A | N |
| ANISOU | 1190 | N | VAL | A | 203 | 1365 | 1462 | 1394 | −51 | 36 | −15 | A | N |
| ATOM | 1191 | CA | VAL | A | 203 | −36.601 | −22.472 | −13.015 | 1.00 | 12.08 | | A | C |
| ANISOU | 1191 | CA | VAL | A | 203 | 1582 | 1563 | 1445 | −14 | 43 | 4 | A | C |
| ATOM | 1192 | CB | VAL | A | 203 | −35.182 | −21.866 | −12.805 | 1.00 | 12.03 | | A | C |
| ANISOU | 1192 | CB | VAL | A | 203 | 1512 | 1552 | 1507 | −23 | 60 | −50 | A | C |
| ATOM | 1193 | CG1 | VAL | A | 203 | −35.259 | −20.531 | −12.068 | 1.00 | 13.35 | | A | C |
| ANISOU | 1193 | CG1 | VAL | A | 203 | 1885 | 1554 | 1632 | −96 | 8 | −114 | A | C |
| ATOM | 1194 | CG2 | VAL | A | 203 | −34.270 | −22.820 | −12.063 | 1.00 | 15.76 | | A | C |
| ANISOU | 1194 | CG2 | VAL | A | 203 | 2081 | 1920 | 1987 | 78 | 16 | 124 | A | C |
| ATOM | 1195 | C | VAL | A | 203 | −36.525 | −23.655 | −13.986 | 1.00 | 12.37 | | A | C |
| ANISOU | 1195 | C | VAL | A | 203 | 1637 | 1548 | 1516 | 23 | −15 | −24 | A | C |
| ATOM | 1196 | O | VAL | A | 203 | −36.919 | −23.528 | −15.145 | 1.00 | 13.23 | | A | O |
| ANISOU | 1196 | O | VAL | A | 203 | 1708 | 1714 | 1603 | 140 | 1 | 30 | A | O |
| ATOM | 1197 | N | ALA | A | 204 | −36.018 | −24.796 | −13.511 | 1.00 | 13.12 | | A | N |
| ANISOU | 1197 | N | ALA | A | 204 | 1730 | 1578 | 1678 | 0 | 5 | −28 | A | N |
| ATOM | 1198 | CA | ALA | A | 204 | −35.904 | −26.001 | −14.341 | 1.00 | 14.01 | | A | C |
| ANISOU | 1198 | CA | ALA | A | 204 | 1825 | 1758 | 1739 | 43 | 0 | −64 | A | C |
| ATOM | 1199 | CB | ALA | A | 204 | −34.617 | −26.756 | −14.020 | 1.00 | 14.15 | | A | C |
| ANISOU | 1199 | CB | ALA | A | 204 | 1789 | 1807 | 1779 | 18 | −11 | −47 | A | C |
| ATOM | 1200 | C | ALA | A | 204 | −37.111 | −26.916 | −14.159 | 1.00 | 14.74 | | A | C |
| ANISOU | 1200 | C | ALA | A | 204 | 1844 | 1900 | 1857 | −4 | −7 | −47 | A | C |
| ATOM | 1201 | O | ALA | A | 204 | −37.591 | −27.105 | −13.037 | 1.00 | 16.66 | | A | O |
| ANISOU | 1201 | O | ALA | A | 204 | 2104 | 2177 | 2049 | 60 | 28 | 56 | A | O |
| ATOM | 1202 | N | TRP | A | 205 | −37.588 | −27.486 | −15.266 | 1.00 | 14.96 | | A | N |
| ANISOU | 1202 | N | TRP | A | 205 | 1886 | 1924 | 1875 | 27 | −24 | −3 | A | N |
| ATOM | 1203 | CA | TRP | A | 205 | −38.736 | −28.394 | −15.254 | 1.00 | 15.22 | | A | C |
| ANISOU | 1203 | CA | TRP | A | 205 | 1929 | 1985 | 1870 | 6 | −17 | 5 | A | C |
| ATOM | 1204 | CB | TRP | A | 205 | −39.268 | −28.578 | −16.681 | 1.00 | 15.26 | | A | C |
| ANISOU | 1204 | CB | TRP | A | 205 | 1931 | 1988 | 1881 | −25 | −30 | −45 | A | C |
| ATOM | 1205 | CG | TRP | A | 205 | −40.327 | −29.627 | −16.839 | 1.00 | 14.96 | | A | C |
| ANISOU | 1205 | CG | TRP | A | 205 | 1896 | 1967 | 1823 | 6 | 21 | −2 | A | C |
| ATOM | 1206 | CD1 | TRP | A | 205 | −41.633 | −29.554 | −16.434 | 1.00 | 15.36 | | A | C |
| ANISOU | 1206 | CD1 | TRP | A | 205 | 1899 | 1934 | 2003 | 17 | 18 | −6 | A | C |
| ATOM | 1207 | NE1 | TRP | A | 205 | −42.300 | −30.710 | −16.773 | 1.00 | 15.07 | | A | N |
| ANISOU | 1207 | NE1 | TRP | A | 205 | 1848 | 1924 | 1954 | 54 | −2 | 17 | A | N |
| ATOM | 1208 | CE2 | TRP | A | 205 | −41.429 | −31.550 | −17.417 | 1.00 | 15.65 | | A | C |
| ANISOU | 1208 | CE2 | TRP | A | 205 | 1980 | 1926 | 2039 | −8 | 64 | 12 | A | C |
| ATOM | 1209 | CD2 | TRP | A | 205 | −40.175 | −30.899 | −17.476 | 1.00 | 14.84 | | A | C |
| ANISOU | 1209 | CD2 | TRP | A | 205 | 1813 | 1944 | 1883 | 44 | −8 | −18 | A | C |
| ATOM | 1210 | CE3 | TRP | A | 205 | −39.101 | −31.553 | −18.096 | 1.00 | 13.59 | | A | C |
| ANISOU | 1210 | CE3 | TRP | A | 205 | 1657 | 1834 | 1672 | 82 | −74 | 22 | A | C |
| ATOM | 1211 | CZ3 | TRP | A | 205 | −39.307 | −32.823 | −18.624 | 1.00 | 14.90 | | A | C |
| ANISOU | 1211 | CZ3 | TRP | A | 205 | 1864 | 1883 | 1913 | 21 | −27 | −12 | A | C |
| ATOM | 1212 | CH2 | TRP | A | 205 | −40.568 | −33.445 | −18.545 | 1.00 | 16.23 | | A | C |
| ANISOU | 1212 | CH2 | TRP | A | 205 | 2029 | 1991 | 2145 | −30 | 32 | −32 | A | C |
| ATOM | 1213 | CZ2 | TRP | A | 205 | −41.637 | −32.826 | −17.949 | 1.00 | 15.89 | | A | C |
| ANISOU | 1213 | CZ2 | TRP | A | 205 | 2013 | 1999 | 2027 | −27 | 27 | −24 | A | C |
| ATOM | 1214 | C | TRP | A | 205 | −38.382 | −29.748 | −14.635 | 1.00 | 15.77 | | A | C |
| ANISOU | 1214 | C | TRP | A | 205 | 2003 | 2022 | 1965 | −2 | 4 | 28 | A | C |
| ATOM | 1215 | O | TRP | A | 205 | −37.366 | −30.358 | −14.977 | 1.00 | 15.54 | | A | O |
| ANISOU | 1215 | O | TRP | A | 205 | 1934 | 2028 | 1943 | 12 | −6 | 46 | A | O |
| ATOM | 1216 | OXT | TRP | A | 205 | −39.114 | −30.263 | −13.784 | 1.00 | 15.61 | | A | O |
| ANISOU | 1216 | OXT | TRP | A | 205 | 2000 | 2033 | 1897 | 7 | 42 | 50 | A | O |
| TER | | | | | | | | | | | | | |
| ATOM | 1217 | N | SER | B | 202 | −12.218 | −4.255 | 8.791 | 1.00 | 26.34 | | B | N |
| ANISOU | 1217 | N | SER | B | 202 | 3332 | 3314 | 3363 | −9 | −8 | −13 | B | N |
| ATOM | 1218 | CA | SER | B | 202 | −12.959 | −5.151 | 9.696 | 1.00 | 26.10 | | B | C |
| ANISOU | 1218 | CA | SER | B | 202 | 3262 | 3321 | 3332 | 0 | −11 | −10 | B | C |
| ATOM | 1219 | CB | SER | B | 202 | −12.667 | −4.800 | 11.162 | 1.00 | 26.41 | | B | C |
| ANISOU | 1219 | CB | SER | B | 202 | 3306 | 3383 | 3347 | 12 | −3 | −9 | B | C |
| ATOM | 1220 | OG | SER | B | 202 | −11.294 | −4.924 | 11.487 | 1.00 | 26.33 | | B | O |
| ANISOU | 1220 | OG | SER | B | 202 | 3256 | 3354 | 3394 | 0 | −34 | 0 | B | O |
| ATOM | 1221 | C | SER | B | 202 | −14.465 | −5.102 | 9.454 | 1.00 | 25.75 | | B | C |
| ANISOU | 1221 | C | SER | B | 202 | 3234 | 3245 | 3303 | −17 | 3 | 0 | B | C |
| ATOM | 1222 | O | SER | B | 202 | −15.057 | −4.027 | 9.460 | 1.00 | 25.89 | | B | O |
| ANISOU | 1222 | O | SER | B | 202 | 3239 | 3267 | 3331 | −1 | 22 | 31 | B | O |
| ATOM | 1223 | N | VAL | B | 203 | −15.054 | −6.283 | 9.289 | 1.00 | 24.73 | | B | N |
| ANISOU | 1223 | N | VAL | B | 203 | 3062 | 3174 | 3159 | −5 | 19 | −3 | B | N |
| ATOM | 1224 | CA | VAL | B | 203 | −16.490 | −6.439 | 9.028 | 1.00 | 23.69 | | B | C |
| ANISOU | 1224 | CA | VAL | B | 203 | 3004 | 3029 | 2969 | 10 | 40 | 6 | B | C |
| ATOM | 1225 | CB | VAL | B | 203 | −16.723 | −7.626 | 8.069 | 1.00 | 23.92 | | B | C |
| ANISOU | 1225 | CB | VAL | B | 203 | 3026 | 3031 | 3030 | 25 | 7 | 4 | B | C |
| ATOM | 1226 | CG1 | VAL | B | 203 | −18.179 | −7.854 | 7.804 | 1.00 | 24.83 | | B | C |
| ANISOU | 1226 | CG1 | VAL | B | 203 | 3132 | 3157 | 3144 | −16 | −14 | 0 | B | C |
| ATOM | 1227 | CG2 | VAL | B | 203 | −15.969 | −7.390 | 6.761 | 1.00 | 24.95 | | B | C |
| ANISOU | 1227 | CG2 | VAL | B | 203 | 3167 | 3203 | 3109 | −12 | 27 | 0 | B | C |
| ATOM | 1228 | C | VAL | B | 203 | −17.283 | −6.659 | 10.325 | 1.00 | 22.55 | | B | C |
| ANISOU | 1228 | C | VAL | B | 203 | 2869 | 2815 | 2883 | 67 | 50 | −12 | B | C |
| ATOM | 1229 | O | VAL | B | 203 | −17.193 | −7.729 | 10.926 | 1.00 | 23.38 | | B | O |
| ANISOU | 1229 | O | VAL | B | 203 | 2996 | 2917 | 2970 | 55 | 71 | −10 | B | O |
| ATOM | 1230 | N | TRP | B | 204 | −18.049 | −5.652 | 10.748 | 1.00 | 19.59 | | B | N |
| ANISOU | 1230 | N | TRP | B | 204 | 2447 | 2561 | 2435 | 1 | 65 | 33 | B | N |
| ATOM | 1231 | CA | TRP | B | 204 | −18.837 | −5.775 | 11.972 | 1.00 | 17.14 | | B | C |
| ANISOU | 1231 | CA | TRP | B | 204 | 2109 | 2166 | 2238 | −4 | −25 | −1 | B | C |
| ATOM | 1232 | CB | TRP | B | 204 | −18.304 | −4.851 | 13.088 | 1.00 | 18.13 | | B | C |
| ANISOU | 1232 | CB | TRP | B | 204 | 2330 | 2344 | 2213 | −26 | −4 | −43 | B | C |
| ATOM | 1233 | CG | TRP | B | 204 | −16.827 | −4.950 | 13.412 | 1.00 | 18.57 | | B | C |
| ANISOU | 1233 | CG | TRP | B | 204 | 2359 | 2420 | 2277 | 9 | 65 | −23 | B | C |
| ATOM | 1234 | CD1 | TRP | B | 204 | −15.924 | −3.938 | 13.392 | 1.00 | 19.14 | | B | C |
| ANISOU | 1234 | CD1 | TRP | B | 204 | 2416 | 2483 | 2373 | −28 | 19 | −21 | B | C |
| ATOM | 1235 | NE1 | TRP | B | 204 | −14.693 | −4.389 | 13.759 | 1.00 | 20.89 | | B | N |
| ANISOU | 1235 | NE1 | TRP | B | 204 | 2622 | 2585 | 2729 | 44 | −6 | 47 | B | N |
| ATOM | 1236 | CE2 | TRP | B | 204 | −14.779 | −5.722 | 14.031 | 1.00 | 19.28 | | B | C |
| ANISOU | 1236 | CE2 | TRP | B | 204 | 2422 | 2532 | 2371 | 7 | −13 | −19 | B | C |
| ATOM | 1237 | CD2 | TRP | B | 204 | −16.115 | −6.103 | 13.843 | 1.00 | 19.32 | | B | C |
| ANISOU | 1237 | CD2 | TRP | B | 204 | 2480 | 2469 | 2390 | 12 | 56 | 15 | B | C |
| ATOM | 1238 | CE3 | TRP | B | 204 | −16.466 | −7.433 | 14.055 | 1.00 | 19.90 | | B | C |
| ANISOU | 1238 | CE3 | TRP | B | 204 | 2597 | 2455 | 2509 | 20 | 11 | 0 | B | C |
| ATOM | 1239 | CZ3 | TRP | B | 204 | −15.487 | −8.311 | 14.452 | 1.00 | 20.20 | | B | C |
| ANISOU | 1239 | CZ3 | TRP | B | 204 | 2547 | 2580 | 2549 | 30 | −22 | 21 | B | C |
| ATOM | 1240 | CH2 | TRP | B | 204 | −14.180 | −7.901 | 14.643 | 1.00 | 20.07 | | B | C |
| ANISOU | 1240 | CH2 | TRP | B | 204 | 2565 | 2507 | 2554 | 42 | 37 | 11 | B | C |
| ATOM | 1241 | CZ2 | TRP | B | 204 | −13.802 | −6.611 | 14.437 | 1.00 | 20.52 | | B | C |
| ANISOU | 1241 | CZ2 | TRP | B | 204 | 2628 | 2544 | 2626 | 33 | 27 | 31 | B | C |
| ATOM | 1242 | C | TRP | B | 204 | −20.336 | −5.540 | 11.828 | 1.00 | 15.25 | | B | C |
| ANISOU | 1242 | C | TRP | B | 204 | 1984 | 1867 | 1942 | −32 | −17 | 63 | B | C |
| ATOM | 1243 | O | TRP | B | 204 | −21.102 | −6.115 | 12.546 | 1.00 | 14.65 | | B | O |
| ANISOU | 1243 | O | TRP | B | 204 | 1726 | 1911 | 1929 | 13 | 25 | 70 | B | O |
| ATOM | 1244 | N | ILE | B | 205 | −20.743 | −4.652 | 10.934 | 1.00 | 12.92 | | B | N |
| ANISOU | 1244 | N | ILE | B | 205 | 1654 | 1592 | 1664 | −6 | −86 | −62 | B | N |
| ATOM | 1245 | CA | ILE | B | 205 | −22.173 | −4.324 | 10.896 | 1.00 | 12.06 | | B | C |
| ANISOU | 1245 | CA | ILE | B | 205 | 1560 | 1491 | 1532 | −80 | −23 | −42 | B | C |
| ATOM | 1246 | CB | ILE | B | 205 | −22.519 | −3.054 | 10.071 | 1.00 | 13.07 | | B | C |
| ANISOU | 1246 | CB | ILE | B | 205 | 1665 | 1620 | 1680 | 35 | −33 | −42 | B | C |
| ATOM | 1247 | CG1 | ILE | B | 205 | −23.989 | −2.686 | 10.302 | 1.00 | 14.88 | | B | C |
| ANISOU | 1247 | CG1 | ILE | B | 205 | 1736 | 1857 | 2062 | 20 | 66 | 18 | B | C |
| ATOM | 1248 | CD1 | ILE | B | 205 | −24.344 | −1.290 | 9.940 | 1.00 | 16.48 | | B | C |
| ANISOU | 1248 | CD1 | ILE | B | 205 | 2090 | 1986 | 2187 | 59 | −23 | 51 | B | C |
| ATOM | 1249 | CG2 | ILE | B | 205 | −22.230 | −3.248 | 8.592 | 1.00 | 13.15 | | B | C |
| ANISOU | 1249 | CG2 | ILE | B | 205 | 1676 | 1753 | 1569 | −53 | 30 | 72 | B | C |
| ATOM | 1250 | C | ILE | B | 205 | −22.996 | −5.543 | 10.466 | 1.00 | 11.30 | | B | C |
| ANISOU | 1250 | C | ILE | B | 205 | 1461 | 1455 | 1377 | −60 | 1 | −12 | B | C |
| ATOM | 1251 | O | ILE | B | 205 | −22.688 | −6.173 | 9.452 | 1.00 | 10.83 | | B | O |
| ANISOU | 1251 | O | ILE | B | 205 | 1473 | 1288 | 1355 | 0 | 52 | −55 | B | O |
| ATOM | 1252 | N | PRO | B | 206 | −23.998 | −5.919 | 11.281 | 1.00 | 10.01 | | B | N |
| ANISOU | 1252 | N | PRO | B | 206 | 1304 | 1292 | 1207 | 2 | 3 | −105 | B | N |
| ATOM | 1253 | CA | PRO | B | 206 | −24.786 | −7.106 | 10.960 | 1.00 | 9.82 | | B | C |
| ANISOU | 1253 | CA | PRO | B | 206 | 1197 | 1290 | 1245 | −59 | 19 | −6 | B | C |
| ATOM | 1254 | CB | PRO | B | 206 | −25.863 | −7.110 | 12.047 | 1.00 | 9.81 | | B | C |
| ANISOU | 1254 | CB | PRO | B | 206 | 1294 | 1355 | 1079 | 10 | −13 | −17 | B | C |
| ATOM | 1255 | CG | PRO | B | 206 | −25.188 | −6.443 | 13.212 | 1.00 | 9.12 | | B | C |
| ANISOU | 1255 | CG | PRO | B | 206 | 1178 | 1161 | 1128 | −70 | −47 | −64 | B | C |
| ATOM | 1256 | CD | PRO | B | 206 | −24.402 | −5.333 | 12.577 | 1.00 | 9.52 | | B | C |
| ANISOU | 1256 | CD | PRO | B | 206 | 1191 | 1295 | 1132 | 3 | 11 | 0 | B | C |
| ATOM | 1257 | C | PRO | B | 206 | −25.424 | −7.091 | 9.574 | 1.00 | 9.41 | | B | C |
| ANISOU | 1257 | C | PRO | B | 206 | 1232 | 1134 | 1210 | −46 | 7 | −23 | B | C |
| ATOM | 1258 | O | PRO | B | 206 | −25.744 | −6.023 | 9.019 | 1.00 | 10.16 | | B | O |
| ANISOU | 1258 | O | PRO | B | 206 | 1283 | 1287 | 1291 | −5 | −97 | −37 | B | O |
| ATOM | 1259 | N | VAL | B | 207 | −25.597 | −8.290 | 9.028 | 1.00 | 9.57 | | B | N |
| ANISOU | 1259 | N | VAL | B | 207 | 1229 | 1228 | 1179 | −54 | −53 | −50 | B | N |
| ATOM | 1260 | CA | VAL | B | 207 | −26.267 | −8.479 | 7.751 | 1.00 | 9.38 | | B | C |
| ANISOU | 1260 | CA | VAL | B | 207 | 1204 | 1196 | 1164 | −1 | −46 | −39 | B | C |
| ATOM | 1261 | CB | VAL | B | 207 | −26.438 | −9.994 | 7.442 | 1.00 | 8.93 | | B | C |
| ANISOU | 1261 | CB | VAL | B | 207 | 1253 | 1067 | 1072 | −8 | 5 | −13 | B | C |
| ATOM | 1262 | CG1 | VAL | B | 207 | −27.376 | −10.680 | 8.450 | 1.00 | 12.21 | | B | C |
| ANISOU | 1262 | CG1 | VAL | B | 207 | 1419 | 1691 | 1530 | −21 | 75 | 154 | B | C |
| ATOM | 1263 | CG2 | VAL | B | 207 | −26.923 | −10.209 | 6.028 | 1.00 | 9.91 | | B | C |
| ANISOU | 1263 | CG2 | VAL | B | 207 | 1159 | 1406 | 1201 | −57 | −101 | −8 | B | C |
| ATOM | 1264 | C | VAL | B | 207 | −27.615 | −7.737 | 7.719 | 1.00 | 9.35 | | B | C |
| ANISOU | 1264 | C | VAL | B | 207 | 1241 | 1142 | 1171 | 47 | 2 | −3 | B | C |
| ATOM | 1265 | O | VAL | B | 207 | −28.354 | −7.722 | 8.716 | 1.00 | 9.54 | | B | O |
| ANISOU | 1265 | O | VAL | B | 207 | 1255 | 1234 | 1136 | 47 | −18 | 117 | B | O |
| ATOM | 1266 | N | ASN | B | 208 | −27.879 | −7.082 | 6.585 | 1.00 | 9.61 | | B | N |
| ANISOU | 1266 | N | ASN | B | 208 | 1286 | 1272 | 1094 | 78 | 14 | −4 | B | N |
| ATOM | 1267 | CA | ASN | B | 208 | −29.161 | −6.423 | 6.275 | 1.00 | 9.61 | | B | C |
| ANISOU | 1267 | CA | ASN | B | 208 | 1250 | 1169 | 1232 | 10 | 3 | −31 | B | C |
| ATOM | 1268 | CB | ASN | B | 208 | −30.342 | −7.397 | 6.394 | 1.00 | 9.46 | | B | C |
| ANISOU | 1268 | CB | ASN | B | 208 | 1229 | 1149 | 1217 | −34 | 12 | −43 | B | C |
| ATOM | 1269 | CG | ASN | B | 208 | −30.405 | −8.393 | 5.252 | 1.00 | 9.53 | | B | C |
| ANISOU | 1269 | CG | ASN | B | 208 | 1310 | 1179 | 1132 | 47 | 58 | −15 | B | C |
| ATOM | 1270 | OD1 | ASN | B | 208 | −29.924 | −8.131 | 4.144 | 1.00 | 9.50 | | B | O |
| ANISOU | 1270 | OD1 | ASN | B | 208 | 1026 | 1368 | 1217 | 0 | 26 | −66 | B | O |
| ATOM | 1271 | ND2 | ASN | B | 208 | −31.022 | −9.539 | 5.513 | 1.00 | 11.56 | | B | N |
| ANISOU | 1271 | ND2 | ASN | B | 208 | 1548 | 1328 | 1517 | −89 | 115 | −19 | B | N |
| ATOM | 1272 | C | ASN | B | 208 | −29.498 | −5.127 | 7.012 | 1.00 | 9.14 | | B | C |
| ANISOU | 1272 | C | ASN | B | 208 | 1172 | 1196 | 1106 | −8 | 30 | −49 | B | C |
| ATOM | 1273 | O | ASN | B | 208 | −30.617 | −4.625 | 6.880 | 1.00 | 9.37 | | B | O |
| ANISOU | 1273 | O | ASN | B | 208 | 1200 | 1151 | 1211 | 29 | −71 | −5 | B | O |
| ATOM | 1274 | N | GLU | B | 209 | −28.558 | −4.575 | 7.777 | 1.00 | 9.37 | | B | N |
| ANISOU | 1274 | N | GLU | B | 209 | 1282 | 1158 | 1119 | −10 | −44 | −11 | B | N |
| ATOM | 1275 | CA | GLU | B | 209 | −28.843 | −3.323 | 8.488 | 1.00 | 8.53 | | B | C |
| ANISOU | 1275 | CA | GLU | B | 209 | 1174 | 1051 | 1017 | 43 | −83 | 21 | B | C |
| ATOM | 1276 | CB | GLU | B | 209 | −27.710 | −2.952 | 9.444 | 1.00 | 8.27 | | B | C |
| ANISOU | 1276 | CB | GLU | B | 209 | 1123 | 950 | 1069 | −97 | −71 | 22 | B | C |
| ATOM | 1277 | CG | GLU | B | 209 | −27.663 | −3.826 | 10.687 | 1.00 | 8.89 | | B | C |
| ANISOU | 1277 | CG | GLU | B | 209 | 1131 | 1267 | 978 | −20 | 57 | 89 | B | C |
| ATOM | 1278 | CD | GLU | B | 209 | −28.824 | −3.555 | 11.629 | 1.00 | 8.85 | | B | C |
| ANISOU | 1278 | CD | GLU | B | 209 | 1050 | 1255 | 1056 | 7 | −6 | −39 | B | C |
| ATOM | 1279 | OE1 | GLU | B | 209 | −28.771 | −2.539 | 12.356 | 1.00 | 8.75 | | B | O |
| ANISOU | 1279 | OE1 | GLU | B | 209 | 1185 | 1017 | 1122 | −57 | −35 | 104 | B | O |
| ATOM | 1280 | OE2 | GLU | B | 209 | −29.779 | −4.365 | 11.647 | 1.00 | 10.20 | | B | O |
| ANISOU | 1280 | OE2 | GLU | B | 209 | 1427 | 1197 | 1251 | −93 | −58 | 58 | B | O |
| ATOM | 1281 | C | GLU | B | 209 | −29.089 | −2.205 | 7.488 | 1.00 | 8.66 | | B | C |
| ANISOU | 1281 | C | GLU | B | 209 | 1182 | 1082 | 1026 | −4 | −36 | 7 | B | C |
| ATOM | 1282 | O | GLU | B | 209 | −28.200 | −1.850 | 6.723 | 1.00 | 9.65 | | B | O |
| ANISOU | 1282 | O | GLU | B | 209 | 1201 | 1199 | 1265 | 0 | 43 | 62 | B | O |
| ATOM | 1283 | N | GLY | B | 210 | −30.312 | −1.684 | 7.479 | 1.00 | 8.63 | | B | N |
| ANISOU | 1283 | N | GLY | B | 210 | 1186 | 1102 | 992 | 45 | −39 | 15 | B | N |
| ATOM | 1284 | CA | GLY | B | 210 | −30.707 | −0.656 | 6.514 | 1.00 | 8.72 | | B | C |
| ANISOU | 1284 | CA | GLY | B | 210 | 1166 | 1112 | 1037 | 22 | −2 | 28 | B | C |
| ATOM | 1285 | C | GLY | B | 210 | −31.044 | −1.166 | 5.118 | 1.00 | 9.63 | | B | C |
| ANISOU | 1285 | C | GLY | B | 210 | 1329 | 1207 | 1122 | 20 | 8 | 11 | B | C |
| ATOM | 1286 | O | GLY | B | 210 | −31.222 | −0.361 | 4.198 | 1.00 | 9.32 | | B | O |
| ANISOU | 1286 | O | GLY | B | 210 | 1234 | 1173 | 1134 | 99 | −38 | 82 | B | O |
| ATOM | 1287 | N | ALA | B | 211 | −31.129 | −2.489 | 4.955 | 1.00 | 9.12 | | B | N |
| ANISOU | 1287 | N | ALA | B | 211 | 1258 | 1198 | 1010 | 63 | −3 | −11 | B | N |
| ATOM | 1288 | CA | ALA | B | 211 | −31.401 | −3.098 | 3.644 | 1.00 | 9.06 | | B | C |
| ANISOU | 1288 | CA | ALA | B | 211 | 1180 | 1163 | 1100 | −19 | −41 | −46 | B | C |
| ATOM | 1289 | CB | ALA | B | 211 | −30.811 | −4.499 | 3.559 | 1.00 | 10.07 | | B | C |
| ANISOU | 1289 | CB | ALA | B | 211 | 1313 | 1284 | 1229 | 11 | −17 | −23 | B | C |
| ATOM | 1290 | C | ALA | B | 211 | −32.889 | −3.128 | 3.314 | 1.00 | 9.89 | | B | C |
| ANISOU | 1290 | C | ALA | B | 211 | 1272 | 1333 | 1152 | −8 | −37 | −26 | B | C |
| ATOM | 1291 | O | ALA | B | 211 | −33.739 | −3.230 | 4.210 | 1.00 | 10.72 | | B | O |
| ANISOU | 1291 | O | ALA | B | 211 | 1298 | 1606 | 1171 | −1 | 7 | 34 | B | O |
| ATOM | 1292 | N | SER | B | 212 | −33.188 | −3.060 | 2.019 | 1.00 | 8.81 | | B | N |
| ANISOU | 1292 | N | SER | B | 212 | 1166 | 1089 | 1091 | −8 | −63 | 19 | B | N |
| ATOM | 1293 | CA | SER | B | 212 | −34.557 | −3.127 | 1.517 | 1.00 | 8.42 | | B | C |
| ANISOU | 1293 | CA | SER | B | 212 | 1109 | 1046 | 1045 | 12 | 5 | −28 | B | C |
| ATOM | 1294 | CB | SER | B | 212 | −35.282 | −1.806 | 1.778 | 1.00 | 8.64 | | B | C |
| ANISOU | 1294 | CB | SER | B | 212 | 1176 | 1047 | 1061 | 66 | −20 | −28 | B | C |
| ATOM | 1295 | OG | SER | B | 212 | −34.782 | −0.776 | 0.935 | 1.00 | 8.67 | | B | O |
| ANISOU | 1295 | OG | SER | B | 212 | 1130 | 1132 | 1032 | −50 | 17 | −13 | B | O |
| ATOM | 1296 | C | SER | B | 212 | −34.520 | −3.379 | 0.016 | 1.00 | 8.18 | | B | C |
| ANISOU | 1296 | C | SER | B | 212 | 1075 | 1051 | 983 | −25 | 7 | 7 | B | C |
| ATOM | 1297 | O | SER | B | 212 | −33.441 | −3.407 | −0.588 | 1.00 | 8.37 | | B | O |
| ANISOU | 1297 | O | SER | B | 212 | 1050 | 1119 | 1012 | 70 | 12 | −83 | B | O |
| ATOM | 1298 | N | THR | B | 213 | −35.696 | −3.550 | −0.586 | 1.00 | 8.69 | | B | N |
| ANISOU | 1298 | N | THR | B | 213 | 1132 | 1048 | 1123 | −14 | −27 | −17 | B | N |
| ATOM | 1299 | CA | THR | B | 213 | −35.820 | −3.481 | −2.042 | 1.00 | 9.11 | | B | C |
| ANISOU | 1299 | CA | THR | B | 213 | 1203 | 1113 | 1144 | −40 | −21 | 11 | B | C |
| ATOM | 1300 | CB | THR | B | 213 | −37.188 | −3.993 | −2.536 | 1.00 | 10.69 | | B | C |
| ANISOU | 1300 | CB | THR | B | 213 | 1304 | 1295 | 1461 | −69 | −46 | 5 | B | C |
| ATOM | 1301 | OG1 | THR | B | 213 | −38.227 | −3.161 | −2.003 | 1.00 | 13.40 | | B | O |
| ANISOU | 1301 | OG1 | THR | B | 213 | 1393 | 1750 | 1948 | 72 | −7 | −62 | B | O |
| ATOM | 1302 | CG2 | THR | B | 213 | −37.418 | −5.438 | −2.121 | 1.00 | 9.46 | | B | C |
| ANISOU | 1302 | CG2 | THR | B | 213 | 1179 | 1234 | 1182 | −38 | −63 | 20 | B | C |
| ATOM | 1303 | C | THR | B | 213 | −35.665 | −2.020 | −2.478 | 1.00 | 9.18 | | B | C |
| ANISOU | 1303 | C | THR | B | 213 | 1206 | 1098 | 1183 | −36 | 30 | 2 | B | C |
| ATOM | 1304 | O | THR | B | 213 | −35.740 | −1.102 | −1.654 | 1.00 | 9.80 | | B | O |
| ANISOU | 1304 | O | THR | B | 213 | 1425 | 1144 | 1154 | −103 | 75 | −30 | B | O |
| ATOM | 1305 | N | SER | B | 214 | −35.459 | −1.800 | −3.770 | 1.00 | 9.33 | | B | N |
| ANISOU | 1305 | N | SER | B | 214 | 1235 | 1135 | 1175 | −56 | 1 | 3 | B | N |
| ATOM | 1306 | CA | SER | B | 214 | −35.339 | −0.436 | −4.286 | 1.00 | 9.41 | | B | C |
| ANISOU | 1306 | CA | SER | B | 214 | 1240 | 1106 | 1230 | 0 | 17 | 9 | B | C |
| ATOM | 1307 | CB | SER | B | 214 | −34.632 | −0.432 | −5.639 | 1.00 | 9.49 | | B | C |
| ANISOU | 1307 | CB | SER | B | 214 | 1219 | 1209 | 1179 | 12 | −12 | 16 | B | C |
| ATOM | 1308 | OG | SER | B | 214 | −35.445 | −1.035 | −6.626 | 1.00 | 9.77 | | B | O |
| ANISOU | 1308 | OG | SER | B | 214 | 1284 | 1292 | 1136 | −34 | 37 | −73 | B | O |
| ATOM | 1309 | C | SER | B | 214 | −36.691 | 0.261 | −4.417 | 1.00 | 11.48 | | B | C |
| ANISOU | 1309 | C | SER | B | 214 | 1398 | 1448 | 1514 | 66 | −27 | 3 | B | C |
| ATOM | 1310 | O | SER | B | 214 | −36.757 | 1.490 | −4.457 | 1.00 | 12.35 | | B | O |
| ANISOU | 1310 | O | SER | B | 214 | 1459 | 1534 | 1698 | −17 | 22 | −33 | B | O |
| ATOM | 1311 | N | GLY | B | 215 | −37.758 | −0.527 | −4.501 | 1.00 | 13.83 | | B | N |
| ANISOU | 1311 | N | GLY | B | 215 | 1643 | 1736 | 1875 | −56 | 39 | 9 | B | N |
| ATOM | 1312 | CA | GLY | B | 215 | −39.087 | 0.009 | −4.795 | 1.00 | 17.70 | | B | C |
| ANISOU | 1312 | CA | GLY | B | 215 | 2048 | 2223 | 2454 | 73 | −31 | 17 | B | C |
| ATOM | 1313 | C | GLY | B | 215 | −39.342 | 0.177 | −6.286 | 1.00 | 21.31 | | B | C |
| ANISOU | 1313 | C | GLY | B | 215 | 2684 | 2735 | 2679 | 19 | −44 | 39 | B | C |
| ATOM | 1314 | O | GLY | B | 215 | −40.440 | 0.568 | −6.691 | 1.00 | 23.00 | | B | O |
| ANISOU | 1314 | O | GLY | B | 215 | 2747 | 2974 | 3019 | 73 | −24 | 44 | B | O |
| ATOM | 1315 | N | MET | B | 216 | −38.332 | −0.122 | −7.104 | 1.00 | 23.06 | | B | N |
| ANISOU | 1315 | N | MET | B | 216 | 2853 | 2955 | 2955 | 42 | 9 | 21 | B | N |
| ATOM | 1316 | CA | MET | B | 216 | −38.442 | −0.027 | −8.564 | 1.00 | 25.50 | | B | C |
| ANISOU | 1316 | CA | MET | B | 216 | 3283 | 3253 | 3152 | 18 | −14 | 12 | B | C |
| ATOM | 1317 | CB | MET | B | 216 | −37.054 | −0.022 | −9.214 | 1.00 | 26.35 | | B | C |
| ANISOU | 1317 | CB | MET | B | 216 | 3348 | 3400 | 3264 | 75 | 34 | 47 | B | C |
| ATOM | 1318 | CG | MET | B | 216 | −36.147 | 1.137 | −8.803 | 1.00 | 30.19 | | B | C |
| ANISOU | 1318 | CG | MET | B | 216 | 3766 | 3861 | 3845 | −81 | −42 | −118 | B | C |
| ATOM | 1319 | SD | MET | B | 216 | −36.215 | 2.556 | −9.914 | 1.00 | 36.26 | | B | S |
| ANISOU | 1319 | SD | MET | B | 216 | 4764 | 4465 | 4549 | −27 | 14 | 188 | B | S |
| ATOM | 1320 | CE | MET | B | 216 | −35.453 | 1.874 | −11.388 | 1.00 | 37.40 | | B | C |
| ANISOU | 1320 | CE | MET | B | 216 | 4742 | 4744 | 4724 | −8 | 20 | −24 | B | C |
| ATOM | 1321 | C | MET | B | 216 | −39.267 | −1.182 | −9.125 | 1.00 | 26.30 | | B | C |
| ANISOU | 1321 | C | MET | B | 216 | 3364 | 3312 | 3315 | −9 | −5 | −2 | B | C |
| ATOM | 1322 | O | MET | B | 216 | −39.044 | −2.345 | −8.778 | 1.00 | 27.09 | | B | O |
| ANISOU | 1322 | O | MET | B | 216 | 3492 | 3355 | 3446 | 15 | 3 | −27 | B | O |
| TER | | | | | | | | | | | | | |
| ATOM | 1323 | O | HOH | W | 1 | −17.577 | −2.208 | −15.264 | 1.00 | 17.35 | | W | O |
| ANISOU | 1323 | O | HOH | W | 1 | 2356 | 2136 | 2102 | −102 | 143 | −68 | W | O |
| ATOM | 1324 | O | HOH | W | 2 | −35.594 | −10.276 | 2.592 | 1.00 | 13.91 | | W | O |
| ANISOU | 1324 | O | HOH | W | 2 | 1942 | 1918 | 1424 | 13 | 114 | 54 | W | O |
| ATOM | 1325 | O | HOH | W | 3 | −35.642 | −20.809 | −3.292 | 1.00 | 12.21 | | W | O |
| ANISOU | 1325 | O | HOH | W | 3 | 1460 | 1644 | 1536 | −77 | 0 | 16 | W | O |
| ATOM | 1326 | O | HOH | W | 4 | −35.424 | −25.035 | −10.598 | 1.00 | 21.40 | | W | O |
| ANISOU | 1326 | O | HOH | W | 4 | 3001 | 2529 | 2600 | 136 | 8 | −42 | W | O |
| ATOM | 1327 | O | HOH | W | 5 | −24.926 | −5.147 | 6.517 | 1.00 | 9.57 | | W | O |
| ANISOU | 1327 | O | HOH | W | 5 | 1355 | 1165 | 1118 | −28 | 22 | −18 | W | O |
| ATOM | 1328 | O | HOH | W | 6 | −21.611 | −13.148 | −0.059 | 1.00 | 11.46 | | W | O |
| ANISOU | 1328 | O | HOH | W | 6 | 1603 | 1565 | 1186 | 43 | 42 | −59 | W | O |
| ATOM | 1329 | O | HOH | W | 7 | −28.218 | 1.256 | −17.427 | 1.00 | 20.49 | | W | O |
| ANISOU | 1329 | O | HOH | W | 7 | 2734 | 2939 | 2114 | −154 | −80 | 22 | W | O |
| ATOM | 1330 | O | HOH | W | 8 | −25.220 | 13.789 | −0.043 | 1.00 | 12.25 | | W | O |
| ANISOU | 1330 | O | HOH | W | 8 | 1643 | 1317 | 1696 | −4 | 36 | −62 | W | O |
| ATOM | 1331 | O | HOH | W | 9 | −30.539 | −6.374 | 9.900 | 1.00 | 8.90 | | W | O |
| ANISOU | 1331 | O | HOH | W | 9 | 1262 | 1047 | 1071 | −15 | 59 | −72 | W | O |
| ATOM | 1332 | O | HOH | W | 10 | −25.439 | −2.441 | 6.588 | 1.00 | 8.37 | | W | O |
| ANISOU | 1332 | O | HOH | W | 10 | 1283 | 936 | 963 | −43 | 47 | 118 | W | O |
| ATOM | 1333 | O | HOH | W | 11 | −39.528 | −20.928 | −6.391 | 1.00 | 10.80 | | W | O |
| ANISOU | 1333 | O | HOH | W | 11 | 1291 | 1531 | 1283 | −34 | 24 | −34 | W | O |
| ATOM | 1334 | O | HOH | W | 12 | −32.547 | 0.470 | 1.879 | 1.00 | 8.79 | | W | O |
| ANISOU | 1334 | O | HOH | W | 12 | 1111 | 1151 | 1077 | −132 | −23 | −50 | W | O |
| ATOM | 1335 | O | HOH | W | 13 | −32.528 | −2.452 | 9.104 | 1.00 | 14.12 | | W | O |
| ANISOU | 1335 | O | HOH | W | 13 | 1651 | 1846 | 1869 | −13 | 136 | 134 | W | O |
| ATOM | 1336 | O | HOH | W | 14 | −32.888 | −5.287 | 8.783 | 1.00 | 15.35 | | W | O |
| ANISOU | 1336 | O | HOH | W | 14 | 1893 | 2080 | 1861 | −98 | 48 | 143 | W | O |
| ATOM | 1337 | O | HOH | W | 15 | −29.948 | −1.948 | 14.783 | 1.00 | 9.36 | | W | O |
| ANISOU | 1337 | O | HOH | W | 15 | 1334 | 1221 | 1000 | 14 | 9 | 123 | W | O |
| ATOM | 1338 | O | HOH | W | 16 | −32.882 | −20.658 | −3.305 | 1.00 | 8.16 | | W | O |
| ANISOU | 1338 | O | HOH | W | 16 | 1130 | 945 | 1024 | −92 | −48 | 27 | W | O |
| ATOM | 1339 | O | HOH | W | 17 | −27.215 | 9.941 | 2.716 | 1.00 | 11.38 | | W | O |
| ANISOU | 1339 | O | HOH | W | 17 | 1529 | 1280 | 1514 | −74 | 65 | −69 | W | O |
| ATOM | 1340 | O | HOH | W | 18 | −35.273 | −4.347 | −9.780 | 1.00 | 13.71 | | W | O |
| ANISOU | 1340 | O | HOH | W | 18 | 1785 | 2067 | 1359 | 112 | 13 | −40 | W | O |
| ATOM | 1341 | O | HOH | W | 19 | −15.860 | −8.015 | −12.860 | 1.00 | 16.32 | | W | O |
| ANISOU | 1341 | O | HOH | W | 19 | 2085 | 2133 | 1981 | 42 | 249 | 22 | W | O |
| ATOM | 1342 | O | HOH | W | 20 | −28.365 | −24.659 | −2.537 | 1.00 | 15.24 | | W | O |
| ANISOU | 1342 | O | HOH | W | 20 | 2142 | 1926 | 1724 | −11 | −78 | 75 | W | O |
| ATOM | 1343 | O | HOH | W | 21 | −31.479 | −8.831 | −12.989 | 1.00 | 10.82 | | W | O |
| ANISOU | 1343 | O | HOH | W | 21 | 1453 | 1464 | 1193 | −180 | 34 | −54 | W | O |
| ATOM | 1344 | O | HOH | W | 22 | −28.639 | −9.218 | 11.256 | 1.00 | 13.05 | | W | O |
| ANISOU | 1344 | O | HOH | W | 22 | 1770 | 1708 | 1479 | 32 | 104 | −187 | W | O |
| ATOM | 1345 | O | HOH | W | 23 | −21.886 | −12.325 | 2.589 | 1.00 | 11.85 | | W | O |
| ANISOU | 1345 | O | HOH | W | 23 | 1542 | 1523 | 1436 | 38 | 49 | −34 | W | O |
| ATOM | 1346 | O | HOH | W | 24 | −38.093 | −5.281 | −12.113 | 1.00 | 32.02 | | W | O |
| ANISOU | 1346 | O | HOH | W | 24 | 4050 | 4058 | 4059 | 42 | 7 | 30 | W | O |
| ATOM | 1347 | O | HOH | W | 25 | −21.736 | −8.315 | 14.027 | 1.00 | 13.19 | | W | O |
| ANISOU | 1347 | O | HOH | W | 25 | 1675 | 1680 | 1658 | 93 | 125 | 99 | W | O |
| ATOM | 1348 | O | HOH | W | 26 | −28.734 | −16.766 | 1.037 | 1.00 | 13.90 | | W | O |
| ANISOU | 1348 | O | HOH | W | 26 | 1885 | 1640 | 1755 | 33 | −45 | 140 | W | O |
| ATOM | 1349 | O | HOH | W | 27 | −25.284 | 15.456 | −2.469 | 1.00 | 17.12 | | W | O |
| ANISOU | 1349 | O | HOH | W | 27 | 2290 | 2092 | 2123 | −15 | 28 | −24 | W | O |
| ATOM | 1350 | O | HOH | W | 28 | −40.841 | −15.176 | −8.930 | 1.00 | 9.10 | | W | O |
| ANISOU | 1350 | O | HOH | W | 28 | 1091 | 1222 | 1146 | −155 | −25 | −10 | W | O |
| ATOM | 1351 | O | HOH | W | 29 | −35.631 | −27.098 | −0.943 | 1.00 | 19.59 | | W | O |
| ANISOU | 1351 | O | HOH | W | 29 | 2488 | 2391 | 2565 | 60 | 37 | 56 | W | O |
| ATOM | 1352 | O | HOH | W | 30 | −38.170 | −9.327 | 2.404 | 1.00 | 16.39 | | W | O |
| ANISOU | 1352 | O | HOH | W | 30 | 1969 | 2260 | 2000 | 14 | 51 | −24 | W | O |
| ATOM | 1353 | O | HOH | W | 31 | −20.495 | 5.307 | −21.147 | 1.00 | 16.72 | | W | O |
| ANISOU | 1353 | O | HOH | W | 31 | 1994 | 2272 | 2088 | 5 | 13 | −123 | W | O |
| ATOM | 1354 | O | HOH | W | 32 | −35.426 | −12.995 | 2.633 | 1.00 | 13.61 | | W | O |
| ANISOU | 1354 | O | HOH | W | 32 | 1846 | 1709 | 1615 | −145 | 143 | −61 | W | O |
| ATOM | 1355 | O | HOH | W | 33 | −32.186 | −24.295 | −2.822 | 1.00 | 16.23 | | W | O |
| ANISOU | 1355 | O | HOH | W | 33 | 2117 | 1959 | 2089 | 15 | 61 | 108 | W | O |
| ATOM | 1356 | O | HOH | W | 34 | −45.956 | −19.042 | −10.604 | 1.00 | 17.65 | | W | O |
| ANISOU | 1356 | O | HOH | W | 34 | 2029 | 2282 | 2394 | 16 | −146 | 90 | W | O |
| ATOM | 1357 | O | HOH | W | 35 | −33.021 | 11.167 | −6.944 | 1.00 | 28.87 | | W | O |
| ANISOU | 1357 | O | HOH | W | 35 | 3762 | 3634 | 3574 | 13 | −8 | 14 | W | O |
| ATOM | 1358 | O | HOH | W | 36 | −14.625 | 3.563 | −6.888 | 1.00 | 15.72 | | W | O |
| ANISOU | 1358 | O | HOH | W | 36 | 1939 | 1996 | 2039 | −106 | −163 | 133 | W | O |
| ATOM | 1359 | O | HOH | W | 37 | −24.167 | −11.590 | 3.937 | 1.00 | 10.67 | | W | O |
| ANISOU | 1359 | O | HOH | W | 37 | 1451 | 1258 | 1344 | −95 | −63 | 24 | W | O |
| ATOM | 1360 | O | HOH | W | 38 | −32.756 | −6.244 | −15.553 | 1.00 | 13.42 | | W | O |
| ANISOU | 1360 | O | HOH | W | 38 | 1598 | 2149 | 1352 | 14 | 119 | 19 | W | O |
| ATOM | 1361 | O | HOH | W | 39 | −15.858 | −14.584 | −9.559 | 1.00 | 17.50 | | W | O |
| ANISOU | 1361 | O | HOH | W | 39 | 2273 | 2077 | 2300 | 11 | 210 | −98 | W | O |
| ATOM | 1362 | O | HOH | W | 40 | −21.845 | −6.027 | 6.765 | 1.00 | 11.35 | | W | O |
| ANISOU | 1362 | O | HOH | W | 40 | 1489 | 1425 | 1398 | 41 | 36 | −24 | W | O |
| ATOM | 1363 | O | HOH | W | 41 | −16.117 | 9.727 | −16.186 | 1.00 | 15.67 | | W | O |
| ANISOU | 1363 | O | HOH | W | 41 | 1854 | 2053 | 2046 | −49 | 16 | 91 | W | O |
| ATOM | 1364 | O | HOH | W | 42 | −37.898 | 18.234 | −0.349 | 1.00 | 17.94 | | W | O |
| ANISOU | 1364 | O | HOH | W | 42 | 2225 | 2281 | 2312 | −102 | −32 | 6 | W | O |
| ATOM | 1365 | O | HOH | W | 43 | −26.699 | 10.900 | 5.367 | 1.00 | 15.87 | | W | O |
| ANISOU | 1365 | O | HOH | W | 43 | 2104 | 1830 | 2095 | 18 | −84 | −42 | W | O |
| ATOM | 1366 | O | HOH | W | 44 | −17.856 | 13.967 | −15.991 | 1.00 | 18.20 | | W | O |
| ANISOU | 1366 | O | HOH | W | 44 | 2520 | 2142 | 2255 | 5 | −38 | 116 | W | O |
| ATOM | 1367 | O | HOH | W | 45 | −26.845 | −18.369 | −0.073 | 1.00 | 10.51 | | W | O |
| ANISOU | 1367 | O | HOH | W | 45 | 1443 | 1278 | 1271 | −16 | 118 | −86 | W | O |
| ATOM | 1368 | O | HOH | W | 46 | −28.467 | 6.084 | −15.718 | 1.00 | 13.99 | | W | O |
| ANISOU | 1368 | O | HOH | W | 46 | 1735 | 1678 | 1903 | −40 | 119 | 59 | W | O |
| ATOM | 1369 | O | HOH | W | 47 | −33.866 | −16.919 | −16.127 | 1.00 | 16.58 | | W | O |
| ANISOU | 1369 | O | HOH | W | 47 | 2119 | 2153 | 2028 | 80 | −121 | −31 | W | O |
| ATOM | 1370 | O | HOH | W | 48 | −38.316 | −11.213 | −11.870 | 1.00 | 18.45 | | W | O |
| ANISOU | 1370 | O | HOH | W | 48 | 2405 | 2394 | 2212 | 105 | 24 | 104 | W | O |
| ATOM | 1371 | O | HOH | W | 49 | −33.941 | −13.483 | −17.134 | 1.00 | 16.80 | | W | O |
| ANISOU | 1371 | O | HOH | W | 49 | 2065 | 2389 | 1931 | −101 | 3 | 184 | W | O |
| ATOM | 1372 | O | HOH | W | 50 | −22.404 | −10.184 | 12.149 | 1.00 | 18.83 | | W | O |
| ANISOU | 1372 | O | HOH | W | 50 | 2481 | 2294 | 2380 | 51 | −110 | −61 | W | O |
| ATOM | 1373 | O | HOH | W | 51 | −23.364 | −19.986 | −2.433 | 1.00 | 14.73 | | W | O |
| ANISOU | 1373 | O | HOH | W | 51 | 1895 | 1758 | 1943 | −2 | −68 | 85 | W | O |
| ATOM | 1374 | O | HOH | W | 52 | −25.212 | 14.790 | 2.470 | 1.00 | 20.54 | | W | O |
| ANISOU | 1374 | O | HOH | W | 52 | 2668 | 2743 | 2393 | −78 | 21 | −149 | W | O |
| ATOM | 1375 | O | HOH | W | 53 | −20.246 | 3.243 | −16.893 | 1.00 | 14.04 | | W | O |
| ANISOU | 1375 | O | HOH | W | 53 | 1934 | 1702 | 1699 | 100 | 17 | −154 | W | O |
| ATOM | 1376 | O | HOH | W | 54 | −32.491 | −21.446 | −0.561 | 1.00 | 14.89 | | W | O |
| ANISOU | 1376 | O | HOH | W | 54 | 1759 | 1957 | 1940 | −71 | −30 | 201 | W | O |
| ATOM | 1377 | O | HOH | W | 55 | −31.780 | −18.709 | −16.472 | 1.00 | 16.34 | | W | O |
| ANISOU | 1377 | O | HOH | W | 55 | 2114 | 2049 | 2046 | 7 | −106 | 110 | W | O |
| ATOM | 1378 | O | HOH | W | 56 | −14.644 | 9.747 | −3.176 | 1.00 | 14.24 | | W | O |
| ANISOU | 1378 | O | HOH | W | 56 | 1736 | 1823 | 1851 | 17 | −59 | −41 | W | O |
| ATOM | 1379 | O | HOH | W | 57 | −28.567 | −4.470 | −17.889 | 1.00 | 13.04 | | W | O |
| ANISOU | 1379 | O | HOH | W | 57 | 1753 | 1840 | 1362 | −80 | −9 | 90 | W | O |
| ATOM | 1380 | O | HOH | W | 58 | −31.086 | 10.207 | 2.626 | 1.00 | 21.18 | | W | O |
| ANISOU | 1380 | O | HOH | W | 58 | 2560 | 2613 | 2873 | 17 | 52 | −70 | W | O |
| ATOM | 1381 | O | HOH | W | 59 | −35.331 | −15.627 | 4.012 | 1.00 | 22.99 | | W | O |
| ANISOU | 1381 | O | HOH | W | 59 | 2831 | 2974 | 2929 | 28 | 66 | −111 | W | O |
| ATOM | 1382 | O | HOH | W | 60 | −13.578 | 12.770 | −10.122 | 1.00 | 19.99 | | W | O |
| ANISOU | 1382 | O | HOH | W | 60 | 2444 | 2514 | 2638 | −112 | −54 | 30 | W | O |
| ATOM | 1383 | O | HOH | W | 61 | −25.363 | −22.141 | −1.606 | 1.00 | 15.76 | | W | O |
| ANISOU | 1383 | O | HOH | W | 61 | 1912 | 2231 | 1844 | 85 | 88 | 12 | W | O |
| ATOM | 1384 | O | HOH | W | 62 | −34.201 | −1.533 | 6.338 | 1.00 | 25.60 | | W | O |
| ANISOU | 1384 | O | HOH | W | 62 | 3230 | 3301 | 3197 | −47 | 32 | −180 | W | O |
| ATOM | 1385 | O | HOH | W | 63 | −28.843 | 16.175 | −0.888 | 1.00 | 13.71 | | W | O |
| ANISOU | 1385 | O | HOH | W | 63 | 1766 | 1603 | 1840 | 2 | −77 | −130 | W | O |
| ATOM | 1386 | O | HOH | W | 64 | −32.349 | −10.317 | −15.467 | 1.00 | 15.26 | | W | O |
| ANISOU | 1386 | O | HOH | W | 64 | 1965 | 1919 | 1915 | −29 | 13 | 88 | W | O |
| ATOM | 1387 | O | HOH | W | 65 | −15.843 | 12.358 | −17.044 | 1.00 | 19.47 | | W | O |
| ANISOU | 1387 | O | HOH | W | 65 | 2548 | 2380 | 2469 | −105 | −5 | 136 | W | O |
| ATOM | 1388 | O | HOH | W | 66 | −22.682 | 13.344 | 4.939 | 1.00 | 29.16 | | W | O |
| ANISOU | 1388 | O | HOH | W | 66 | 3643 | 3693 | 3744 | 8 | 32 | −52 | W | O |
| ATOM | 1389 | O | HOH | W | 67 | −24.719 | −10.533 | 10.728 | 1.00 | 12.50 | | W | O |
| ANISOU | 1389 | O | HOH | W | 67 | 1794 | 1533 | 1422 | −53 | −53 | 40 | W | O |
| ATOM | 1390 | O | HOH | W | 68 | −17.609 | −7.463 | −2.544 | 1.00 | 16.67 | | W | O |
| ANISOU | 1390 | O | HOH | W | 68 | 2053 | 2007 | 2275 | −2 | −7 | 50 | W | O |
| ATOM | 1391 | O | HOH | W | 69 | −19.998 | −12.784 | 6.418 | 1.00 | 17.72 | | W | O |
| ANISOU | 1391 | O | HOH | W | 69 | 2454 | 2175 | 2104 | 61 | −34 | 51 | W | O |
| ATOM | 1392 | O | HOH | W | 70 | −25.511 | 16.648 | −5.834 | 1.00 | 24.98 | | W | O |
| ANISOU | 1392 | O | HOH | W | 70 | 3132 | 3142 | 3218 | 27 | 50 | 102 | W | O |
| ATOM | 1393 | O | HOH | W | 71 | −30.370 | −10.707 | −17.365 | 1.00 | 13.92 | | W | O |
| ANISOU | 1393 | O | HOH | W | 71 | 1689 | 1765 | 1834 | −34 | −20 | 81 | W | O |
| ATOM | 1394 | O | HOH | W | 72 | −19.452 | −12.124 | 3.846 | 1.00 | 16.88 | | W | O |
| ANISOU | 1394 | O | HOH | W | 72 | 1903 | 2525 | 1984 | 103 | −188 | 103 | W | O |
| ATOM | 1395 | O | HOH | W | 73 | −33.633 | −5.506 | 6.145 | 1.00 | 19.28 | | W | O |
| ANISOU | 1395 | O | HOH | W | 73 | 2556 | 2560 | 2211 | 8 | 76 | 102 | W | O |
| ATOM | 1396 | O | HOH | W | 74 | −24.032 | −12.611 | 6.439 | 1.00 | 17.76 | | W | O |
| ANISOU | 1396 | O | HOH | W | 74 | 2359 | 2169 | 2220 | 4 | −23 | −2 | W | O |
| ATOM | 1397 | O | HOH | W | 75 | −38.807 | −7.560 | 4.267 | 1.00 | 28.03 | | W | O |
| ANISOU | 1397 | O | HOH | W | 75 | 3560 | 3598 | 3494 | 31 | 73 | −52 | W | O |
| ATOM | 1398 | O | HOH | W | 76 | −38.015 | −3.860 | 1.333 | 1.00 | 16.98 | | W | O |
| ANISOU | 1398 | O | HOH | W | 76 | 1874 | 2178 | 2399 | −160 | 101 | −36 | W | O |
| ATOM | 1399 | O | HOH | W | 77 | −29.048 | 8.682 | −15.205 | 1.00 | 13.39 | | W | O |
| ANISOU | 1399 | O | HOH | W | 77 | 1807 | 1731 | 1551 | 16 | 75 | −37 | W | O |
| ATOM | 1400 | O | HOH | W | 78 | −20.384 | 17.139 | −16.774 | 1.00 | 37.38 | | W | O |
| ANISOU | 1400 | O | HOH | W | 78 | 4783 | 4692 | 4728 | −23 | −13 | 18 | W | O |
| ATOM | 1401 | O | HOH | W | 79 | −24.430 | 0.007 | −17.942 | 0.50 | 14.64 | | W | O |
| ANISOU | 1401 | O | HOH | W | 79 | 1867 | 1909 | 1786 | 0 | 0 | 169 | W | O |
| ATOM | 1402 | O | HOH | W | 80 | −32.935 | 7.930 | −9.055 | 1.00 | 25.33 | | W | O |
| ANISOU | 1402 | O | HOH | W | 80 | 3234 | 3315 | 3075 | −28 | −101 | 8 | W | O |
| ATOM | 1403 | O | HOH | W | 81 | −19.049 | −9.564 | 11.396 | 1.00 | 21.81 | | W | O |
| ANISOU | 1403 | O | HOH | W | 81 | 2812 | 2658 | 2818 | −51 | 12 | −36 | W | O |
| ATOM | 1404 | O | HOH | W | 82 | −36.583 | 6.046 | −17.526 | 1.00 | 24.91 | | W | O |
| ANISOU | 1404 | O | HOH | W | 82 | 3053 | 3195 | 3218 | 27 | −17 | −7 | W | O |
| ATOM | 1405 | O | HOH | W | 83 | −32.166 | −3.340 | 12.148 | 1.00 | 26.37 | | W | O |
| ANISOU | 1405 | O | HOH | W | 83 | 3182 | 3418 | 3419 | 16 | 29 | −90 | W | O |
| ATOM | 1406 | O | HOH | W | 84 | −19.553 | −4.605 | 6.544 | 1.00 | 17.63 | | W | O |
| ANISOU | 1406 | O | HOH | W | 84 | 2104 | 2385 | 2209 | 6 | −3 | 14 | W | O |
| ATOM | 1407 | O | HOH | W | 85 | −34.283 | −2.655 | −13.801 | 1.00 | 25.53 | | W | O |
| ANISOU | 1407 | O | HOH | W | 85 | 3362 | 3279 | 3059 | 104 | −53 | 160 | W | O |
| ATOM | 1408 | O | HOH | W | 86 | −22.754 | −14.753 | 3.938 | 1.00 | 17.89 | | W | O |
| ANISOU | 1408 | O | HOH | W | 86 | 2066 | 2435 | 2297 | −56 | −170 | 83 | W | O |
| ATOM | 1409 | O | HOH | W | 87 | −37.426 | −17.164 | −14.510 | 1.00 | 18.90 | | W | O |
| ANISOU | 1409 | O | HOH | W | 87 | 2487 | 2453 | 2243 | −102 | −83 | −55 | W | O |
| ATOM | 1410 | O | HOH | W | 88 | −8.317 | −0.021 | −4.957 | 1.00 | 25.19 | | W | O |
| ANISOU | 1410 | O | HOH | W | 88 | 3012 | 3175 | 3383 | 0 | 15 | −12 | W | O |
| ATOM | 1411 | O | HOH | W | 89 | −15.400 | 8.594 | −18.670 | 1.00 | 18.92 | | W | O |
| ANISOU | 1411 | O | HOH | W | 89 | 2551 | 2432 | 2207 | −39 | 104 | 52 | W | O |
| ATOM | 1412 | O | HOH | W | 90 | −35.748 | 2.314 | 0.706 | 1.00 | 24.11 | | W | O |
| ANISOU | 1412 | O | HOH | W | 90 | 2899 | 3219 | 3044 | −2 | 90 | −87 | W | O |
| ATOM | 1413 | O | HOH | W | 91 | −13.793 | −11.720 | −9.912 | 1.00 | 35.53 | | W | O |
| ANISOU | 1413 | O | HOH | W | 91 | 4426 | 4610 | 4464 | 20 | 34 | −13 | W | O |
| ATOM | 1414 | O | HOH | W | 92 | −32.293 | −25.005 | −9.762 | 1.00 | 27.57 | | W | O |
| ANISOU | 1414 | O | HOH | W | 92 | 3560 | 3421 | 3495 | 13 | 7 | −54 | W | O |
| ATOM | 1415 | O | HOH | W | 93 | −25.119 | −24.432 | −16.225 | 1.00 | 26.44 | | W | O |
| ANISOU | 1415 | O | HOH | W | 93 | 3476 | 3246 | 3324 | 46 | −34 | 0 | W | O |
| ATOM | 1416 | O | HOH | W | 94 | −23.288 | 14.915 | −8.603 | 1.00 | 24.56 | | W | O |
| ANISOU | 1416 | O | HOH | W | 94 | 3271 | 3026 | 3036 | 34 | 90 | 12 | W | O |
| ATOM | 1417 | O | HOH | W | 95 | −39.205 | −6.810 | −7.586 | 1.00 | 19.40 | | W | O |
| ANISOU | 1417 | O | HOH | W | 95 | 2316 | 2669 | 2386 | 80 | 82 | 108 | W | O |
| ATOM | 1418 | O | HOH | W | 96 | −17.494 | 6.819 | −21.327 | 1.00 | 20.51 | | W | O |
| ANISOU | 1418 | O | HOH | W | 96 | 2543 | 2542 | 2708 | −51 | 29 | −90 | W | O |
| ATOM | 1419 | O | HOH | W | 97 | −14.851 | −11.603 | −3.482 | 1.00 | 24.36 | | W | O |
| ANISOU | 1419 | O | HOH | W | 97 | 2971 | 3335 | 2948 | −72 | −93 | 17 | W | O |
| ATOM | 1420 | O | HOH | W | 98 | −25.386 | 13.341 | 4.826 | 1.00 | 19.48 | | W | O |
| ANISOU | 1420 | O | HOH | W | 98 | 2611 | 2440 | 2349 | 29 | −15 | 29 | W | O |
| ATOM | 1421 | O | HOH | W | 99 | −12.094 | −6.045 | −4.402 | 1.00 | 27.97 | | W | O |
| ANISOU | 1421 | O | HOH | W | 99 | 3373 | 3669 | 3587 | 16 | −67 | −43 | W | O |
| ATOM | 1422 | O | HOH | W | 100 | −18.788 | 16.271 | −2.585 | 1.00 | 27.23 | | W | O |
| ANISOU | 1422 | O | HOH | W | 100 | 3384 | 3390 | 3572 | −18 | −1 | 65 | W | O |
| ATOM | 1423 | O | HOH | W | 101 | −18.181 | −18.672 | 2.543 | 1.00 | 37.98 | | W | O |
| ANISOU | 1423 | O | HOH | W | 101 | 4803 | 4890 | 4737 | 32 | −24 | 17 | W | O |
| ATOM | 1424 | O | HOH | W | 102 | −14.157 | −9.082 | −10.112 | 1.00 | 36.38 | | W | O |
| ANISOU | 1424 | O | HOH | W | 102 | 4628 | 4584 | 4610 | 6 | 20 | 4 | W | O |
| ATOM | 1425 | O | HOH | W | 103 | −18.953 | −15.029 | 2.825 | 1.00 | 23.49 | | W | O |
| ANISOU | 1425 | O | HOH | W | 103 | 2940 | 3082 | 2904 | 79 | −70 | −62 | W | O |
| ATOM | 1426 | O | HOH | W | 104 | −25.758 | −24.902 | −9.194 | 1.00 | 22.26 | | W | O |
| ANISOU | 1426 | O | HOH | W | 104 | 2912 | 2822 | 2722 | 55 | 13 | 1 | W | O |
| ATOM | 1427 | O | HOH | W | 105 | −35.360 | −7.421 | 6.412 | 1.00 | 39.45 | | W | O |
| ANISOU | 1427 | O | HOH | W | 105 | 5075 | 4972 | 4943 | −21 | −8 | −10 | W | O |
| ATOM | 1428 | O | HOH | W | 106 | −31.705 | 7.844 | 0.951 | 1.00 | 22.98 | | W | O |
| ANISOU | 1428 | O | HOH | W | 106 | 2846 | 3014 | 2870 | −48 | 51 | −32 | W | O |
| ATOM | 1429 | O | HOH | W | 107 | −33.789 | 5.332 | −10.444 | 1.00 | 23.75 | | W | O |
| ANISOU | 1429 | O | HOH | W | 107 | 2956 | 2984 | 3083 | 31 | 95 | 44 | W | O |
| ATOM | 1430 | O | HOH | W | 108 | −37.766 | 8.496 | −15.873 | 1.00 | 18.33 | | W | O |
| ANISOU | 1430 | O | HOH | W | 108 | 2280 | 2400 | 2283 | 56 | 58 | 96 | W | O |
| ATOM | 1431 | O | HOH | W | 109 | −16.849 | 10.178 | −20.326 | 1.00 | 21.01 | | W | O |
| ANISOU | 1431 | O | HOH | W | 109 | 2291 | 2795 | 2898 | 86 | 104 | 147 | W | O |
| ATOM | 1432 | O | HOH | W | 110 | −16.652 | 14.848 | −7.843 | 1.00 | 31.69 | | W | O |
| ANISOU | 1432 | O | HOH | W | 110 | 4168 | 3973 | 3900 | −88 | 46 | 1 | W | O |
| ATOM | 1433 | O | HOH | W | 111 | −33.495 | 2.286 | 3.683 | 1.00 | 23.52 | | W | O |
| ANISOU | 1433 | O | HOH | W | 111 | 2995 | 2923 | 3019 | 104 | −52 | −102 | W | O |
| ATOM | 1434 | O | HOH | W | 112 | −14.233 | −13.021 | −1.395 | 1.00 | 35.05 | | W | O |
| ANISOU | 1434 | O | HOH | W | 112 | 4390 | 4414 | 4513 | −18 | −6 | 46 | W | O |
| ATOM | 1435 | O | HOH | W | 113 | −18.928 | −6.369 | 4.488 | 1.00 | 20.99 | | W | O |
| ANISOU | 1435 | O | HOH | W | 113 | 2668 | 2757 | 2549 | −17 | 12 | 69 | W | O |
| ATOM | 1436 | O | HOH | W | 114 | −16.254 | 13.811 | −10.287 | 1.00 | 26.01 | | W | O |
| ANISOU | 1436 | O | HOH | W | 114 | 3424 | 3287 | 3171 | −123 | −35 | 31 | W | O |
| ATOM | 1437 | O | HOH | W | 115 | −44.321 | −22.041 | −13.782 | 1.00 | 23.76 | | W | O |
| ANISOU | 1437 | O | HOH | W | 115 | 3036 | 3123 | 2869 | 15 | −125 | 14 | W | O |
| ATOM | 1438 | O | HOH | W | 116 | −10.049 | 6.870 | −15.858 | 1.00 | 29.52 | | W | O |
| ANISOU | 1438 | O | HOH | W | 116 | 3695 | 3816 | 3704 | 28 | 24 | −34 | W | O |
| ATOM | 1439 | O | HOH | W | 117 | −16.198 | −8.979 | −0.865 | 1.00 | 27.27 | | W | O |
| ANISOU | 1439 | O | HOH | W | 117 | 3351 | 3563 | 3448 | 67 | −1 | 67 | W | O |
| ATOM | 1440 | O | HOH | W | 118 | −15.461 | −14.732 | −6.170 | 1.00 | 23.22 | | W | O |
| ANISOU | 1440 | O | HOH | W | 118 | 3067 | 2849 | 2908 | 163 | −34 | −15 | W | O |
| ATOM | 1441 | O | HOH | W | 119 | −15.355 | −18.077 | −1.105 | 1.00 | 33.10 | | W | O |
| ANISOU | 1441 | O | HOH | W | 119 | 4121 | 4209 | 4247 | 59 | −65 | −37 | W | O |
| ATOM | 1442 | O | HOH | W | 120 | −16.785 | 16.156 | −14.795 | 1.00 | 37.45 | | W | O |
| ANISOU | 1442 | O | HOH | W | 120 | 4779 | 4643 | 4808 | −31 | −17 | −20 | W | O |
| ATOM | 1443 | O | HOH | W | 121 | −11.446 | −1.822 | −9.921 | 1.00 | 39.00 | | W | O |
| ANISOU | 1443 | O | HOH | W | 121 | 4874 | 4936 | 5007 | 20 | −7 | −7 | W | O |
| ATOM | 1444 | O | HOH | W | 122 | −36.066 | −28.974 | −10.892 | 1.00 | 32.42 | | W | O |
| ANISOU | 1444 | O | HOH | W | 122 | 4233 | 4097 | 3989 | 1 | 49 | −39 | W | O |
| ATOM | 1445 | O | HOH | W | 123 | −28.814 | 13.128 | −9.171 | 1.00 | 37.88 | | W | O |
| ANISOU | 1445 | O | HOH | W | 123 | 4909 | 4684 | 4800 | −19 | 55 | 0 | W | O |
| ATOM | 1446 | O | HOH | W | 124 | −23.858 | −10.006 | −19.805 | 1.00 | 17.03 | | W | O |
| ANISOU | 1446 | O | HOH | W | 124 | 2000 | 2302 | 2167 | 65 | 58 | −51 | W | O |
| ATOM | 1447 | O | HOH | W | 125 | −35.853 | −21.706 | −0.604 | 1.00 | 20.83 | | W | O |
| ANISOU | 1447 | O | HOH | W | 125 | 2571 | 2757 | 2585 | 91 | 12 | −42 | W | O |
| ATOM | 1448 | O | HOH | W | 126 | −26.086 | 13.362 | −10.040 | 1.00 | 31.69 | | W | O |
| ANISOU | 1448 | O | HOH | W | 126 | 3975 | 3998 | 4069 | −19 | 0 | −35 | W | O |
| ATOM | 1449 | O | HOH | W | 127 | −12.675 | −5.116 | −14.495 | 1.00 | 16.99 | | W | O |
| ANISOU | 1449 | O | HOH | W | 127 | 2019 | 2183 | 2255 | −6 | −34 | 41 | W | O |
| ATOM | 1450 | O | HOH | W | 128 | −12.525 | 2.687 | −8.526 | 1.00 | 31.55 | | W | O |
| ANISOU | 1450 | O | HOH | W | 128 | 3907 | 3899 | 4181 | −51 | 27 | −27 | W | O |
| ATOM | 1451 | O | HOH | W | 129 | −13.185 | −9.266 | −3.758 | 1.00 | 32.19 | | W | O |
| ANISOU | 1451 | O | HOH | W | 129 | 4089 | 4077 | 4065 | −21 | −31 | −46 | W | O |
| ATOM | 1452 | O | HOH | W | 130 | −20.837 | −20.749 | −2.546 | 1.00 | 20.30 | | W | O |
| ANISOU | 1452 | O | HOH | W | 130 | 2548 | 2495 | 2669 | −20 | −63 | 46 | W | O |
| ATOM | 1453 | O | HOH | W | 131 | −42.049 | −6.173 | −0.979 | 1.00 | 34.99 | | W | O |
| ANISOU | 1453 | O | HOH | W | 131 | 4453 | 4452 | 4391 | 79 | −14 | −10 | W | O |
| ATOM | 1454 | O | HOH | W | 132 | −30.910 | 10.260 | 5.460 | 1.00 | 25.98 | | W | O |
| ANISOU | 1454 | O | HOH | W | 132 | 3353 | 3229 | 3291 | 20 | 45 | 8 | W | O |
| ATOM | 1455 | O | HOH | W | 133 | −16.355 | −12.508 | −12.425 | 1.00 | 34.47 | | W | O |
| ANISOU | 1455 | O | HOH | W | 133 | 4344 | 4443 | 4310 | 4 | 19 | −31 | W | O |
| ATOM | 1456 | O | HOH | W | 134 | −33.787 | −23.690 | −0.535 | 1.00 | 20.72 | | W | O |
| ANISOU | 1456 | O | HOH | W | 134 | 2709 | 2607 | 2556 | −41 | 94 | 58 | W | O |
| ATOM | 1457 | O | HOH | W | 135 | −17.202 | 14.602 | 1.995 | 1.00 | 32.29 | | W | O |
| ANISOU | 1457 | O | HOH | W | 135 | 3975 | 4193 | 4102 | −35 | 6 | 27 | W | O |
| ATOM | 1458 | O | HOH | W | 136 | −36.490 | −3.479 | 4.425 | 1.00 | 25.34 | | W | O |
| ANISOU | 1458 | O | HOH | W | 136 | 3265 | 3153 | 3209 | 3 | 54 | 7 | W | O |
| ATOM | 1459 | O | HOH | W | 137 | −13.537 | −3.668 | −13.089 | 1.00 | 20.02 | | W | O |
| ANISOU | 1459 | O | HOH | W | 137 | 2513 | 2592 | 2501 | −52 | −17 | 93 | W | O |
| ATOM | 1460 | O | HOH | W | 138 | −10.714 | −2.940 | −2.231 | 1.00 | 25.50 | | W | O |
| ANISOU | 1460 | O | HOH | W | 138 | 3152 | 3252 | 3283 | 6 | 24 | 12 | W | O |
| ATOM | 1461 | O | HOH | W | 139 | −15.629 | −6.832 | 1.930 | 1.00 | 40.10 | | W | O |
| ANISOU | 1461 | O | HOH | W | 139 | 5034 | 5119 | 5085 | 1 | 6 | 15 | W | O |
| ATOM | 1463 | O | HOH | W | 140 | −12.895 | 9.296 | −19.096 | 1.00 | 25.38 | | W | O |
| ANISOU | 1463 | O | HOH | W | 140 | 3138 | 3277 | 3228 | −41 | 62 | 81 | W | O |
| ATOM | 1464 | O | HOH | W | 141 | −38.445 | 14.541 | 9.688 | 1.00 | 35.55 | | W | O |
| ANISOU | 1464 | O | HOH | W | 141 | 4504 | 4437 | 4567 | −8 | 13 | −16 | W | O |
| ATOM | 1465 | O | HOH | W | 142 | −12.063 | −10.830 | −6.318 | 1.00 | 43.73 | | W | O |
| ANISOU | 1465 | O | HOH | W | 142 | 5511 | 5527 | 5577 | 22 | 1 | 0 | W | O |
| ATOM | 1466 | O | HOH | W | 143 | −37.663 | −3.372 | −6.214 | 1.00 | 31.56 | | W | O |
| ANISOU | 1466 | O | HOH | W | 143 | 4017 | 3925 | 4049 | −31 | −30 | 12 | W | O |
| ATOM | 1467 | O | HOH | W | 144 | −32.757 | 12.064 | 1.336 | 1.00 | 18.33 | | W | O |
| ANISOU | 1467 | O | HOH | W | 144 | 2347 | 2338 | 2278 | −7 | −14 | −14 | W | O |
| ATOM | 1468 | O | HOH | W | 145 | −6.861 | 1.812 | −7.652 | 1.00 | 39.81 | | W | O |
| ANISOU | 1468 | O | HOH | W | 145 | 5041 | 5026 | 5060 | −11 | 4 | 9 | W | O |
| ATOM | 1469 | O | HOH | W | 146 | −31.942 | −14.561 | −18.798 | 1.00 | 17.94 | | W | O |
| ANISOU | 1469 | O | HOH | W | 146 | 2197 | 2389 | 2232 | 0 | −43 | −3 | W | O |
| ATOM | 1470 | O | HOH | W | 147 | −9.446 | −2.670 | −4.750 | 1.00 | 36.51 | | W | O |
| ANISOU | 1470 | O | HOH | W | 147 | 4572 | 4622 | 4678 | 32 | 7 | 13 | W | O |
| ATOM | 1471 | O | HOH | W | 148 | −15.375 | 1.722 | −17.052 | 1.00 | 35.42 | | W | O |
| ANISOU | 1471 | O | HOH | W | 148 | 4536 | 4467 | 4455 | 9 | −2 | 20 | W | O |
| ATOM | 1472 | O | HOH | W | 149 | −38.919 | −7.779 | −17.024 | 1.00 | 28.36 | | W | O |
| ANISOU | 1472 | O | HOH | W | 149 | 3582 | 3617 | 3578 | −27 | −50 | 36 | W | O |
| ATOM | 1473 | O | HOH | W | 150 | −15.030 | −16.764 | −4.420 | 1.00 | 33.51 | | W | O |
| ANISOU | 1473 | O | HOH | W | 150 | 4240 | 4207 | 4286 | 32 | −26 | 29 | W | O |
| ATOM | 1474 | O | HOH | W | 151 | −39.973 | −4.759 | −4.928 | 1.00 | 35.85 | | W | O |
| ANISOU | 1474 | O | HOH | W | 151 | 4541 | 4532 | 4549 | 3 | −24 | 23 | W | O |
| ATOM | 1475 | O | HOH | W | 152 | −8.412 | 6.752 | 0.251 | 1.00 | 32.26 | | W | O |
| ANISOU | 1475 | O | HOH | W | 152 | 4073 | 4073 | 4110 | 37 | −75 | 19 | W | O |
| ATOM | 1476 | O | HOH | W | 153 | −13.023 | −8.221 | −7.782 | 1.00 | 32.89 | | W | O |
| ANISOU | 1476 | O | HOH | W | 153 | 4180 | 4143 | 4174 | −3 | 60 | −2 | W | O |
| ATOM | 1477 | O | HOH | W | 154 | −13.208 | 11.082 | −21.898 | 1.00 | 40.04 | | W | O |
| ANISOU | 1477 | O | HOH | W | 154 | 4970 | 5090 | 5152 | −37 | 12 | −26 | W | O |
| ATOM | 1478 | O | HOH | W | 155 | −19.149 | 4.119 | −19.317 | 1.00 | 19.86 | | W | O |
| ANISOU | 1478 | O | HOH | W | 155 | 2509 | 2559 | 2477 | 194 | −63 | 18 | W | O |
| ATOM | 1479 | O | HOH | W | 156 | −20.492 | 18.216 | −1.534 | 1.00 | 32.66 | | W | O |
| ANISOU | 1479 | O | HOH | W | 156 | 4148 | 4088 | 4175 | 22 | 24 | 5 | W | O |
| ATOM | 1480 | O | HOH | W | 157 | −35.300 | −5.432 | −16.094 | 1.00 | 32.36 | | W | O |
| ANISOU | 1480 | O | HOH | W | 157 | 4062 | 4127 | 4105 | 53 | −28 | 13 | W | O |
| ATOM | 1481 | O | HOH | W | 158 | −13.547 | 13.164 | −15.727 | 1.00 | 29.38 | | W | O |
| ANISOU | 1481 | O | HOH | W | 158 | 3685 | 3704 | 3775 | 10 | −23 | 10 | W | O |
| ATOM | 1482 | O | HOH | W | 159 | −22.959 | 17.330 | −2.703 | 1.00 | 38.75 | | W | O |
| ANISOU | 1482 | O | HOH | W | 159 | 4869 | 4973 | 4880 | 32 | −16 | 47 | W | O |
| ATOM | 1483 | O | HOH | W | 160 | −19.480 | 15.893 | −8.204 | 1.00 | 40.98 | | W | O |
| ANISOU | 1483 | O | HOH | W | 160 | 5240 | 5157 | 5173 | 6 | −41 | −3 | W | O |
| ATOM | 1484 | O | HOH | W | 161 | −12.130 | −6.363 | −12.581 | 1.00 | 30.57 | | W | O |
| ANISOU | 1484 | O | HOH | W | 161 | 3848 | 3881 | 3886 | 51 | 15 | −22 | W | O |
| ATOM | 1485 | O | HOH | W | 162 | −21.519 | 15.984 | −5.415 | 1.00 | 34.15 | | W | O |
| ANISOU | 1485 | O | HOH | W | 162 | 4372 | 4281 | 4324 | 1 | 22 | −3 | W | O |
| ATOM | 1486 | O | HOH | W | 163 | −10.642 | −3.655 | −7.326 | 1.00 | 30.22 | | W | O |
| ANISOU | 1486 | O | HOH | W | 163 | 3734 | 3886 | 3861 | −18 | −35 | −19 | W | O |
| ATOM | 1487 | O | HOH | W | 164 | −16.003 | 15.845 | −11.987 | 1.00 | 33.56 | | W | O |
| ANISOU | 1487 | O | HOH | W | 164 | 4323 | 4156 | 4273 | −51 | −3 | 46 | W | O |
| ATOM | 1488 | O | HOH | W | 165 | −41.661 | −5.375 | −8.117 | 1.00 | 48.81 | | W | O |
| ANISOU | 1488 | O | HOH | W | 165 | 6200 | 6191 | 6153 | −2 | −7 | 7 | W | O |
| ATOM | 1489 | O | HOH | W | 166 | −11.258 | 10.250 | −4.887 | 1.00 | 29.05 | | W | O |
| ANISOU | 1489 | O | HOH | W | 166 | 3706 | 3696 | 3636 | −33 | −60 | 10 | W | O |
| ATOM | 1490 | O | HOH | W | 167 | −9.835 | 12.511 | −10.978 | 1.00 | 34.26 | | W | O |
| ANISOU | 1490 | O | HOH | W | 167 | 4285 | 4324 | 4407 | −94 | −5 | −49 | W | O |
| ATOM | 1491 | O | HOH | W | 168 | −21.373 | −17.739 | −15.785 | 1.00 | 30.47 | | W | O |
| ANISOU | 1491 | O | HOH | W | 168 | 3866 | 3824 | 3888 | 11 | 37 | 4 | W | O |
| ATOM | 1492 | O | HOH | W | 169 | −13.692 | 3.837 | −10.947 | 1.00 | 41.82 | | W | O |
| ANISOU | 1492 | O | HOH | W | 169 | 5256 | 5276 | 5356 | −5 | 31 | 10 | W | O |
| ATOM | 1493 | O | HOH | W | 170 | −26.343 | −14.724 | 10.918 | 1.00 | 22.06 | | W | O |
| ANISOU | 1493 | O | HOH | W | 170 | 2786 | 2785 | 2810 | 13 | −40 | −106 | W | O |
| ATOM | 1494 | O | HOH | W | 171 | −38.666 | −0.338 | −0.808 | 1.00 | 29.54 | | W | O |
| ANISOU | 1494 | O | HOH | W | 171 | 3749 | 3739 | 3734 | 109 | 51 | −60 | W | O |
| ATOM | 1495 | O | HOH | W | 172 | −46.649 | −10.554 | −10.990 | 1.00 | 29.25 | | W | O |
| ANISOU | 1495 | O | HOH | W | 172 | 3652 | 3731 | 3729 | 20 | 24 | −57 | W | O |
| ATOM | 1496 | O | HOH | W | 173 | −35.288 | 9.861 | −5.432 | 1.00 | 25.29 | | W | O |
| ANISOU | 1496 | O | HOH | W | 173 | 3273 | 3302 | 3033 | 23 | 38 | −38 | W | O |
| ATOM | 1497 | O | HOH | W | 174 | −22.002 | −14.472 | 6.571 | 1.00 | 25.70 | | W | O |
| ANISOU | 1497 | O | HOH | W | 174 | 3178 | 3435 | 3150 | 33 | −16 | 7 | W | O |
| ATOM | 1498 | O | HOH | W | 175 | −39.622 | −24.724 | −14.785 | 1.00 | 26.21 | | W | O |
| ANISOU | 1498 | O | HOH | W | 175 | 3239 | 3549 | 3169 | −40 | −90 | −39 | W | O |
| ATOM | 1499 | O | HOH | W | 176 | −33.595 | −20.281 | 1.934 | 1.00 | 26.47 | | W | O |
| ANISOU | 1499 | O | HOH | W | 176 | 3307 | 3394 | 3357 | 30 | −11 | −40 | W | O |
| ATOM | 1500 | O | HOH | W | 177 | −20.591 | −12.285 | 10.684 | 1.00 | 37.24 | | W | O |
| ANISOU | 1500 | O | HOH | W | 177 | 4791 | 4685 | 4673 | 23 | −1 | 32 | W | O |
| ATOM | 1501 | O | HOH | W | 178 | −15.209 | 17.130 | −7.820 | 1.00 | 37.87 | | W | O |
| ANISOU | 1501 | O | HOH | W | 178 | 4819 | 4745 | 4825 | −35 | −18 | 23 | W | O |
| ATOM | 1502 | O | HOH | W | 179 | −24.544 | −12.959 | 9.249 | 1.00 | 17.12 | | W | O |
| ANISOU | 1502 | O | HOH | W | 179 | 2550 | 2092 | 1862 | 3 | 48 | 50 | W | O |
| ATOM | 1503 | O | HOH | W | 180 | −13.182 | 3.720 | −19.624 | 1.00 | 27.68 | | W | O |
| ANISOU | 1503 | O | HOH | W | 180 | 3398 | 3539 | 3579 | −23 | 37 | 33 | W | O |
| ATOM | 1504 | O | HOH | W | 181 | −32.038 | 5.011 | −16.449 | 1.00 | 22.74 | | W | O |
| ANISOU | 1504 | O | HOH | W | 181 | 2906 | 2861 | 2875 | −61 | −52 | 81 | W | O |
| ATOM | 1505 | O | HOH | W | 182 | −16.553 | −2.372 | 5.336 | 1.00 | 32.54 | | W | O |
| ANISOU | 1505 | O | HOH | W | 182 | 4054 | 4144 | 4164 | −36 | 22 | 32 | W | O |
| ATOM | 1506 | O | HOH | W | 183 | −30.110 | 2.507 | −17.061 | 1.00 | 34.18 | | W | O |
| ANISOU | 1506 | O | HOH | W | 183 | 4413 | 4381 | 4193 | −79 | −5 | 17 | W | O |
| ATOM | 1507 | O | HOH | W | 184 | −32.092 | 8.417 | 7.086 | 1.00 | 29.00 | | W | O |
| ANISOU | 1507 | O | HOH | W | 184 | 3689 | 3625 | 3703 | 12 | −36 | 65 | W | O |
| ATOM | 1508 | O | HOH | W | 185 | −9.701 | 5.909 | −13.328 | 1.00 | 34.86 | | W | O |
| ANISOU | 1508 | O | HOH | W | 185 | 4426 | 4388 | 4431 | −12 | −38 | 0 | W | O |
| ATOM | 1509 | O | HOH | W | 186 | −38.067 | −26.038 | −8.364 | 1.00 | 25.03 | | W | O |
| ANISOU | 1509 | O | HOH | W | 186 | 3272 | 2997 | 3243 | −73 | −24 | 30 | W | O |
| ATOM | 1510 | O | HOH | W | 187 | −36.886 | 14.089 | 11.945 | 1.00 | 37.28 | | W | O |
| ANISOU | 1510 | O | HOH | W | 187 | 4701 | 4713 | 4750 | 5 | −8 | 37 | W | O |
| ATOM | 1511 | O | HOH | W | 188 | −12.458 | 5.260 | −9.288 | 1.00 | 40.97 | | W | O |
| ANISOU | 1511 | O | HOH | W | 188 | 5135 | 5172 | 5259 | 36 | −25 | −12 | W | O |
| ATOM | 1512 | O | HOH | W | 189 | −16.715 | −18.732 | −8.679 | 1.00 | 29.83 | | W | O |
| ANISOU | 1512 | O | HOH | W | 189 | 3648 | 3914 | 3773 | −2 | 25 | −51 | W | O |
| ATOM | 1513 | O | HOH | W | 190 | −16.253 | 12.848 | −19.583 | 1.00 | 28.21 | | W | O |
| ANISOU | 1513 | O | HOH | W | 190 | 3690 | 3545 | 3483 | −45 | 27 | 46 | W | O |
| ATOM | 1514 | O | HOH | W | 191 | −12.648 | −13.510 | −6.689 | 1.00 | 40.82 | | W | O |
| ANISOU | 1514 | O | HOH | W | 191 | 5107 | 5196 | 5205 | −2 | 0 | 7 | W | O |
| ATOM | 1515 | O | HOH | W | 192 | −33.188 | −19.092 | 4.270 | 1.00 | 12.92 | | W | O |
| ANISOU | 1515 | O | HOH | W | 192 | 1694 | 1561 | 1655 | 121 | 70 | 47 | W | O |
| ATOM | 1516 | O | HOH | W | 193 | −32.033 | −27.045 | −3.388 | 1.00 | 16.55 | | W | O |
| ANISOU | 1516 | O | HOH | W | 193 | 2364 | 1872 | 2053 | 89 | 127 | 15 | W | O |
| ATOM | 1517 | O | HOH | W | 194 | −33.034 | −27.618 | −5.837 | 1.00 | 28.01 | | W | O |
| ANISOU | 1517 | O | HOH | W | 194 | 3477 | 3704 | 3462 | 18 | −37 | −71 | W | O |
| ATOM | 1518 | O | HOH | W | 195 | −29.766 | −12.924 | −19.046 | 1.00 | 19.43 | | W | O |
| ANISOU | 1518 | O | HOH | W | 195 | 2679 | 2256 | 2448 | 61 | −9 | −37 | W | O |
| ATOM | 1519 | O | HOH | W | 196 | −36.209 | −14.072 | −18.601 | 1.00 | 18.48 | | W | O |
| ANISOU | 1519 | O | HOH | W | 196 | 2204 | 2554 | 2264 | −15 | −17 | 65 | W | O |
| ATOM | 1520 | O | HOH | W | 197 | −19.632 | −14.809 | −18.078 | 1.00 | 31.61 | | W | O |
| ANISOU | 1520 | O | HOH | W | 197 | 3966 | 4003 | 4040 | −20 | 53 | 33 | W | O |
| ATOM | 1521 | O | HOH | W | 198 | −35.965 | −19.560 | 1.308 | 1.00 | 19.15 | | W | O |
| ANISOU | 1521 | O | HOH | W | 198 | 2387 | 2481 | 2407 | −40 | 102 | 24 | W | O |
| ATOM | 1522 | O | HOH | W | 199 | −19.023 | −16.574 | 5.048 | 1.00 | 34.92 | | W | O |
| ANISOU | 1522 | O | HOH | W | 199 | 4450 | 4403 | 4414 | 8 | −48 | 19 | W | O |
| ATOM | 1523 | O | HOH | W | 200 | −32.264 | 1.311 | −17.693 | 1.00 | 36.00 | | W | O |
| ANISOU | 1523 | O | HOH | W | 200 | 4581 | 4589 | 4507 | −42 | 24 | 13 | W | O |
| ATOM | 1524 | O | HOH | W | 201 | −10.883 | 0.717 | −9.050 | 1.00 | 34.47 | | W | O |
| ANISOU | 1524 | O | HOH | W | 201 | 4350 | 4375 | 4371 | 18 | 34 | 49 | W | O |
| ATOM | 1525 | O | HOH | W | 202 | −13.114 | 14.491 | −13.396 | 1.00 | 37.67 | | W | O |
| ANISOU | 1525 | O | HOH | W | 202 | 4739 | 4727 | 4846 | −7 | 10 | −10 | W | O |
| ATOM | 1526 | O | HOH | W | 203 | −11.401 | 3.972 | −12.449 | 1.00 | 26.95 | | W | O |
| ANISOU | 1526 | O | HOH | W | 203 | 3391 | 3424 | 3424 | 2 | 48 | 72 | W | O |
| ATOM | 1527 | O | HOH | W | 204 | −12.921 | 15.175 | −17.404 | 1.00 | 41.19 | | W | O |
| ANISOU | 1527 | O | HOH | W | 204 | 5215 | 5194 | 5240 | −11 | −14 | 10 | W | O |
| ATOM | 1528 | O | HOH | W | 205 | −16.185 | −18.758 | −11.756 | 1.00 | 31.45 | | W | O |
| ANISOU | 1528 | O | HOH | W | 205 | 3942 | 3975 | 4032 | 10 | −48 | 15 | W | O |
| ATOM | 1529 | O | HOH | W | 206 | −28.178 | −14.281 | −20.942 | 1.00 | 23.21 | | W | O |
| ANISOU | 1529 | O | HOH | W | 206 | 2840 | 3032 | 2946 | −7 | −46 | 127 | W | O |
| ATOM | 1530 | O | HOH | W | 207 | −41.608 | −26.172 | −15.525 | 1.00 | 20.83 | | W | O |
| ANISOU | 1530 | O | HOH | W | 207 | 2559 | 2694 | 2661 | 88 | 30 | 4 | W | O |
| ATOM | 1531 | O | HOH | W | 208 | −31.007 | −28.137 | −7.582 | 1.00 | 33.07 | | W | O |
| ANISOU | 1531 | O | HOH | W | 208 | 4150 | 4277 | 4139 | 10 | −3 | 0 | W | O |
| ATOM | 1532 | O | HOH | W | 209 | −26.717 | −16.429 | −21.309 | 1.00 | 40.46 | | W | O |
| ANISOU | 1532 | O | HOH | W | 209 | 5097 | 5127 | 5149 | 9 | −6 | −39 | W | O |
| ATOM | 1533 | O | HOH | W | 210 | −14.590 | −6.202 | −14.654 | 1.00 | 24.63 | | W | O |
| ANISOU | 1533 | O | HOH | W | 210 | 3087 | 3169 | 3102 | −48 | −23 | −51 | W | O |
| ATOM | 1534 | O | HOH | W | 211 | −18.534 | 14.288 | −20.243 | 1.00 | 33.59 | | W | O |
| ANISOU | 1534 | O | HOH | W | 211 | 4241 | 4262 | 4261 | 44 | −18 | 40 | W | O |
| ATOM | 1535 | O | HOH | W | 212 | −34.991 | −9.772 | 5.173 | 1.00 | 24.50 | | W | O |
| ANISOU | 1535 | O | HOH | W | 212 | 3186 | 3142 | 2980 | −98 | 31 | 43 | W | O |
| ATOM | 1536 | O | HOH | W | 213 | −29.145 | −17.690 | −20.575 | 1.00 | 36.77 | | W | O |
| ANISOU | 1536 | O | HOH | W | 213 | 4717 | 4669 | 4584 | −12 | 10 | −37 | W | O |
| ATOM | 1537 | O | HOH | W | 214 | −21.647 | −14.722 | −16.446 | 1.00 | 14.03 | | W | O |
| ANISOU | 1537 | O | HOH | W | 214 | 1889 | 1799 | 1642 | 128 | 85 | −42 | W | O |
| ATOM | 1538 | O | HOH | W | 215 | −37.973 | −12.129 | 0.160 | 1.00 | 11.11 | | W | O |
| ANISOU | 1538 | O | HOH | W | 215 | 1540 | 1427 | 1253 | −1 | −9 | −18 | W | O |
| ATOM | 1539 | O | HOH | W | 216 | −34.433 | −11.523 | −21.527 | 1.00 | 18.28 | | W | O |
| ANISOU | 1539 | O | HOH | W | 216 | 2122 | 2311 | 2514 | −79 | −15 | −79 | W | O |
| ATOM | 1540 | O | HOH | W | 217 | −45.244 | −11.118 | −13.348 | 1.00 | 24.99 | | W | O |
| ANISOU | 1540 | O | HOH | W | 217 | 3218 | 2972 | 3306 | 5 | 107 | −62 | W | O |
| ATOM | 1541 | O | HOH | W | 218 | −11.731 | 6.833 | −19.982 | 1.00 | 30.42 | | W | O |
| ANISOU | 1541 | O | HOH | W | 218 | 3846 | 3957 | 3755 | 56 | −19 | 21 | W | O |
| ATOM | 1542 | O | HOH | W | 219 | −40.740 | −7.456 | −19.159 | 1.00 | 34.03 | | W | O |
| ANISOU | 1542 | O | HOH | W | 219 | 4380 | 4300 | 4250 | −3 | −41 | 6 | W | O |
| ATOM | 1543 | O | HOH | W | 220 | −15.766 | −2.407 | −13.856 | 1.00 | 21.48 | | W | O |
| ANISOU | 1543 | O | HOH | W | 220 | 2767 | 2693 | 2702 | 71 | 111 | 57 | W | O |
| ATOM | 1544 | O | HOH | W | 221 | −40.009 | 11.889 | 3.227 | 1.00 | 34.24 | | W | O |
| ANISOU | 1544 | O | HOH | W | 221 | 4371 | 4376 | 4261 | −25 | 43 | 12 | W | O |
| ATOM | 1545 | O | HOH | W | 222 | −11.482 | 12.602 | −8.555 | 1.00 | 32.40 | | W | O |
| ANISOU | 1545 | O | HOH | W | 222 | 3999 | 4135 | 4178 | −35 | −12 | −19 | W | O |
| ATOM | 1546 | O | HOH | W | 223 | −39.301 | −8.137 | −13.677 | 1.00 | 28.89 | | W | O |
| ANISOU | 1546 | O | HOH | W | 223 | 3672 | 3662 | 3641 | 36 | 74 | −21 | W | O |
| ATOM | 1547 | O | HOH | W | 224 | −9.153 | 7.193 | −20.136 | 1.00 | 38.80 | | W | O |
| ANISOU | 1547 | O | HOH | W | 224 | 4871 | 4925 | 4946 | −9 | 7 | 4 | W | O |
| ATOM | 1548 | O | HOH | W | 225 | −10.660 | 3.993 | −21.569 | 1.00 | 31.87 | | W | O |
| ANISOU | 1548 | O | HOH | W | 225 | 4006 | 4034 | 4069 | 39 | −16 | 41 | W | O |
| ATOM | 1549 | O | HOH | W | 226 | −14.607 | −3.251 | 2.355 | 1.00 | 31.52 | | W | O |
| ANISOU | 1549 | O | HOH | W | 226 | 4008 | 3990 | 3978 | −25 | −4 | −29 | W | O |
| ATOM | 1550 | O | HOH | W | 227 | −32.099 | −9.968 | 8.246 | 1.00 | 21.51 | | W | O |
| ANISOU | 1550 | O | HOH | W | 227 | 2911 | 2703 | 2558 | −135 | 89 | 41 | W | O |
| ATOM | 1551 | O | HOH | W | 228 | −18.947 | −18.450 | −15.806 | 1.00 | 35.58 | | W | O |
| ANISOU | 1551 | O | HOH | W | 228 | 4477 | 4498 | 4544 | 11 | 23 | −19 | W | O |
| ATOM | 1552 | O | HOH | W | 229 | −14.617 | −4.337 | 5.879 | 1.00 | 32.84 | | W | O |
| ANISOU | 1552 | O | HOH | W | 229 | 4091 | 4198 | 4188 | −3 | 24 | −54 | W | O |
| ATOM | 1553 | O | HOH | W | 230 | −18.632 | −3.056 | 9.142 | 1.00 | 18.31 | | W | O |
| ANISOU | 1553 | O | HOH | W | 230 | 2308 | 2415 | 2233 | −181 | −110 | 211 | W | O |
| ATOM | 1554 | O | HOH | W | 231 | −19.875 | 0.003 | 8.973 | 0.50 | 29.26 | | W | O |
| ANISOU | 1554 | O | HOH | W | 231 | 3656 | 3717 | 3745 | 0 | 0 | −21 | W | O |
| ATOM | 1556 | O | HOH | W | 232 | −18.267 | −0.042 | −16.681 | 1.00 | 31.38 | | W | O |
| ANISOU | 1556 | O | HOH | W | 232 | 4059 | 3901 | 3962 | 46 | −15 | −13 | W | O |
| ATOM | 1557 | O | HOH | W | 233 | −17.524 | −0.238 | 7.354 | 1.00 | 31.55 | | W | O |
| ANISOU | 1557 | O | HOH | W | 233 | 4057 | 4035 | 3897 | −24 | 8 | 35 | W | O |
| ATOM | 1558 | O | HOH | W | 234 | −31.339 | 12.412 | 8.912 | 1.00 | 36.68 | | W | O |
| ANISOU | 1558 | O | HOH | W | 234 | 4620 | 4657 | 4660 | −1 | 37 | 7 | W | O |
| ATOM | 1559 | O | HOH | W | 235 | −39.183 | −1.353 | 1.588 | 1.00 | 42.01 | | W | O |
| ANISOU | 1559 | O | HOH | W | 235 | 5236 | 5367 | 5358 | 32 | 15 | −2 | W | O |
| ATOM | 1560 | O | HOH | W | 236 | −19.646 | −23.489 | −7.613 | 1.00 | 34.45 | | W | O |
| ANISOU | 1560 | O | HOH | W | 236 | 4371 | 4389 | 4328 | 79 | −10 | 5 | W | O |
| ATOM | 1561 | O | HOH | W | 237 | −19.777 | 17.123 | −13.589 | 1.00 | 43.45 | | W | O |
| ANISOU | 1561 | O | HOH | W | 237 | 5492 | 5467 | 5551 | 9 | −1 | −6 | W | O |
| ATOM | 1562 | O | HOH | W | 238 | −9.204 | 12.782 | −15.255 | 1.00 | 46.73 | | W | O |
| ANISOU | 1562 | O | HOH | W | 238 | 5930 | 5931 | 5896 | −9 | −6 | −1 | W | O |
| END | | | | | | | | | | | | | |

The upstream region (Ser²⁰² to Asn²⁰⁸) of vIRF4, which binds to the HAUSP TRAF domain in a novel extended conformation, participates in extensive interactions with the other side of the β-sheet of the TRAF domain, especially the 136 strand (FIGS. 1b and d). The TRAF Arg¹⁵³ appears to play a decisive and unique role in the interaction with the upstream region of the vIRF4 peptide: the TRAF Agr¹⁵³ side chain amino group engages in hydrogen bonding with the vIRF4 Ile²⁰⁵ backbone oxygen and also participates in water molecule-mediated backbone-backbone interactions with the vIRF4 Pro²⁰⁶ (FIG. 1c). In addition, the β-carbon of TRAF Arg¹⁵³ participates in hydrophobic interactions with the vIRF4 Val²⁰⁷ α-carbon and side chain, and the backbone oxygen of TRAF Arg¹⁵³ directly interacts with the backbone nitrogen of peptide Asn²⁰⁸ (FIG. 1c). The vIRF4 Asn²⁰⁸ side chain participates in additional interactions with the TRAF Ser¹⁵⁵ backbone amide and Trp¹⁶⁵ indole nitrogen (FIG. 1c). On the other hand, the Glu²⁰⁹ and Gly²¹⁰ residues located in the middle of the vIRF4 peptide grasp the TRAF β6 and β7 strands (FIGS. 1b and d): the vIRF4 Glu²⁰⁹ backbone oxygen interacts with the Arg¹⁵² side chain amino group on the TRAF β6 strand (FIG. 1c), and the vIRF4 Gly²¹⁰ backbone oxygen forms a water molecule-mediated hydrogen bond with the Ser¹⁶⁸ side chain hydroxyl group and backbone nitrogen on the TRAF β7 strand. Finally, the vIRF4 Ser²⁰² backbone amide participates in water molecule-mediated interactions with the TRAF Ser¹⁵¹ backbone nitrogen and Ser¹⁴⁹ backbone oxygen, and the vIRF4 Trp²⁰⁴ and Ile²⁰⁵ backbone nitrogens participate in water molecule-mediated interactions with the TRAF Ser¹⁵¹ backbone oxygen. These results indicate that the upstream region of the vIRF4 peptide may be vital for stabilizing its interaction with the HAUSP TRAF domain. In fact, ITC analysis demonstrated that the deletion of this upstream region (vIRF4²⁰⁹⁻²¹⁶) resulted in a 25-fold decrease in TRAF binding affinity compared with the vIRF4²⁰²⁻²¹⁶ peptide (Table 1a).

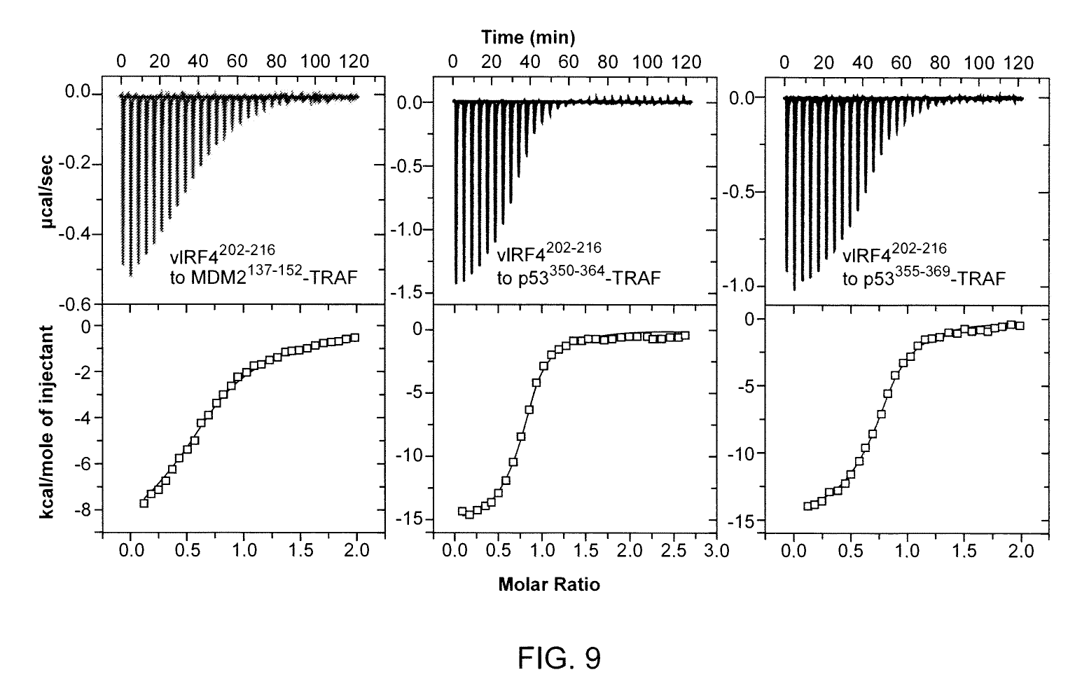

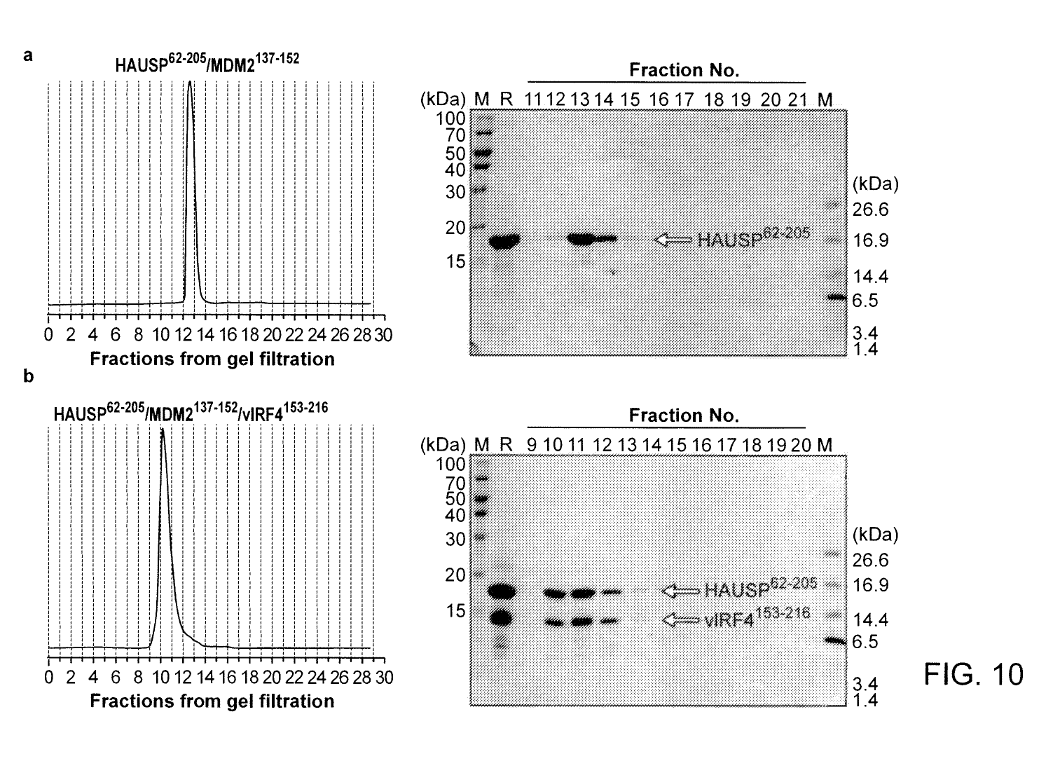

ITC binding affinity study indicates that the vIRF4²⁰²⁻²¹⁶ peptide exhibits 28-40-fold tighter binding (K_{d}=0.39 μM) to the TRAF domain compared to the peptides derived from MDM2 and p53 (Table 1a). To evaluate whether vIRF4 competes with cellular substrates for binding to the HAUSP TRAF domain, each peptide (MDM2¹³⁷⁻¹⁵², p53³⁵⁰⁻³⁶⁴, and p53³⁵⁵⁻³⁶⁹) was first titrated into the HAUSP TRAF domain, resulting in association constants (Kₐ) of 9.1×10⁴ M⁻¹, 6.5×10⁴ M⁻¹, and 6.7×10⁴ M⁻¹, respectively. When vIRF4²⁰²⁻²¹⁶ was subsequently titrated against HAUSP cellular substrates as a competitor, the association constant (K_{obs}) of each titration was markedly increased to 10.9×10⁶ M⁻¹, 44.2×10⁶ M⁻¹, and 35.8×10⁶ M⁻¹, respectively, indicating a considerably tighter interaction between HAUSP TRAF domain and vIRF4²⁰²⁻²¹⁶ compared to its cellular substrates, MDM2 and p53 (Table 1b and FIG. 9). To further gauge the competitive nature of vIRF4 binding, the TRAF domain was incubated with an equal molar amount of vIRF4¹⁵³⁻²¹⁶ in the presence of a 5-fold molar excess of MDM2¹³⁷⁻¹⁵², and the mixture was then analyzed by size exclusion chromatography. The vIRF4²⁰²⁻²¹⁶ peptide formed a stable complex with the TRAF even in the presence of a 5-fold molar excess of MDM2 peptide (FIG. 10). This competitive nature of vIRF4²⁰²⁻²¹⁶ peptide binding is likely due to its upstream region, since the upstream-region-deleted vIRF4²⁰⁹⁻²¹⁶ peptide exhibited comparable binding affinity with cellular substrates (Table 1a).

ITC analysis also showed that while vIRF4¹⁵³⁻²¹⁶ exhibited comparable TRAF binding affinity (K_{d}=0.54 μM) to that of vIRF4²⁰²⁻²¹⁶, the C-terminally extended vIRF4¹⁵³⁻²⁵⁶ exhibited a 7-fold higher affinity (K_{d}=0.076 μM) for the HAUSP TRAF domain. Deletion of residues 202-216 of vIRF4 (vIRF4^{153-256/}Δ²⁰²⁻²¹⁶, K_{d}=3.45 μM) led to a significant reduction of TRAF binding affinity, whereas an additional deletion of residues 237-256 (vIRF4^{153-256/}Δ^{202-216/}Δ²³⁷⁻²⁵⁶, K_{d}=4.03 μM) did not further affect TRAF binding affinity (Table 1a). These indicate that besides the vIRF4²⁰²⁻²¹⁶ residues, the vIRF4²¹⁷⁻²³⁶ sequence also plays an important role in TRAF binding. To further investigate this, this study analyzed NMR chemical shift perturbations of vIRF4¹⁵³⁻²⁵⁶ in the presence of HAUSP⁶²⁻²⁰⁵ (TRAF domain) or HAUSP⁶²⁻⁵⁶⁰ (TRAF-Catalytic-domain). FIG. 2a shows the superposition of 2D ¹H-¹⁵N correlation spectra of vIRF4¹⁵³⁻²⁵⁶ in the absence and presence of each HAUSP fragment. Signal changes (denoted by blue triangles) of vIRF4¹⁵³⁻²⁵⁶ were observed upon the binding of HAUSP⁶²⁻²⁰⁵ (red contours) compared to free vIRF4¹⁵³⁻²⁵⁶ (black contours), and additional changes (denoted by orange triangles) were detected upon the binding of HAUSP⁶²⁻⁵⁶⁰ (blue contours).

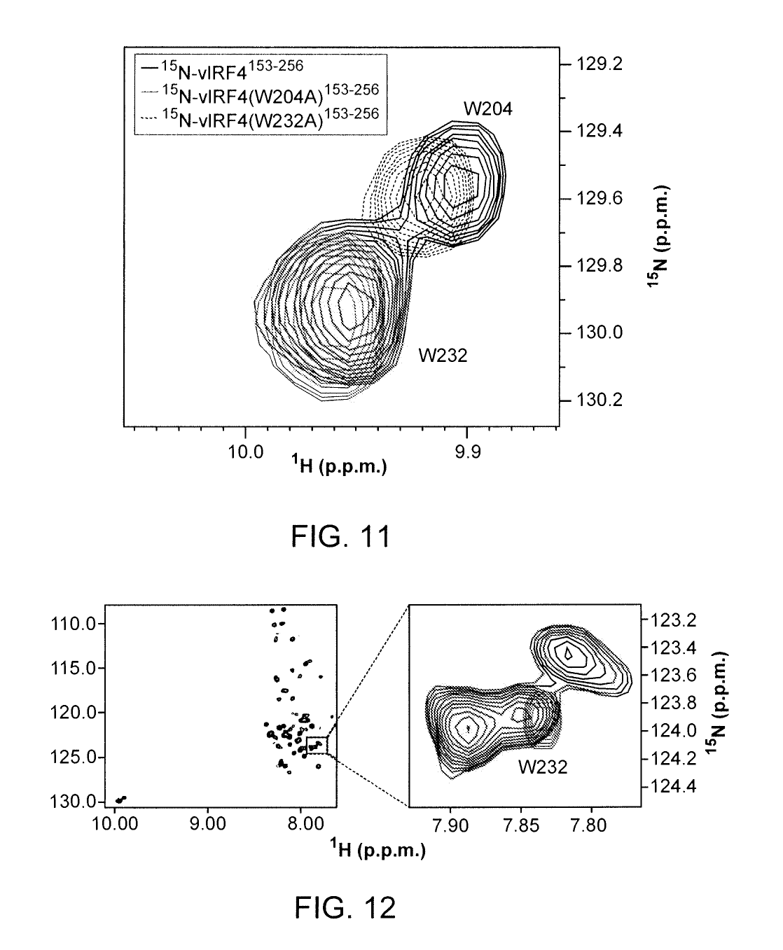

2D ¹H-¹⁵N correlation spectra and mutational analysis revealed that the ε-NH proton of Trp²⁰⁴ changed dramatically upon binding of TRAF-containing HAUSP⁶²⁻²⁰⁵ (FIG. 2c, light gray contours and FIG. 11), consistent with crystal structure data showing that Trp²⁰⁴ is located in the TRAF binding region of vIRF4 (aa202-216). On the other hand, the ε-NH signal of Trp²³² was evidently perturbed by binding of the TRAF-Catalytic-domain-containing HAUSP⁶²⁻⁵⁶⁰, but not by binding of the TRAF-containing HAUSP⁶²⁻²⁰⁵ (FIG. 2c, dark gray contours), suggesting that Trp²³² is involved in vIRF4 interaction with the HAUSP catalytic domain. Selective isotope (¹⁵N) labeling of Trp²³² was further carried out to identify backbone amide signals derived from residues located near the Trp²³² by comparing vIRF4 wild-type (wt) and W232A mutant (FIG. 2b, denoted by light gray arrows and FIG. 12). This also indicates that the residues near the Trp²³² are involved in binding to the HAUSP catalytic domain (FIG. 2a). Furthermore, the two short sequences of vIRF4, vIRF4²⁰²⁻²¹⁶ and vIRF4²²⁰⁻²³⁶, were individually capable of interacting with full length HAUSP in vivo as efficiently as vIRF4²⁰²⁻²⁵⁶, whereas vIRF4²³⁷⁻²⁵⁶ showed little or no HAUSP binding. The experiment used cells that were single transfected with the indicated vIRF4 constructs. The cells were harvested, followed by GST-pulldown and IB with an anti-HAUSP antibody. 1% of the WCL was used as the input. Based on these results, this example postulated a bilateral mode of interaction between vIRF4 and HAUSP (FIG. 2d): vIRF4²⁰²⁻²¹⁶ interacts with the HAUSP TRAF domain (primarily β6 and β7) with an unusually high binding affinity, while vIRF4²¹⁷⁻²³⁶ contacts the catalytic domain of HAUSP.

To investigate the bilateral mode of interaction between vIRF4 and HAUSP and the biological relevance of this interaction, this study first tested the effect of vIRF4 on HAUSP enzymatic activity. 293T cells were transfected with Flag-HAUSP together with V5-vIRF4 (wt) or vIRF4Δ²⁰²⁻²⁵⁶ mutant incapable of binding HAUSP. Here, 293T cells were co-transfected with HAUSP and the wt or mutant forms of vIRF4, followed by IP with anti-Flag (M2) agarose beads and a Flag (M2) peptide was used to elute purified complexes. Purified HAUSP complexes were incubated with K48-linked ubiquitin chains at 37° C. for the indicated times and IB with an anti-ubiquitin antibody. 1% of the WCL was used as the input. Further, in vitro DUB assay of immuno-purified Flag-HAUSP complexes with K48- or K63-linked 3-7 ubiquitin chains showed that vIRF4 effectively suppressed HAUSP DUB activity in a binding dependent manner. To further test the effects of vIRF4 short sequences on HAUSP enzymatic activity, the vIRF4 peptides corresponding to aa202-216 (called vif1 peptide) and aa220-236 (called vif2 peptide), were mixed with 0.25 μM purified HAUSP for 5 min and then subjected to an in vitro DUB assay with K48- or K63-linked 3-7 polyubiquitin chains. An “Amp” peptide derived from the amphipathic helix sequence of herpesvirus saimiri Tip protein was included as a negative control. This assay showed that vif2 peptide markedly suppressed HAUSP DUB activity, whereas vif1 peptide's inhibition was minimal. In this experiment, purified HAUSP (0.25 μM) was pre-mixed with the vif1, vif2, or Amp (nonspecific peptide) peptide for 5 min and then subjected to an in vitro deubiquitination assay with K48-linked 3-7 polyubiquitin chains and IB with an anti-ubiquitin antibody. Also, purified MDM2 (also, E3 ligase) was incubated with E1, E2 (Ubc H5b), and ubiquitin (in vitro MDM2 ubiquitination assay). Pre-mixed HAUSP and peptide were incubated with ubiquitinated MDM2 and IB with an anti-MDM2 antibody. It was observed that vif1/2 peptides block HAUSP activity via different manner in vitro.

By contrast, neither vif1 peptide nor vif2 peptide was capable of inhibiting USP8 DUB activity, demonstrating the specificity of vif1- and vif2-mediated inhibition of HAUSP activity. Purified USP8 (0.25 μM) was pre-mixed with the vif1, vif2, or Amp (non-specific peptide) peptide for 5 min and then subjected to an in vitro deubiquitination assay with K48- or K63-linked 3-7 polyubiquitin chain and IB with an anti-ubiquitin antibody. It was shown that vif1/2 peptides can not inhibit the USP8 de-ubiquitinase enzymatic activity in vitro. Comparative kinetic analysis showed that while vif1 peptide weakly attenuated HAUSP DUB activity, vif2 peptide completely suppressed HAUSP DUB activity (FIG. 2e). These results strongly suggest that vif2 peptide corresponding to the vIRF4²²⁰⁻²³⁶ may directly contact the catalytic domain of HAUSP and hence inhibit its DUB activity.

This study then investigated whether vif1 and vif2 peptides can inhibit HAUSP DUB activity against ubiquitinated substrates through substrate binding competition. To this end, this study first generated ubiquitinated MDM2 using purified E1 (UBE1), E2 (UbCH5b), and E3 (MDM2) proteins, and then performed an in vitro DUB assay employing purified HAUSP alone or HAUSP preincubated with increasing amounts of each peptide (FIG. 2f). vif2 peptide efficiently blocked HAUSP enzymatic activity against ubiquitinated MDM2 and polyubiquitin chains (FIG. 2e and f). In striking contrast to its ineffectiveness against K48- or K63-linked polyubiquitin chains, vif1 peptide efficiently blocked HAUSP DUB activity when ubiquitinated MDM2 was used as a substrate (FIG. 2f). To further delineate the vIRF4 peptides' action in vivo, the vif1 and vif2 peptides were fused with the HIV-1 TAT protein transduction domain for intracellular delivery (Ref 11, 12) and tested for their potential effects on in vivo HAUSP DUB activity. At 24 h post-transfection with Flag-HAUSP, 293T cells were incubated with 100 μM of each TAT-conjugated peptide for an additional 12 h, followed by immunopurification of Flag-HAUSP, which was then used for an in vitro DUB assay. Consistent with the previous in vitro DUB assay, TAT-vif2 peptide showed the strongest inhibitory activity toward ex vivo HAUSP enzymatic activity, but TAT-vif1 peptide showed no effect under the same conditions (FIG. 2g). These suggest that vif1 interferes with HAUSP substrate binding, while vif2 inhibits HAUSP DUB activity.

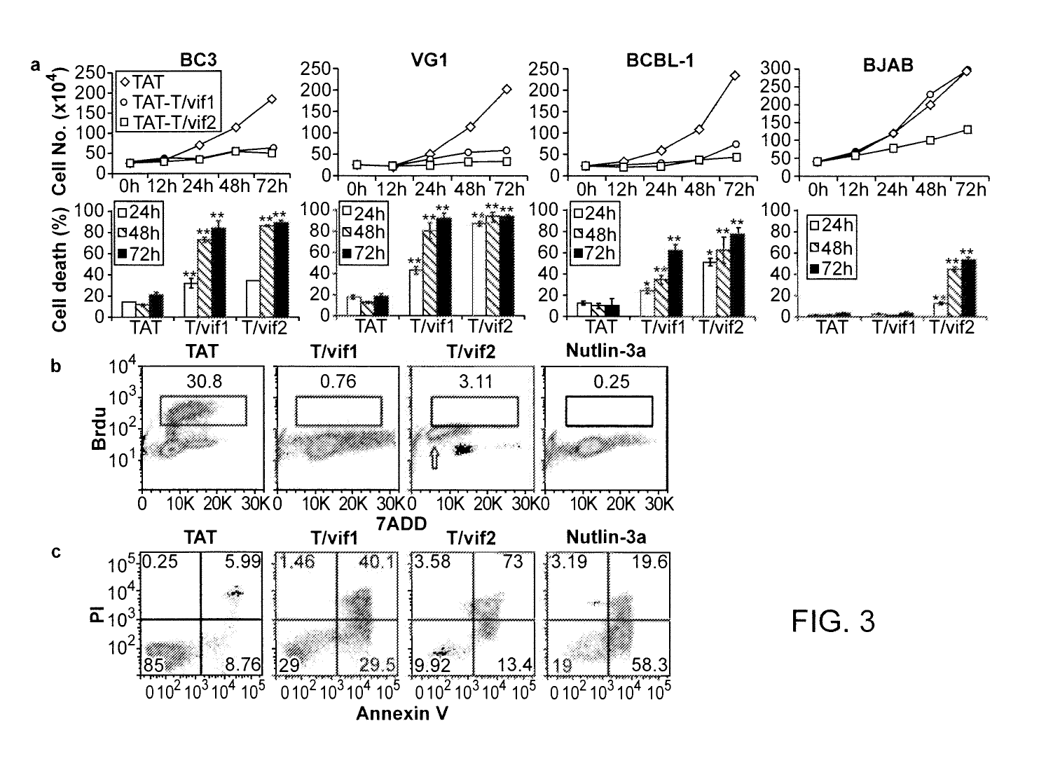

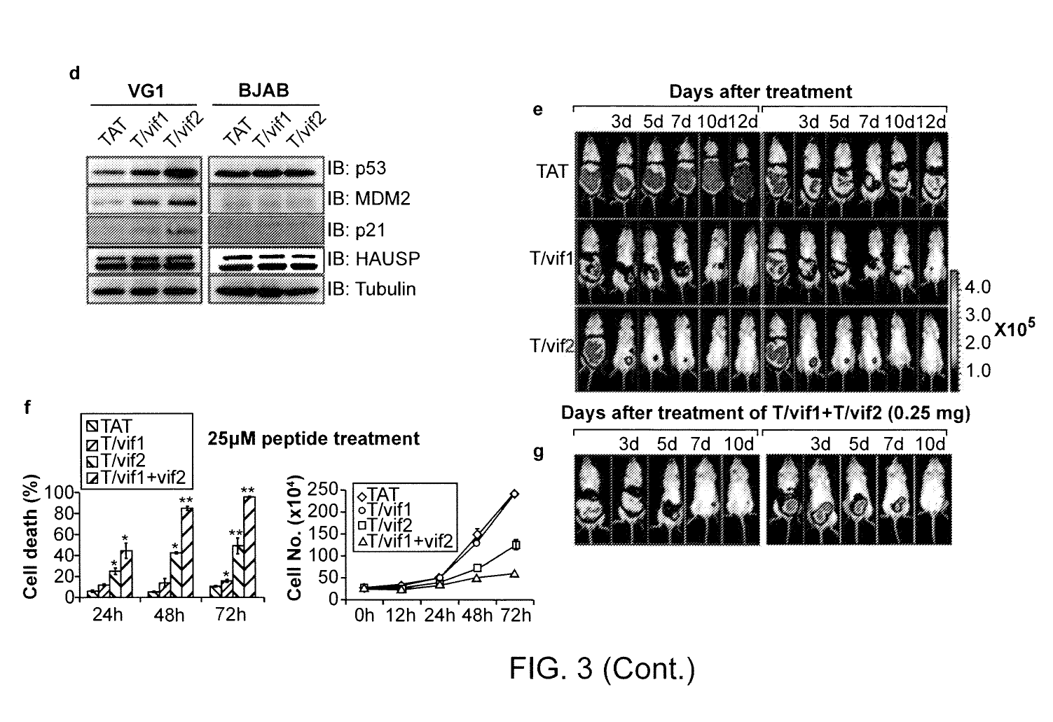

Since HAUSP plays a pivotal role in the regulation of the p53 pathway (Ref. 2, 3), this study investigated the potential effect of each peptide on KSHV-induced primary effusion lymphoma (PEL) tumor cell lines harboring p53 (wt) (Ref. 13, 14). Cell lines with mutated, non-functional p53 were included as controls. This showed that time-dependent antiproliferative and cytotoxic activity differed depending on p53 status. Incubation of PELs with various concentrations (25, 50, or 100 μM) of the TAT-vif2 peptide not only robustly suppressed cell proliferation, but also induced profound cell death, whereas TAT-vif1 peptide showed much weaker activity than TAT-vif2 peptide (FIG. 3a). In contrast, treatment with HIV-1 TAT showed no effect on cell proliferation and cell death (FIG. 3a). Three different human cancer cell lines, SJSA-1 (MDM2 amplification), MCF7 (MDMX amplification), and LnCap (HAUSP overexpression), were treated with 100 μM of TAT, TAT-vif1, or TATvif2. Cell viability was measured for the indicated times after treatment of each peptide using a WST-1 assay. Relative cell growth was determined by calculating the OD450ₙₘ at each time point relative to t=0. Results are expressed as the mean±SD of triplicate cultures and are representative of at least 3 independent experiments. Three prostate cancer cell lines harboring different p53 status, LnCap (p53 wt/wt), DU145 (p53 m/m), and PC3 (p53−/−), were used for WST-1 assays. It was observed that vif1/2 peptides induced significant growth inhibition in HAUSP overexpression cell lines and in a p53 (wt) dependent manner.

Significantly, BJAB Burkitt lymphoma tumor cells carrying mutant p53 (Ref. 15) continued to proliferate in the presence of TAT-vif1 and only minor growth reduction and cell death in the presence of TAT-vif2 peptide (FIG. 3a). When prostate cancer cells, LnCap (p53^{wt/wt}), PC3 (p53^{-/-}), and DU145 (p53^{m/m}) (Ref. 16) carrying different functional p53 genotypes were subjected to peptide treatment, the p53-dependence of TAT-vif1 and TAT-vif2 peptides was also evident: LnCap cells, but not PC3 and DU145 cells, were highly susceptible to the TAT-vif1- and TAT-vif2-mediated cell growth inhibition. Additionally, the high level of HAUSP expression in LnCap cells likely contributed to the strong susceptibility to TAT-vif1- and TAT-vif2-mediated cell growth inhibition since SJSA-1 and MCF7 tumor cells carrying high levels of MDM2 and MDMX expression, respectively, did not show as robust a susceptibility to vif1- and vif2-mediated cell growth inhibition as LnCap cells. These data collectively demonstrate that both vif1 and vif2 peptides have vigorous cell killing activities against p53 (wt)-containing tumor cells.

One of the main cellular consequences of p53 activation in proliferating cells is cell cycle arrest through transcriptional upregulation of the cyclin-dependent kinase inhibitor p21, which causes G₁-S or G₂-M cell cycle arrest (Ref 17). Indeed, treatment of PEL cells with the TAT-vif1 or TAT-vif2 peptide markedly increased the G₁ and G₂/M phase fraction and nearly completely depleted S-phase cells (FIG. 3b). Asynchronously growing PEL cells (BC-1, VG1, and BC3) and mutant p53 cells (BJAB) were treated with 100 μM of either vif1 or vif2 peptide for the indicated time periods. 10 μM Nutlin-3a was used as a positive control. Cells were pulse-labeled with BrdU and analyzed for DNA content by flow cytometry. BrdU incorporation during the S phase is indicated as percentage of stained cells. The sub-G1 populations are denoted by arrow. Data are representative of 3 independent experiments. The results show that vif1/2 peptides induce cell cycle arrest in p53 (wt) harboring cells.

Interestingly, this study also observed significant sub-G₁ accumulation, reflecting cell death, in TAT-vif2 treated cells compared to TAT or TAT-vif1 treated cells (FIG. 3b) Annexin V and propidium iodide (PI) staining assay showed that TAT-vif1 or TAT-vif2 peptide treatment effectively induced apoptotic cell death in PEL cells carrying p53 (wt) compared with TAT treatment where TAT-vif2 peptide more rapidly and dramatically induced apoptotic cell death compared with TAT-vif1 (FIG. 3c). Apoptosis in BC-1, VG1, BC3, and BJAB cells was assessed at the indicated time after treatment with 100 μM each peptide or 10 μM Nutlin-3a by Annexin V-FITC/PI binding and subjected to flow cytometry analysis; lower right quadrants represent early apoptotic cells (Annexin V-positive, PI-negative) demonstrating cytoplasmic membrane intergrity; upper right quadrants represent non-viable, late apoptotic cells (Annexin V- and PI-positive). Numbers indicate the percentage of cells in each phase. Shown is one representative experiment of three. It was shown that vif1/2 peptides cause cell death by apoptosis.

Treatment of Nutlin-3a, which blocks the interaction between MDM2 and p53 and thus induces extensive apoptosis, also led to considerable cell death, comparable to either peptide treatment. As the inhibition of HAUSP enzymatic activity results in the stabilization and activation of p53, this study examined the effect of each peptide on the intracellular levels of p53 and its transcriptional targets p21 and MDM2. Incubation of exponentially growing PELs cells with either peptide for 6 h led to increased levels of p53, p21, and MDM2 (FIG. 3d). Cells with wt and mutant p53 were incubated for the indicated times in the presence of each peptide. WCL were subjected to SDS-PAGE followed by Western blotting and analyzed for p53, MDM2, p21, and HAUSP expression. Tubulin immunoblot is shown as a loading control. vif1/2 peptides, as the data shows, activated p53 and its target genes.

In contrast, BJAB cells exposed to the same conditions showed no detectable changes in p53, MDM2, and p21. Neither vif1 nor vif2 peptide treatment altered HAUSP levels (FIG. 3d). These results demonstrate that TAT-vif1 and TAT-vif2 peptides activate the p53 pathway primarily in cancer cells with functional p53 (wt).

To evaluate the in vivo anti-tumor activity of vif1 and vif2 peptides, this study utilized NOD/SCID xenografted mice intraperitoneally injected with BCBL-1 cells expressing the luciferase gene as a traceable bioluminescence reporter and evaluated these mice for the development of PEL, as shown in references 18-20. After being injected with the tumor cells, all of the mice developed PEL, with evident distention and ascites in the peritoneal cavity as well as markedly increased luminescence (data not shown). Mice with advanced PEL were challenged with 1 mg (equivalent to ˜100 μM) TAT, TAT-vif1, or TAT-vif2 peptide on days 3, 5, 7, and twice weekly for subsequent weeks by intraperitoneal injection. Treatment with TAT-vif1 or TAT-vif2 peptide led to little or no traceable luminescence, showing marked tumor regression (FIG. 3e). In particular, TAT-vif2 peptide caused efficient and powerful tumor regression. By contrast, tumors continuously advanced in mice that received TAT peptide injection (FIG. 3e). After PEL establishment, mice were challenged with intraperitoneal injections of 1 mg of the TAT, TAT-vif1, or TAT-vif2 peptide. Tumors were monitored via bioluminescence imaging. Tumor regression was observed to be induced by the vif1/2 peptides.

BCBL-1 cells were treated with different doses of vif1 (25 or 50 μM), vif2 (12.5 or 25 μM) or together with each combination of peptides for the indicated time periods. The percentage of dead cells was determined by trypan blue staining Data are mean±s. e.m.; n=200-300 cells from three independent experiments; *p<0.05; **p<0.01. Synergistic effect of the addition of vif2 peptide to vif1 peptide was observed.

None of the mice showed significant weight-loss, nor any gross abnormalities upon necropsy at the end of the treatment. In addition, the combination of 25 μM of the TAT-vif1 and TAT-vif2 peptide were highly effective in inducing p53-dependent growth suppression as well as cell death, concordant with the 100 μM peptide treatment results (FIG. 3f). Furthermore, when NOD/SCID mice with advanced PEL were challenged with the combination of TAT-vif1 and TAT-vif2 peptide each at a dose of 0.25 mg, this too led to marked tumor regression (FIG. 3g).

Combining vif1 and vif2 peptide leads to synergistic effect on cell cycle arrest, further data shows. BCBL-1 cells were treated with 100 μM of either vif1 or vif2 peptide for the indicated time periods. 10 μM Nutlin-3a was used as a positive control. BCBL-1 cells were treated with 25 μM vif1 peptide alone, vif2 peptide alone, or vif1 and vif2 peptide together for the indicated time periods. Cells were pulse-labeled with BrdU and analyzed for DNA content by flow cytometry. BrdU incorporation during the S phase is indicated as percentage of stained cells. The sub-G1 populations are denoted by arrow. Data are representative of 3 independent experiments.

It was also observed that combining vif1 and vif2 peptide leads to synergistic effect on apoptosis. In this respect, BCBL-1 cells were treated with 100 μM of either vif1 or vif2 peptide for the indicated time periods and then subjected to Annexin V and PI staining, followed by FACS analysis. 10 μM Nutlin-3a was used as a positive control. BCBL-1 cells were treated with 25 μM vif1 peptide alone, vif2 peptide alone, or vif1 and vif2 peptide together for the indicated time periods and then subjected to Annexin V and PI staining, followed by FACS analysis.

Also observed was the synergistic effect of vif1 and vif2 peptides on the activation of p53 and its target genes. BCBL-1 cell were incubated for the indicated times with 25 μM vif1 peptide alone, vif2 peptide alone, or vif1 and vif2 peptide together. WCL were subjected to SDS-PAGE followed by Western blotting and analyzed for p53, MDM2, p21, and HAUSP expression. Tubulin immunoblot is shown as a loading control. Still further observed was the synergistic effect of vif1 and vif2 peptides on tumor regression. After PEL establishment, mice were challenged with intraperitoneal injections of 0.25 mg of TAT-vif1 and TAT-vif2 peptide together. Tumors were monitored via bioluminescence imaging.

These results collectively indicate that TAT-vif1 and TAT-vif2 peptides robustly suppress HAUSP DUB enzymatic activity, ultimately leading to p53-mediated anti-cancer activity.

Recent accumulated observations suggest that the re-introduction of functional p53 can robustly induce tumor regression (Ref 21, 22), and p53 is also essential for effective chemo- or radio-therapy (Ref. 23). Thus, any small molecule or peptide that can activate p53 would be a valuable cancer therapeutic reagent. Along the same lines, it is unquestionable that inhibitors of HAUSP are therapeutically beneficial against p53 (wt) tumors as a very recent paper reported that HAUSP knockout embryos showed p53 stabilization and cell growth arrest (Ref 24). Due to the high binding affinity between HAUSP and MDM2, coupled with the observation that MDM2 is highly destabilized in the absence of HAUSP, blocking HAUSP activity should have a net effect of robust p53 stabilization. This study shows that vIRF4-derived short vif1 and vif2 peptides posses two provocative and effective strategies, thereby acting as specific and robust HAUSP antagonists: the vif1 peptide binds to the TRAF domain of HAUSP with a higher affinity than any other reported substrate, blocking its binding to other substrates, whereas the vif2 peptide appears to loosely bind the TRAF domain and the active site of the catalytic domain of HAUSP, suppressing its DUB enzymatic activity. Consequently, the vIRF4-derived short vif1/2 peptides comprehensively suppress HAUSP activity, effectively restoring p53-dependent apoptosis in wild-type p53-carrying cancer cells and suppressing tumor growth in mouse xenograft models. Of especial importance, this study herein reports that vif1/2 peptides represent potential novel chemotherapeutic molecules for anti-cancer therapies.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Hu, M. et al. Structural basis of competitive recognition of p53 and MDM2 by HAUSP/USP7: implications for the regulation of the p53-MDM2 pathway. PLoS Biol 4, e27 (2006).
2. Cummins, J. M. et al. Tumour suppression: disruption of HAUSP gene stabilizes p53. Nature 428, 1 p following 486 (2004).
3. Cummins, J. M. & Vogelstein, B. HAUSP is required for p53 destabilization. Cell Cycle 3, 689-692 (2004).
4. Li, M. et al. Deubiquitination of p53 by HAUSP is an important pathway for p53 stabilization. Nature 416, 648-653 (2002).
5. Everett, R. D. et al. A novel ubiquitin-specific protease is dynamically associated with the PML nuclear domain and binds to a herpesvirus regulatory protein. EMBO J 16, 1519-1530 (1997).
6. Uetz, P. et al. Herpesviral protein networks and their interaction with the human proteome. Science 311, 239-242 (2006).
7. Sarkari, F. et al. Further insight into substrate recognition by USP7: structural and biochemical analysis of the HdmX and Hdm2 interactions with USP7. J Mol Biol 402, 825-837.
8. Saridakis, V. et al. Structure of the p53 binding domain of HAUSP/USP7 bound to Epstein-Barr nuclear antigen 1 implications for EBV-mediated immortalization. Mol Cell 18, 25-36 (2005).
9. Sheng, Y. et al. Molecular recognition of p53 and MDM2 by USP7/HAUSP. Nat Struct Mol Biol 13, 285-291 (2006).
10. Dong, A. et al. In situ proteolysis for protein crystallization and structure determination. Nat Methods 4, 1019-1021 (2007).
11. Wadia, J. S., Stan, R. V. & Dowdy, S. F. Transducible TAT-HA fusogenic peptide enhances escape of TAT-fusion proteins after lipid raft macropinocytosis. Nat Med 10, 310-315 (2004).
12. Gump, J. M. & Dowdy, S. F. TAT transduction: the molecular mechanism and therapeutic prospects. Trends Mol Med 13, 443-448 (2007).
13. Katano, H., Sato, Y. & Sata, T. Expression of p53 and human herpesvirus-8 (HHV-8)-encoded latency-associated nuclear antigen with inhibition of apoptosis in HHV-8-associated malignancies. Cancer 92, 3076-3084 (2001).
14. Petre, C. E., Sin, S. H. & Dittmer, D. P. Functional p53 signaling in Kaposi's sarcoma-associated herpesvirus lymphomas: implications for therapy. J Virol 81, 1912-1922 (2007).
15. Bhatia, K. et al. Hemi- or homozygosity: a requirement for some but not other p53 mutant proteins to accumulate and exert a pathogenetic effect. FASEB J 7, 951-956 (1993).
16. Zhang, R., Wang, H. & Agrawal, S, Novel antisense anti-MDM2 mixed-backbone oligonucleotides: proof of principle, in vitro and in vivo activities, and mechanisms. Curr Cancer Drug Targets 5, 43-49 (2005).
17. Bunz, F. et al. Requirement for p53 and p21 to sustain G2 arrest after DNA damage. Science 282, 1497-1501 (1998).
18. Keller, S. A. et al. NF-kappaB is essential for the progression of KSHV- and EBV-infected lymphomas in vivo. Blood 107, 3295-3302 (2006).
19. Wu, W., Rochford, R., Toomey, L., Harrington, W., Jr. & Feuer, G Inhibition of HHV-8/KSHV infected primary effusion lymphomas in NOD/SCID mice by azidothymidine and interferon-alpha. Leuk Res 29, 545-555 (2005).
20. Lee, J. S. et al. FLIP-mediated autophagy regulation in cell death control. Nat Cell Biol 11, 1355-1362 (2009).
21. Ringshausen, I., O'Shea, C. C., Finch, A. J., Swigart, L. B. & Evan, G. I. Mdm2 is critically and continuously required to suppress lethal p53 activity in vivo. Cancer Cell 10, 501-514 (2006).
22. Martins, C. P., Brown-Swigart, L. & Evan, G. I. Modeling the therapeutic efficacy of p53 restoration in tumors. Cell 127, 1323-1334 (2006).
23. El-Deiry, W. S. The role of p53 in chemosensitivity and radiosensitivity. Oncogene 22, 7486-7495 (2003).
24. Kon, N. et al. Inactivation of HAUSP in vivo modulates p53 function. Oncogene 29, 1270-1279.

1. A purified, isolated or recombinant vIRF4 peptide fragment, wherein the fragment comprises an amino acid sequence of the group: vIRF4 as 153-256; vIRF4 as 608-758; vIRF4 as 202-208; vIRF4 as 211-216; vIRF4 as 202-216 (vif1); vIRF4 as 209-216; vIRF4 as 153-216; vIRF4 as 217-236; or vIRF4 as 220-236 (vif2), or a biological equivalent of each thereof.

2. A purified, isolated or recombinant vIRF4 peptide comprising two non-contiguous vIRF4 peptide fragments of claim 1.

3. The peptide of claim 1, further comprising a cell penetrating domain.

4. A purified, isolated or recombinant retro-inverso peptide of claim 1.

5. The peptide of claim 3, wherein the cell penetrating domain comprises a HIV TAT peptide.

6. A purified, isolated or recombinant polynucleotide encoding the peptide of claim 1.

7. A composition comprising the purified, isolated or recombinant peptide of claim 1.

8. The composition of claim 7, wherein the carrier is a pharmaceutically acceptable carrier.

9. An antibody that specifically binds a peptide of claim 1.

10. A composition comprising the antibody of claim 9, and a carrier.

11. The composition of claim 10, wherein the carrier is a pharmaceutically acceptable carrier.

12. A method of increasing or inducing apoptosis in a cell, comprising contacting the cell with an effective amount of a vIRF4 peptide fragment of claim 1, thereby increasing or inducing apoptosis in the cell.

13. A method of increasing p53 activity in a cell with functional p53, comprising contacting the cell with an effective amount of a vIRF4 peptide fragment of claim 1, thereby increasing p53 activity in the cell.

14. A method of increasing MDM2 activity in a cell, comprising contacting the cell with an effective amount of a vIRF4 peptide fragment of claim 1, thereby increasing MDM2 activity in the cell.

15. A method of decreasing HAUSP activity in a cell, comprising contacting the cell with an effective amount of a vIRF4 peptide fragment of claim 1, thereby decreasing HAUSP activity in the cell.

16. A method of inhibiting enzyme substrate interaction between p53 and MDM2 in a cell, comprising contacting the cell with an effective amount of a vIRF4 peptide fragment of claim 1, thereby inhibiting enzyme substrate interaction between p53 and MDM2 in the cell.

17. A method of competitively blocking substrate binding of the TRAF domain of HAUSP in a cell, comprising contacting the cell with an effective amount of a vIRF4 peptide fragment of claim 1, thereby competitively blocking substrate binding of the TRAF domain of HAUSP in the cell.

18. A method for suppressing deubiquitination activity of HAUSP in a cell comprising contacting the cell with an effective amount of a vIRF4 peptide fragment of claim 1, thereby suppressing deubiquitination activity of HAUSP in a cell.

19. A method of inhibiting the growth of a cancer cell, comprising contacting the cell with an effective amount of a vIRF4 peptide fragment of claim 1, thereby inhibiting the growth of the cancer cell.

20. A method for tuning p53-mediated anti-tumor activity in a cell with functional p53, comprising contacting the cell with an effective amount of a vIRF4 peptide fragment of claim 1, thereby tuning p53-mediated anti-tumor activity in a cell.

21. The method of claim 12, wherein the contacting is in vitro or in vivo.

22. An isolated host cell comprising the purified or isolated vIRF4 peptide fragment of claim 1.

23. A method for expressing a polynucleotide encoding a vIRF4 peptide fragment comprising growing a host cell comprising a polynucleotide encoding the peptide fragment of claim 1, under conditions that favor expression of the polynucleotide.

24. The method of claim 23, further comprising isolating the expressed vIRF4 peptide fragment from the host cell.

25. The antibody of claim 9, wherein the antibody is a monoclonal antibody or a derivative or fragment thereof.

26. A composition comprising the antibody of claim 9 and a carrier.

27. The composition of claim 26, wherein the carrier is a pharmaceutically acceptable carrier.

28. A method for treating cancer in a subject in need of such treatment, comprising administering an effective amount of one or more of the vIRF4 peptide fragment of claim 1, thereby treating cancer in the subject.

29. (canceled)

30. A screen for a possible therapeutic agent, comprising contacting the agent with the catalytic domain of HAUSP (206-560) and comparing the physical interaction of the therapeutic agent to the HAUSP catalytic domain to the interaction of an isolated or purified vIRF4 peptide fragment to the HAUSP catalytic domain, wherein an interaction that is substantially similar or greater than the interaction of vIRF4 peptide interaction identifies the agent as a possible therapeutic agent.

31. A computer-implemented method for identifying an agent that binds herpes virus-associated ubiquitin-specific protease (HAUSP), comprising positioning a three-dimensional structure of a candidate agent against a three-dimensional structure of a HAUSP fragment, wherein the three-dimensional structure of the HAUSP fragment is based on X, Y and Z atomic structure coordinates determined from a crystalline form of the HAUSP fragment, wherein interaction of the agent with the HAUSP fragment at two or more HAUSP amino acids selected from R104, R152, R153, S155, D164, W165 or G166 as represented in SEQ ID NO: 4, or the equivalent of each, identifies that the agent binds HAUSP.

32. The method of claim 31, wherein interaction of the agent with the HAUSP fragment at three or more HAUSP amino acids selected from R104, R152, R153, S155, D164, W165 or G166 or the equivalent of each, identifies that the agent binds HAUSP.

33. The method of claim 31, wherein interaction of the agent with the HAUSP fragment at five or more HAUSP amino acids selected from R104, R152, R153, S155, D164, W165 or G166 or the equivalent of each identifies that the agent binds HAUSP.

34. The method of claim 31, wherein the X, Y and Z atomic structure coordinates are determined from a crystalline form of a vIRF4-HAUSP TRAF domain complex.

35. The method of claim 34, wherein the X, Y and Z atomic structure coordinates comprise the coordinates for HAUSP as set forth in Table 3.

36. The method of claim 31, wherein the X, Y and Z atomic structure coordinates are determined from a crystalline form of a free HAUSP TRAF domain.

37. The method of claim 36, wherein the X, Y and Z atomic structure coordinates comprise the coordinates as set forth in Protein Data Bank (PDB) Accession No.: 2F1W.

38. The method of claim 31, wherein the three-dimensional structure of the HAUSP fragment is further based on X, Y and Z atomic structure coordinates of a HAUSP catalytic domain and wherein interaction of the candidate agent with the HAUSP fragment at two or more HAUSP amino acids selected from R104, R152, R153, S155, D164, W165 or G166 or the equivalent of each and at two or more HAUSP amino acids selected from C223, D481 or H464, as represented in SEQ ID NO: 4, or equivalent of each, identifies that the agent binds HAUSP.

39. The method of claim 38, wherein interaction of the agent with the HAUSP fragment at three or more HAUSP amino acids selected from R104, R152, R153, S155, D164, W165 or G166 or the equivalent of each and at all HAUSP amino acids selected from C223, D481 or H464 or the equivalent of each identifies that the agent binds HAUSP.

40. The method of claim 38, wherein interaction of the agent with the HAUSP fragment at three or more HAUSP amino acids selected from R104, R152, R153, S155, D164, W165 or G166 or the equivalent of each, at all HAUSP amino acids selected from C223, D481 or H464 or the equivalent of each and at one or more HAUSP amino acids selected from N218, N226, D295, D482 or H456 or the equivalent of each identifies that the agent binds HAUSP.

41. The method of claim 38, wherein the X, Y and Z atomic structure coordinates of the HAUSP catalytic domain are set forth in Protein Data Bank (PDB) Accession No.: 2F1Z.

42. The method of claim 31, further comprising analyzing the ability of the candidate agent to bind to HAUSP in an in vitro or in vivo assay.

43. The method of claim 31, wherein the agent is a small molecule.

44. The method of claim 31, wherein the agent is a polypeptide, an antibody, an antibody fragment, or combinations or mixtures thereof.

45. The method of claim 31, wherein the agent inhibits the activity of HAUSP, inhibits cell growth, promotes cell cycle arrest, promotes apoptosis or promotes cell death.

46. An agent that binds HAUSP identified by the method claim 31.

47. A computer-implemented method for identifying an agent that interacts with herpes virus-associated ubiquitin-specific protease (HAUSP), comprising positioning a three-dimensional structure of a candidate agent against a three-dimensional structure of a HAUSP fragment, wherein the three-dimensional structure of the HAUSP fragment is based on X, Y and Z atomic structure coordinates determined from a crystalline form of the HAUSP fragment, wherein interaction of the agent with the HAUSP fragment at two or more HAUSP amino acids selected from R104, R152, R153, S155, D164, W165 or G166 identifies that the candidate agent interacts with HAUSP.

48. A computer-implemented method for identifying an agent suitable for inhibiting the activity of herpes virus-associated ubiquitin-specific protease (HAUSP), inhibiting cell growth, promoting cell cycle arrest, promoting apoptosis or promoting cell death, comprising positioning a three-dimensional structure of a candidate agent against a three-dimensional structure of a HAUSP fragment, wherein the three-dimensional structure of the HAUSP fragment is based on X, Y and Z atomic structure coordinates determined from a crystalline form of the HAUSP fragment, wherein interaction of the agent with the HAUSP fragment at two or more HAUSP amino acids selected from R104, R152, R153, S155, D164, W165 or G166 as represented in SEQ ID NO: 4 or equivalent of each identifies the candidate agent as suitable for inhibiting the activity of HAUSP, inhibiting cell growth, promoting cell cycle arrest, promoting apoptosis or promoting cell death.

49. An agent suitable for inhibiting the activity of HAUSP, inhibiting cell growth, promoting cell cycle arrest, promoting apoptosis or promoting cell death, identified by the method of claim 48.

50. A method for identifying an agent that binds herpes virus-associated ubiquitin-specific protease (HAUSP) or is suitable for inhibiting the activity of HAUSP, inhibiting cell growth, promoting cell cycle arrest, promoting apoptosis or promoting cell death, in a custom computing apparatus, the custom computing apparatus comprising at least one processor and a memory, the method comprising:
receiving, in the memory, X, Y and Z atomic structure coordinates determined from a crystalline form of the HAUSP;
accessing, by the at least one processor the X, Y and Z atomic structure coordinates;
positioning, by the at least one processor, a three-dimensional structure of a candidate agent against a three-dimensional structure of a HAUSP fragment based on the X, Y and Z atomic structure coordinates, wherein interaction of the agent with the HAUSP fragment at two or more HAUSP amino acids selected from R104, R152, R153, S155, D164, W165 or G166 as represented in SEQ ID NO: 4 or equivalent of each identifies that the agent binds HAUSP, or is suitable for inhibiting the activity of HAUSP, inhibiting cell growth, promoting cell cycle arrest, promoting apoptosis or promoting cell death.

51. A custom computing apparatus comprising:
at least one processor;
a memory coupled to the at least one processor;
a storage medium in communication with the memory and the at least one processor, the storage medium containing a set of processor executable instructions that, when executed by the processor configure the custom computing apparatus to identify an agent that binds herpes virus-associated ubiquitin-specific protease (HAUSP) or is suitable for inhibiting the activity of HAUSP, inhibiting cell growth, promoting cell cycle arrest, promoting apoptosis or promoting cell death, wherein the configuration comprises:
positioning a three-dimensional structure of a candidate agent against a three-dimensional structure of a HAUSP fragment based on the X, Y and Z atomic structure coordinates, wherein interaction of the agent with the HAUSP fragment at two or more HAUSP amino acids selected from R104, R152, R153, S155, D164, W165 or G166 as represented in SEQ ID NO: 4 or equivalent of each identifies that the agent binds HAUSP, or is suitable for inhibiting the activity of HAUSP, inhibiting cell growth, promoting cell cycle arrest, promoting apoptosis or promoting cell death.

52. A non-transitory computer medium comprising a set of processor executable instructions that, when executed by a processor, identify an agent that binds herpes virus-associated ubiquitin-specific protease (HAUSP), or is suitable for inhibiting the activity of HAUSP, inhibiting cell growth, promoting cell cycle arrest, promoting apoptosis or promoting cell death by positioning a three-dimensional structure of a candidate agent against a three-dimensional structure of a HAUSP fragment based on the X, Y and Z atomic structure coordinates, wherein interaction of the agent with the HAUSP fragment at two or more HAUSP amino acids selected from R104, R152, R153, S155, D164, W165 or G166 as represented in SEQ ID NO: 4 or equivalent of each identifies that the agent binds HAUSP, or is as suitable for inhibiting the activity of HAUSP, inhibiting cell growth, promoting cell cycle arrest, promoting apoptosis or promoting cell death.
